(19) United States
(12) Patent Application Publication
(10) Pub. No.: US 2026/0365754 A1
(43) Pub. Date: Nov. 19, 2026

(54) IMMUNE CELLS WITH PAIRED RECEPTORS AND USES THEREOF

(71) Applicant: A2 Biotherapeutics, Inc., Agoura Hills, CA (US)

(72) Inventors: Timothy P. Riley, Moorpark, CA (US); Richele Martha Bruno, Agoura Hills, CA (US); Mark Erik Daris, Newbury Park, CA (US)

(21) Appl. No.: 19/485,143

(22) PCT Filed: May 17, 2024
(86) PCT No.: PCT/US2024/030032

§ 371 Date: Nov. 17, 2025

Related U.S. Application Data
(60) Provisional application No. 63/503,133, filed on May 18, 2023.
(60) Provisional application No. 63/608,567, filed on Dec. 11, 2023.

(51) Int. Cl. C07K 16/30; A61K 40/11; A61K 40/31; A61K 40/42; C07K 14/485; C07K 14/705; C07K 16/28; C12N 5/0783

(52) U.S. Cl. CPC C07K 16/30; A61K 40/11; A61K 40/31; A61K 40/4211; A61K 40/4255; C07K 14/485; C07K 14/70521; C07K 16/2803; C12N 5/0636; C07K 2317/53; C07K 2317/622; C07K 2319/03; C12N 2510/00

(57) ABSTRACT

The disclosure provides immune cells with paired receptors, and methods of making and using same for the treatment of cancer.

### CROSS REFERENCE TO RELATED APPLICATIONS

This application claims priority to U.S. provisional patent applications 63/503,133, filed on May 18, 2023, and 63/608,567, filed on Dec. 11, 2023, the contents of each of which are incorporated herein by reference in its entireties.

### SEQUENCE LISTING

The instant application contains a Sequence Listing which has been submitted electronically in XML format and is hereby incorporated by reference in its entirety. Said XML copy created on May 16, 2024, is named 061250-554001WO.xml and is 540 KB in size.

### TECHNICAL FIELD

The disclosure relates to the fields of adoptive cell therapy and cancer therapeutics.

### BACKGROUND

Engineered immune cells have emerged as a powerful platform to reprogram immune system functions in the body. Chimeric antigen receptors (CARs) have been particularly effective at redirecting T cells to eradicate B cell malignancies, with recent efforts to expand this approach into solid tumor indications. However, a persistent obstacle in oncology is the lack of tractable targets that differentiate tumors from normal tissues. Several approaches have sought to circumvent this obstacle by implementing logic-gated systems that respond to antigen profiles, rather than single antigens (see for recent review Labanieh and Mackall, 2023). An example of such an approach is the NOT-gated Tmod platform (see for review DiAndreth et al., 2022). By pairing an activating CAR or TCR with an engineered inhibitory receptor (i.e., blocker), Tmod cells provide a safety switch to spare normal cells that express an antigen not typically found on tumor cells.

In principle, Tmod receptors can accommodate a wide diversity of therapeutic targets. Tmod activators and blockers are modular, and Tmod constructs directed at many different activator antigens (A-Antigens; including CD19, CEA, and MSLN) have demonstrated tumor selectivity in preclinical experiments in vitro and in vivo (DiAndreth et al., 2022). Beyond the requirement for a few core components (e.g., ligand-binding, transmembrane, and signaling domains), CARs can tolerate significant structural variation (Xu et al., 2020). The Tmod blocker derived from the LIR-1 (LILRB1) immune inhibitory receptor is less well studied, though it also displays a degree of modularity (Hamburger et al., 2020).

One extensively studied class of blocker antigens (B-Antigens) is HLA-I. Apart from being the natural ligand of LIR-1, HLA-I has a variety of features ideal for use in the Tmod platform to focus the immune response on selected tumors. HLA-I loci are expressed at high levels in all nucleated cells, are polymorphic, and undergo clonal LOH in a large subset of tumors (Huang et al., 2021; McGlanahan et al.). Thus, blockers that specifically target one HLA-I allele, e.g., HLA-A*02, can be used for heterozygous HLA-A*02 patients whose tumors have LOH. In this situation, Tmod cells can be engineered to target activator antigens (A-Antigens) such as MSLN expressed in tumors with HLA-A*02 LOH. In principle, expression of HLA-A*02 antigen on normal cells enables Tmod cells to destroy MSLN (+) HLA-A*02(−) tumors while avoiding MSLN (+) HLA-A*02(+) normal cells in the mesothelial lining of the heart and lung (Tokatlian et al., 2022). Though robust and extensively validated in preclinical experiments, this approach is limited to patients with somatic HLA-I LOH in their tumors.

To extend the Tmod system beyond HLA-I LOH, two additional requirements must be satisfied: (i)non-HLA B-antigens must be present on vulnerable healthy tissues, but consistently not expressed in tumors; and (ii) the B-antigen, together with the A-antigen that comprise the Tmod target antigens, must direct the Tmod cells to achieve effective control over cytotoxicity.

Accordingly, there exists a need in the art for compositions and methods that can expand the number of cancer targets available for tumor-selective cell therapy.

### SUMMARY

Provided herein are compositions and methods related to immune cells with paired activating and inhibitory receptors. Advantageously, the compositions and methods disclosed herein may exploit the hinge length of paired receptors to address any loss of activity of one or both receptors of the pair. The compositions and methods disclosed herein may, in some cases, avoid systemic toxicity to normal tissues by pairing a targeted activator receptor with a blocker receptor. Without being bound by theory, the difference in length of an activator receptor and an inhibitory receptor may confer high selectivity for targeting cells expressing two antigens.

The disclosure provides a method of producing an engineered cell responsive to targets cells expressing a first antigen and not a second antigen. The method includes identifying a first antigen and a second antigen having epitopes of defined heights; selecting one or more hinge elements based on the relative heights of the epitopes of the first antigen and the second antigen; generating polynucleotide sequences encoding: an activator receptor with a defined receptor height that specifically binds the epitope of the first antigen, an inhibitory receptor with a defined receptor height that specifically binds the epitope of the second antigen, wherein either the activator receptor or the inhibitor receptor, or both, comprise at least one hinge element such that the combined height of the inhibitory receptor and second antigen epitope is less than or approximately equal to the combined height of the activator receptor and first antigen epitope; and introducing the polynucleotide sequences into a cell, thereby generating the engineered cell responsive to targets cells expressing the first antigen and not the second antigen and nonresponsive to cells expressing both the first and second antigen.

The disclosure also provides a method of rescuing activity of paired inhibitory and activating receptors in an engineered cell. The method includes generating polynucleotide sequences encoding: an activator receptor that specifically binds an epitope of a first antigen, and an inhibitory receptor that specifically binds an epitope of a second antigen, wherein the activator receptor comprises at least one hinge element such that the combined height of the activator receptor and first antigen epitope is greater than the combined height of the inhibitory receptor and second antigen epitope; and introducing the polynucleotide sequences into a cell, thereby rescuing the activity of inhibitory and activating receptors in the engineered cell.

In some embodiments, the cell is an immune cell or a cell capable of differentiating into an immune cell.

In some embodiments, the activator receptor and the inhibitory receptor each comprise at least one selected hinge element such that the combined height of the inhibitory receptor and second antigen epitope is approximately equal to the combined height of the activator receptor and first antigen.

In some embodiments, the activator receptor comprises a plurality of selected hinge elements such that the combined height of the activator receptor and the first antigen epitope is larger than the combined height of the inhibitory receptor and second antigen epitope.

In some embodiments, the height of the inhibitory receptor is reduced such that the combined height of the inhibitory receptor and second antigen epitope is less than the combined height of the activator receptor and first antigen epitope.

The disclosure also provides an engineered cell selectively responsive to target cells expressing a first antigen and not a second antigen, but nonresponsive cells expressing both the first and second antigen, the engineered cell comprising: an activator receptor that specifically binds the first antigen, and an inhibitory receptor that specifically binds the second antigen, wherein the receptor that binds the shorter of the epitopes of two antigens comprises one or more hinge elements such that said receptor has a greater height than the other receptor.

In some embodiments, the cell is an immune cell or a cell capable of differentiating into an immune cell.

In some embodiments, the activator receptor and the inhibitory receptor each comprises one or more hinge elements.

In some embodiments, the one or more hinge elements are derived from polypeptides encoding a G4Q hinge, a thrombospondin (TSP) hinge, an Immunoglobulin (Ig) hinge, an Epidermal Growth Factor (EGF) hinge, a fibronectin III (FNIII) hinge, or combinations thereof. In some embodiments, the one or more hinge elements comprises a hinge element comprising a sequence of SEQ ID NOs: 1-22, or a sequence having at least 85%, at least 90%, at least 95%, at least 97% or at least 99% identity thereto.

In some embodiments, the height of the inhibitory receptor is approximately equal to or less than the epitope height of the activator receptor.

In some embodiments, the first antigen is selected from MSLN, CD19, and CEA.

In some embodiments, the second antigen is selected from MSLN, LRRN4, and HLA-A2.

In some embodiments, the activator receptor is a chimeric antigen receptor (CAR). In some embodiments, the activator receptor comprises an scFv. In some embodiments, the scFv comprises a sequence of SEQ ID NO: 29-31, or a sequence having at least 85%, at least 90%, at least 95%, at least 97% or at least 99% identity thereto.

In some embodiments, the CAR comprises a CD8 or CD28 transmembrane domain.

In some embodiments, the CAR comprises CD28 and/or 4-1BB intracellular domains and a CD3z intracellular domain.

In some embodiments, the activator receptor comprises a sequence having at least 85%, at least 90%, at least 95%, at least 97%, at least 99% identity to SEQ ID NOs: 84-104.

In some embodiments, the inhibitory receptor is a chimeric antigen receptor (CAR). In some embodiments, inhibitory receptor comprises an scFv. In some embodiments, the scFv comprises a sequence of SEQ ID NO: 28, 29, or 31, or a sequence having at least 85%, at least 90%, at least 95%, at least 97% or at least 99% identity thereto.

In some embodiments, the inhibitory receptor comprises a LILRB1 intracellular domain or a functional variant thereof.

In some embodiments, the inhibitory receptor comprises one or more EGF hinge elements. In some embodiments, the inhibitory receptor comprises 1, 2, 3, or 4 EGF hinge elements.

In some embodiments, the inhibitory receptor comprises a sequence having at least 85%, at least 90%, at least 95%, at least 97%, at least 99% identity to SEQ ID NOs: 50-72.

The disclosure also provides an engineered immune cell made by the process described herein.

The disclosure also provides a pharmaceutical composition, comprising a therapeutically effective amount of the engineered cells as described herein. In some embodiments, the pharmaceutical composition further comprises a pharmaceutically acceptable carrier, diluent, or excipient.

The disclosure further provides a method of selectively killing targets cells expressing a first antigen and not a second antigen, comprising contacting target cells with the engineered immune cells as described herein.

The disclosure also provides a method of treating or preventing cancer in a subject, comprising administering to the subject the engineered cells as described herein.

The disclosure further provides an engineered cell selectively responsive to target cells expressing a first antigen and not LRRN4. The engineered cell includes an activator receptor that specifically binds the first antigen, optionally MSLN, and an inhibitory receptor that specifically binds LRRN4.

In some embodiments, the cell is an immune cell or a cell capable of differentiating into an immune cell.

In some embodiments, the inhibitory receptor comprises one or more hinge elements, such that the combined height of the inhibitory receptor and LRRN4 epitope is less than or approximately equivalent to the combined height of the activator receptor and first antigen epitope.

In some embodiments, the activator receptor and the inhibitory receptor each comprises one or more hinge elements, such that the combined height of the inhibitory receptor and LRRN4 epitope is less than or approximately equal to the combined height of the activator receptor and first antigen epitope. In some embodiments, the one or more hinge elements are selected from a G4Q hinge, a TSP hinge, an Ig hinge, an EGF hinge, a FNIII hinge, or combinations thereof. In some embodiments, the one or more hinge elements comprises a hinge element comprising a sequence of SEQ ID NOs: 1-22, or a sequence having at least 85%, at least 90%, at least 95%, at least 97% or at least 99% identity thereto.

In some embodiments, the activator receptor is a chimeric antigen receptor (CAR). In some embodiments, the activator receptor comprises an scFv. In some embodiments, the scFv comprises a sequence of SEQ ID NO: 29-31, or a sequence having at least 85%, at least 90%, at least 95%, at least 97% or at least 99% identity thereto. In some embodiments, the CAR comprises a CD8 or CD28 transmembrane domain. In some embodiments, the CAR comprises CD28 and 4-1BB intracellular domains and a CD3z intracellular domain.

In some embodiments, the activator receptor comprises a sequence having at least 85%, at least 90%, at least 95%, at least 97%, at least 99% identity to SEQ ID NOs.: 84-104.

In some embodiments, the inhibitory receptor is a chimeric antigen receptor (CAR). In some embodiments, the inhibitory receptor comprises an scFv. In some embodiments, the scFv comprises a sequence of SEQ ID NO: 29, or a sequence having at least 85%, at least 90%, at least 95%, at least 97% or at least 99% identity thereto.

In some embodiments, the inhibitory receptor comprises a LILRB1 intracellular domain or a functional variant thereof.

In some embodiments, the inhibitory receptor comprises one or more EGF hinge elements.

In some embodiments, the inhibitory receptor comprises 5, 6, or 7 EGF hinge elements.

In some embodiments, the inhibitory receptor comprises a sequence having at least 85%, at least 90%, at least 95%, at least 97%, at least 99% identity to SEQ ID NO. 50.

The disclosure also provides an engineered cell comprising an activator receptor and an inhibitory receptor wherein the activator receptor comprises one or more hinge elements. In some embodiments, the hinge element is derived from polypeptides encoding a G4Q hinge, a thrombospondin (TSP) hinge, an Immunoglobulin (Ig) hinge, an Epidermal Growth Factor (EGF) hinge, a fibronectin III (FNIII) hinge, or combinations thereof. In some embodiments, the one or more hinge elements comprises a hinge element comprising a sequence of SEQ ID NOs: 1-22, or a sequence having at least 85%, at least 90%, at least 95%, at least 97% or at least 99% identity thereto.

The disclosure also provides a pharmaceutical composition, comprising a therapeutically effective amount of the engineered cell as described herein. In some embodiments, the pharmaceutical composition further comprises a pharmaceutically acceptable carrier, diluent, or excipient.

The disclosure also provides a method of selectively killing targets cells expressing a first antigen, optionally MSLN and not LRRN4, comprising contacting target cells with the engineered immune cell as described herein.

The disclosure also provides a method of treating or preventing cancer in a subject, comprising administering to the subject the engineered cells as described herein.

Any engineered cell, pharmaceutical composition, or method disclosed herein is applicable to any herein-disclosed engineered cell, pharmaceutical composition, or method. In other words, any aspect or embodiment described herein can be combined with any other aspect or embodiment as disclosed herein.

### DESCRIPTION OF THE DRAWINGS

FIG. 1 shows a schematic of antigen and receptor heights, and the ideal relationship between activator and blocker (inhibitory) receptors.

FIG. 2 shows the rationale for hinge engineering. Targeting specific antigens or epitopes can result in unideal geometry between activator/blocking receptors, resulting in one or the other being excluded from the T cell synapse. Engineered hinges aim to address this challenge on the effector side by optimizing the activator and the blocker with respect to their target antigens.

FIG. 3 shows graphs illustrating the activation and inhibition parameters for the receptors described herein. Equations for these parameters are located in the abbreviations section.

FIG. 4 shows the use of effector cell height estimates and target cell height estimates for determination of best blocking activity.

FIG. 5 shows illustrations of targetable cell surface B-antigens. The different targetable B-antigens vary broadly in size and length.

FIG. 6 shows next generation hinge designs. Multiple hinge elements were explored that could serve as spacers to extend the ligand binding domain from the membrane surface to a set distance. We considered domains that serve as natural spacers throughout native biological proteins, and domains were ranked based on a unit length/amino acid ratio. Since these domains are so prevalent throughout biology, segments from template proteins with multiple domain repeats could be repurposed as hinges to mitigate immunogenicity concerns. Other unexplored possibility: Fibronectin III domains, which has similar properties as Ig (~100 residues/40 A).

FIG. 7A shows polypeptide sequences of MSLN activators with hinge examples. Example hinge sequences are underlined and bolded.

FIG. 7B shows polypeptide sequences of MSLN activators with hinge examples. Example hinge sequences are underlined and bolded.

FIG. 7C shows polypeptide sequences of MSLN activators with hinge examples. Example hinge sequences are underlined and bolded.

FIG. 8 shows a diagram of hinge design and rationale for LRRN4 blocker. The size dependency of Tmod targets suggested that modifications of the standard Tmod designs may be necessary to pair CAR activators with LRRN4 blockers. As a first step, we considered variations in blocker hinge design. Using the 1J21 scFv characterized in various blocker studies, we designed new hinges derived from relatively structural spacers seen in various native biological proteins. Without being bound by theory, unlike flexible hinge elements, repeated hinge elements may allow an indirect estimation of the distance between the T cell membrane surface and the ligand-binding domain based on the number of repeats. Thus, we designed a series of activator and inhibitor receptors with rigid hinge elements, such as seven EGF-like domain repeats (extracted from LRP-1; uniprotID: Q07954, aa 4147-4409) to increase the distance between the ligand binding domain and surface of the T cell in approximately 20-30 Å increments. Although limited experimental data is available for these large proteins, Monte Carlo simulations of structural models suggested that even the largest hinge maintained a relatively extended conformation.

FIG. 9 shows screening of hinge element for expression. We screened various hinge elements for transient expression in Jurkat cell lines (a T cell line). Each hinge element was incorporated into a MSLN activator and paired with an LRRN4 blocker of equivalent length. We co-expressed each construct and evaluated module designs based on transfection efficiency, expression, and uniform behavior. Surface expression was detected by ability of activator and blocker to bind labeled antigens, demonstrating that the hinge did not disrupt the ligand binding domain.

FIG. 10 shows the estimated length of engineered receptors.

FIG. 11 shows the height of the respective receptors, determined by hinge element identity and repetition, is a tunable functional parameter. When targeting a small, membrane proximal A-antigen (CD19) and a larger, membrane distal B-antigen (MSLN), blocking is generally poor. By manipulating the combined epitope and receptor height of the activator and the blocker simultaneously, we can create scenarios where both receptor-ligand complexes are of approximate similar height or broadly disparate in height. Jurkat coculture assays suggest inhibition of the activation signal can be enhanced in this scenario by either increasing the height of the activator or by decreasing the height of the blocker.

FIG. 12 shows approximating preferred synapse relationships. Using the conversion table shown in FIG. 10, we can estimate the synapse sizes for each receptor combination. Mapping these distances on the experimental heatmap suggests blocking is most effective when the blocker synapse (blocker+B antigen) is smaller than the activation synapse (activator+A antigen).

FIG. 13 shows blocking MSLN with LRRN4 has different hinge requirements. Modular hinges can also be used to improve scenarios where the B-antigen (LRRN4) is significantly larger than the A-antigen (MSLN), but instead targets a membrane proximal epitope.

Although the complete structure of LRRN4 is unknown, it is likely substantially larger than MSLN, as the primary protein structure contains twice the amino acids. Structural models derived from Alphafold and Rosetta predict LRRN4 consists of a membrane-distal Leucine-rich repeat domain and a membrane-proximal Fibronectin type III domain, and size exclusion chromatography suggests the ectodomain exists as a dimer. Simulations suggest LRRN4 extends ~175 A from the membrane surface.

Jurkat coculture assays revealed that both long blocker and activator hinges resulted in the most effective blocking, although the longer activators were also associated with a reduction in max Jurkat activation. We hypothesized this was a result of the blocker needing to extend beyond the large ectodomain of the LRRN4 protein in order to access the membrane-proximal epitope. Considering the 175 A length of the LRRN4 extracellular domain, only the long blocker hinges (EGF 5&7) resulted in a successful block when paired with an equally long activator.

FIG. 14 shows hinges can be used interchangeably to assess size requirements. The longest EGF hinge explored has a max length of ~180 Å, which limited the assessment of the EGF hinges targeting LRRN4. To explore the impact of longer hinges with LRRN4 blockers, we leveraged TSP hinges which are approximately 2-3× larger per unit than EGF-like domains. Thus, LRRN4 blockers with a TSP2 hinge are approximately the same length as the blockers with EGF5 hinges. Comparing TSP2, TSP4, and TSP6 hinged LRRN4 blockers reveals that continued extension beyond ~150 Å decreases the ability of the blocker to inhibit the activation signal. *(G4Q)3 hinge used to mimic short blocker hinge in TSP experiment. **EGF5 activator transfection failed for TSP experiments due to unexpected plasmid mutation.

FIG. 15 shows extending the activator alone is enough to improve performance of the blocker. To explore the ability of these modular hinges to influence blocking, we tested the effect of hinge length on blocker function. A series of 4 blocker constructs with 1-7 EGF-like domains were tested in Jurkat cell assays. Unfortunately, none of these Tmod blocker hinge variants demonstrated any measurable functional activity when paired with a CD8 hinged activator. Poor expression or improper scFv folding could not explain the poor functional performance, as surface-staining with labeled recombinant LRRN4 detected high receptor levels. This result suggested that blockers with hinges composed of multiple domains may be partially incompatible with the membrane proximal LRRN4 epitope.

Alternatively, we hypothesized the structure of the activator complex may be another avenue to modulate Tmod-blocker function. Therefore, we also tested the effects of incorporating EGF-like modules into the hinge of the MSLN activator. As before, we co-cultured Jurkat effector cells with HeLa target cells in which target antigen levels were varied by mRNA titration. Surprisingly, systematically increasing the hinge length of the activator resulted in improved blocker sensitivities as the number of EGF-like domains increased from 1 to 7, with the most sensitive blocking achieved with the largest activators. Indeed, Tmod constructs with the longest activator hinges resulted in a significant blocking improvement compared to the Tmod construct with the unmodified CD8 activator hinge. Quantification of blocker activity showed an IC50 of ~15,000 molecules/cell for the MSLN (7 EGF)/LRRN4 constructs containing the canonical LIR-1 backbone/hinge. This IC50 was ~10× left-shifted (i.e., more sensitive) than the original MSLN/LRRN4 Tmod construct and was similar to the reported IC50 of the benchmark MSLN/A*02 Tmod pair.

FIG. 16A shows a schematic of structural comparisons of CD19 A-Antigen (green) and three B-Antigens (HLA-A*02, MSLN, and ICAM-1; red).

FIG. 16B shows quantification of the percent activation of Jurkat T cells expressing the CD19 activator and a blocker targeting one of the three B-antigens (HLA-A*02, MSLN, and ICAM-1) co-cultured with K562(CD19+) target cells transfected with increasing amounts of B-antigen mRNA.

FIG. 17A shows the scFv binding affinities for the B antigens shown in FIG. 19.

FIG. 17B shows FACs plots quantifying the expression of the B-antigen on target cells that were transfected with increasing amounts of mRNA.

FIG. 17C shows the relative expression of B-antigens quantified by QIFI.

FIG. 18A shows a schematic of the structural parameters that impact the overall receptor/ligand complex height (C_{B}). X_{B} is the length of the epitope distal region on the blocker antigen. E_{B} is the proximity of the B-antigen epitope.

FIG. 18B shows a schematic of the MSLN epitopes that are membrane proximal, membrane distal, or truncated compared to the CD19 epitope.

FIG. 18C shows the quantification of the percent activation of Jurkat T cells expressing the CD19 activator and a blocker targeting either a MSLN-distal epitope (N=17) or a MSLN-proximal epitope (N=11) co-cultured with K562(CD19+) target cells transfected with increasing amount of MSLN mRNA. Average trace for each epitope bin shown in black line (p<0.001).

FIG. 18D shows the quantification of the percent activation of Jurkat T cells expressing the CD19 activator and a membrane proximal MSLN blocker co-cultured with K562(CD19+) target cells transfected with mRNA encoding either the full-length or the truncated form of MSLN.

FIG. 19 shows a schematic of the comparison of hinge length and flexibility relative to TCR:pMHC complex. (100 Monte Carlo simulations for each hinge were overlayed to assess flexibility.)

FIG. 20 shows the functional assessment of MSLN CARs responding to full length or truncated MSLN. (If a CAR did not respond to truncated form, it was classified as a distal binder)

FIG. 21A shows a schematic of EGF-like hinges that were used to manipulate receptor length. Extension from the cell membrane is estimated to be ~20-30 Å per subunit. See abbreviations for parameter definitions.

FIG. 21B shows the quantification of the percent activation of Jurkat T cells expressing each hinge combination of CD19 activator plus MSLN blocker that were co-cultured with either K562(MSLN−) or K562(MSLN+) target cells. The difference in activation between the two conditions was converted into Imax (%) and presented as a heatmap.

FIG. 21C shows the quantification of the percent activation of Jurkat T cells expressing 7EGF Activator: 2EGF Blocker that were co-cultured with K562 target cells expressing increasing amounts of MSLN in co-culture assay (left), with a diagram illustrating approximate activator and blocker interactions (right).

FIG. 21D shows the quantification of the percent activation of Jurkat T cells expressing 1EGF Activator: 7EGF Blocker that were co-cultured with to K562 target cells expressing increasing amounts of MSLN in co-culture assay (left), with a diagram illustrating approximate activator and blocker lengths (right).

FIG. 21E shows quantification of the killing of target cells expressing CD19 by PBMCs expressing a short CD19 activator and long MSLN blocker.

FIG. 21F shows quantification of the killing of target cells expressing CD19 by PBMCs expressing a long activator and short blocker.

FIG. 22A shows FACS quantification of the expression levels of the CD19 activator/MSLN blocker with different lengths due to incorporation of EGF domains.

FIG. 22B shows quantification of the relative expression levels of the activator (CD19 CAR).

FIG. 22C shows quantification of activator activity in T-cells based on the binding of their activating receptor. Activating receptor size was manipulated by insertion or removal of EGF domains.

FIG. 23 shows a schematic of the geometric parameters that regulate blocker receptor performance.

FIG. 24A shows a schematic of the structural comparison of the PSMA A-antigen (with proposed epitope in green) and a series of B-antigens with an N-terminal FLAG tag epitope (red), with axial length approximations for each antigen.

FIG. 24B shows a comparison of the NFAT activation signal from the Jurkat T cells co-culture assays for each blocker antigen as a function of axial length (TB), using CD8/LIR1 hinge configuration.

FIG. 24C shows a correlation analysis between Imax and the estimated difference in complex height between activator and blocker (ΔCB-A) using modular EGF hinges.

FIG. 25A shows the antigen density for FLAG-antigens and compares it to Imax.

FIG. 25B shows the blocking profile for the indicated receptors when expanding the R_{A} or R_{B} with EGF modular hinges.

FIG. 25C shows a plot of the Imax from 26B versus total estimates of ΔC.

### DETAILED DESCRIPTION

Provided herein are compositions and methods for targeting specific antigens or epitopes comprising an optimized two receptor system responsive to differences in gene expression of a ligand between a target cell (e.g., a cancer cell) and normal (i.e., healthy or wild type) cells. The differences in expression can be because the gene is not expressed in target cells or is expressed in target cells at a lower level than normal cells. The two-receptor system can be expressed in immune cells, for example immune cells used in adoptive cell therapy, and targets activity of these immune cells to target cells exhibiting expression differences. In this two receptor system, the first receptor (an activator receptor, sometimes referred to herein as an A module) activates, or promotes activation of the immune cells, while the second receptor (an inhibitory receptor, sometimes referred to herein as a blocker, inhibitor receptor, or B module) acts to inhibit activation of the immune cells by the first receptor. Each receptor contains a ligand-binding domain (LBD) that binds a specific ligand. Signals from the two receptors upon ligand binding are integrated by the immune cell. Differential expression of ligands for the first and second receptors in target and normal cells, or differences in transcription levels, mediates activation of immune cells by target cells that express the first activator ligand but not the second inhibitory ligand.

The two-receptor system described herein employs one receptor to activate, for example, T cells exposed to tumor-antigen-positive tumor cells (sometimes referred to as an “activator module”), and a second receptor to prevent activation of the immune cells in the presence of a surface blocker antigen such as LRRN4 protein. The dual-receptor system described herein (sometimes referred to herein as “Tmod”) possesses other advantageous properties as a cell therapy, including but not limited to reversible activation/blockade of immune cells, and selectivity in mixtures of tumor and “normal” cells.

However, targeting of specific antigens or epitopes in this context requires an ideal geometry between the activating and blocking receptors. This ideal geometry allows the activating and blocking receptors to be adjacent each to each other in the T cell synapse and result in successful blocking. In cases where an unideal geometry between the activating and blocking receptors exists, the blocking receptor, for example, can be excluded from the T cell synapse and result in weak or absent blocking activity. To address this problem, the immune cells with paired receptors described herein employs engineered hinge elements in one or both of the activating and blocking receptors in order to optimize the activator and blocker with respect to each of their target antigens.

In particular embodiments of the compositions and methods provided herein, immune cells comprising the two-receptor system described herein are used to treat cancers. In some embodiments, the cancer is a Mesothelin (MSLN) positive cancer. This includes mesothelioma cancer, ovarian cancer, cervical cancer, colorectal cancer, esophageal cancer, head and neck cancer, kidney cancer, uterine cancer, gastric cancer, pancreatic cancer, lung cancer, colorectal cancer, or cholangiocarcinoma. In some embodiments, the cancer has relapsed in a subject. In some embodiments, the cancer is refractory to one or more prior administered anticancer therapies. In some embodiments, the cancer is metastatic. In the case of MSLN-positive cancers, the target antigen of the activator receptor is MSLN, or a peptide antigen thereof, in a complex with a major histocompatibility complex class I (MHC-I). MSLN is expressed in normal adipose, fallopian tube, lung and salivary gland tissues, among others. Because of its expression in certain tumors, MSLN is an attractive tumor-specific antigen that could mediate selective killing of MSLN+ tumors if these cancer cells could be specifically targeted with an appropriate therapeutic. However, normal MSLN expression in non-cancer (non-target) cells has prevented the effective use of MSLN for targeted therapies such as adoptive cell therapies. Previously, Tmod cells have been engineered to target activator antigens (A-Antigens) such as MSLN expressed in tumors with HLA-A*02 loss of heterozygosity (LOH). In principle, expression of HLA-A*02 antigen on normal cells enables Tmod cells to destroy MSLN (+) HLA-A*02(−) tumors while avoiding MSLN (+) HLA-A*02(+) normal cells in the mesothelial lining of the heart and lung. Though robust and extensively validated in preclinical experiments, this approach is limited to patients with somatic HLA-I LOH in their tumors. Thus, as described herein, the optimized Tmod system has been extended to include additional activator and blocker pairings that, under normal circumstances, would exhibit weak or absent blocking due to the geometry between the activating and blocking receptor. By pairing an MSLN activator receptor, which has been engineered with optimized hinge elements, with an optimized inhibitory receptor, the methods provided herein increase the specificity of adoptive cell therapies and decrease harmful effects associated with these therapies, such as dose-limited toxicity. The methods provided herein also expand the number of cancer targets available for tumor-selective cell therapy.

In variations, the compositions and methods described herein may be used to kill target cells and/or treat subjects in which expression of the second antigen is partially or completely decreased by causes other than loss of heterozygosity, including but not limited to partial gene deletion, epigenetic silencing, and point mutations or truncating mutations in the sequence encoding the second antigen.

The methods and compositions described in PCT/US2021/060607, PCT/US2023/011698, US 20230029341, US 20220370497, US 20220273721, and US 20220054551, are incorporated herein by reference in their entirety.

### DEFINITIONS

Prior to setting forth this disclosure in more detail, it may be helpful to an understanding thereof to provide definitions of certain terms to be used herein.

Unless defined otherwise, all technical and scientific terms used herein have the same meaning as commonly understood by those of ordinary skill in the art to which the disclosure belongs. Although any methods and materials similar or equivalent to those described herein can be used in the practice or testing of particular embodiments, preferred embodiments of compositions, methods and materials are described herein. For the purposes of the present disclosure, the following terms are defined below. Additional definitions are set forth throughout this disclosure.

As used herein, the singular forms “a”, “an”, and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps. As also used herein, in any instance or embodiment described herein, “comprising” may be replaced with “consisting essentially of” and/or “consisting of” used herein, in any instance or embodiment described.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

As used herein, the “administering” of an agent, e.g., an engineered immune cell, to a subject or subject includes any route of introducing or delivering to a subject a compound to perform its intended function. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated and target cell or tissue. Non-limiting examples of route of administration include parenteral, enteral, and topical routes of administration. Administration includes self-administration and the administration by another. It is also to be appreciated that the various modes of treatment or prevention of medical conditions as described are intended to mean “substantial”, which includes total but also less than total treatment or prevention, and wherein some biologically or medically relevant result is achieved.

As used herein, the term “isolated” means material that is substantially or essentially free from components that normally accompany it in its native state.

In embodiments, the term “obtained” or “derived” is used synonymously with isolated. In embodiments, the terms “obtained” or “derived” mean selecting a known amino acid sequence and including the known amino acid sequence into an antigen binding domain, ligand binding domain, construct, receptor, polynucleotide, or polypeptide of the present disclosure. As an example, the expression a “ligand binding domain may be derived from commercially available antibodies” may be interpreted as incorporating the amino acid sequence for one or more complement determining regions (CDRs) of the commercially available antibodies into an antigen binding domain, ligand binding domain, construct, receptor, or polypeptide of the present disclosure. Additionally, a VH or VL of one or more commercially available antibodies may be incorporated into an antigen binding domain, ligand binding domain, construct, receptor, or polypeptide of the present disclosure. A known amino acid sequence comprises a non-antigen binding domain of a commercially available antibody; here the non-antigen binding domain is incorporated into a construct, receptor, or polypeptide of the present disclosure. In some embodiments, “incorporating” comprises including the nucleic acid sequence encoding the known amino acid sequence into a polynucleotide that encodes an antigen binding domain, ligand binding domain, construct, receptor, or polypeptide of the present disclosure.

The terms “subject,” “patient” and “individual” are used interchangeably herein to refer to a vertebrate, preferably a mammal, more preferably a human. Tissues, cells, and their progeny of a biological entity obtained in vivo or cultured in vitro are also encompassed. A “subject,” “patient” or “individual” as used herein, includes any animal that exhibits pain that can be treated with the vectors, compositions, and methods contemplated herein. Suitable subjects (e.g., patients) include laboratory animals (such as mouse, rat, rabbit, or guinea pig), farm animals, and domestic animals or pets (such as a cat or dog). Non-human primates and, preferably, human patients, are included.

As used herein “treatment” or “treating,” includes any beneficial or desirable effect, and may include even minimal improvement in symptoms. “Treatment” does not necessarily indicate complete eradication or cure of the disease or condition, or associated symptoms thereof.

As used herein, “prevent,” and similar words such as “prevented,” “preventing” etc., indicate an approach for preventing, inhibiting, or reducing the likelihood of a symptom of disease. It also refers to delaying the onset or recurrence of a disease or condition or delaying the occurrence or recurrence of the symptoms of a disease. As used herein, “prevention” and similar words also includes reducing the intensity, effect, symptoms and/or burden of disease prior to onset or recurrence.

As used herein, the term “amount” refers to “an amount effective” or “an effective amount” of a virus to achieve a beneficial or desired prophylactic or therapeutic result, including clinical results.

A “therapeutically effective amount” of a virus or cell may vary according to factors such as the disease state, age, sex, and weight of the individual, and the ability of the virus or cell to elicit a desired response in the individual. A therapeutically effective amount is also one in which any toxic or detrimental effects of the virus or cell are outweighed by the therapeutically beneficial effects. The term “therapeutically effective amount” includes an amount that is effective to “treat” a subject (e.g., a patient).

An “increased” or “enhanced” amount of a physiological response, e.g., electrophysiological activity or cellular activity, is typically a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the level of activity in an untreated cell.

A “decreased” or “reduced” amount of a physiological response, e.g., electrophysiological activity or cellular activity, is typically a “statistically significant” amount, and may include an decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the level of activity in an untreated cell.

By “maintain,” or “preserve,” or “maintenance,” or “no change,” or “no substantial change,” or “no substantial decrease” refers generally to a physiological response that is comparable to a response caused by either vehicle, or a control molecule/composition. A comparable response is one that is not significantly different or measurable different from the reference response.

In general, “sequence identity” or “sequence homology” refers to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby and comparing these sequences to a second nucleotide or amino acid sequence. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequences and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA 87:2264-2268 (1990) and as discussed in Altschul, et al., J. Mol. Biol. 215:403-410 (1990); Karlin And Altschul, Proc. Natl. Acad. Sci. USA 90:5873-5877 (1993); and Altschul et al., Nucleic Acids Res. 25:3389-3402 (1997). Briefly, the BLAST program defines identity as the number of identical aligned symbols (generally nucleotides or amino acids), divided by the total number of symbols in the shorter of the two sequences. The program may be used to determine percent identity over the entire length of the proteins being compared. Default parameters are provided to optimize searches with short query sequences in, for example, with the blastp program. The program also allows use of an SEG filter to mask-off segments of the query sequences as determined by the SEG program of Wootton and Federhen, Computers and Chemistry 17:149-163 (1993). Ranges of desired degrees of sequence identity are approximately 80% to 100% and integer values therebetween. Typically, the percent identities between a disclosed sequence and a claimed sequence are at least 80%, at least 85%, at least 90%, at least 95%, or at least 98%.

As used herein, a “polynucleotide system” refers to one or more polynucleotides. The one or more polynucleotides may be designed to work in concert for a particular application, or to produce a desired transformed cell.

The term “exogenous” is used herein to refer to any molecule, including nucleic acids, protein or peptides, small molecular compounds, and the like that originate from outside the organism. In contrast, the term “endogenous” refers to any molecule that originates from inside the organism (i.e., naturally produced by the organism).

In the present description, any concentration range, percentage range, ratio range, or integer range is to be understood to include the value of any integer within the recited range and, when appropriate, fractions thereof (such as one tenth and one hundredth of an integer), unless otherwise indicated. The term “about”, when immediately preceding a number or numeral, means that the number or numeral ranges plus or minus 10%.

The term “MOP” is used herein to refer to multiplicity of infection, which is the ratio of agents (e.g., viral particles) to infection targets (e.g., cells).

As used herein, the term “antigen” refers to a compound, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides), phospholipids, and proteins. Common categories of antigens include, but are not limited to, viral antigens, bacterial antigens, fungal antigens, protozoa and other parasitic antigens, tumor antigens, antigens involved in autoimmune disease, allergy and graft rejection, toxins, and other miscellaneous antigens.

As used herein, “binding affinity” refers to the tendency of one molecule to bind (typically non-covalently) with another molecule, such as the tendency of a member of a specific binding pair for another member of a specific binding pair. A binding affinity can be measured as a dissociation constant, which for a specific binding pair (such as an antibody/antigen pair) can be lower than 1×10⁻⁵ M, lower than 1×10⁻⁶ M, lower than 1×10⁻⁷ M, lower than 1×10⁻⁸ M, lower than 1×10⁻⁹ M, lower than 1×10⁻¹⁰M, lower than 1×10⁻¹¹ M or lower than 1×10⁻¹²M. In one aspect, binding affinity is calculated by a modification of the Scatchard method described by Frankel et al., Mol. Immunol., 16:101-106, 1979. In another aspect, binding affinity is measured by a binding constant. In another aspect, binding affinity is measured by an antigen/antibody dissociation rate. In yet another aspect, a high binding affinity is measured by a competition radioimmunoassay.

The term “human antibody” as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis or by somatic mutation in vivo). However, the term “human antibody” as used herein, is not intended to include antibodies in which CDR sequences derived from the germline of another mammalian species, such as a rabbit, have been grafted onto human framework sequences. Thus, as used herein, the term “human antibody” refers to an antibody in which substantially every part of the protein (e.g., CDR, framework, C_{L}, C_{H} domains (e.g., C_{H1}, C_{H2}, C_{H3}), hinge, V_{L}, V_{H}) is substantially non-immunogenic in humans, with only minor sequence changes or variations. Similarly, antibodies designated primate (monkey, baboon, chimpanzee, etc.), rodent (mouse, rat, rabbit, guinea pig, hamster, and the like) and other mammals designate such species, sub-genus, genus, sub-family, family specific antibodies. Further, chimeric antibodies include any combination of the above. Such changes or variations optionally and preferably retain or reduce the immunogenicity in humans or other species relative to non-modified antibodies. Thus, a human antibody is distinct from a chimeric or humanized antibody. It is pointed out that a human antibody can be produced by a non-human animal or prokaryotic or eukaryotic cell that is capable of expressing functionally rearranged human immunoglobulin (e.g., heavy chain and/or light chain) genes. Further, when a human antibody is a single chain antibody, it can comprise a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin.

As used herein, a “target cell” refers to cell that is targeted by an adoptive cell therapy. For example, a target cell can be cancer cell, which can be killed by the transplanted T cells of the adoptive cell therapy. Target cells of the disclosure express a target antigen, as described herein, and do not express a non-target antigen.

As used herein, a “non-target cell” refers to cell that is not targeted by an adoptive cell therapy. For example, in an adoptive cell targeting cancer cells, normal, healthy, non-cancerous cells are non-target cells. Some, or all, non-target cells in a subject may express both the target antigen and the non-target antigen. Non-target cells in a subject may express the non-target antigen irrespective of whether or not these cells also express the target antigen.

As used herein, HLA-A*02 antibody” and “anti-HLA-A*02 antibody” are used interchangeably and refer to an antibody that specifically binds to an HLA-A*02 polypeptide. Similarly, the term “HLA-A*02”, as mentioned in “HLA-A*02 binding”, “HLA-A*02 targeting”, “HLA-A*02 specific”, “HLA-A*02 expression” or other similar terms here, refers to HLA-A*02 polypeptide. HLA-A*02 antibodies of the disclosure may bind to proteins falling within the HLA-A*02 allele group (for example HLA-A*02:01, HLA-A*02:02 and the like), and may not bind, or bind with lower affinity, to proteins falling within other HLA-A allele groups (for example, HLA-A*01, HLA-A*11, and the like). The person of ordinary skill in the art will recognize that some cross-reactivity with other HLA antigens may exist, but that the HLA-A*02 antibodies of the disclosure will still be considered to be specific to HLA-A*02. In some cases, specificity is considered in the context of the subject to be treated with an HLA-A*02 antibody or receptor of the disclosure. When the subject has both an HLA-A*02 allele and a second HLA-A allele not recognized, or only poorly recognized, by the HLA-A*02 antibody or receptor comprising an equivalent antigen binding domain, the HLA-A*02 antibody is specific to the HLA-A*02 allele of the subject.

As used herein, “HLA-A*03 antibody” and “anti-HLA-A*03 antibody” are used interchangeably and refer to an antibody that specifically binds to an HLA-A*03 polypeptide. Similarly, the term “HLA-A*03”, as mentioned in “HLA-A*03 binding”, “HLA-A*03 targeting”, “HLA-A*03 specific”, “HLA-A*03 expression” or other similar terms here, refers to HLA-A*03 polypeptide. HLA-A*03 antibodies of the disclosure may bind to proteins falling within the HLA-A*03 allele group (for example HLA-A*03:01, HLA-A*03:02 and the like), and may not bind, or bind with lower affinity, to proteins falling within other HLA-A allele groups (for example, HLA-A*02, HLA-A*11, and the like). The person of ordinary skill in the art will recognize that some cross-reactivity with other HLA antigens may exist, but that the HLA-A*03 antibodies of the disclosure will still be considered to be specific to HLA-A*03. In some cases, specificity is considered in the context of the subject to be treated with an HLA-A*03 antibody or receptor of the disclosure. When the subject has both an HLA-A*03 allele and a second HLA-A allele not recognized, or only poorly recognized, by the HLA-A*03 antibody or receptor comprising an equivalent antigen binding domain, the HLA-A*03 antibody is specific to the HLA-A*03 allele of the subject.

As used herein, the term “monoclonal antibody” refers to an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

As used herein, a “non-target allelic variant” refers to an allele of a gene whose product is expressed by non-target cells but is not expressed by target cells. For example, a non-target allelic variant is an allele of a gene that is expressed by normal, non-cancer cells of subject, but not expressed by cancer cells of the subject. The expression of the non-target allelic variant can be lost in the cancer cells by any mechanism, including, but not limited to, loss of heterozygosity, mutation, or epigenetic modification of the gene encoding the non-target allelic variant.

As used herein, a “TCR”, sometimes also called a “TCR complex” or “TCR/CD3 complex” refers to a protein complex comprising a TCR alpha chain, a TCR beta chain, and one or more of the invariant CD3 chains (zeta, gamma, delta and epsilon), sometimes referred to as subunits. The TCR alpha and beta chains can be disulfide-linked to function as a heterodimer to bind to peptide-MHC complexes. Once the TCR alpha/beta heterodimer engages peptide-MHC, conformational changes in the TCR complex in the associated invariant CD3 subunits are induced, which leads to their phosphorylation and association with downstream proteins, thereby transducing a primary stimulatory signal. In an exemplary TCR complex, the TCR alpha and TCR beta polypeptides form a heterodimer, CD3 epsilon and CD3 delta form a heterodimer, CD3 epsilon and CD3 gamma for a heterodimer, and two CD3 zeta form a homodimer.

As used herein, “specific to” or “specifically binds to” when used with respect to a ligand binding domain, such as an antigen binding domain, refers to a ligand binding domain that has a high specificity for a named target. Antibody specificity can be viewed as a measure of the goodness of fit between the ligand binding domain and the corresponding ligand, or the ability of the ligand binding domain to discriminate between similar or even dissimilar ligands. In comparison with specificity, affinity is a measure of the strength of the binding between the ligand binding domain and ligand, such that a low-affinity ligand binding domain binds weakly and high-affinity ligand binding domain binds firmly. The person of skill in the art will appreciate that a ligand binding domain can be said to be specific to a particular target, and yet still have low levels of binding to one or more additional targets that do not affect its function in the receptor systems described herein.

As used herein, a “target antigen,” whether referred to using the term antigen or the name of a specific antigen, refers to an antigen expressed by a target cell, such as a cancer cell. Expression of target antigen is not limited to target cells. Target antigens may be expressed by both cancer cells and normal, non-cancer cells in a subject.

As used herein, a “non-target antigen” (or “blocker antigen”) whether referred to using the term antigen or the name of a specific antigen, refers to an antigen that is expressed by normal, non-cancer cells and is not expressed in cancer cells. This difference in expression allows the inhibitory receptor to inhibit immune cell activation in the presence of non-target cells, but not in the presence of target cells.

Polymorphism refers to the presence of two or more variants of a nucleotide sequence in a population. A polymorphism may comprise one or more base changes, an insertion, a repeat, or a deletion. A polymorphism includes e.g., a simple sequence repeat (SSR) and a single nucleotide polymorphism (SNP), which is a variation, occurring when a single nucleotide of adenine (A), thymine (T), cytosine (C) or guanine (G) is altered.

As used herein, “affinity” refers to strength of binding of a ligand to a single ligand binding site on a receptor, for example an antigen for the antigen binding domain of any of the receptors described herein. Ligand binding domains can have a weaker interaction (low affinity) with their ligand, or a stronger interaction (high affinity).

Kd, or dissociation constant, is a type of equilibrium constant that measures the propensity of a larger object to separate reversibly into smaller components, such as, for example, when a macromolecular complex comprising receptor and its cognate ligand separates into the ligand and the receptor. When the Kd is high, it means that a high concentration of ligand is needed to occupy the receptor, and the affinity of the receptor for the ligand is low. Conversely, a low Kd means that the ligand has a high affinity for the receptor.

As used herein, a receptor that is “responsive” or “responsive to” refers to a receptor comprising an intracellular domain, that when bound by a ligand (i.e., antigen) generates a signal corresponding to the known function of the intracellular domain. An activator receptor bound to a target antigen can generate a signal that causes activation of an immune cell expressing the activator receptor. An inhibitory receptor bound to a non-target antigen can generate an inhibitory signal that prevents or reduces an activation of an immune cell expressing the activator receptor. Responsiveness of receptors, and their ability to activate or inhibit immune cells expressing the receptors, can be assayed by any means known in the art and described herein, including, but not limited to, reporter assays and cytotoxicity assays.

As used herein, “activation” of an immune cell or an immune cell that is “activated” refers to an immune cell that can carry out one or more functions characteristic of an immune response. These functions include proliferation, release of cytokines, and cytotoxicity, i.e., killing of a target cell. Activated immune cells express markers that will be apparent to persons of skill in the art. For example, activated T cells can express one or more of CD69, CD71, CD25 and HL A-DR. An immune cell expressing an activator receptor (e.g., a MSLN CAR) can be activated by the activator receptor when it becomes responsive to the binding of the receptor to a target antigen (e.g., MSLN) expressed by the target cell. A “target antigen” can also be referred to an “activator antigen” and may be isolated or expressed by a target cell. Activation of an immune cell expressing an inhibitory receptor can be prevented when the inhibitory receptor becomes responsive to a non-target antigen (e.g., LRRN4), even when the activator receptor is bound to the target activator ligand. A “non-target antigen” can also be referred to as an “inhibitory ligand” or a “blocker” and may be isolated or expressed by a target cell.

Receptor expression on an immune cell can be verified by assays that report the presence of the activator receptors and inhibitory receptors described herein. For example, a population of immune cells can be stained with a labeled molecule (e.g., a fluorophore labeled receptor-specific antibody or a fluorophore-labeled receptor-specific ligand) and quantified using fluorescence activated cell sorting (FACS) flow cytometry. This method allows a percentage of immune cells in a population of immune cells to be characterized as expressing an activator receptor, an inhibitory receptor, or both receptors. The ratio of activator receptor and inhibitory receptors expressed by the immune cells described herein can be determined by, for example, digital droplet PCR. These approaches can be used to characterize the population of cells for the production and manufacturing of the immune cells, pharmaceutical compositions, and kits described herein. For the immune cells, pharmaceutical compositions, and kits described herein, it is understood that a suitable percentage of immune cells expressing both an activator receptor and an inhibitory receptor is determined specifically for the methods described herein. For example, a suitable percentage of immune cells expressing both an activator receptor and in inhibitory receptor can be at least 50%, at least 55%, at least 60%, at least 65%, at least 70%, at least 75%, at least 80%, at least 85%, at least 90%, or at least 95%. As a further example, between 50% and 99%, between 60% and 95%, between 65% and 95%, between 65% and 90%, between 70% and 90%, between 75% and 90%, between 75% and 85%, between 80% and 99%, between 85% and 99%, between 90% and 99% or between 95% and 99% of immune cells can express both the activator receptor and the inhibitory receptor. For example, a suitable ratio of activator receptor and inhibitory receptor in an immune cell can be about 5:1, about 4:1, about 3:1, about 2:1, about 1:1, about 1:2, about 1:3, about 1:4, or about 1:5. It is understood that purification, enrichment, and/or depletion steps can be used on populations of immune cells to meet suitable values for the immune cells, pharmaceutical compositions, and kits described herein.

A responsive receptor expressed by the immune cells described herein can be verified by assays that measure the generation of a signal expected to be generated by the intracellular domain of the receptor. Reporter cell lines, such as Jurkat-Luciferase NFAT cells (Jurkat cells), can be used to characterize a responsive receptor. Jurkat cells are derived from T cells and comprise a stably integrated nuclear factor of activated T-cells (NFAT)-inducible luciferase reporter system. NFAT is a family of transcription factors required for immune cell activation, whose activation can be used as a signaling marker for T cell activation. Jurkat cells can be transduced or transfected with the activator receptors and/or inhibitory receptors described herein. The activator receptor is responsive to the binding of a ligand if the Jurkat cell expresses a luciferase reporter gene, and the level of responsiveness can be determined by the level of reporter gene expression. The presence of luciferase can be determined using any known luciferase detection reagent, such as luciferin. An inhibitory receptor is responsive to the binding of a ligand if, when co-expressed with an activator receptor in Jurkat cells, it prevents a normally responsive immune cell from expressing luciferase in response to the activator receptor. For example, the responsiveness of an inhibitory receptor can be determined and quantified in a Jurkat cell expressing both an activator and an inhibitor by observing the following: 1) the Jurkat cell expresses luciferase in the presence of activator receptor ligand and absence of inhibitory receptor ligand; and 2) luciferase expression in the Jurkat cell is reduced or eliminated in the presence of both an activator receptor ligand and an inhibitory receptor ligand. This approach can be used to determine the sensitivity, potency, and selectivity of activator receptors and specific pairs of activator receptors and inhibitory receptors. The sensitivity, potency, and selectivity can be quantified by EC50 or IC50 values using dose-response experiments, where an activator receptor ligand and/or inhibitory receptor ligand is titrated into a culture of Jurkat cells expressing an activator receptor or a specific pair of activator and inhibitory receptors. Alternatively, the EC50 and IC50 values can be determined in a co-culture of immune cells (e.g., Jurkat cells or primary immune cells) expressing an activator receptor or a specific pair of activator and inhibitory receptors and target cells expressing an increasing amount of an activator ligand or inhibitor ligand. An increasing amount of activator ligand or inhibitor ligand can be accomplished in the target cell by, for example, titration of activator ligand or inhibitor ligand encoding mRNA into target cells, or use of target cells that naturally express different levels of the target ligands. Exemplary suitable EC50 and IC50 values for the activator and inhibitory receptors as determined used target cells expressing varying amounts of the target and non-target ligands include an EC50 of 10 transcripts per million (TPM) or less for the activator receptor, for example an EC50 of between 2-10 TPM, and an IC50 of 25 TPM or less for the inhibitory receptor, for example an IC50 of 5-21 TPM.

Activation of the immune cells described herein that express an activator receptor or specific pairs of activator and inhibitory receptors can be further determined by assays that measure the viability of a target cell following co-incubation with said immune cells. The immune cells, sometimes referred to as effector cells, are co-incubated with target cells that express an activator receptor ligand, an inhibitory receptor ligand, or both an activator and inhibitory receptor ligand. Following co-incubation, viability of the target cell is measured using any method to measure viability in a cell culture. For example, viability can be determined using a mitochondrial function assay that uses a tetrazolium salt substrate to measure active mitochondrial enzymes. Viability can also be determined using imaging-based methods. Target cells can express a fluorescent protein, such as green fluorescent protein or red fluorescent protein. Reduction in total cell fluorescence indicates a reduction in viability of the target cell. A reduction in viability of the target cell following incubation with immune cells expressing an activator receptor or a specific pair of activator and inhibitory receptors is interpreted as target cell-mediated activation of the immune cell. A measure of the selectivity of the immune cells can also be determined using this approach. The immune cell expressing a pair of activator and inhibitory receptors is selective if the following is observed: 1) viability is reduced in target cells expressing the activator receptor ligand but not the inhibitory receptor ligand; 2) viability is not reduced in target cells expressing both an activator receptor ligand and an inhibitory receptor ligand. From these measurements, a “specific killing” value can be derived that quantifies the percentage of immune cell activation based on the reduction in viability of target cell as a percentage of a negative control (immune cells that do not express an activator receptor). Further, from these measurements a “selectivity ratio” value can be derived that represents the ratio of the specific killing observed in target cells expressing an activator receptor ligand in the absence of inhibitory receptor ligand to the specific killing observed in target cells expressing both an activator receptor ligand and an inhibitory receptor ligand. This approach can be used to characterize the population of cells for the production and manufacturing of the immune cells, pharmaceutical compositions, and kits described herein. A suitable specific killing value for the immune cells, pharmaceutical compositions, and kits can be, for example, the following criteria: 1) at least 40%, at least 50%, at least 60%, at least 70%, at least 80%, at least 90%, at least 95%, at least 97% or at least 99% specific killing following a 48 hour co-incubation of immune cells and target cells expressing activator receptor ligand in the absence of inhibitory receptor ligand; and 2) less than or equal to 40%, less than or equal to 35%, less than or equal to 30%, less than or equal to 25%, less than or equal to 20%, less than or equal to 15%, less than or equal to 10%, less than or equal to 5%, less than or equal to 3% or less than or equal to 1% specific killing of target cell expressing both an activator receptor ligand and an inhibitory receptor ligand.

As a further example, a suitable specific killing value for the immune cells, pharmaceutical compositions and kits can be the following criteria: 1) between 30% and 99%, between 40% and 99%, between 50% and 99%, between 55% and 95%, between 60% and 95%, between 60% and 90%, between 50% and 80%, between 50% and 70% or between 50% and 60% of target cells expressing the activator ligand but not the inhibitor ligand are killed; and 2), between 1% and 40%, between 3% and 40%, between 5% and 40%, between 5% and 30%, between 10% and 30%, between 15% and 30% or between 5% and 20% of target cells expressing the activator ligand and the inhibitor ligand are killed. As a still further example, a suitable specific killing value for the immune cells, pharmaceutical compositions, and kits can be, for example, the following criteria: 1) at least 50% specific killing following a 48 hour co-incubation of immune cells and target cells expressing activator receptor ligand in the absence of inhibitory receptor ligand; and 2) less than or equal to 20% specific killing of target cell expressing both an activator receptor ligand and an inhibitory receptor ligand. As a further example, the immune cells are capable of killing at least 30%, at least 40%, at least 50%, at least 60%, at least 70%, at least 80%, at least 90%, at least 95%, at least 97% or at least 99% of target cells expressing the activator ligand and not the inhibitor ligand over a period of 6 hours, 12 hours, 18 hours, 24 hours, 30 hours, 36 hours, 42 hours, 48 hours, 54 hours, or 60 hours, while killing less than 40%, less than 30%, less than 20%, less than 10%, less than 5%, less than 3% or less than 1% of target cells expressing the activator and inhibitor ligands over the same time period.

A suitable specific killing value of the target cell expressing an activator ligand in the absence of an inhibitory ligand value for the immune cells, pharmaceutical compositions, and kits can be, for example, at least about 50% to at least about 95%. A suitable specific killing value of the target cell expressing an activator ligand in the absence of an inhibitory ligand value for the immune cells, pharmaceutical compositions, and kits can be, for example, at least about 50%, at least about 55%, at least about 60%, at least about 65%, at least about 70%, at least about 75%, at least about 80%, at least about 85%, at least about 90%, or at least about 95%. A suitable specific killing value of the target cell expressing an activator ligand in the absence of an inhibitory ligand value for the immune cells, pharmaceutical compositions, and kits can be, for example, at most about 50%, at most about 55%, at most about 60%, at most about 65%, at most about 70%, at most about 75%, at most about 80%, at most about 85%, at most about 90%, or at most about 95%. A suitable specific killing value of target cells expressing both an activator receptor ligand and an inhibitory receptor ligand for the immune cells, pharmaceutical compositions, and kits can be less than about 50%, less than about 45%, less than about 40%, less than about 35%, less than about 30%, less than about 25%, less than about 20%, less than about 15%, less than about 10%, or less than about 5%. The suitable specific killing value for the immune cells, pharmaceutical compositions, and kits can be determined following about 6 hours, about 12 hours, about 18 hours, about 24, about 30 hours, about 36 hours, about 42 hours, about 48 hours, about 54 hours, about 60 hours, about 66 hours, or about 72 hours of co-incubation of immune cells with target cells.

A suitable specific killing value of the target cell expressing an activator ligand in the absence of an inhibitory ligand value for the immune cells, pharmaceutical compositions, and kits can be, for example, at least about 50% to at least about 95%. A suitable specific killing value of the target cell expressing an activator ligand in the absence of an inhibitory ligand value for the immune cells, pharmaceutical compositions, and kits can be, for example, at least about 50%, at least about 55%, at least about 60%, at least about 65%, at least about 70%, at least about 75%, at least about 80%, at least about 85%, at least about 90%, or at least about 95%. A suitable specific killing value of the target cell expressing an activator ligand in the absence of an inhibitory ligand value for the immune cells, pharmaceutical compositions, and kits can be, for example, at most about 50%, at most about 55%, at most about 60%, at most about 65%, at most about 70%, at most about 75%, at most about 80%, at most about 85%, at most about 90%, or at most about 95%. A suitable specific killing value of target cells expressing both an activator receptor ligand and an inhibitory receptor ligand for the immune cells, pharmaceutical compositions, and kits can be can be less than about 50%, less than about 45%, less than about 40%, less than about 35%, less than about 30%, less than about 25%, less than about 20%, less than about 15%, less than about 10%, or less than about 5%. The suitable specific killing value for the immune cells, pharmaceutical compositions, and kits can be can be determined following about 6 hours, about 12 hours, about 18 hours, about 24, about 30 hours, about 36 hours, about 42 hours, about 48 hours, about 54 hours, about 60 hours, about 66 hours, or about 72 hours of co-incubation of immune cells with target cells.

As used herein, the term “functional variant” refers to a protein that has one or more amino-acid substitutions, insertions, or deletions as compared to a parental protein, and which retains one or more desired activities of the parental protein. A functional variant may be a fragment of the protein (i.e., a variant having N- and/or C-terminal deletions) that retain the one or more desired activities of the parental protein.

All publications and patents mentioned herein are hereby incorporated by reference in their entirety as if each individual publication or patent was specifically and individually indicated to be incorporated by reference. In case of conflict, the present application, including any definitions herein, will control. However, mention of any reference, article, publication, patent, patent publication, and patent application cited herein is not, and should not be taken as an acknowledgment, or any form of suggestion, that they constitute valid prior art or form part of the common general knowledge in any country in the world.

### Hinge Elements

As described above, the immune cells with paired receptors described herein employs engineered hinge elements in one or both of the activating and blocking receptors in order to optimize the activator and blocker with respect to each of their target antigens.

The disclosure provides activator and inhibitory receptors, of which one or both comprise one or more selected hinge elements.

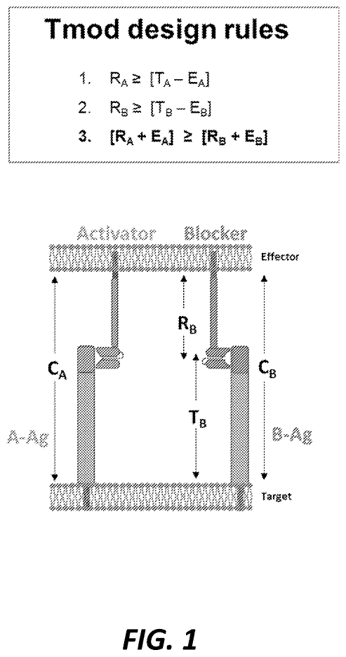

As described herein, the one or more hinge elements are selected based on the relative heights of an identified first antigen and second antigen. The “height” of an antigen refers to the distance, along an axis perpendicular to the cell membrane, from the surface of the cell membrane to the centroid of the epitope specifically bound by a cognate antibody, antibody fragment, or receptor. This is depicted in FIG. 1, which describes T_{A} as the total height of an A antigen (e.g., a first antigen) from a target cell membrane, and T_{B} as the total height of a B antigen (e.g., a second antigen) from a target cell membrane. As shown in FIG. 1, the epitope height of the A antigen (as defined above) is indicated as E_{A}, and the epitope height of the B antigen is indicated as E_{B}. Similarly, the “height” of a receptor refers to the distance, along an axis perpendicular to the cell membrane, from the surface of the effector cell membrane to the centroid of the site of receptor of the receptor that specifically binds the antigen. This is depicted in FIG. 1, which describes R_{A} as the height of a CAR extracellular region (e.g., an activator receptor) from an effector cell membrane, and R_{B} as the height of a blocker extracellular region (e.g., an inhibitory receptor) from an effector cell membrane Exemplary first and second antigens as well as activator and blocker receptors for use herein are described in more detail below.

A variety of hinge elements can be used in the methods and compositions of the disclosure. As used herein, a “hinge element” is any polypeptide segment that can serve as a spacer to extend the ligand binding domain of an activator or inhibitory receptor from the membrane surface a set distance. In some embodiments, the hinge element is a flexible hinge element. In some embodiments, the hinge element is a structured hinge element with tertiary structure. Exemplary hinge elements that can be used in the methods and compositions of the disclosure include, without limitation, hinge elements derived from polypeptides encoding a G4Q hinge, a thrombospondin (TSP) hinge, an Immunoglobulin (Ig) hinge, an Epidermal Growth Factor (EGF) hinge, a fibronectin III (FNIII) hinge, or combinations thereof. In some embodiments, the TSP hinge is derived from human Properdin (UnitProt P27918). In some embodiments, the Ig hinge is derived from human CEACAM5 (UnitProt P06731). In some embodiments, the EGF hinge is derived from LRP1 (UnitProt Q07954). In some embodiments, the FNIII hinge is derived from human PTPRC (UnitProt P08575). Characteristics and sequences of exemplary hinge elements are shown in Table 1 below.

TABLE 1

| | Estimated | | | SEQ |
|---|---|---|---|---|
| | Height | No. | | ID |
| Hinge | (Angstroms) | residues | Sequence | NO: |
| G4Q | — | 5n | GGGGQ | 1 |
| TSP1 | 60 | 49-84n | DPVLCFTQYEESSGKCKGLLGGGVSVEDCC | 2 |
| | | | LNTAFAYQKRSGGLCQPCR | |
| TSP2 | 60 | 49-84n | SPRWSLWSTWAPCSVTCSEGSQLRYRRCVG | 3 |
| | | | WNGQCSGKVAPGTLEWQLQACEDQQCCP | |
| TSP3 | 60 | 49-84n | HGAWATWGPWTPCSASCHGGPHEPKETRSR | 4 |
| | | | KCSAPEPSQKPPGKPCPGLAYEQRRCTGLP | |
| | | | PCP | |
| TSP4 | 60 | 49-84n | AGGWGPWGPVSPCPVTCGLGQTMEQRTCNH | 5 |
| | | | PVPQHGGPFCAGDATRTHICNTAVPCP | |
| TSP5 | 60 | 49-84n | DGEWDSWGEWSPCIRRNMKSISCQEIPGQQ | 6 |
| | | | SRGRTCRGRKFDGHRCAGQQQDIRHCYSIQ | |
| | | | HCP | |
| TSP6 | 60 | 49-84n | KGSWSEWSTWGLCMPPCGPNPTRARQRLCT | 7 |
| | | | PLLPKYPPTVSMVEGQGEKNVTFWGRPLPR | |
| | | | ECELQGQKLVVEEKRPCLHVPACK | |
| Ig1 | 30 | 76-88n | PDTPIISPPDSSYLSGANLNLSCHSASNPS | 8 |
| | | | PQYSWRINGIPQQHTQVLFIAKITPNNNGT | |
| | | | YACFVSNLATGRNNSIVKSITVS | |
| Ig2 | 30 | 76-88n | PKPSISSNNSKPVEDKDAVAFTCEPEAQNT | 9 |
| | | | TYLWWVNGQSLPVSPRLQLSNGNRTLTLFN | |
| | | | VTRNDARAYVCGIQNSVSANRSDPVTLD | |
| Ig3 | 30 | 76-88n | PTISPSYTYYRPGVNLSLSCHAASNPPAQY | 10 |
| | | | SWLIDGNIQQHTQELFISNITEKNSGLYTC | |
| | | | QANNSASGHSRTTVKTIT | |
| Ig4 | 30 | 76-88n | PKPFITSNNSNPVEDEDAVALTCEPEIQNT | 11 |
| | | | TYLWWVNNQSLPVSPRLQLSNDNRTLTLLS | |
| | | | VTRNDVGPYECGIQNELSVDHSDPVILN | |
| Ig5 | 30 | 76-88n | PTISPLNTSYRSGENLNLSCHAASNPPAQY | 12 |
| | | | SWFVNGTFQQSTQELFIPNITVNNSGSYTC | |
| | | | QAHNSDTGLNRTTVTT | |
| Ig6 | 30 | 76-88n | PKPSISSNNSKPVEDKDAVAFTCEPETQDA | 13 |
| | | | TYLWWVNNQSLPVSPRLQLSNGNRTLTLFN | |
| | | | VTRNDTASYKCETQNPVSARRSDSVILN | |
| EGF1 | 30 | 36-37n | LTCVGHCSNGGSCTMNSKMMPECQCPPHMT | 14 |
| | | | GPRCE | |
| EGF2 | 30 | 36-37n | EVNKCSRCLEGACVVNKQSGDVTCNCTDGR | 15 |
| | | | VAPSCL | |
| EGF3 | 30 | 36-37n | QYRQCSGYCENFGTCQMAADGSRQCRCTAY | 16 |
| | | | FEGSRCE | |
| EGF4 | 30 | 36-37n | TQQVCAGYCANNSTCTVNQGNQPQCRCLPG | 17 |
| | | | FLGDRCQ | |
| EGF5 | 30 | 36-37n | ELDQCWEHCRNGGTCAASPSGMPTCRCPTG | 18 |
| | | | FTGPKCT | |
| EGF6 | 30 | 36-37n | RPGTCNLQCFNGGSCFLNARRQPKCRCQPR | 19 |
| | | | YTGDKCE | |
| EGF7 | 30 | 36-37n | VTNPCDRKKCEWLCLLSPSGPVCTCPNGKR | 20 |
| | | | LDNGTCV | |
| FN31 | 40 | 93n | PPSQVWNMTVSMTSDNSMHVKCRPPRDRNG | 21 |
| | | | PHERYHLEVEAGNTLVRNESHKNCDFRVKD | |
| | | | LQYSTDYTFKAYFHNGDYPGEPFILHHSTS | |
| | | | YNS | |
| FN32 | 40 | 93n | SPGEPQIIFCRSEAAHQGVITWNPPQRSFH | 22 |
| | | | NFTLCYIKETEKDCLNLDKNLIKYDLQNLK | |
| | | | PYTKYVLSLHAYIIAKVQRNGSAAMCHFTT | |
| | | | KSA | |

In some embodiments, the one or more hinge elements comprises a sequence of SEQ ID NOs: 1-22, or a sequence having at least 85%, at least 90%, at least 95%, at least 97% or at least 99% identity thereto.

In some embodiments, 1, 2, 3, 4, 5, 6, or 7 hinge elements are selected and incorporated into one or both of the activator receptor and the inhibitory receptor. In some embodiments, the hinge element is an EGF hinge element.

In some embodiments, the hinge elements can be integrated into one or both of the activator receptor and the inhibitory receptor such that the combined height of the inhibitory receptor and second antigen epitope is less than or approximately equal to the combined height of the activator receptor and first antigen epitope.

In some embodiments, the activator receptor comprises a plurality of selected hinge elements such that the combined height of the activator receptor and the first antigen epitope is larger than the combined height of the inhibitory receptor and second antigen epitope.

In some embodiments, the height of the inhibitory receptor is-reduced such that the combined height of the inhibitory receptor and second antigen epitope is less than the combined height of the activator receptor and first antigen epitope.

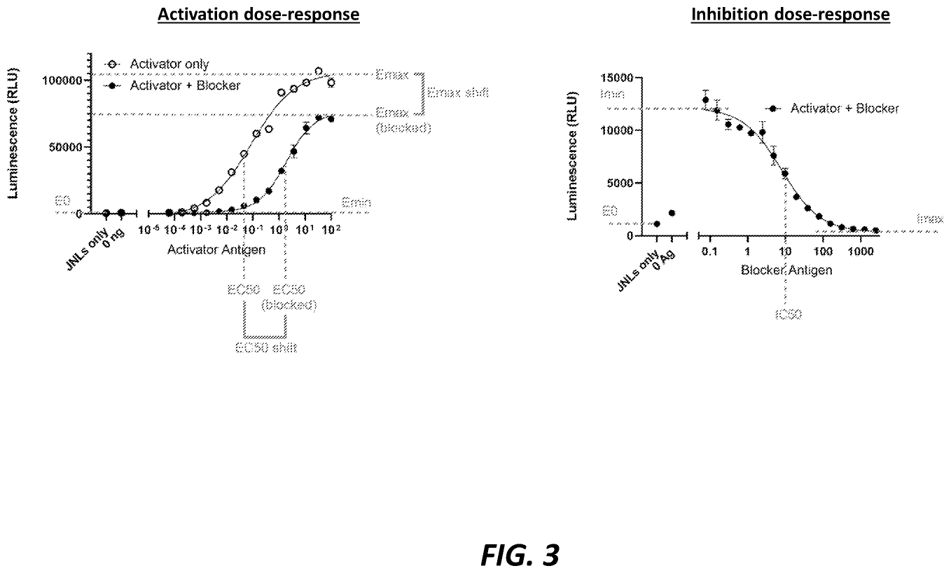

In some embodiments, the height of the activator receptor and the inhibitory receptor are such that the maximum activation signal (Emax) and maximum inhibition signal (Imax) are achieved. This is demonstrated in FIG. 3, which shows the determination of activation and inhibition parameters.

In some embodiments, the relationship between an inhibitor receptor and an activator receptor can be calculated using one of the following parameters as shown in FIG. 1:

(R_{B}+E_{B})≤(R_{A}+E_{A})

R_{A}≥[T_{A}−E_{A}]

R_{B}≥[T_{B}−E_{B}]

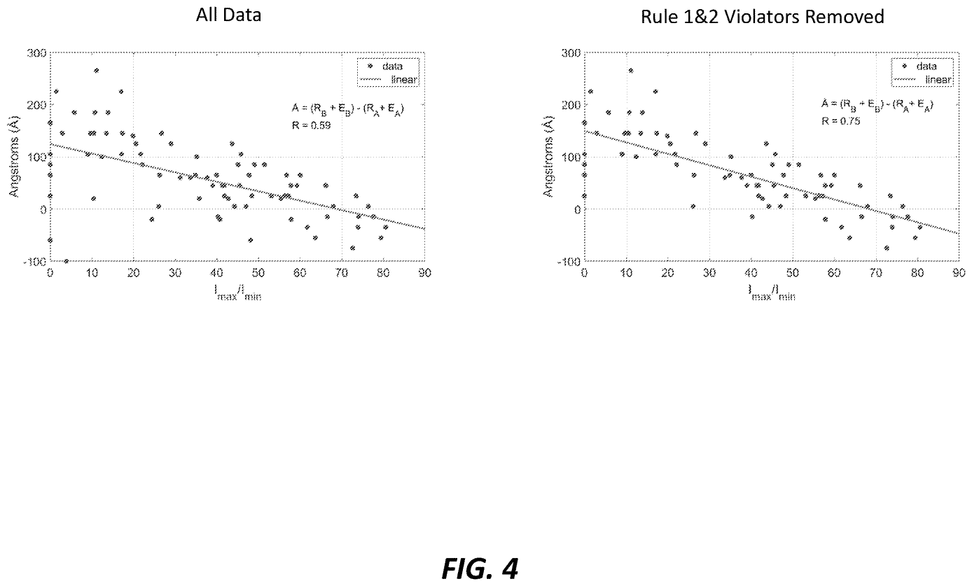

The use of the parameters described herein to determine relationships between an inhibitor receptor and an activator receptor is shown in FIG. 4.

In some embodiments, provided herein is an activator receptor and an inhibitory receptor wherein the activator receptor comprises one or more hinge elements.

### Activator Receptors

The disclosure provides a first antigen, comprising an epitope of a specific height, and an activator receptor with a defined receptor height that specifically binds the epitope of the first antigen. The disclosure further provides a second antigen, comprising an epitope of a specific height, and an inhibitory receptor with a defined receptor height that specifically binds the epitope of the second antigen. As described above, in some embodiments the activator receptor includes one or more hinge elements. In some embodiments, the inhibitory receptor contains one or more hinge elements. In some embodiments, both the activator receptor and inhibitory receptor contain one or more hinge elements. The activator receptor mediates activation of a cell expressing the receptor upon binding of the first antigen by the extracellular ligand binding domain of the activator receptor. The first receptor is responsive to the first antigen. For example, when the first antigen binds to or contacts the activator receptor, the activator receptor is responsive and activates a cell expressing the activator receptor upon binding of the first antigen by the extracellular ligand binding domain of the activator receptor. In some embodiments, the first receptor is a chimeric antigen receptor (CAR). In some embodiments, the first receptor is a T cell receptor (TCR).

In some embodiments, the first receptor is humanized. As used herein, “humanized” refers to the replacement of a sequence or a subsequence in a transgene that has been isolated or derived from a non-human species with a homologous, or functionally equivalent, human sequence. For example, a humanized antibody can be created by grafting mouse CDRs into human framework sequences, followed by back substitution of certain human framework residues for the corresponding mouse residues from the source antibody.

It will be appreciated by the person of ordinary skill that first, activator ligand binding domains for the activator receptor may be isolated or derived from any source known in the art, including, but not limited to, art recognized T cell receptors, chimeric antigen receptors and antibody binding domains. For example, the first ligand binding domain may be derived from commercially available antibodies, including but not limited to Urelumab, Utomilumab, Oleclumab, Naptumomab, Ascrinvacumab, Tacatuzumab, Nesvacumab, Vanucizumab, Belimumab, Tabalumab, Tibulizumab, Belantamab, Igovomab, Oregovomab, Sofituzumab, Mogamulizumab, Talacotuzumab, Tavolimab, Vonlerolizumab, Ipilimumab, Duvortuxizumab, Blinatumomab, Coltuximab, Denintuzumab, Inebilizumab, Loncastuximab, Taplitumomab, Ibritumomab, Obinutuzumab, Ocaratuzumab, Ocrelizumab, Ofatumumab, Rituximab, Tositumomab, Veltuzumab, Samalizumab, Bectumomab, Epratuzumab, Inotuzumab, Moxetumomab, Pinatuzumab, Gomiliximab, Lumiliximab, Camidanlumab, Basiliximab, Inolimomab, Daclizumab, Varlilumab, Enoblituzumab, Omburtamab, Brentuximab, Iratumumab, Gemtuzumab, Lintuzumab, Vadastuximab, Lilotomab, Otlertuzumab, Tetulomab, Daratumumab, Isatuximab, Bivatuzumab, Abituzumab, Intetumumab, Lorvotuzumab, Itolizumab, Cusatuzumab, Vorsetuzumab, Milatuzumab, Polatuzumab, Iladatuzumab, Galiximab, Altumomab, Arcitumomab, Labetuzumab, Cibisatamab, Zolbetuximab, Lacnotuzumab, Cabiralizumab, Emactuzumab, Gimsilumab, Lenzilumab, Otilimab, Mavrilimumab, Tremelimumab, Ulocuplumab, Tepoditamab, Rovalpituzumab, Demcizumab, Drozitumab, Parsatuzumab, Cetuximab, Depatuxizumab, Futuximab, Imgatuzumab, Laprituximab, Matuzumab, Necitumumab, Nimotuzumab, Panitumumab, Zalutumumab, Modotuximab, Amivantamab, Tomuzotuximab, Losatuxizumab, Adecatumumab, Citatuzumab, Edrecolomab, Oportuzumab, Solitomab, Tucotuzumab, Catumaxomab, Ifabotuzumab, Duligotuzumab, Elgemtumab, Lumretuzumab, Patritumab, Seribantumab, Zenocutuzumab, Aprutumab, Bemarituzumab, Vantictumab, Dinutuximab, Ecromeximab, Mitumomab, Codrituzumab, Glembatumumab, Zatuximab, Ertumaxomab, Margetuximab, Timigutuzumab, Gancotamab, Pertuzumab, Trastuzumab, Ficlatuzumab, Rilotumumab, Telisotuzumab, Emibetuzumab, Cixutumumab, Dalotuzumab, Figitumumab, Ganitumab, Robatumumab, Teprotumumab, Flotetuzumab, Bermekimab, Cergutuzumab, Volociximab, Etaracizumab, Relatlimab, Carlumab, Amatuximab, Clivatuzumab, Gatipotuzumab, Pemtumomab, Cantuzumab, Pankomab, Racotumomab, Brontictuzumab, Tarextumab, Vesencumab, Camrelizumab, Cetrelimab, Nivolumab, Pembrolizumab, Pidilizumab, Cemiplimab, Spartalizumab, Atezolizumab, Avelumab, Durvalumab, Cirmtuzumab, Tenatumomab, Fresolimumab, Brolucizumab, Bevacizumab, Ranibizumab, Varisacumab, Faricimab, Icrucumab, Alacizumab or Ramucirumab. The first ligand binding domain may bind to an antigen, including but not limited to TNF receptor superfamily member 9 (4-1BB, CD137), 5′-nucleotidase, trophoblast glycoprotein (5T4), activin receptor-like kinase 1, alpha-fetoprotein, angiopoietin 2, TNF superfamily member 13b (BAFF), TNF receptor superfamily member 17 (BCMA), mucin 16, cell surface associated (CA-125), C—C motif chemokine receptor 4 (CCR4), interleukin 3 receptor subunit alpha (CD123), TNF receptor superfamily member 4 (CD134), cytotoxic T-lymphocyte associated protein 4 (CD152), CD19 molecule (CD19), membrane spanning 4-domains A1 (CD20), CD200 molecule (CD200), CD22 molecule (CD22), Fc fragment of IgE receptor II (CD23, IgE receptor), interleukin 2 receptor subunit alpha (CD25), CD27 molecule (CD27), CD276 molecule (CD276), TNF receptor superfamily member 8 (CD30, TNFRSF8), CD33 molecule (CD33), CD37 molecule (CD37), CD38 molecule (CD38), CD44 molecule v6 (CD44 v6), integrin subunit alpha V (CD51), neural cell adhesion molecule 1 (CD56), CD6 molecule (CD6), CD70 molecule (CD70), CD74 molecule (CD74), CD79B molecule (CD79B), CD80 molecule (CD80), CEA cell adhesion molecule 5 (CEA), Claudin 18 Isoform 2, Colony stimulating factor 1 (CSF1), colony stimulating factor 1 receptor (CSF1R), Colony stimulating factor 2 (CSF2), cytotoxic T-lymphocyte associated protein 4 (CTLA-4), CXCR4 (CD184), dendritic cell-associated lectin 2, delta like canonical Notch ligand 3 (DLL3), delta like canonical Notch ligand 4 (DLL4), TNF receptor superfamily member 10b (DR5), EGF like domain multiple 7 (EGFL7), epidermal growth factor receptor (EGFR), epithelial cell adhesion molecule (EpCAM), EPH receptor A3 (EPHA3), erb-b2 receptor tyrosine kinase 3 (ERBB3, HER3), fibroblast growth factor receptor (FGFR2), Frizzled receptor, GD2 ganglioside, GD3 ganglioside, GD3 ganglioside, glypican 3, glycoprotein nmb (GPNMB), epidermal growth factor receptor (HER1), erb-b2 receptor tyrosine kinase 2 (HER2), hepatocyte growth factor (HGF), MET proto-oncogene, receptor tyrosine kinase (HGFR), IGF-1 receptor (CD221), Interleukin 3 receptor, Interleukin 1 alpha (IL1A), Interleukin 2 (IL2), integrin α5β1, integrin αvβ, lymphocyte activating 3 (LAG3), C—C motif chemokine ligand 2 (MCP-1), mesothelin, Mucin 1, NGNA ganglioside, Notch 1, Notch receptor, neuropilin 1 (NRP1), programmed cell death 1 (PD-1), CD274 molecule (PD-L1), receptor tyrosine kinase like orphan receptor 1 (ROR1), tenascin C, transforming growth factor beta 1 (TGF-β), VEGF-A, VEGFR-1 and VEGFR2. Accordingly, the immune cells comprising the two receptor system described can be used to treat diseases and disorders, including but not limited to cancer, diffuse large B-cell lymphoma, pancreatic and colorectal cancer, non-small cell lung carcinoma, renal cell carcinoma, autoimmune disorders, multiple myeloma, ovarian cancer, melanoma, multiple sclerosis, leukemias and lymphomas, solid tumors and hematologic malignancies, Hodgkin's lymphoma, squamous cell carcinoma, psoriasis, B-cell lymphoma, gastric cancer, head & neck cancers, urothelial cancer, small cell lung cancer, glioblastoma multiforme, neuroblastoma, malignant melanoma, small cell lung carcinoma, breast cancer, and prostate cancer.

### First Antigens

In some embodiments, the first antigen for the activator receptor is a cancer cell specific antigen. Any cell surface molecule expressed by the target cancer cells may be a suitable target antigen for the activator receptor ligand binding domain. For example, a cell adhesion molecule, a cell-cell signaling molecule, an extracellular domain, a molecule involved in chemotaxis, a glycoprotein, a G protein-coupled receptor, a transmembrane, a receptor for a neurotransmitter or a voltage gated ion channel can be used as a target antigen.

In some embodiments, the cancer specific antigen is EGFR, HER2, or a blood cancer antigen.

In some embodiments, the target antigen is a peptide antigen of a cancer cell-specific antigen in a complex with a major histocompatibility complex class I (MHC-I). Any molecule expressed by the target cancer cells and presented by the major histocompatibility complex class I (MHC-I) on the cancer cell surface as a peptide antigen (pMHC) may be a suitable target antigen for the first receptor extracellular ligand binding domain.

In some embodiments, the first antigen is selected from MSLN, CD19, and CEA. Exemplary first antigens for use in the methods and compositions described herein are described in PCT/US2021/060607, PCT/US2023/011698, US 20230029341, US 20220370497, US 20220273721, and US 20220054551, are incorporated herein by reference in their entirety.

In some embodiments, the cancer cell-specific antigen is Mesothelin (MSLN), or a peptide antigen thereof in a complex with a major histocompatibility complex class I (IHC-I).

The major histocompatibility complex class I (IHC-I) is a protein complex that displays antigens to cells of the immune system, triggering an immune response. The Human Leukocyte Antigens (HLAs) corresponding to MHC-I are HLA-A, HLA-B and HLA-C.

Cancer cell-specific pMHC antigens comprising any of HLA-A, HLA-B, HLA-C, HLA-E, HLA-F or HLA-G are envisaged as within the scope of the disclosure. In some embodiments, the cancer cell-specific antigen comprises HLA-A. HLA-A receptors are heterodimers comprising a heavy a chain and smaller p chain. The a chain is encoded by a variant of HLA-A, while the R chain (02-microglobulin) is an invariant. There are several thousand variant HLA-A genes, all of which fall within the scope of the instant disclosure. In some embodiments, the MHC-I comprises a human leukocyte antigen A*02 allele (HLA-A*02).

In some embodiments, the cancer cell-specific antigen comprises HLA-B. Hundreds of versions (alleles) of the HLA-B gene are known, each of which is given a particular number (such as HLA-B*27).

In some embodiments, the cancer cell-specific antigen comprises HLA-C. HLA-C belongs to the HLA class I heavy chain paralogues. This class I molecule is a heterodimer consisting of a heavy chain and a light chain (beta-2 microglobulin). Over one hundred HLA-C alleles are known in the art.

In some embodiments, the cancer cell-specific antigen is an ovarian cancer antigen, a pancreatic cancer antigen, a lung cancer antigen, a colorectal cancer antigen or a mesothelioma antigen. In some embodiments, the cancer cell-specific antigen is a colorectal cancer antigen. In some embodiments, the cancer cell-specific antigen is MSLN or a peptide antigen thereof.

In some embodiments, the cancer cell-specific antigen is MSLN, or a peptide antigen thereof in a complex with a major histocompatibility complex class I (MHC-I). MSLN is a 40 KDa protein that is normally expressed in mesothelial cells, as well as lung, fallopian tube, salivary gland and adipose tissues. MSLN is expressed in multiple human tumor types, including mesothelioma cancer, ovarian cancer, cervical cancer, colorectal cancer, esophageal cancer, head and neck cancer, kidney cancer, uterine cancer, gastric cancer, pancreatic cancer, lung cancer, colorectal cancer, or cholangiocarcinoma. In some embodiments, the cancer has relapsed in a subject. In some embodiments, the cancer is refractory to one or more prior administered anticancer therapies. In some embodiments, the cancer is metastatic.

All isoforms of MSLN are envisaged as cancer cell-specific antigens of the disclosure. MSLN isoform 1 preprotein is described in NCBI record number NP_005814.2, the contents of which are incorporated by reference herein. In some embodiments, MSLN comprises an amino acid sequence of
1 MALPTARPLL GSCGTPALGS LLFLLFSLGW VQPSRTLAGE TGQEAAPLDG VLANPPNISS
61 LSPRQLLGFP CAEVSGLSTE RVRELAVALA QKNVKLSTEQ LRCLAHRLSE PPEDLDALPL
121 DLLLFLNPDA FSGPQACTRF FSRITKANVD LLPRGAPERQ RLLPAALACW GVRGSLLSEA
181 DVRALGGLAC DLPGRFVAES AEVLLPRLVS CPGPLDQDQQ EAARAALQGG GPPYGPPSTW
241 SVSTMDALRG LLPVLGQPII RSIPQGIVAA WRQRSSRDPS WRQPERTILR PRFRREVEKT
301 ACPSGKKARE IDESLIFYKK WELEACVDAA LLATQMDRVN AIPFTYEQLD VLKHKLDELY
361 PQGYPESVIQ HLGYLFLKMS PEDIRKWNVT SLETLKALLE VNKGHEMSPQ VATLIDRFVK
421 GRGQLDKDTL DTLTAFYPGY LCSLSPEELS SVPPSSIWAV RPQDLDTCDP RQLDVLYPKA
481 RLAFQNMNGS EYFVKIQSFL GGAPTEDLKA LSQQNVSMDL ATFMKLRTDA VLPLTVAEVQ
541 KLLGPHVEGL KAEERHRPVR DWILRQRQDD LDTLGLGLQG GIPNGYLVLD LSMQEALSGT
601 PCLLGPGPVL TVLALLLAST LA (SEQ ID NO: 23).

In some embodiments, MSLN comprises a sequence that shares at least 80%, at least 85%, at least 90%, at least 95%, at least 96%, at least 97%, at least 98%, or at least 99% identity to SEQ ID NO: 23.

MSLN isoform 2 preprotein is described in NCBI record number NP_037536.2, the contents of which are incorporated by reference herein. In some embodiments, MSLN comprises an amino acid sequence of:

(SEQ ID NO: 24)

| | |
|---|---|
| 1 | MALPTARPLL GSCGTPALGS LLFLLESLGW VQPSRTLAGE |
| | TGQEAAPLDG VLANPPNISS |
| 61 | LSPRQLLGFP CAEVSGLSTE RVRELAVALA QKNVKLSTEQ |
| | LRCLAHRLSE PPEDLDALPL |
| 121 | DLLLELNPDA FSGPQACTRE FSRITKANVD LLPRGAPERQ |
| | RLLPAALACW GVRGSLLSEA |
| 181 | DVRALGGLAC DLPGREVAES AEVLLPRLVS CPGPLDQDQQ |
| | EAARAALQGG GPPYGPPSTW |
| 241 | SVSTMDALRG LLPVLGQPII RSIPQGIVAA WRQRSSRDPS |
| | WRQPERTILR PRERREVEKT |
| 301 | ACPSGKKARE IDESLIFYKK WELEACVDAA LLATQMDRVN |
| | AIPFTYEQLD VLKHKLDELY |
| 361 | PQGYPESVIQ HLGYLFLKMS PEDIRKWNVT SLETLKALLE |
| | VNKGHEMSPQ APRRPLPQVA |
| 421 | TLIDREVKGR GQLDKDTLDT LTAFYPGYLC SLSPEELSSV |
| | PPSSIWAVRP QDLDTCDPRQ |
| 481 | LDVLYPKARL AFQNMNGSEY FVKIQSELGG APTEDLKALS |
| | QQNVSMDLAT FMKLRTDAVL |
| 541 | PLTVAEVQKL LGPHVEGLKA EERHRPVRDW ILRQRQDDLD |
| | TLGLGLQGGI PNGYLVLDLS |
| 601 | MQEALSGTPC LLGPGPVLIV LALLLASTLA. |

In some embodiments, MSLN comprises a sequence that shares at least 80%, at least 85%, at least 90%, at least 95%, at least 96%, at least 97%, at least 98%, or at least 99% identity to SEQ ID NO: 24.

In some embodiments, the cancer cell-specific antigen is a peptide antigen derived from MSLN. In some embodiments, the peptide antigen comprises a sequence having at least 80%, at least 85%, at least 90%, at least 95%, at least 96%, at least 97%, at least 98%, or at least 99% identity to a subsequence of SEQ ID NO: 23 and/or SEQ ID NO: 24. In some embodiments, the peptide antigen comprises a sequence identical to a subsequence of SEQ ID NO: 23 and/or SEQ ID NO: 24.

### Antigen Binding of the Activator Receptor

The disclosure provides an activator receptor that specifically bind to a first antigen. In some embodiments, the first antigen comprises a cancer cell-specific antigen.

In some embodiments, the cancer cell-specific antigen is MSLN, or a MSLN-derived peptide antigen complexed with MHC-I, and the activator receptor recognizes and binds to the MSLN antigen.

Any type of antigen binding domain that can regulate the activity of a receptor in a ligand dependent manner is envisaged as within the scope of the instant disclosure. Exemplary antigen binding domains include, inter alia, scFv, SdAb, Vβ-only domains, and TCR antigen binding domains derived from the TCR α and β chain variable domains.

For example, the antigen binding domain of the activator receptor may be part of a contiguous polypeptide chain including, for example, a Vβ-only domain, a single domain antibody fragment (sdAb) or heavy chain antibodies HCAb, a single chain antibody (scFv) derived from a murine, humanized or human antibodies (Harlow et al., 1999, In: Using Antibodies: A Laboratory Manual, Cold Spring Harbor Laboratory Press, N.Y.; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In some aspects, the antigen binding domain comprises an antibody fragment. In further aspects, the antigen binding domain comprises an antibody fragment that comprises a scFv or an sdAb.

The term “antibody,” as used herein, collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits and mice, as well as non-mammalian species, such as shark immunoglobulins. The term “antibody” includes intact immunoglobulins and “antibody fragments” or “antigen binding fragments” that specifically bind to a molecule of interest (or a group of highly similar molecules of interest) to the substantial exclusion of binding to other molecules (for example, antibodies and antibody fragments that have a binding constant for the molecule of interest that is at least 103 M-1 greater, at least 104 M-1 greater or at least 105 M-1 greater than a binding constant for other molecules in a biological sample). The term “antibody” also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology 3^{rd}, Ed., W.H. Freeman & Co., New York, 1997. The term antibody herein is used in the broadest sense and specifically covers intact monoclonal antibodies, polyclonal antibodies, multispecific antibodies (e.g., bispecific antibodies) formed from at least two intact antibodies, and antibody fragments, so long as they exhibit the desired biological activity.

The terms “antibody fragment” or “antibody binding domain” refer to at least one portion of an antibody, or recombinant variants thereof, that contains the antigen binding domain, i.e., an antigenic determining variable region of an intact antibody, that is sufficient to confer recognition and specific binding of the antibody fragment to a target, such as an antigen and its defined epitope. Examples of antibody fragments include, but are not limited to, Fab, Fab′, F(ab′)2, and Fv fragments, single-chain (sc)Fv (“scFv”) antibody fragments, linear antibodies, single domain antibodies (abbreviated “sdAb”) (either VL or VH), camelid VHH domains, and multi-specific antibodies formed from antibody fragments.

The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single polypeptide chain, and wherein the scFv retains the specificity of the intact antibody from which it is derived.

“Heavy chain variable region” or “VH” (or, in the case of single domain antibodies, e.g., nanobodies, “VHH”) with regard to an antibody refers to the fragment of the heavy chain that contains three complement determining regions (CDRs) interposed between flanking stretches known as framework regions, these framework regions are generally more highly conserved than the CDRs and form a scaffold to support the CDRs.

Unless specified, as used herein a scFv may have the VL and VH variable regions in either order, e.g., with respect to the N-terminal and C-terminal ends of the polypeptide, the scFv may comprise VL-linker-VH or may comprise VH-linker-VL.

In some embodiments, the antigen binding domain of the activator and/or inhibitory receptor comprises an scFv. In some embodiments, the scFv comprises a VL and VH region joined by a linker. In some embodiments, the linker comprises a glycine serine linker, for example GGGGSGGGGSGGGGSGG (SEQ ID NO: 25). In some embodiments, the scFv further comprises a signal sequence at the N terminus of the scFv. Exemplary signal sequences include MDMRVPAQLLGLLLLWLRGARC (SEQ ID NO: 26), which is encoded by ATGGACATGAGGGTCCCCGCTCAGCTCCTGGGGCTCCTGCTACTCTGGCTCCGA GGTGCCAGATGT (SEQ ID NO: 27).

The term “antibody light chain,” refers to the smaller of the two types of polypeptide chains present in antibody molecules in their naturally occurring conformations. Kappa (“K”) and lambda (“ ”) light chains refer to the two major antibody light chain isotypes.

The term “recombinant antibody” refers to an antibody that is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

The term “Vβ domain”, “Vβ-only domain”, “p chain variable domain” or “single variable domain TCR (svd-TCR)” refers to an antigen binding domain that consists essentially of a single T Cell Receptor (TCR) beta variable domain that specifically binds to an antigen in the absence of a second TCR variable domain. The Vβ-only domain engages antigen using complementarity-determining regions (CDRs). Each Vβ-only domain contains three complement determining regions (CDR1, CDR2, and CDR3). Additional elements may be combined provided that the Vβ domain is configured to bind the epitope in the absence of a second TCR variable domain.

In some embodiments, the antigen binding domain of the activator receptor comprises an antibody fragment, a single chain Fv antibody fragment (scFv), or a β chain variable domain (Vβ).

In some embodiments, the antigen binding domain of the activator receptor comprises a TCR α chain variable domain and a TCR β chain variable domain.

In some embodiments, the antigen binding domain of the activator receptor comprises an scFv antigen binding domain.

In some embodiments, the antigen binding domain of the activator receptor is an scFv. In some embodiments, the scFv domain binds to MSLN. In some embodiments, the scFv is the ligand binding domain of a CAR.

Exemplary scFv are described in PCT/US2021/060607, PCT/US2023/011698, US 20230029341, US 20220370497, US 20220273721, and US 20220054551, are incorporated herein by reference in their entirety. Exemplary scFv sequences are shown in Table 2 below (complementarity determining regions (CDRs) underlined).

TABLE 2

| scFv | Protein Sequence |
|---|---|
| MSLN | QVQLQESGPGLVKPSQTLSLTCTVSGGSISSGDYYWSWIRQPPGK |
| scFv | GLEWIGYIYYSGSTYYNPSLKSRVTISVDTSKNQFSLKLSSVTAA |
| 4174 | DTAVYYCAREDVVKGAFDIWGQGTMVTVSSGGGGSGGGGSGGGGS |
| | GGDIQMTQSPSSLSASVGDRVTITCRASQSISSYLNWYQQKPGKA |
| | PKLLIYAASSLQSGVPSRFSGSGSGTDFTLTISSLQPEDFATYYC |
| | QQSYSTPLTFGGGTKVEIK (SEQ ID NO: 28) |
| LRRN4 | QVQLKESGPGLVAPSQSLSITCTVSGFSLTNYAVHWVRQSPGKGL |
| scvFv | EWLGAIWSDGSTDYNAAFISRLSISKDNSKSQVFFKMNSLQADDT |
| 1J21 | AIYYCARKDYSNYVFDYWGQGTTLTVSSGGGGSGGGGSGGGGSGG |
| | EIVLTQSPAITAASLGQKVTISCSASSSVSYMHWYQQKSGTSPKP |
| | WIYDISKLASGVPARFSGSGSGTSYSLTISSMEAEDAAIYYCQQW |
| | SYPLITFGAGTKLELK (SEQ ID NO: 29) |
| Cd19 | DIQMTQTTSSLSASLGDRVTISCRASQDISKYLNWYQQKPDGTVK |
| scFv | LLIYHTSRLHSGVPSRFSGSGSGTDYSLTISNLEQEDIATYFCQQ |
| FMC63 | GNTLPYTFGGGTKLEITGGGGSGGGGSGGGGSGGEVKLQESGPGL |
| | VAPSQSLSVTCTVSGVSLPDYGVSWIRQPPRKGLEWLGVIWGSET |
| | TYYNSALKSRLTIIKDNSKSQVFLKMNSLQTDDTAIYYCAKHYYY |
| | GGSYAMDYWGQGTSVTVSS (SEQ ID NO: 30) |
| MSLN | QVQLVQSGAEVKKPGASVKVSCKASGYSFTGYTMNWVRQAPGQRL |
| scFv | EWMGLITPYNGASSYNQKFRGRVTITRDTSASTAYMELSSLRSED |
| SS1 | TAVYYCARGGYDGRGFDYWGQGTTVTVSSGGGGSGGGGSGGGGSG |
| | GDIQMTQSPSSLSASVGDRVTITCSASSSVSYMHWYQQKPGKAPK |
| | RLIYDTSKLASGVPSRFSGSGSGTEFTLTISSLQPEDFATYYCQQ |
| | WSGYPLTFGQGTKLEIK (SEQ ID NO: 31) |

In some embodiments, the activator receptor comprises an scFv having at least 80% identity, at least 85% identity, at least 90% identity, at least 95% identity, at least 97% identity or at least 99% identity to a sequence of SEQ ID NOS: 28-31.

In some embodiments, the activator receptor comprises a sequence having at least 80% identity, at least 85% identity, at least 90% identity, at least 95% identity, at least 97% identity or at least 99% identity to a sequence of SEQ ID NOS: 84-104 as shown in Table 3 below.

TABLE 3

| Receptor | Sequence |
|---|---|
| MSLN | QVQLQESGPGLVKPSQTLSLTCTVSGGSISSGDYYWSWIRQ |
| 1EGF | PPGKGLEWIGYIYYSGSTYYNPSLKSRVTISVDTSKNQFSL |
| | KLSSVTAADTAVYYCAREDVVKGAFDIWGQGTMVTVSSGGG |
| | GSGGGGSGGGGSGGDIQMTQSPSSLSASVGDRVTITCRASQ |
| | SISSYLNWYQQKPGKAPKLLIYAASSLQSGVPSRFSGSGSG |
| | TDFTLTISSLQPEDFATYYCQQSYSTPLTFGGGTKVEIKGG |
| | GGLTCVGHCSNGGSCTMNSKMMPECQCPPHMTGPRCEVFWV |
| | LVVVGGVLACYSLLVTVAFIIFWVRSKRSRLLHSDYMNMTP |
| | RRPGPTRKHYQPYAPPRDFAAYRSKRGRKKLLYIFKQPFMR |
| | PVQTTQEEDGCSCRFPEEEEGGCELRVKFSRSADAPAYKQG |
| | QNQLYNELNLGRREEYDVLDKRRGRDPEMGGKPRRKNPQEG |
| | LYNELQKDKMAEAYSEIGMKGERRRGKGHDGLYQGLSTATK |
| | DTYDALHMQALPPR (SEQ ID NO: 84) |
| MSLN | QVQLQESGPGLVKPSQTLSLTCTVSGGSISSGDYYWSWIRQ |
| 3EGF | PPGKGLEWIGYIYYSGSTYYNPSLKSRVTISVDTSKNQFSL |
| | KLSSVTAADTAVYYCAREDVVKGAFDIWGQGTMVTVSSGGG |
| | GSGGGGSGGGGSGGDIQMTQSPSSLSASVGDRVTITCRASQ |
| | SISSYLNWYQQKPGKAPKLLIYAASSLQSGVPSRFSGSGSG |
| | TDFTLTISSLQPEDFATYYCQQSYSTPLTFGGGTKVEIKGG |
| | GGQYRQCSGYCENFGTCQMAADGSRQCRCTAYFEGSRCEVN |
| | KCSRCLEGACVVNKQSGDVTCNCTDGRVAPSCLTCVGHCSN |
| | GGSCTMNSKMMPECQCPPHMTGPRCEVFWVLVVVGGVLACY |
| | SLLVTVAFIIFWVRSKRSRLLHSDYMNMTPRRPGPTRKHYQ |
| | PYAPPRDFAAYRSKRGRKKLLYIFKQPFMRPVQTTQEEDGC |
| | SCRFPEEEEGGCELRVKFSRSADAPAYKQGQNQLYNELNLG |
| | RREEYDVLDKRRGRDPEMGGKPRRKNPQEGLYNELQKDKMA |
| | EAYSEIGMKGERRRGKGHDGLYQGLSTATKDTYDALHMQAL |
| | PPR (SEQ ID NO: 85) |
| MSLN | QVQLQESGPGLVKPSQTLSLTCTVSGGSISSGDYYWSWIRQ |
| 5EGF | PPGKGLEWIGYIYYSGSTYYNPSLKSRVTISVDTSKNQFSL |
| | KLSSVTAADTAVYYCAREDVVKGAFDIWGQGTMVTVSSGGG |
| | GSGGGGSGGGGSGGDIQMTQSPSSLSASVGDRVTITCRASQ |
| | SISSYLNWYQQKPGKAPKLLIYAASSLQSGVPSRFSGSGSG |
| | TDFTLTISSLQPEDFATYYCQQSYSTPLTFGGGTKVEIKGG |
| | GGELDQCWEHCRNGGTCAASPSGMPTCRCPTGFTGPKCTQQ |
| | VCAGYCANNSTCTVNQGNQPQCRCLPGFLGDRCQYRQCSGY |
| | CENFGTCQMAADGSRQCRCTAYFEGSRCEVNKCSRCLEGAC |
| | VVNKQSGDVTCNCTDGRVAPSCLTCVGHCSNGGSCTMNSKM |
| | MPECQCPPHMTGPRCEVFWVLVVVGGVLACYSLLVTVAFII |
| | FWVRSKRSRLLHSDYMNMTPRRPGPTRKHYQPYAPPRDFAA |
| | YRSKRGRKKLLYIFKQPFMRPVQTTQEEDGCSCRFPEEEEG |
| | GCELRVKFSRSADAPAYKQGQNQLYNELNLGRREEYDVLDK |
| | RRGRDPEMGGKPRRKNPQEGLYNELQKDKMAEAYSEIGMKG |
| | ERRRGKGHDGLYQGLSTATKDTYDALHMQALPPR (SEQ |
| | ID NO: 86) |
| MSLN | QVQLQESGPGLVKPSQTLSLTCTVSGGSISSGDYYWSWIRQ |
| 7EGF | PPGKGLEWIGYIYYSGSTYYNPSLKSRVTISVDTSKNQFSL |
| | KLSSVTAADTAVYYCAREDVVKGAFDIWGQGTMVTVSSGGG |
| | GSGGGGSGGGGSGGDIQMTQSPSSLSASVGDRVTITCRASQ |
| | SISSYLNWYQQKPGKAPKLLIYAASSLQSGVPSRFSGSGSG |
| | TDFTLTISSLQPEDFATYYCQQSYSTPLTFGGGTKVEIKGG |
| | GGVTNPCDRKKCEWLCLLSPSGPVCTCPNGKRLDNGTCVPV |
| | PSPTPPPDAPRPGTCNLQCFNGGSCFLNARRQPKCRCQPRY |
| | TGDKCELDQCWEHCRNGGTCAASPSGMPTCRCPTGFTGPKC |
| | TQQVCAGYCANNSTCTVNQGNQPQCRCLPGFLGDRCQYRQC |
| | SGYCENFGTCQMAADGSRQCRCTAYFEGSRCEVNKCSRCLE |
| | GACVVNKQSGDVTCNCTDGRVAPSCLTCVGHCSNGGSCTMN |
| | SKMMPECQCPPHMTGPRCEVFWVLVVVGGVLACYSLLVTVA |
| | FIIFWVRSKRSRLLHSDYMNMTPRRPGPTRKHYQPYAPPRD |
| | FAAYRSKRGRKKLLYIFKQPFMRPVQTTQEEDGCSCRFPEE |
| | EEGGCELRVKFSRSADAPAYKQGQNQLYNELNLGRREEYDV |
| | LDKRRGRDPEMGGKPRRKNPQEGLYNELQKDKMAEAYSEIG |
| | MKGERRRGKGHDGLYQGLSTATKDTYDALHMQALPPR |
| | (SEQ ID NO: 87) |
| MSLNG4Q3 | QVQLQESGPGLVKPSQTLSLTCTVSGGSISSGDYYWSWIRQ |
| | PPGKGLEWIGYIYYSGSTYYNPSLKSRVTISVDTSKNQFSL |
| | KLSSVTAADTAVYYCAREDVVKGAFDIWGQGTMVTVSSGGG |
| | GSGGGGSGGGGSGGDIQMTQSPSSLSASVGDRVTITCRASQ |
| | SISSYLNWYQQKPGKAPKLLIYAASSLQSGVPSRFSGSGSG |
| | TDFTLTISSLQPEDFATYYCQQSYSTPLTFGGGTKVEIKGG |
| | GGQGGGGQGGGGQFWVLVVVGGVLACYSLLVTVAFIIFWVR |
| | SKRSRLLHSDYMNMTPRRPGPTRKHYQPYAPPRDFAAYRSK |
| | RGRKKLLYIFKQPFMRPVQTTQEEDGCSCRFPEEEEGGCEL |
| | RVKFSRSADAPAYKQGQNQLYNELNLGRREEYDVLDKRRGR |
| | DPEMGGKPRRKNPQEGLYNELQKDKMAEAYSEIGMKGERRR |
| | GKGHDGLYQGLSTATKDTYDALHMQALPPR (SEQ ID |
| | NO: 88) |
| MSLN | QVQLQESGPGLVKPSQTLSLTCTVSGGSISSGDYYWSWIRQ |
| G4Q6 | PPGKGLEWIGYIYYSGSTYYNPSLKSRVTISVDTSKNQFSL |
| | KLSSVTAADTAVYYCAREDVVKGAFDIWGQGTMVTVSSGGG |
| | GSGGGGSGGGGSGGDIQMTQSPSSLSASVGDRVTITCRASQ |
| | SISSYLNWYQQKPGKAPKLLIYAASSLQSGVPSRFSGSGSG |
| | TDFTLTISSLQPEDFATYYCQQSYSTPLTFGGGTKVEIKGG |
| | GGQGGGGQGGGGQGGGGQGGGGQGGGGQFWVLVVVGGVLAC |
| | YSLLVTVAFIIFWVRSKRSRLLHSDYMNMTPRRPGPTRKHY |
| | QPYAPPRDFAAYRSKRGRKKLLYIFKQPFMRPVQTTQEEDG |
| | CSCRFPEEEEGGCELRVKFSRSADAPAYKQGQNQLYNELNL |
| | GRREEYDVLDKRRGRDPEMGGKPRRKNPQEGLYNELQKDKM |
| | AEAYSEIGMKGERRRGKGHDGLYQGLSTATKDTYDALHMQA |
| | LPPR (SEQ ID NO: 89) |
| MSLN | QVQLQESGPGLVKPSQTLSLTCTVSGGSISSGDYYWSWIRQ |
| G4Q10 | PPGKGLEWIGYIYYSGSTYYNPSLKSRVTISVDTSKNQFSL |
| | KLSSVTAADTAVYYCAREDVVKGAFDIWGQGTMVTVSSGGG |
| | GSGGGGSGGGGSGGDIQMTQSPSSLSASVGDRVTITCRASQ |
| | SISSYLNWYQQKPGKAPKLLIYAASSLQSGVPSRFSGSGSG |
| | TDFTLTISSLQPEDFATYYCQQSYSTPLTFGGGTKVEIKGG |
| | GGQGGGGQGGGGQGGGGQGGGGQGGGGQGGGGQGGGGQGGG |
| | GQGGGGQFWVLVVVGGVLACYSLLVTVAFIIFWVRSKRSRL |
| | LHSDYMNMTPRRPGPTRKHYQPYAPPRDFAAYRSKRGRKKL |
| | LYIFKQPFMRPVQTTQEEDGCSCRFPEEEEGGCELRVKFSR |
| | SADAPAYKQGQNQLYNELNLGRREEYDVLDKRRGRDPEMGG |
| | KPRRKNPQEGLYNELQKDKMAEAYSEIGMKGERRRGKGHDG |
| | LYQGLSTATKDTYDALHMQALPPR (SEQ ID NO: 90) |
| MSLN | QVQLQESGPGLVKPSQTLSLTCTVSGGSISSGDYYWSWIRQ |
| G4Q12 | PPGKGLEWIGYIYYSGSTYYNPSLKSRVTISVDTSKNQFSL |
| | KLSSVTAADTAVYYCAREDVVKGAFDIWGQGTMVTVSSGGG |
| | GSGGGGSGGGGSGGDIQMTQSPSSLSASVGDRVTITCRASQ |
| | SISSYLNWYQQKPGKAPKLLIYAASSLQSGVPSRFSGSGSG |
| | TDFTLTISSLQPEDFATYYCQQSYSTPLTFGGGTKVEIKGG |
| | GGQGGGGQGGGGQGGGGQGGGGQGGGGQGGGGQGGGGQGGG |
| | GQGGGGQGGGGQGGGGQFWVLVVVGGVLACYSLLVTVAFII |
| | FWVRSKRSRLLHSDYMNMTPRRPGPTRKHYQPYAPPRDFAA |
| | YRSKRGRKKLLYIFKQPFMRPVQTTQEEDGCSCRFPEEEEG |
| | GCELRVKFSRSADAPAYKQGQNQLYNELNLGRREEYDVLDK |
| | RRGRDPEMGGKPRRKNPQEGLYNELQKDKMAEAYSEIGMKG |
| | ERRRGKGHDGLYQGLSTATKDTYDALHMQALPPR (SEQ |
| | ID NO: 91) |
| MSLN | QVQLQESGPGLVKPSQTLSLTCTVSGGSISSGDYYWSWIRQ |
| TSP2 | PPGKGLEWIGYIYYSGSTYYNPSLKSRVTISVDTSKNQFSL |
| | KLSSVTAADTAVYYCAREDVVKGAFDIWGQGTMVTVSSGGG |
| | GSGGGGSGGGGSGGDIQMTQSPSSLSASVGDRVTITCRASQ |
| | SISSYLNWYQQKPGKAPKLLIYAASSLQSGVPSRFSGSGSG |
| | TDFTLTISSLQPEDFATYYCQQSYSTPLTFGGGTKVEIKGG |
| | GGSPRWSLWSTWAPCSVTCSEGSQLRYRRCVGWNGQCSGKV |
| | APGTLEWQLQACEDQQCCPEMGGWSGWGPWEPCSVTCSKGT |
| | RTRRRACNHPAPKCGGHCPGQAQESEACDTQQVCPFWVLVV |
| | VGGVLACYSLLVTVAFIIFWVRSKRSRLLHSDYMNMTPRRP |
| | GPTRKHYQPYAPPRDFAAYRSKRGRKKLLYIFKQPFMRPVQ |
| | TTQEEDGCSCRFPEEEEGGCELRVKFSRSADAPAYKQGQNQ |
| | LYNELNLGRREEYDVLDKRRGRDPEMGGKPRRKNPQEGLYN |
| | ELQKDKMAEAYSEIGMKGERRRGKGHDGLYQGLSTATKDTY |
| | DALHMQALPPR (SEQ ID NO: 92) |
| MSLN | QVQLQESGPGLVKPSQTLSLTCTVSGGSISSGDYYWSWIRQ |
| TSP4 | PPGKGLEWIGYIYYSGSTYYNPSLKSRVTISVDTSKNQFSL |
| | KLSSVTAADTAVYYCAREDVVKGAFDIWGQGTMVTVSSGGG |
| | GSGGGGSGGGGSGGDIQMTQSPSSLSASVGDRVTITCRASQ |
| | SISSYLNWYQQKPGKAPKLLIYAASSLQSGVPSRFSGSGSG |
| | TDFTLTISSLQPEDFATYYCQQSYSTPLTFGGGTKVEIKGG |
| | GGSPRWSLWSTWAPCSVTCSEGSQLRYRRCVGWNGQCSGKV |
| | APGTLEWQLQACEDQQCCPEMGGWSGWGPWEPCSVTCSKGT |
| | RTRRRACNHPAPKCGGHCPGQAQESEACDTQQVCPTHGAWA |
| | TWGPWTPCSASCHGGPHEPKETRSRKCSAPEPSQKPPGKPC |
| | PGLAYEQRRCTGLPPCPVAGGWGPWGPVSPCPVTCGLGQTM |
| | EQRTCNHPVPQHGGPFCAGDATRTHICNTAVPCPFWVLVVV |
| | GGVLACYSLLVTVAFIIFWVRSKRSRLLHSDYMNMTPRRPG |
| | PTRKHYQPYAPPRDFAAYRSKRGRKKLLYIFKQPFMRPVQT |
| | TQEEDGCSCRFPEEEEGGCELRVKFSRSADAPAYKQGQNQL |
| | YNELNLGRREEYDVLDKRRGRDPEMGGKPRRKNPQEGLYNE |
| | LQKDKMAEAYSEIGMKGERRRGKGHDGLYQGLSTATKDTYD |
| | ALHMQALPPR (SEQ ID NO: 93) |
| MSLN | QVQLQESGPGLVKPSQTLSLTCTVSGGSISSGDYYWSWIRQ |
| TSP6 | PPGKGLEWIGYIYYSGSTYYNPSLKSRVTISVDTSKNQFSL |
| | KLSSVTAADTAVYYCAREDVVKGAFDIWGQGTMVTVSSGGG |
| | GSGGGGSGGGGSGGDIQMTQSPSSLSASVGDRVTITCRASQ |
| | SISSYLNWYQQKPGKAPKLLIYAASSLQSGVPSRFSGSGSG |
| | TDFTLTISSLQPEDFATYYCQQSYSTPLTFGGGTKVEIKGG |
| | GGSPRWSLWSTWAPCSVTCSEGSQLRYRRCVGWNGQCSGKV |
| | APGTLEWQLQACEDQQCCPEMGGWSGWGPWEPCSVTCSKGT |
| | RTRRRACNHPAPKCGGHCPGQAQESEACDTQQVCPTHGAWA |
| | TWGPWTPCSASCHGGPHEPKETRSRKCSAPEPSQKPPGKPC |
| | PGLAYEQRRCTGLPPCPVAGGWGPWGPVSPCPVTCGLGQTM |
| | EQRTCNHPVPQHGGPFCAGDATRTHICNTAVPCPVDGEWDS |
| | WGEWSPCIRRNMKSISCQEIPGQQSRGRTCRGRKFDGHRCA |
| | GQQQDIRHCYSIQHCPLKGSWSEWSTWGLCMPPCGPNPTRA |
| | RQRLCTPLLPKYPPTVSMVEGQGEKNVTFWGRPLPRCEELQ |
| | GQKLVVEEKRPCLHVPACKFWVLVVVGGVLACYSLLVTVAF |
| | IIFWVRSKRSRLLHSDYMNMTPRRPGPTRKHYQPYAPPRDF |
| | AAYRSKRGRKKLLYIFKQPFMRPVQTTQEEDGCSCRFPEEE |
| | EGGCELRVKFSRSADAPAYKQGQNQLYNELNLGRREEYDVL |
| | DKRRGRDPEMGGKPRRKNPQEGLYNELQKDKMAEAYSEIGM |
| | KGERRRGKGHDGLYQGLSTATKDTYDALHMQALPPR (SEQ |
| | ID NO: 94) |
| MSLN | QVQLQESGPGLVKPSQTLSLTCTVSGGSISSGDYYWSWIRQ |
| Ig2 | PPGKGLEWIGYIYYSGSTYYNPSLKSRVTISVDTSKNQFSL |
| | KLSSVTAADTAVYYCAREDVVKGAFDIWGQGTMVTVSSGGG |
| | GSGGGGSGGGGSGGDIQMTQSPSSLSASVGDRVTITCRASQ |
| | SISSYLNWYQQKPGKAPKLLIYAASSLQSGVPSRFSGSGSG |
| | TDFTLTISSLQPEDFATYYCQQSYSTPLTFGGGTKVEIKGG |
| | GGPKPSISSNNSKPVEDKDAVAFTCEPEAQNTTYLWWVNGQ |
| | SLPVSPRLQLSNGNRTLTLFNVTRNDARAYVCGIQNSVSAN |
| | RSDPVTLDVLYGPDTPIISPPDSSYLSGANLNLSCHSASNP |
| | SPQYSWRINGIPQQHTQVLFIAKITPNNNGTYACFVSNLAT |
| | GRNNSIVKSITVSFWVLVVVGGVLACYSLLVTVAFIIFWVR |
| | SKRSRLLHSDYMNMTPRRPGPTRKHYQPYAPPRDFAAYRSK |
| | RGRKKLLYIFKQPFMRPVQTTQEEDGCSCRFPEEEEGGCEL |
| | RVKFSRSADAPAYKQGQNQLYNELNLGRREEYDVLDKRRGR |
| | DPEMGGKPRRKNPQEGLYNELQKDKMAEAYSEIGMKGERRR |
| | GKGHDGLYQGLSTATKDTYDALHMQALPPR (SEQ ID |
| | NO: 95) |
| MSLN | QVQLQESGPGLVKPSQTLSLTCTVSGGSISSGDYYWSWIRQ |
| Ig4 | PPGKGLEWIGYIYYSGSTYYNPSLKSRVTISVDTSKNQFSL |
| | KLSSVTAADTAVYYCAREDVVKGAFDIWGQGTMVTVSSGGG |
| | GSGGGGSGGGGSGGDIQMTQSPSSLSASVGDRVTITCRASQ |
| | SISSYLNWYQQKPGKAPKLLIYAASSLQSGVPSRFSGSGSG |
| | TDFTLTISSLQPEDFATYYCQQSYSTPLTFGGGTKVEIKGG |
| | GGPKPFITSNNSNPVEDEDAVALTCEPEIQNTTYLWWVNNQ |
| | SLPVSPRLQLSNDNRTLTLLSVTRNDVGPYECGIQNELSVD |
| | HSDPVILNVLYGPDDPTISPSYTYYRPGVNLSLSCHAASNP |
| | PAQYSWLIDGNIQQHTQELFISNITEKNSGLYTCQANNSAS |
| | GHSRTTVKTITVSAELPKPSISSNNSKPVEDKDAVAFTCEP |
| | EAQNTTYLWWVNGQSLPVSPRLQLSNGNRTLTLFNVTRNDA |
| | RAYVCGIQNSVSANRSDPVTLDVLYGPDTPIISPPDSSYLS |
| | GANLNLSCHSASNPSPQYSWRINGIPQQHTQVLFIAKITPN |
| | NNGTYACFVSNLATGRNNSIVKSITVSFWVLVVVGGVLACY |
| | SLLVTVAFIIFWVRSKRSRLLHSDYMNMTPRRPGPTRKHYQ |
| | PYAPPRDFAAYRSKRGRKKLLYIFKQPFMRPVQTTQEEDGC |
| | SCRFPEEEEGGCELRVKFSRSADAPAYKQGQNQLYNELNLG |
| | RREEYDVLDKRRGRDPEMGGKPRRKNPQEGLYNELQKDKMA |
| | EAYSEIGMKGERRRGKGHDGLYQGLSTATKDTYDALHMQAL |
| | PPR (SEQ ID NO: 96) |
| MSLN | QVQLQESGPGLVKPSQTLSLTCTVSGGSISSGDYYWSWIRQ |
| Ig6 | PPGKGLEWIGYIYYSGSTYYNPSLKSRVTISVDTSKNQFSL |
| | KLSSVTAADTAVYYCAREDVVKGAFDIWGQGTMVTVSSGGG |
| | GSGGGGSGGGGSGGDIQMTQSPSSLSASVGDRVTITCRASQ |
| | SISSYLNWYQQKPGKAPKLLIYAASSLQSGVPSRFSGSGSG |
| | TDFTLTISSLQPEDFATYYCQQSYSTPLTFGGGTKVEIKGG |
| | GGPKPSISSNNSKPVEDKDAVAFTCEPETQDATYLWWVNNQ |
| | SLPVSPRLQLSNGNRTLTLFNVTRNDTASYKCETQNPVSAR |
| | RSDSVILNVLYGPDAPTISPLNTSYRSGENLNLSCHAASNP |
| | PAQYSWFVNGTFQQSTQELFIPNITVNNSGSYTCQAHNSDT |
| | GLNRTTVTTITVYAEPPKPFITSNNSNPVEDEDAVALTCEP |
| | EIQNTTYLWWVNNQSLPVSPRLQLSNDNRTLTLLSVTRNDV |
| | GPYECGIQNELSVDHSDPVILNVLYGPDDPTISPSYTYYRP |
| | GVNLSLSCHAASNPPAQYSWLIDGNIQQHTQELFISNITEK |
| | NSGLYTCQANNSASGHSRTTVKTITVSAELPKPSISSNNSK |
| | PVEDKDAVAFTCEPEAQNTTYLWWVNGQSLPVSPRLQLSNG |
| | NRTLTLFNVTRNDARAYVCGIQNSVSANRSDPVTLDVLYGP |
| | DTPIISPPDSSYLSGANLNLSCHSASNPSPQYSWRINGIPQ |
| | QHTQVLFIAKITPNNNGTYACFVSNLATGRNNSIVKSITVS |
| | FWVLVVVGGVLACYSLLVTVAFIIFWVRSKRSRLLHSDYMN |
| | MTPRRPGPTRKHYQPYAPPRDFAAYRSKRGRKKLLYIFKQP |
| | FMRPVQTTQEEDGCSCRFPEEEEGGCELRVKFSRSADAPAY |
| | KQGQNQLYNELNLGRREEYDVLDKRRGRDPEMGGKPRRKNP |
| | QEGLYNELQKDKMAEAYSEIGMKGERRRGKGHDGLYQGLST |
| | ATKDTYDALHMQALPPR (SEQ ID NO: 97) |
| CD19 | DIQMTQTTSSLSASLGDRVTISCRASQDISKYLNWYQQKPD |
| EGF1 | GTVKLLIYHTSRLHSGVPSRFSGSGSGTDYSLTISNLEQED |
| | IATYFCQQGNTLPYTFGGGTKLEITGGGGSGGGGSGGGGSG |
| | GEVKLQESGPGLVAPSQSLSVTCTVSGVSLPDYGVSWIRQP |
| | PRKGLEWLGVIWGSETTYYNSALKSRLTIIKDNSKSQVFLK |
| | MNSLQTDDTAIYYCAKHYYYGGSYAMDYWGQGTSVTVSSGG |
| | GGLTCVGHCSNGGSCTMNSKMMPECQCPPHMTGPRCEVFWV |
| | LVVVGGVLACYSLLVTVAFIIFWVRSKRSRLLHSDYMNMTP |
| | RRPGPTRKHYQPYAPPRDFAAYRSKRGRKKLLYIFKQPFMR |
| | PVQTTQEEDGCSCRFPEEEEGGCELRVKFSRSADAPAYKQG |
| | QNQLYNELNLGRREEYDVLDKRRGRDPEMGGKPRRKNPQEG |
| | LYNELQKDKMAEAYSEIGMKGERRRGKGHDGLYQGLSTATK |
| | DTYDALHMQALPPR (SEQ ID NO: 98) |
| CD19 | DIQMTQTTSSLSASLGDRVTISCRASQDISKYLNWYQQKPD |
| EGF2 | GTVKLLIYHTSRLHSGVPSRFSGSGSGTDYSLTISNLEQED |
| | IATYFCQQGNTLPYTFGGGTKLEITGGGGSGGGGSGGGGSG |
| | GEVKLQESGPGLVAPSQSLSVTCTVSGVSLPDYGVSWIRQP |
| | PRKGLEWLGVIWGSETTYYNSALKSRLTIIKDNSKSQVFLK |
| | MNSLQTDDTAIYYCAKHYYYGGSYAMDYWGQGTSVTVSSGG |
| | GGEVNKCSRCLEGACVVNKQSGDVTCNCTDGRVAPSCLTCV |
| | GHCSNGGSCTMNSKMMPECQCPPHMTGPRCEVFWVLVVVGG |
| | VLACYSLLVTVAFIIFWVRSKRSRLLHSDYMNMTPRRPGPT |
| | RKHYQPYAPPRDFAAYRSKRGRKKLLYIFKQPFMRPVQTTQ |
| | EEDGCSCRFPEEEEGGCELRVKFSRSADAPAYKQGQNQLYN |
| | ELNLGRREEYDVLDKRRGRDPEMGGKPRRKNPQEGLYNELQ |
| | KDKMAEAYSEIGMKGERRRGKGHDGLYQGLSTATKDTYDAL |
| | HMQALPPR (SEQ ID NO: 99) |
| CD19 | DIQMTQTTSSLSASLGDRVTISCRASQDISKYLNWYQQKPD |
| EGF3 | GTVKLLIYHTSRLHSGVPSRFSGSGSGTDYSLTISNLEQED |
| | IATYFCQQGNTLPYTFGGGTKLEITGGGGSGGGGSGGGGSG |
| | GEVKLQESGPGLVAPSQSLSVTCTVSGVSLPDYGVSWIRQP |
| | PRKGLEWLGVIWGSETTYYNSALKSRLTIIKDNSKSQVFLK |
| | MNSLQTDDTAIYYCAKHYYYGGSYAMDYWGQGTSVTVSSGG |
| | GGQYRQCSGYCENFGTCQMAADGSRQCRCTAYFEGSRCEVN |
| | KCSRCLEGACVVNKQSGDVTCNCTDGRVAPSCLTCVGHCSN |
| | GGSCTMNSKMMPECQCPPHMTGPRCEVFWVLVVVGGVLACY |
| | SLLVTVAFIIFWVRSKRSRLLHSDYMNMTPRRPGPTRKHYQ |
| | PYAPPRDFAAYRSKRGRKKLLYIFKQPFMRPVQTTQEEDGC |
| | SCRFPEEEEGGCELRVKFSRSADAPAYKQGQNQLYNELNLG |
| | RREEYDVLDKRRGRDPEMGGKPRRKNPQEGLYNELQKDKMA |
| | EAYSEIGMKGERRRGKGHDGLYQGLSTATKDTYDALHMQAL |
| | PPR (SEQ ID NO: 100) |
| CD19 | DIQMTQTTSSLSASLGDRVTISCRASQDISKYLNWYQQKPD |
| EGF4 | GTVKLLIYHTSRLHSGVPSRFSGSGSGTDYSLTISNLEQED |
| | IATYFCQQGNTLPYTFGGGTKLEITGGGGSGGGGSGGGGSG |
| | GEVKLQESGPGLVAPSQSLSVTCTVSGVSLPDYGVSWIRQP |
| | PRKGLEWLGVIWGSETTYYNSALKSRLTIIKDNSKSQVFLK |
| | MNSLQTDDTAIYYCAKHYYYGGSYAMDYWGQGTSVTVSSGG |
| | GGTQQVCAGYCANNSTCTVNQGNQPQCRCLPGFLGDRCQYR |
| | QCSGYCENFGTCQMAADGSRQCRCTAYFEGSRCEVNKCSRC |
| | LEGACVVNKQSGDVTCNCTDGRVAPSCLTCVGHCSNGGSCT |
| | MNSKMMPECQCPPHMTGPRCEVFWVLVVVGGVLACYSLLVT |
| | VAFIIFWVRSKRSRLLHSDYMNMTPRRPGPTRKHYQPYAPP |
| | RDFAAYRSKRGRKKLLYIFKQPFMRPVQTTQEEDGCSCRFP |
| | EEEEGGCELRVKFSRSADAPAYKQGQNQLYNELNLGRREEY |
| | DVLDKRRGRDPEMGGKPRRKNPQEGLYNELQKDKMAEAYSE |
| | IGMKGERRRGKGHDGLYQGLSTATKDTYDALHMQALPPR |
| | (SEQ ID NO: 101) |
| CD19 | DIQMTQTTSSLSASLGDRVTISCRASQDISKYLNWYQQKPD |
| EGF5 | GTVKLLIYHTSRLHSGVPSRFSGSGSGTDYSLTISNLEQED |
| | IATYFCQQGNTLPYTFGGGTKLEITGGGGSGGGGSGGGGSG |
| | GEVKLQESGPGLVAPSQSLSVTCTVSGVSLPDYGVSWIRQP |
| | PRKGLEWLGVIWGSETTYYNSALKSRLTIIKDNSKSQVFLK |
| | MNSLQTDDTAIYYCAKHYYYGGSYAMDYWGQGTSVTVSSGG |
| | GGELDQCWEHCRNGGTCAASPSGMPTCRCPTGFTGPKCTQQ |
| | VCAGYCANNSTCTVNQGNQPQCRCLPGFLGDRCQYRQCSGY |
| | CENFGTCQMAADGSRQCRCTAYFEGSRCEVNKCSRCLEGAC |
| | VVNKQSGDVTCNCTDGRVAPSCLTCVGHCSNGGSCTMNSKM |
| | MPECQCPPHMTGPRCEVFWVLVVVGGVLACYSLLVTVAFII |
| | FWVRSKRSRLLHSDYMNMTPRRPGPTRKHYQPYAPPRDFAA |
| | YRSKRGRKKLLYIFKQPFMRPVQTTQEEDGCSCRFPEEEEG |
| | GCELRVKFSRSADAPAYKQGQNQLYNELNLGRREEYDVLDK |
| | RRGRDPEMGGKPRRKNPQEGLYNELQKDKMAEAYSEIGMKG |
| | ERRRGKGHDGLYQGLSTATKDTYDALHMQALPPR (SEQ |
| | ID NO: 102) |
| CD19 | DIQMTQTTSSLSASLGDRVTISCRASQDISKYLNWYQQKPD |
| EGF6 | GTVKLLIYHTSRLHSGVPSRFSGSGSGTDYSLTISNLEQED |
| | IATYFCQQGNTLPYTFGGGTKLEITGGGGSGGGGSGGGGSG |
| | GEVKLQESGPGLVAPSQSLSVTCTVSGVSLPDYGVSWIRQP |
| | PRKGLEWLGVIWGSETTYYNSALKSRLTIIKDNSKSQVFLK |
| | MNSLQTDDTAIYYCAKHYYYGGSYAMDYWGQGTSVTVSSGG |
| | GGRPGTCNLQCFNGGSCFLNARRQPKCRCQPRYTGDKCELD |
| | QCWEHCRNGGTCAASPSGMPTCRCPTGFTGPKCTQQVCAGY |
| | CANNSTCTVNQGNQPQCRCLPGFLGDRCQYRQCSGYCENFG |
| | TCQMAADGSRQCRCTAYFEGSRCEVNKCSRCLEGACVVNKQ |
| | SGDVTCNCTDGRVAPSCLTCVGHCSNGGSCTMNSKMMPECQ |
| | CPPHMTGPRCEVFWVLVVVGGVLACYSLLVTVAFIIFWVRS |
| | KRSRLLHSDYMNMTPRRPGPTRKHYQPYAPPRDFAAYRSKR |
| | GRKKLLYIFKQPFMRPVQTTQEEDGCSCRFPEEEEGGCELR |
| | VKFSRSADAPAYKQGQNQLYNELNLGRREEYDVLDKRRGRD |
| | PEMGGKPRRKNPQEGLYNELQKDKMAEAYSEIGMKGERRRG |
| | KGHDGLYQGLSTATKDTYDALHMQALPPR (SEQ ID NO: |
| | 103) |
| CD19 | DIQMTQTTSSLSASLGDRVTISCRASQDISKYLNWYQQKPD |
| EGF7 | GTVKLLIYHTSRLHSGVPSRFSGSGSGTDYSLTISNLEQED |
| | IATYFCQQGNTLPYTFGGGTKLEITGGGGSGGGGSGGGGSG |
| | GEVKLQESGPGLVAPSQSLSVTCTVSGVSLPDYGVSWIRQP |
| | PRKGLEWLGVIWGSETTYYNSALKSRLTIIKDNSKSQVFLK |
| | MNSLQTDDTAIYYCAKHYYYGGSYAMDYWGQGTSVTVSSGG |
| | GGVTNPCDRKKCEWLCLLSPSGPVCTCPNGKRLDNGTCVPV |
| | PSPTPPPDAPRPGTCNLQCFNGGSCFLNARRQPKCRCQPRY |
| | TGDKCELDQCWEHCRNGGTCAASPSGMPTCRCPTGFTGPKC |
| | TQQVCAGYCANNSTCTVNQGNQPQCRCLPGFLGDRCQYRQC |
| | SGYCENFGTCQMAADGSRQCRCTAYFEGSRCEVNKCSRCLE |
| | GACVVNKQSGDVTCNCTDGRVAPSCLTCVGHCSNGGSCTMN |
| | SKMMPECQCPPHMTGPRCEVFWVLVVVGGVLACYSLLVTVA |
| | FIIFWVRSKRSRLLHSDYMNMTPRRPGPTRKHYQPYAPPRD |
| | FAAYRSKRGRKKLLYIFKQPFMRPVQTTQEEDGCSCRFPEE |
| | EEGGCELRVKFSRSADAPAYKQGQNQLYNELNLGRREEYDV |
| | LDKRRGRDPEMGGKPRRKNPQEGLYNELQKDKMAEAYSEIG |
| | MKGERRRGKGHDGLYQGLSTATKDTYDALHMQALPPR |
| | (SEQ ID NO: 104) |

### Chimeric Antigen Receptors (CARs)

The disclosure provides an activator receptor and engineered cells comprising same. In some embodiments, the activator receptor is a chimeric antigen receptor.

The term “chimeric antigen receptors (CARs)” as used herein, may refer to artificial receptors derived from T-cell receptors and encompasses engineered receptors that graft an artificial specificity onto a particular immune effector cell. CARs may be employed to impart the specificity of a monoclonal antibody onto a T cell, thereby allowing a large number of specific T cells to be generated, for example, for use in adoptive cell therapy. In specific embodiments, CARs direct specificity of the cell to a tumor associated antigen, for example. Exemplary CARs comprise an intracellular activation domain, a transmembrane domain, and an extracellular domain comprising a tumor associated antigen binding region. In some embodiments, CARs further comprise a hinge domain. In particular aspects, CARs comprise fusions of single-chain variable fragments (scFv) derived from monoclonal antibodies, fused to a CD3 transmembrane domain and endodomain. The specificity of other CAR designs may be derived from ligands of receptors (e.g., peptides). In certain cases, CARs comprise domains for additional co-stimulatory signaling, such as CD3, 4-1BB, FcR, CD27, CD28, CD137, DAP10, and/or OX40. In some cases, molecules can be co-expressed with the CAR, including co-stimulatory molecules, reporter genes for imaging, gene products that conditionally ablate the T cells upon addition of a pro-drug, homing receptors, cytokines, and cytokine receptors.

In some embodiments, the antigen binding domain of the activator receptor is fused to the extracellular domain of a CAR.

In some embodiments, the CARs of the present disclosure comprise an extracellular hinge region. Hinge elements contemplated for use in the present disclosure are described in detail above. In some embodiments, a hinge is selected such that the combined height of the activator receptor and first antigen epitope is greater than the combined height of the inhibitory receptor and second antigen epitope.

The CARs of the present disclosure can be designed to comprise a transmembrane domain that is fused to the extracellular domain of the CAR. In some embodiments, the transmembrane domain that naturally is associated with one of the domains in the CAR is used. For example, a CAR comprising a CD28 co-stimulatory domain might also use a CD28 transmembrane domain. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein. Transmembrane regions may be isolated or derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, or from an immunoglobulin such as IgG4. Alternatively, the transmembrane domain may be synthetic, in which case it will comprise predominantly hydrophobic residues such as leucine and valine. In some embodiments, a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

In some embodiments of the CARs of the disclosure, the CARs comprise a CD28 transmembrane domain. In some embodiments, the CD28 transmembrane domain comprises an amino acid sequence having at least 80% identity, at least 90% identity, at least 95% identity, at least 99% identity or is identical to a sequence of FWVLVVVGGVLACYSLLVTVAFIIFWV (SEQ ID NO: 32). In some embodiments, the CD28 transmembrane domain comprises or consists essentially of SEQ ID NO: 32. In some embodiments, the CD28 transmembrane domain is encoded by a nucleotide sequence having at least 80% identity, at least 90% identity, at least 95% identity, at least 99% identity or is identical to a sequence of TTCTGGGTGCTGGTCGTTGTGGGCGGCGTGCTGGCCTGCTACAGCCTGCTGGTGA CAGTGGCCTTCATCATCTTTTGGGTG (SEQ ID NO: 33). In some embodiments, the CD28 transmembrane domain is encoded by SEQ ID NO: 33.

In some embodiments of the CARs of the disclosure, the CARs comprise an IL-2Rbeta transmembrane domain. In some embodiments, the IL-2Rbeta transmembrane domain comprises an amino acid sequence having at least 80% identity, at least 90% identity, at least 95% identity, at least 99% identity or is identical to a sequence of IPWLGHLLVGLSGAFGFIILVYLLI (SEQ ID NO: 34). In some embodiments, the IL-2Rbeta transmembrane domain comprises or consists essentially of SEQ ID NO: 34. In some embodiments, the TL-2Rbeta transmembrane domain is encoded by a nucleotide sequence having at least 80% identity, at least 90% identity, at least 95% identity, at least 99% identity or is identical to a sequence of ATTCCGTGGC TCGGCCACCT CCTCGTGGGC CTCAGCGGGG CTTTTGGCTT CATCATCTTA GTGTACTTGC TGATC (SEQ ID NO: 35). In some embodiments, the IL-2Rbeta transmembrane domain is encoded by SEQ ID NO: 35.

The cytoplasmic domain or otherwise the intracellular signaling domain of the CARs of the instant disclosure is responsible for activation of at least one of the normal effector functions of the immune cell in which the CAR has been placed. The term “effector function” refers to a specialized function of a cell. Thus, the term “intracellular signaling domain” refers to the portion of a protein which transduces the effector function signal and directs the cell to perform a specialized function. While usually the entire intracellular signaling domain can be employed, in many cases it is not necessary to use the entire domain. To the extent that a truncated portion of the intracellular signaling domain is used, such truncated portion may be used in place of the intact chain as long as it transduces the effector function signal. In some cases, multiple intracellular domains can be combined to achieve the desired functions of the CAR-T cells of the instant disclosure. The term intracellular signaling domain is thus meant to include any truncated portion of one or more intracellular signaling domains sufficient to transduce the effector function signal.

Examples of intracellular signaling domains for use in the CARs of the instant disclosure include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

Accordingly, the intracellular domain of CARs of the instant disclosure comprises at least one cytoplasmic activation domain. In some embodiments, the intracellular activation domain ensures that there is T-cell receptor (TCR) signaling necessary to activate the effector functions of the CAR T-cell. In some embodiments, the at least one cytoplasmic activation is a CD247 molecule (CD3ζ) activation domain, a stimulatory killer immunoglobulin-like receptor (KIR) KIR2DS2 activation domain, or a DNAX-activating protein of 12 kDa (DAP12) activation domain.

In some embodiments, the CD3ζ activation domain comprises an amino acid sequence having at least 80% identity, at least 90% identity, at least 95% identity, at least 99% identity or is identical to a sequence of

(SEQ ID NO: 36)
RVKFSRSADAPAYKQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

In some embodiments, the CD3ζ activation domain comprises or consists essentially of SEQ ID NO: 36. In some embodiments, the CD3ζ activation domain is encoded by a nucleotide sequence having at least 80% identity, at least 90% identity, at least 95% identity, at least 99% identity or is identical to a sequence of: AGAGTGAAGTTCAGCAGGAGCGCAGACGCCCCCGCGTACAAGCAGGGCCAGAA CCAGCTCTATAACGAGCTCAATCTAGGACGAAGAGAGGAGTACGATGTTTTGGA CAAGCGTAGAGGCCGGGACCCTGAGATGGGGGGAAAGCCGAGAAGGAAGAAC CCTCAGGAAGGCCTGTACAATGAACTGCAGAAAGATAAGATGGCGGAGGCCTA CAGTGAGATTGGGATGAAAGGCGAGCGCCGGAGGGGCAAGGGGCACGATGGCC TTTACCAGGGACTCAGTACAGCCACCAAGGACACCTACGACGCCCTTCACATGC AGGCCCTGCCCCCTCGC (SEQ ID NO: 37). In some embodiments, the CD3ζ activation domain is encoded by SEQ ID NO: 37).

It is known that signals generated through the TCR alone are often insufficient for full activation of the T cell and that a secondary or co-stimulatory signal is also required. Thus, T cell activation can be said to be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

Primary cytoplasmic signaling sequences regulate primary activation of the TCR complex either in a stimulatory way, or in an inhibitory way. Primary cytoplasmic signaling sequences that act in a stimulatory manner may contain signaling motifs, which are known as immunoreceptor tyrosine-based activation motifs or ITAMs. In some embodiments, the ITAM contains a tyrosine separated from a leucine or an isoleucine by any two other amino acids (YxxL/I (SEQ ID NO: 38)). In some embodiments, the cytoplasmic domain contains 1, 2, 3, 4 or 5 ITAMs. An exemplary ITAM containing cytoplasmic domain is the CD3ζ activation domain. Further examples of ITAM containing primary cytoplasmic signaling sequences that can be used in the CARs of the instant disclosure include those derived from TCRζ, FcRγ, FcRβ, CD3γ, CD3δ, CD3ε, CD3ζ, CD5, CD22, CD79a, CD79b, and CD66d.

In some embodiments, the CD3ζ activation domain comprising a single ITAM comprises an amino acid sequence having at least 80% identity, at least 90% identity, at least 95% identity, at least 99% identity or is identical to a sequence of RVKFSRSADAPAYQQGQNQLYNELNLGRREEYDVLHMQALPPR (SEQ ID NO: 39). In some embodiments, the CD3ζ activation domain comprises SEQ ID NO: 39. In some embodiments, the CD3ζ activation domain comprising a single ITAM consists essentially of an amino acid sequence of: RVKFSRSADAPAYQQGQNQLYNELNLGRREEYDVLHMQALPPR (SEQ ID NO: 39. In some embodiments, the CD3ζ activation domain comprising a single ITAM is encoded by a nucleotide sequence having at least 80% identity, at least 90% identity, at least 95% identity, at least 99% identity or is identical to a sequence of: AGAGTGAAGT TCAGCAGGAG CGCAGACGCC CCCGCGTACC AGCAGGGCCA GAACCAGCTC TATAACGAGC TCAATCTAGG ACGAAGAGAG GAGTACGATG TTTTGCACAT GCAGGCCCTG CCCCCTCGC (SEQ ID NO: 40). In some embodiments, the CD3ζ activation domain is encoded by SEQ ID NO: 40.

In some embodiments, the cytoplasmic domain of the CAR can be designed to comprise the CD3ζ signaling domain by itself or combined with any other desired cytoplasmic domain(s) useful in the context of the CAR of the instant disclosure. For example, the cytoplasmic domain of the CAR can comprise a CD3ζ chain portion and a co-stimulatory domain. The co-stimulatory domain refers to a portion of the CAR comprising the intracellular domain of a costimulatory molecule. A costimulatory molecule is a cell surface molecule other than an antigen receptor or its ligands that is required for an efficient response of lymphocytes to an antigen. Examples of such molecules include the co-stimulatory domain is selected from the group consisting of IL-2Rβ, Fc Receptor gamma (FcRγ), Fc Receptor beta (FcRβ), CD3g molecule gamma (CD3γ), CD3δ, CD3ε, CD5 molecule (CD5), CD22 molecule (CD22), CD79a molecule (CD79a), CD79b molecule (CD79b), carcinoembryonic antigen related cell adhesion molecule 3 (CD66d), CD27 molecule (CD27), CD28 molecule (CD28), TNF receptor superfamily member 9 (4-1BB), TNF receptor superfamily member 4 (OX40), TNF receptor superfamily member 8 (CD30), CD40 molecule (CD40), programmed cell death 1 (PD-1), inducible T cell costimulatory (ICOS), lymphocyte function-associated antigen-1 (LFA-1), CD2 molecule (CD2), CD7 molecule (CD7), TNF superfamily member 14 (LIGHT), killer cell lectin like receptor C2 (NKG2C) and CD276 molecule (B7-H3) c-stimulatory domains, or functional variants thereof. In some embodiments, the intracellular domains of CARs of the instant disclosure comprise at least one co-stimulatory domain. In some embodiments, the co-stimulatory domain is isolated or derived from CD28.

In some embodiments, the intracellular domains of CARs of the instant disclosure comprise at least one co-stimulatory domain. In some embodiments, the co-stimulatory domain is isolated or derived from CD28. In some embodiments, the CD28 co-stimulatory domain comprises an amino acid sequence having at least 80% identity, at least 90% identity, at least 95% identity, at least 99% identity or is identical to a sequence of: RSKRSRLLHSDYMNMTPRRPGPTRKHYQPYAPPRDFAAYRS (SEQ ID NO: 41). In some embodiments, the CD28 co-stimulatory domain comprises or consists essentially of SEQ ID NO: 41. In some embodiments, the CD28 co-stimulatory domain is encoded by a nucleotide sequence having at least 80% identity, at least 90% identity, at least 95% identity, at least 99% identity or is identical to a sequence of: AGGAGCAAGCGGAGCAGACTGCTGCACAGCGACTACATGAACATGACCCCCCG GAGGCCTGGCCCCACCCGGAAGCACTACCAGCCCTACGCCCCTCCCAGGGATTT CGCCGCCTACCGGAGC (SEQ ID NO: 42). In some embodiments, the CD28 co-stimulatory domain is encoded by SEQ ID NO: 42.

In some embodiments, the co-stimulatory domain is isolated or derived from 4-1BB. In some embodiments, the 4-1BB co-stimulatory domain comprises an amino acid sequence having at least 80% identity, at least 90% identity, at least 95% identity, at least 99% identity or is identical to a sequence of KRGRKKLLYIFKQPFMRPVQTTQEEDGCSCRFPEEEEGGCEL (SEQ ID NO: 43). In some embodiments, the 4-1BB co-stimulatory domain comprises or consists essentially of KRGRKKLLYIFKQPFMRPVQTTQEEDGCSCRFPEEEEGGCEL (SEQ ID NO: 43). In some embodiments, the 4-1BB co-stimulatory domain is encoded by a nucleotide sequence having at least 80% identity, at least 90% identity, at least 95% identity, at least 99% identity or is identical to a sequence of

(SEQ ID NO: 44)
AAACGGGGCAGAAAGAAACTCCTGTATATATTCAAACAACCATTTATGA
GGCCAGTACAAACTACTCAAGAGGAAGATGGCTGTAGCTGCCGATTTCC
AGAAGAAGAAGAAGGAGGATGTGAACTG.

In some embodiments, the intracellular domain of the CAR comprises a CD28 co-stimulatory domain, a 4-1BB costimulatory domain, and a CD3ζ activation domain. In some embodiments, the intracellular domain of the CAR comprises a sequence of RSKRSRLLHSDYMNMTPRRPGPTRKHYQPYAPPRDFAAYRSKRGRKKLLYIFKQPF MRPVQTTQEEDGCSCRFPEEEEGGCELRVKFSRSADAPAYKQGQNQLYNELNLGRR EEYDVLDKRRGRDPEMGGKPRRKNPQEGLYNELQKDKMAEAYSEIGMKGERRRG KGHDGLYQGLSTATKDTYDALHMQALPPR (SEQ ID NO: 45), or a sequence having at least 80% identity, at least 90% identity, at least 95% identity, at least 99% identity thereto.

The cytoplasmic domains within the cytoplasmic signaling portion of the CARs of the instant disclosure may be linked to each other in a random or specified order. Optionally, a short oligo- or polypeptide linker, for example between 2 and 10 amino acids in length may form the linkage. A glycine-serine doublet provides an example of a suitable linker. An exemplary linker comprises a sequence of GGGGSGGGGSGGGGSGG (SEQ ID NO: 46).

The cytoplasmic domains within the cytoplasmic signaling portion of the CARs of the instant disclosure may be linked to each other in a random or specified order. Optionally, a short oligo- or polypeptide linker, for example between 2 and 10 amino acids in length may form the linkage. A glycine-serine doublet provides an example of a suitable linker.

The cytoplasmic domains within the cytoplasmic signaling portion of the CARs of the instant disclosure may be linked to each other in a random or specified order. Optionally, a short oligo- or polypeptide linker, for example between 2 and 10 amino acids in length may form the linkage. A glycine-serine doublet provides an example of a suitable linker.

### T Cell Receptors (TCRs)

The disclosure provides an activator receptor and immune cells comprising same. In some embodiments, the activator receptor is a T cell receptor (TCR).

As used herein, a “TCR”, sometimes also called a “TCR complex” or “TCR/CD3 complex” refers to a protein complex comprising a TCR alpha chain, a TCR beta chain, and one or more of the invariant CD3 chains (zeta, gamma, delta and epsilon), sometimes referred to as subunits. The TCR alpha and beta chains can be disulfide-linked to function as a heterodimer to bind to peptide-MHC complexes. Once the TCR alpha/beta heterodimer engages peptide-MHC, conformational changes in the TCR complex in the associated invariant CD3 subunits are induced, which leads to their phosphorylation and association with downstream proteins, thereby transducing a primary stimulatory signal. In an exemplary TCR complex, the TCR alpha and TCR beta polypeptides form a heterodimer, CD3 epsilon and CD3 delta form a heterodimer, CD3 epsilon and CD3 gamma for a heterodimer, and two CD3 zeta form a homodimer.

Any suitable ligand binding domain may be fused to an extracellular domain, hinge domain or transmembrane of the TCRs described herein. For example, the ligand binding domain can be an antigen binding domain of an antibody or TCR, or comprise an antibody fragment, a Vβ only domain, a linear antibody, a single-chain variable fragment (scFv), or a single domain antibody (sdAb).

In some embodiments, the ligand binding domain is fused to one or more extracellular domains or transmembrane domains of one or more TCR subunits. The TCR subunit can be TCR alpha, TCR beta, CD3 delta, CD3 epsilon, CD3 gamma or CD3 zeta. For example, the ligand binding domain can be fused to TCR alpha, or TCR beta, or portions of the ligand binding can be fused to two subunits, for example portions of the ligand binding domain can be fused to both TCR alpha and TCR beta.

TCR subunits include TCR alpha, TCR beta, CD3 zeta, CD3 delta, CD3 gamma and CD3 epsilon. Any one or more of TCR alpha, TCR beta chain, CD3 gamma, CD3 delta, CD3 epsilon, or CD3 zeta, or fragments or derivative thereof, can be fused to one or more domains capable of providing a stimulatory signal of the disclosure, thereby enhancing TCR function and activity.

TCR transmembrane domains isolated or derived from any source are envisaged as within the scope of the disclosure. The transmembrane domain may be derived either from a natural or from a recombinant source. Where the source is natural, the domain may be derived from any membrane-bound or transmembrane protein.

In some embodiments, the transmembrane domain is capable of signaling to the intracellular domain(s) whenever the TCR complex has bound to a target. A transmembrane domain of particular use in this disclosure may include at least the transmembrane region(s) of e.g., the alpha, beta or zeta chain of the TCR, CD3 delta, CD3 epsilon or CD3 gamma, CD28, CD3 epsilon, CD45, CD4, CD5, CD8, CD9, CD16, CD22, CD33, CD37, CD64, CD80, CD86, CD134, CD137, CD154.

In some embodiments, the transmembrane domain can be attached to the extracellular region of a polypeptide of the TCR, e.g., the antigen binding domain of the TCR alpha or beta chain, via a hinge, e.g., a hinge from a human protein. For example, the hinge can be a human immunoglobulin (Ig) hinge, e.g., an IgG4 hinge, or a CD8a hinge. In some embodiments, the hinge is isolated or derived from CD8a or CD28.

In some embodiments, the extracellular ligand binding domain is attached to one or more transmembrane domains of the TCR. In some embodiments, the transmembrane domain comprises a TCR alpha transmembrane domain, a TCR beta transmembrane domain, or both. In some embodiments, the transmembrane comprises a CD3 zeta transmembrane domain.

A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 or up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 or up to 15 amino acids of the intracellular region).

In some embodiments, the transmembrane domain can be selected or modified by amino acid substitution to avoid binding of such domains to the transmembrane domains of the same or different surface membrane proteins, e.g., to minimize interactions with other members of the receptor complex.

When present, the transmembrane domain may be a natural TCR transmembrane domain, a natural transmembrane domain from a heterologous membrane protein, or an artificial transmembrane domain. The transmembrane domain may be a membrane anchor domain. Without limitation, a natural or artificial transmembrane domain may comprise a hydrophobic a-helix of about 20 amino acids, often with positive charges flanking the transmembrane segment. The transmembrane domain may have one transmembrane segment or more than one transmembrane segment. Prediction of transmembrane domains/segments may be made using publicly available prediction tools (e.g., TMI-MM, Krogh et al. Journal of Molecular Biology 2001; 305(3):567-580; or TMpred, Hofmann & Stoffel Biol. Chem. Hoppe-Seyler 1993; 347: 166). Non-limiting examples of membrane anchor systems include platelet derived growth factor receptor (PDGFR) transmembrane domain, glycosylphosphatidylinositol (GPI) anchor (added post-translationally to a signal sequence) and the like.

In some embodiments, the transmembrane domain comprises a TCR alpha transmembrane domain. In some embodiments, the TCR alpha transmembrane domain comprises an amino acid sequence having at least 85% identity, at least 90% identity, at least 95% identity, at least 96% identity, at least 97% identity, at least 98% identity, at least 99% identity or is identical to a sequence of: VIGFRILLLKVAGFNLLMTLRLW (SEQ ID NO: 126). In some embodiments, the TCR alpha transmembrane domain comprises, or consists essentially of, SEQ ID NO: 127. In some embodiments, the TCR alpha transmembrane domain is encoded by a sequence of:

(SEQ ID NO: 127)
GTGATTGGGTTCCGAATCCTCCTCCTGAAAGTGGCCGGGTTTAATCTGC
TCATGACGCTGCGGCTGTGG.

In some embodiments, the transmembrane domain comprises a TCR beta transmembrane domain. In some embodiments, the TCR beta transmembrane domain comprises an amino acid sequence having at least 85% identity, at least 90% identity, at least 95% identity, at least 96% identity, at least 97% identity, at least 98% identity, at least 99% identity or is identical to a sequence of TILYEILLGKATLYAVLVSALVL (SEQ ID NO: 128). In some embodiments, the TCR beta transmembrane domain comprises, or consists essentially of, SEQ ID NO: 128. In some embodiments, the TCR beta transmembrane domain is encoded by a sequence of

(SEQ ID NO: 129)
ACCATCCTCTATGAGATCTTGCTAGGGAAGGCCACCTTGTATGCCGTGC
TGGTCAGTGCCCTCGTGCTG.

TCRs of the disclosure can comprise one or more intracellular domains. Exemplary TCRs comprising intracellular domains for use in the instant disclosure are described in PCT/US2020/045250 filed on Sep. 6, 2020, the contents of which are incorporated herein by reference. In some embodiments, the intracellular domain comprises one or more domains capable of providing a stimulatory signal to a transmembrane domain. In some embodiments, the intracellular domain comprises a first intracellular domain capable of providing a stimulatory signal and a second intracellular domain capable of providing a stimulatory signal. In other embodiments, the intracellular domain comprises a first, second and third intracellular domain capable of providing a stimulatory signal. The intracellular domains capable of providing a stimulatory signal are selected from the group consisting of a CD28 molecule (CD28) domain, a LCK proto-oncogene, Src family tyrosine kinase (Lck) domain, a TNF receptor superfamily member 9 (4-1BB) domain, a TNF receptor superfamily member 18 (GITR) domain, a CD4 molecule (CD4) domain, a CD8a molecule (CD8a) domain, a FYN proto-oncogene, Src family tyrosine kinase (Fyn) domain, a zeta chain of T cell receptor associated protein kinase 70 (ZAP70) domain, a linker for activation of T cells (LAT) domain, lymphocyte cytosolic protein 2 (SLP76) domain, (TCR) alpha, TCR beta, CD3 delta, CD3 gamma and CD3 epsilon intracellular domains.

In some embodiments, an intracellular domain comprises at least one intracellular signaling domain. An intracellular signaling domain generates a signal that promotes a function a cell, for example an immune effector function of a TCR containing cell, e.g., a TCR-expressing T-cell. In some embodiments, the intracellular domain of the activator receptor of the disclosure includes at least one intracellular signaling domain. For example, the intracellular domains of CD3 gamma, delta or epsilon comprise signaling domains.

In some embodiments, the extracellular domain, transmembrane domain and intracellular domain are isolated or derived from the same protein, for example T-cell receptor (TCR) alpha, TCR beta, CD3 delta, CD3 gamma, CD3 epsilon or CD3 zeta.

Examples of intracellular domains for use in activator receptors of the disclosure include the cytoplasmic sequences of the TCR alpha, TCR beta, CD3 zeta, and 4-1BB, and the intracellular signaling co-receptors that act in concert to initiate signal transduction following antigen receptor engagement, as well as any derivative or variant of these sequences and any recombinant sequence that has the same functional capability.

In some embodiments, the intracellular signaling domain comprises a primary intracellular signaling domain. Exemplary primary intracellular signaling domains include those derived from the proteins responsible for primary stimulation, or antigen dependent stimulation.

In some embodiments, the intracellular domain comprises a CD3 delta intracellular domain, a CD3 epsilon intracellular domain, a CD3 gamma intracellular domain, a CD3 zeta intracellular domain, a TCR alpha intracellular domain or a TCR beta intracellular domain.

In some embodiments, the intracellular domain comprises a TCR alpha intracellular domain. In some embodiments, a TCR alpha intracellular domain comprises Ser-Ser. In some embodiments, a TCR alpha intracellular domain is encoded by a sequence of TCCAGC.

In some embodiments, the intracellular domain comprises a TCR beta intracellular domain. In some embodiments, the TCR beta intracellular domain comprises an amino acid sequence having at least 80% identity, at least 90% identity, or is identical to a sequence of: MAMVKRKDSR (SEQ ID NO: 130). In some embodiments, the TCR beta intracellular domain comprises, or consists essentially of SEQ ID NO: 130. In some embodiments, the TCR beta intracellular domain is encoded by a sequence of:

(SEQ ID NO: 131)
ATGGCCATGGTCAAGAGAAAGGATTCCAGA.

In some embodiments, the intracellular signaling domain comprises at least one stimulatory intracellular domain. In some embodiments, the intracellular signaling domain comprises a primary intracellular signaling domain, such as a CD3 delta, CD3 gamma and CD3 epsilon intracellular domain, and one additional stimulatory intracellular domain, for example a co-stimulatory domain. In some embodiments, the intracellular signaling domain comprises a primary intracellular signaling domain, such as a CD3 delta, CD3 gamma and CD3 epsilon intracellular domain, and two additional stimulatory intracellular domains.

Exemplary co-stimulatory intracellular signaling domains include those derived from proteins responsible for co-stimulatory signals, or antigen independent stimulation. Co-stimulatory molecules include, but are not limited to an MHC class I molecule, BTLA, a Toll ligand receptor, as well as DAP10, DAP12, CD30, LIGHT, OX40, CD2, CD27, CDS, ICAM-1, LFA-1 (CD11a/CD18) 4-1BB (CD137, TNF receptor superfamily member 9), and CD28 molecule (CD28). A co-stimulatory protein can be represented in the following protein families: TNF receptor proteins, Immunoglobulin-like proteins, cytokine receptors, integrins, signaling lymphocytic activation molecules (SLAM proteins), and activating NK cell receptors. Examples of such molecules include CD27, CD28, 4-1BB (CD137), OX40, GITR, CD30, CD40, ICOS, BAFFR, HVEM, lymphocyte function-associated antigen-1 (LFA-1), CD2, CD7, LIGHT, NKG2C, SLAMF7, NKp80, CD160, B7-H3, a ligand that specifically binds with CD83, CD4, and the like. The co-stimulatory domain can comprise the entire intracellular portion, or the entire native intracellular signaling domain, of the molecule from which it is derived, or a functional variant thereof.

In some embodiments, the stimulatory domain comprises a co-stimulatory domain. In some embodiments, the co-stimulatory domain comprises a CD28 or 4-1BB co-stimulatory domain. CD28 and 4-1BB are well characterized co-stimulatory molecules required for full T cell activation and known to enhance T cell effector function. For example, CD28 and 4-1BB have been utilized in chimeric antigen receptors (CARs) to boost cytokine release, cytolytic function, and persistence over the first-generation CAR containing only the CD3 zeta signaling domain. Likewise, inclusion of co-stimulatory domains, for example CD28 and 4-1BB domains, in TCRs can increase T cell effector function and specifically allow co-stimulation in the absence of co-stimulatory ligand, which is typically down-regulated on the surface of tumor cells. In some embodiments, the stimulatory domain comprises a CD28 intracellular domain or a 4-1BB intracellular domain.

### Inhibitory Receptors

The disclosure provides a second antigen, comprising an epitope of a specific height, and an inhibitory receptor with a defined receptor height that specifically binds the epitope of the second antigen. The second antigen can be lost in the cancer cell through any mechanism, such as, without limitation, epigenetic changes that effect antigen expression, mutations to the gene encoding the antigen, disruption of cellular signaling that regulates expression of the antigen, chromosome loss, partial or complete deletion of the genomic locus, gene silencing through modification of nucleic acids or heterochromatin, or loss of expression through other mechanisms. In variations of the compositions and methods disclosed herein, the cells or subject treated may exhibit a loss of expression of the antigen because of non-genetic changes. Accordingly, the disclosure provides compositions and methods for killing cells and/or treating subject lacking expression of the second antigen from any cause.

The disclosure provides an inhibitory receptor, that binds specifically to a non-target antigen selected from intercellular adhesion molecule 1 (ICAM1), MSLN, catechol-O-methyltransferase (COMT), C—X—C motif chemokine ligand 16 (CXCL16), leucine rich repeat neuronal 4 (LRRN4) and uroplakin 3B (UPK3B), or an antigen peptide thereof in a complex with a major histocompatibility complex class I (MHC-I), wherein the non-target antigen may comprise a nonsynonymous, extracellular-domain polymorphism (e.g., in an extracellular domain of ICAM1, COMT, CXCL16), and immune cells comprising same. In some embodiments, the second receptor is an inhibitory chimeric antigen receptor. Alternatively, the second antigen may comprise a protein whose expression is lost in tumors but present in key MSLN-expression normal tissues (e.g., LRRN4, UPK3B).

Exemplary inhibitory receptors are described in PCT/US2020/045228 filed on Sep. 6, 2020, PCT/US2020/064607, filed on Dec. 11, 2020, PCT/US2021/029907, filed on Apr. 29, 2021, and PCT/US2020/059856 filed on Nov. 10, 2020, the contents of each of which are incorporated herein by reference.

In some embodiments, the inhibitory receptor is humanized.

The disclosure provides an inhibitory receptor that can discriminate between single amino-acid variant alleles of a non-target antigen. This ability to discriminate between allelic variants of a non-target antigen allows the second receptor to inhibit activation of immune cells comprising the second receptor in the presence of non-target cells that express that the allele recognized by the ligand binding domain. However, activation of immune cells is not inhibited in the presence of target cells that have lost the allele, for example cancer cells that have lost one allele of a gene through loss of heterozygosity.

The disclosure provides an inhibitory receptor that can discriminate between different levels of expression of a second antigen. This allows the inhibitory receptor to inhibit activation of immune cells comprising the inhibitory receptor in the presence of non-target cells that express the second antigen, but to allow activation of immune cells in the presence of cancer cells that express low levels, or have no expression, of the second antigen.

### Second Antigens

In some embodiments, the second antigen is not expressed by the target cells and is expressed by non-target cells. In some embodiments, the second antigen is expressed by healthy cells, i.e., cells that are not cancer cells. In some embodiments, the target cells are a plurality of cancer cells that have lost expression of the second antigen through loss of heterozygosity (LOH). In some embodiments, the non-target cells are a plurality of healthy cells (i.e., non-cancer cells), that express both the first and the second antigen.

Any cell surface molecule expressed by the non-target cells that is not expressed by target cells may be a suitable second antigen for the inhibitory receptor. For example, a cell adhesion molecule, a cell-cell signaling molecule, an extracellular domain, a molecule involved in chemotaxis, a glycoprotein, a G protein-coupled receptor, a transmembrane, a receptor for a neurotransmitter or a voltage gated ion channel can be used as a non-target antigen.

In some embodiments, the second antigen is EGFR, HER2, or a blood cancer antigen.

In some embodiments, the second antigen is a peptide antigen of a cancer cell-specific antigen in a complex with a major histocompatibility complex class I (MHC-I).

In some embodiments, the second antigen is selected from MSLN, LRRN4, and HLA-A2. Exemplary second antigens for use in the methods and compositions described herein are described in PCT/US2021/060607, PCT/US2023/011698, US 20230029341, US 20220370497, US 20220273721, and US 20220054551, are incorporated herein by reference in their entirety.

In some embodiments, the second antigen is lost in the cancer cells due to loss of heterozygosity. Exemplary non-target antigens lost in cancer cells due to loss of heterozygosity include ICAM1, COMT and CXCL16. In some embodiments, the second antigen is selected from the group consisting of a polymorphic variant of ICAM1, COMT and CXCL16. In some embodiments, the second antigen is an antigen peptide comprising a polymorphic residue of ICAM1, COMT or CXCL16 in a complex with a major histocompatibility complex class I (MHC-I).

In some embodiments, the non-target antigen comprises ICAM1 or an antigen peptide thereof in a complex with MHC-I. Human ICAM1 is frequently lost through LOH in cancer cells.

A wild type Human ICAM1 is described in NCBI record number NP_000192.2 the contents of which are incorporated by reference herein in their entirety. In some embodiments, ICAM1 comprises an amino acid sequence of:

| (SEQ ID NO: 47) | |
|---|---|
| 1 | MAPSSPRPAL PALLVLLGAL FPGPGNAQTS VSPSKVILPR GGSVLVTCST SCDQPKLLGI |
| 61 | ETPLPKKELL LPGNNRKVYE LSNVQEDSQP MCYSNCPDGQ STAKTFLTVY WTPERVELAP |
| 121 | LPSWQPVGKN LTLRCQVEGG APRANLTVVL LRGEKELKRE PAVGEPAEVT TTVLVRRDHH |
| 181 | GANFSCRTEL DLRPQGLELF ENTSAPYQLQ TFVLPATPPQ LVSPRVLEVD TQGTVVCSLD |
| 241 | GLFPVSEAQV HLALGDQRLN PTVTYGNDSF SAKASVSVTA EDEGTQRLTC AVILGNQSQE |
| 301 | TLQTVTIYSF PAPNVILTKP EVSEGTEVTV KCEAHPRAKV TLNGVPAQPL GPRAQLLLKA |
| 361 | TPEDNGRSFS CSATLEVAGQ LIHKNQTREL RVLYGPRLDE RDCPGNWTWP ENSQQTPMCQ |
| 421 | AWGNPLPELK CLKDGTFPLP IGESVTVTRD LEGTYLCRAR STQGEVTRKV TVNVLSPRYE |
| 481 | IVIITVVAAA VIMGTAGLST YLYNRQRKIK KYRLQQAQKG TPMKPNTQAT PP. |

In some embodiments, ICAM1 comprises a sequence that shares at least 80%, at least 85%, at least 90%, at least 95%, at least 96%, at least 97%, at least 98%, or at least 99% identity to SEQ ID NO: 47. Polymorphic residues of ICAM1 are marked as bold and underlined in SEQ ID NO: 47. For example, rs5498 is a polymorphism at position 469 of SEQ ID NO: 47, which can be a K or an E.

In some embodiments, the second antigen comprises a polymorphism of ICAM1. For example, the second antigen comprises a peptide derived from ICAM1 comprising a polymorphic residue of ICAM1. Polymorphic residues of ICAM1 include amino acid residue 469 of SEQ ID NO: 47. In some embodiments, the second antigen comprises a peptide of ICAM1 comprising amino acid 469 of SEQ ID NO: 47. In some embodiments, the second antigen comprises a K at position 469 of SEQ ID NO: 47. In some embodiments, the second antigen comprises an E at position 469 of SEQ ID NO: 47.

In some embodiments, the second antigen comprises an ICAM1 polymorphism with an K at position 469 of SEQ ID NO: 47, and the second receptor comprises a ligand binding domain with a higher affinity for an ICAM1 ligand with an K at position 469 of SEQ ID NO: 47 than for an ICAM1 ligand with an E at position 469 of SEQ ID NO: 47. In some embodiments, the second antigen comprises an ICAM1 polymorphism with an E at position 469 of SEQ ID NO: 47, and the second receptor comprises a ligand binding domain with a higher affinity for an ICAM1 ligand with an E at position 469 of SEQ ID NO: 47 than for an ICAM1 ligand with a K at position 469 of SEQ ID NO: 47.

In some embodiments, the second antigen comprises COMT or an antigen peptide thereof in a complex with MHC-I. Human COMT is frequently lost through LOH in cancer cells.

A wild type Human COMT is described in NCBI record number NP_000192.2, the contents of which are incorporated by reference herein in their entirety. In some embodiments, COMT comprises an amino acid sequence of:

| (SEQ ID NO: 48) | |
|---|---|
| 1 | MPEAPPLLLA AVLLGLVLLV VLLLLLRHWG WGLCLIGWNE FILQPIHNLL MGDTKEQRIL |
| 61 | NHVLQHAEPG NAQSVLEAID TYCEQKEWAM NVGDKKGKIV DAVIQEHQPS VLLELGAYCG |
| 121 | YSAVRMARLL SPGARLITIE INPDCAAITQ RMVDFAGVKD KVTLVVGASQ DIIPQLKKKY |
| 181 | DVDTLDMVFL DHWKDRYLPD TLLLEECGLL RKGTVLLADN VICPGAPDFL AHVRGSSCFE |
| 241 | CTHYQSFLEY REVVDGLEKA IYKGPGSEAG P. |

In some embodiments, COMT comprises a sequence that shares at least 80%, at least 85%, at least 90%, at least 95%, at least 96%, at least 97%, at least 98%, or at least 99% identity to SEQ ID NO: 48. Polymorphic residues of COMT are marked as bold and underlined in SEQ ID NO: 48. For example, V158M is a polymorphism at position 158 of SEQ ID NO: 48, which can be a V or an M.

In some embodiments, the second antigen comprises a polymorphism of COMT. For example, the second antigen comprises a peptide derived from COMT comprising a polymorphic residue of COMT. Polymorphic residues of COMT 1 include amino acid residue 158 of SEQ ID NO: 48. In some embodiments, the second antigen comprises a peptide of COMT comprising amino acid 158 of SEQ ID NO: 48. In some embodiments, the second antigen comprises a V at position 158 of SEQ ID NO: 48. In some embodiments, the second antigen comprises an M at position 158 of SEQ ID NO: 48.

In some embodiments, the second antigen comprises a COMT polymorphism with a V at position 158 of SEQ ID NO: 48, and the second receptor comprises a ligand binding domain with a higher affinity for a COMT ligand with an V at position 158 of SEQ ID NO:48 than for a COMT ligand with an M at position 158 of SEQ ID NO: 48. In some embodiments, the second antigen comprises a COMT polymorphism with a M at position 158 of SEQ ID NO: 48, and the second receptor comprises a ligand binding domain with a higher affinity for a COMT ligand with an M at position 158 of SEQ ID NO: 48 than for a COMT ligand with a V at position 158 of SEQ ID NO: 48.

In some embodiments, the second antigen comprises C—X—C motif chemokine ligand 16 (CXCL16) or an antigen peptide thereof in a complex with MHC-I. Human CXCL16 precursor is described in NCBI record number NP_001094282.1, the contents of which are incorporated by reference herein in their entirety. In some embodiments, CXCL16 comprises an amino acid sequence of:

| (SEQ ID NO: 49) | |
|---|---|
| 1 | MSGSQSEVAP SPQSPRSPEM GRDLRPGSRV LLLLLLLLLV YLTQPGNGNE GSVTGSCYCG |
| 61 | KRISSDSPPS VQFMNRLRKH LRAYHRCLYY TRFQLLSWSV CGGNKDPWVQ ELMSCLDLKE |
| 121 | CGHAYSGIVA HQKHLLPTSP PISQASEGAS SDIHTPAQML LSTLQSTQRP TLPVGSLSSD |
| 181 | KELTRPNETT IHTAGHSLAA GPEAGENQKQ PEKNAGPTAR TSATVPVLCL LAIIFILTAA |
| 241 | LSYVLCKRRR GQSPQSSPDL PVHYIPVAPD SNT. |

In some embodiments, the second antigen comprises a polymorphism of CXCL16. For example, the second antigen comprises a peptide derived from CXCL16 comprising a polymorphic residue of CXCL16. Polymorphic residues of CXCL16 include positions 142 and 200 of SEQ ID NO: XX. In some embodiments, the second antigen comprises a peptide of CXCL16 comprising amino acid 142 or 200 of SEQ ID NO: 49 In some embodiments, the second antigen comprises a peptide of CXCL16 comprising an A at amino acid 200 of SEQ ID NO: 49. In some embodiments, the second antigen comprises a peptide of CXCL16 comprising a V at amino acid 200 of SEQ ID NO: 49. In some embodiments, the second antigen comprises a peptide of CXCL16 comprising an I at amino acid 142 of SEQ ID NO: 49. In some embodiments, the second antigen comprises a peptide of CXCL16 comprising a T at amino acid 142 of SEQ ID NO: 49.

In some embodiments, the second antigen comprises a polymorphism of CXCL16. In some embodiments, the second antigen comprises a peptide of CXCL16 comprising an A at amino acid 200 of SEQ ID NO: 49, and the second receptor comprises a ligand binding domain with a higher affinity for a CXCL16 ligand with an A at position 200 of SEQ ID NO: 49 than for a CXCL16 ligand with a V at position 200 of SEQ ID NO: 49. In some embodiments, the second antigen comprises a peptide of CXCL16 comprising a V at amino acid 200 of SEQ ID NO: 49, and the second receptor comprises a ligand binding domain with a higher affinity for a CXCL16 ligand with a V at position 200 of SEQ ID NO: 49 than for a CXCL16 ligand with an A at position 200 of SEQ ID NO: 49. In some embodiments, the second antigen comprises a peptide of CXCL16 comprising an I at amino acid 142 of SEQ ID NO: 49, and the second receptor comprises a ligand binding domain with a higher affinity for a CXCL16 ligand with an I at position 142 of SEQ ID NO: 49 than for a CXCL16 ligand with a T at position 142 of SEQ ID NO: 49. In some embodiments, the second antigen comprises a peptide of CXCL16 comprising a T at amino acid 142 of SEQ ID NO: 49, and the second receptor comprises a ligand binding domain with a higher affinity for a CXCL16 ligand with a T at position 142 of SEQ ID NO: 49 than for a CXCL16 ligand with an I at position 142 of SEQ ID NO: 49.

In some embodiments, the second antigen comprises HLA-A*01, HLA-A*02, HLA-A*03, HLA-A*11, HLA-B*07 or HLA-C*07.

In some embodiments, the ligand binding domain of the inhibitory receptor comprises an scFv.

Exemplary scFv are described in PCT/US2021/060607 and PCT/US2023/011698, which are incorporated herein by reference in their entirety. Exemplary scFv sequences also include those of Table 2 above.

In some embodiments, the inhibitory receptor comprises an scFv having at least 80% identity, at least 85% identity, at least 90% identity, at least 95% identity, at least 97% identity or at least 99% identity to a sequence of SEQ ID NOS: 28, 29, and 31.

In some embodiments, the inhibitory receptor comprises a sequence having at least 80% identity, at least 85% identity, at least 90% identity, at least 95% identity, at least 97% identity or at least 99% identity to a sequence of SEQ ID NOS: 50-72 as shown in Table 4 below.

TABLE 4

| Receptor | Protein Sequence |
|---|---|
| aLRRN4 | QVQLKESGPGLVAPSQSLSITCTVSGFSLTNYAVHWVRQSPGKGLEW |
| (1J21) | LGAIWSDGSTDYNAAFISRLSISKDNSKSQVFFKMNSLQADDTAIYYC |
| with LIR | ARKDYSNYVFDYWGQGTTLTVSSGGGGSGGGGSGGGGSGGEIVLT |
| hinge | QSPAITAASLGQKVTISCSASSSVSYMHWYQQKSGTSPKPWIYDISKL |
| | ASGVPARFSGSGSGTSYSLTISSMEAEDAAIYYCQQWSYPLITFGAGT |
| | KLELKYGSQSSKPYLLTHPSDPLELVVSGPSGGPSSPTTGPTSTSGPED |
| | QPLTPTGSDPQSGLGRHLGVVIGILVAVILLLLLLLLLFLILRHRRQGK |
| | HWTSTQRKADFQHPAGAVGPEPTDRGLQWRSSPAADAQEENLYAA |
| | VKHTQPEDGVEMDTRSPHDEDPQAVTYAEVKHSRPRREMASPPSPL |
| | SGEFLDTKDRQAEEDRQMDTEAAASEAPQDVTYAQLHSLTLRREAT |
| | EPPPSQEGPSPAVPSIYATLAIH (SEQ ID NO: 50) |
| aLRRN4 | QVQLKESGPGLVAPSQSLSITCTVSGFSLTNYAVHWVRQSPGKGLEW |
| (1J21) | LGAIWSDGSTDYNAAFISRLSISKDNSKSQVFFKMNSLQADDTAIYYC |
| with EGF1 | ARKDYSNYVFDYWGQGTTLTVSSGGGGSGGGGSGGGGSGGEIVLT |
| | QSPAITAASLGQKVTISCSASSSVSYMHWYQQKSGTSPKPWIYDISKL |
| | ASGVPARFSGSGSGTSYSLTISSMEAEDAAIYYCQQWSYPLITFGAGT |
| | KLELKGGGGLTCVGHCSNGGSCTMNSKMMPECQCPPHMTGPRCEV |
| | VIGILVAVILLLLLLLLLFLILRHRRQGKHWTSTQRKADFQHPAGAVG |
| | PEPTDRGLQWRSSPAADAQEENLYAAVKHTQPEDGVEMDTRSPHDE |
| | DPQAVTYAEVKHSRPRREMASPPSPLSGEFLDTKDRQAEEDRQMDT |
| | EAAASEAPQDVTYAQLHSLTLRREATEPPPSQEGPSPAVPSIYATLAI |
| | H (SEQ ID NO: 51) |
| aLRRN4 | QVQLKESGPGLVAPSQSLSITCTVSGFSLTNYAVHWVRQSPGKGLEW |
| (1J21) | LGAIWSDGSTDYNAAFISRLSISKDNSKSQVFFKMNSLQADDTAIYYC |
| with EGF3 | ARKDYSNYVFDYWGQGTTLTVSSGGGGSGGGGSGGGGSGGEIVLT |
| | QSPAITAASLGQKVTISCSASSSVSYMHWYQQKSGTSPKPWIYDISKL |
| | ASGVPARFSGSGSGTSYSLTISSMEAEDAAIYYCQQWSYPLITFGAGT |
| | KLELKGGGGQYRQCSGYCENFGTCQMAADGSRQCRCTAYFEGSRC |
| | EVNKCSRCLEGACVVNKQSGDVTCNCTDGRVAPSCLTCVGHCSNG |
| | GSCTMNSKMMPECQCPPHMTGPRCEVVIGILVAVILLLLLLLLLFLIL |
| | RHRRQGKHWTSTQRKADFQHPAGAVGPEPTDRGLQWRSSPAADAQ |
| | EENLYAAVKHTQPEDGVEMDTRSPHDEDPQAVTYAEVKHSRPRRE |
| | MASPPSPLSGEFLDTKDRQAEEDRQMDTEAAASEAPQDVTYAQLHS |
| | LTLRREATEPPPSQEGPSPAVPSIYATLAIH (SEQ ID NO: 52) |
| aLRRN4 | QVQLKESGPGLVAPSQSLSITCTVSGFSLTNYAVHWVRQSPGKGLEW |
| (1J21) | LGAIWSDGSTDYNAAFISRLSISKDNSKSQVFFKMNSLQADDTAIYYC |
| with EGF5 | ARKDYSNYVFDYWGQGTTLTVSSGGGGSGGGGSGGGGSGGEIVLT |
| hinge | QSPAITAASLGQKVTISCSASSSVSYMHWYQQKSGTSPKPWIYDISKL |
| | ASGVPARFSGSGSGTSYSLTISSMEAEDAAIYYCQQWSYPLITFGAGT |
| | KLELKGGGGELDQCWEHCRNGGTCAASPSGMPTCRCPTGFTGPKCT |
| | QQVCAGYCANNSTCTVNQGNQPQCRCLPGFLGDRCQYRQCSGYCE |
| | NFGTCQMAADGSRQCRCTAYFEGSRCEVNKCSRCLEGACVVNKQS |
| | GDVTCNCTDGRVAPSCLTCVGHCSNGGSCTMNSKMMPECQCPPHM |
| | TGPRCEVVIGILVAVILLLLLLLLLFLILRHRRQGKHWTSTQRKADFQ |
| | HPAGAVGPEPTDRGLQWRSSPAADAQEENLYAAVKHTQPEDGVEM |
| | DTRSPHDEDPQAVTYAEVKHSRPRREMASPPSPLSGEFLDTKDRQAE |
| | EDRQMDTEAAASEAPQDVTYAQLHSLTLRREATEPPPSQEGPSPAVP |
| | SIYATLAIH (SEQ ID NO: 53) |
| aLRRN4 | QVQLKESGPGLVAPSQSLSITCTVSGFSLTNYAVHWVRQSPGKGLEW |
| (1J21) | LGAIWSDGSTDYNAAFISRLSISKDNSKSQVFFKMNSLQADDTAIYYC |
| with EGF7 | ARKDYSNYVFDYWGQGTTLTVSSGGGGSGGGGSGGGGSGGEIVLT |
| hinge | QSPAITAASLGQKVTISCSASSSVSYMHWYQQKSGTSPKPWIYDISKL |
| | ASGVPARFSGSGSGTSYSLTISSMEAEDAAIYYCQQWSYPLITFGAGT |
| | KLELKGGGGVTNPCDRKKCEWLCLLSPSGPVCTCPNGKRLDNGTCV |
| | PVPSPTPPPDAPRPGTCNLQCFNGGSCFLNARRQPKCRCQPRYTGDK |
| | CELDQCWEHCRNGGTCAASPSGMPTCRCPTGFTGPKCTQQVCAGYC |
| | ANNSTCTVNQGNQPQCRCLPGFLGDRCQYRQCSGYCENFGTCQMA |
| | ADGSRQCRCTAYFEGSRCEVNKCSRCLEGACVVNKQSGDVTCNCTD |
| | GRVAPSCLTCVGHCSNGGSCTMNSKMMPECQCPPHMTGPRCEVVIG |
| | ILVAVILLLLLLLLLFLILRHRRQGKHWTSTQRKADFQHPAGAVGPEP |
| | TDRGLQWRSSPAADAQEENLYAAVKHTQPEDGVEMDTRSPHDEDP |
| | QAVTYAEVKHSRPRREMASPPSPLSGEFLDTKDRQAEEDRQMDTEA |
| | AASEAPQDVTYAQLHSLTLRREATEPPPSQEGPSPAVPSIYATLAIH |
| | (SEQ ID NO: 54) |
| aLRRN4 | QVQLKESGPGLVAPSQSLSITCTVSGFSLTNYAVHWVRQSPGKGLEW |
| (1J21) | LGAIWSDGSTDYNAAFISRLSISKDNSKSQVFFKMNSLQADDTAIYYC |
| with G4Q3 | ARKDYSNYVFDYWGQGTTLTVSSGGGGSGGGGSGGGGSGGEIVLT |
| hinge | QSPAITAASLGQKVTISCSASSSVSYMHWYQQKSGTSPKPWIYDISKL |
| | ASGVPARFSGSGSGTSYSLTISSMEAEDAAIYYCQQWSYPLITFGAGT |
| | KLELKGGGGQGGGGQGGGGQVVIGILVAVILLLLLLLLLFLILRHRR |
| | QGKHWTSTQRKADFQHPAGAVGPEPTDRGLQWRSSPAADAQEENL |
| | YAAVKHTQPEDGVEMDTRSPHDEDPQAVTYAEVKHSRPRREMASPP |
| | SPLSGEFLDTKDRQAEEDRQMDTEAAASEAPQDVTYAQLHSLTLRR |
| | EATEPPPSQEGPSPAVPSIYATLAIH (SEQ ID NO: 55) |
| aLRRN4 | QVQLKESGPGLVAPSQSLSITCTVSGFSLTNYAVHWVRQSPGKGLEW |
| (1J21) | LGAIWSDGSTDYNAAFISRLSISKDNSKSQVFFKMNSLQADDTAIYYC |
| with G4Q6 | ARKDYSNYVFDYWGQGTTLTVSSGGGGSGGGGSGGGGSGGEIVLT |
| hinge | QSPAITAASLGQKVTISCSASSSVSYMHWYQQKSGTSPKPWIYDISKL |
| | ASGVPARFSGSGSGTSYSLTISSMEAEDAAIYYCQQWSYPLITFGAGT |
| | KLELKGGGGQGGGGQGGGGQGGGGQGGGGQGGGGQVVIGILVAVI |
| | LLLLLLLLLFLILRHRRQGKHWTSTQRKADFQHPAGAVGPEPTDRGL |
| | QWRSSPAADAQEENLYAAVKHTQPEDGVEMDTRSPHDEDPQAVTY |
| | AEVKHSRPRREMASPPSPLSGEFLDTKDRQAEEDRQMDTEAAASEAP |
| | QDVTYAQLHSLTLRREATEPPPSQEGPSPAVPSIYATLAIH (SEQ ID |
| | NO: 56) |
| aLRRN4 | QVQLKESGPGLVAPSQSLSITCTVSGFSLTNYAVHWVRQSPGKGLEW |
| (1J21) | LGAIWSDGSTDYNAAFISRLSISKDNSKSQVFFKMNSLQADDTAIYYC |
| with | ARKDYSNYVFDYWGQGTTLTVSSGGGGSGGGGSGGGGSGGEIVLT |
| G4Q10 | QSPAITAASLGQKVTISCSASSSVSYMHWYQQKSGTSPKPWIYDISKL |
| hinge | ASGVPARFSGSGSGTSYSLTISSMEAEDAAIYYCQQWSYPLITFGAGT |
| | KLELKGGGGQGGGGQGGGGQGGGGQGGGGQGGGGQGGGGQGGG |
| | GQGGGGQGGGGQVVIGILVAVILLLLLLLLLFLILRHRRQGKHWTST |
| | QRKADFQHPAGAVGPEPTDRGLQWRSSPAADAQEENLYAAVKHTQ |
| | PEDGVEMDTRSPHDEDPQAVTYAEVKHSRPRREMASPPSPLSGEFLD |
| | TKDRQAEEDRQMDTEAAASEAPQDVTYAQLHSLTLRREATEPPPSQ |
| | EGPSPAVPSIYATLAIH (SEQ ID NO: 57) |
| aLRRN4 | QVQLKESGPGLVAPSQSLSITCTVSGFSLTNYAVHWVRQSPGKGLEW |
| (1J21) | LGAIWSDGSTDYNAAFISRLSISKDNSKSQVFFKMNSLQADDTAIYYC |
| with | ARKDYSNYVFDYWGQGTTLTVSSGGGGSGGGGSGGGGSGGEIVLT |
| G4Q12 | QSPAITAASLGQKVTISCSASSSVSYMHWYQQKSGTSPKPWIYDISKL |
| hinge | ASGVPARFSGSGSGTSYSLTISSMEAEDAAIYYCQQWSYPLITFGAGT |
| | KLELKGGGGQGGGGQGGGGQGGGGQGGGGQGGGGQGGGGQGGG |
| | GQGGGGQGGGGQGGGGQGGGGQVVIGILVAVILLLLLLLLLFLILRH |
| | RRQGKHWTSTQRKADFQHPAGAVGPEPTDRGLQWRSSPAADAQEE |
| | NLYAAVKHTQPEDGVEMDTRSPHDEDPQAVTYAEVKHSRPRREMA |
| | SPPSPLSGEFLDTKDRQAEEDRQMDTEAAASEAPQDVTYAQLHSLTL |
| | RREATEPPPSQEGPSPAVPSIYATLAIH (SEQ ID NO: 58) |
| aLRRN4 | QVQLKESGPGLVAPSQSLSITCTVSGFSLTNYAVHWVRQSPGKGLEW |
| (1J21) | LGAIWSDGSTDYNAAFISRLSISKDNSKSQVFFKMNSLQADDTAIYYC |
| with TSP2 | ARKDYSNYVFDYWGQGTTLTVSSGGGGSGGGGSGGGGSGGEIVLT |
| hinge | QSPAITAASLGQKVTISCSASSSVSYMHWYQQKSGTSPKPWIYDISKL |
| | ASGVPARFSGSGSGTSYSLTISSMEAEDAAIYYCQQWSYPLITFGAGT |
| | KLELKGGGGSPRWSLWSTWAPCSVTCSEGSQLRYRRCVGWNGQCS |
| | GKVAPGTLEWQLQACEDQQCCPEMGGWSGWGPWEPCSVTCSKGT |
| | RTRRRACNHPAPKCGGHCPGQAQESEACDTQQVCPVVIGILVAVILL |
| | LLLLLLLFLILRHRRQGKHWTSTQRKADFQHPAGAVGPEPTDRGLQ |
| | WRSSPAADAQEENLYAAVKHTQPEDGVEMDTRSPHDEDPQAVTYA |
| | EVKHSRPRREMASPPSPLSGEFLDTKDRQAEEDRQMDTEAAASEAPQ |
| | DVTYAQLHSLTLRREATEPPPSQEGPSPAVPSIYATLAIH (SEQ ID NO: 59) |
| aLRRN4 | QVQLKESGPGLVAPSQSLSITCTVSGFSLTNYAVHWVRQSPGKGLEW |
| (1J21) | LGAIWSDGSTDYNAAFISRLSISKDNSKSQVFFKMNSLQADDTAIYYC |
| with TSP4 | ARKDYSNYVFDYWGQGTTLTVSSGGGGSGGGGSGGGGSGGEIVLT |
| hinge | QSPAITAASLGQKVTISCSASSSVSYMHWYQQKSGTSPKPWIYDISKL |
| | ASGVPARFSGSGSGTSYSLTISSMEAEDAAIYYCQQWSYPLITFGAGT |
| | KLELKGGGGSPRWSLWSTWAPCSVTCSEGSQLRYRRCVGWNGQCS |
| | GKVAPGTLEWQLQACEDQQCCPEMGGWSGWGPWEPCSVTCSKGT |
| | RTRRRACNHPAPKCGGHCPGQAQESEACDTQQVCPTHGAWATWGP |
| | WTPCSASCHGGPHEPKETRSRKCSAPEPSQKPPGKPCPGLAYEQRRC |
| | TGLPPCPVAGGWGPWGPVSPCPVTCGLGQTMEQRTCNHPVPQHGGP |
| | FCAGDATRTHICNTAVPCPVVIGILVAVILLLLLLLLLFLILRHRRQGK |
| | HWTSTQRKADFQHPAGAVGPEPTDRGLQWRSSPAADAQEENLYAA |
| | VKHTQPEDGVEMDTRSPHDEDPQAVTYAEVKHSRPRREMASPPSPL |
| | SGEFLDTKDRQAEEDRQMDTEAAASEAPQDVTYAQLHSLTLRREAT |
| | EPPPSQEGPSPAVPSIYATLAIH (SEQ ID NO: 60) |
| aLRRN4 | QVQLKESGPGLVAPSQSLSITCTVSGFSLTNYAVHWVRQSPGKGLEW |
| (1J21) | LGAIWSDGSTDYNAAFISRLSISKDNSKSQVFFKMNSLQADDTAIYYC |
| with TSP6 | ARKDYSNYVFDYWGQGTTLTVSSGGGGSGGGGSGGGGSGGEIVLT |
| hinge | QSPAITAASLGQKVTISCSASSSVSYMHWYQQKSGTSPKPWIYDISKL |
| | ASGVPARFSGSGSGTSYSLTISSMEAEDAAIYYCQQWSYPLITFGAGT |
| | KLELKGGGGSPRWSLWSTWAPCSVTCSEGSQLRYRRCVGWNGQCS |
| | GKVAPGTLEWQLQACEDQQCCPEMGGWSGWGPWEPCSVTCSKGT |
| | RTRRRACNHPAPKCGGHCPGQAQESEACDTQQVCPTHGAWATWGP |
| | WTPCSASCHGGPHEPKETRSRKCSAPEPSQKPPGKPCPGLAYEQRRC |
| | TGLPPCPVAGGWGPWGPVSPCPVTCGLGQTMEQRTCNHPVPQHGGP |
| | FCAGDATRTHICNTAVPCPVDGEWDSWGEWSPCIRRNMKSISCQEIP |
| | GQQSRGRTCRGRKFDGHRCAGQQQDIRHCYSIQHCPLKGSWSEWST |
| | WGLCMPPCGPNPTRARQRLCTPLLPKYPPTVSMVEGQGEKNVTFWG |
| | RPLPRCEELQGQKLVVEEKRPCLHVPACKVVIGILVAVILLLLLLLLLF |
| | LILRHRRQGKHWTSTQRKADFQHPAGAVGPEPTDRGLQWRSSPAAD |
| | AQEENLYAAVKHTQPEDGVEMDTRSPHDEDPQAVTYAEVKHSRPR |
| | REMASPPSPLSGEFLDTKDRQAEEDRQMDTEAAASEAPQDVTYAQL |
| | HSLTLRREATEPPPSQEGPSPAVPSIYATLAIH (SEQ ID NO: 61) |
| aLRRN4 | QVQLKESGPGLVAPSQSLSITCTVSGFSLTNYAVHWVRQSPGKGLEW |
| (1J21) | LGAIWSDGSTDYNAAFISRLSISKDNSKSQVFFKMNSLQADDTAIYYC |
| with Ig2 | ARKDYSNYVFDYWGQGTTLTVSSGGGGSGGGGSGGGGSGGEIVLT |
| hinge | QSPAITAASLGQKVTISCSASSSVSYMHWYQQKSGTSPKPWIYDISKL |
| | ASGVPARFSGSGSGTSYSLTISSMEAEDAAIYYCQQWSYPLITFGAGT |
| | KLELKGGGGPKPSISSNNSKPVEDKDAVAFTCEPEAQNTTYLWWVN |
| | GQSLPVSPRLQLSNGNRTLTLFNVTRNDARAYVCGIQNSVSANRSDP |
| | VTLDVLYGPDTPIISPPDSSYLSGANLNLSCHSASNPSPQYSWRINGIP |
| | QQHTQVLFIAKITPNNNGTYACFVSNLATGRNNSIVKSITVSVVIGILV |
| | AVILLLLLLLLLFLILRHRRQGKHWTSTQRKADFQHPAGAVGPEPTD |
| | RGLQWRSSPAADAQEENLYAAVKHTQPEDGVEMDTRSPHDEDPQA |
| | VTYAEVKHSRPRREMASPPSPLSGEFLDTKDRQAEEDRQMDTEAAA |
| | SEAPQDVTYAQLHSLTLRREATEPPPSQEGPSPAVPSIYATLAIH (SEQ |
| | ID NO: 62) |
| aLRRN4 | QVQLKESGPGLVAPSQSLSITCTVSGFSLTNYAVHWVRQSPGKGLEW |
| (1J21) | LGAIWSDGSTDYNAAFISRLSISKDNSKSQVFFKMNSLQADDTAIYYC |
| with Ig4 | ARKDYSNYVFDYWGQGTTLTVSSGGGGSGGGGSGGGGSGGEIVLT |
| hinge | QSPAITAASLGQKVTISCSASSSVSYMHWYQQKSGTSPKPWIYDISKL |
| | ASGVPARFSGSGSGTSYSLTISSMEAEDAAIYYCQQWSYPLITFGAGT |
| | KLELKGGGGPKPFITSNNSNPVEDEDAVALTCEPEIQNTTYLWWVNN |
| | QSLPVSPRLQLSNDNRTLTLLSVTRNDVGPYECGIQNELSVDHSDPVI |
| | LNVLYGPDDPTISPSYTYYRPGVNLSLSCHAASNPPAQYSWLIDGNIQ |
| | QHTQELFISNITEKNSGLYTCQANNSASGHSRTTVKTITVSAELPKPSI |
| | SSNNSKPVEDKDAVAFTCEPEAQNTTYLWWVNGQSLPVSPRLQLSN |
| | GNRTLTLFNVTRNDARAYVCGIQNSVSANRSDPVTLDVLYGPDTPIIS |
| | PPDSSYLSGANLNLSCHSASNPSPQYSWRINGIPQQHTQVLFIAKITPN |
| | NNGTYACFVSNLATGRNNSIVKSITVSVVIGILVAVILLLLLLLLLFLIL |
| | RHRRQGKHWTSTQRKADFQHPAGAVGPEPTDRGLQWRSSPAADAQ |
| | EENLYAAVKHTQPEDGVEMDTRSPHDEDPQAVTYAEVKHSRPRRE |
| | MASPPSPLSGEFLDTKDRQAEEDRQMDTEAAASEAPQDVTYAQLHS |
| | LTLRREATEPPPSQEGPSPAVPSIYATLAIH (SEQ ID NO: 63) |
| aLRRN4 | QVQLKESGPGLVAPSQSLSITCTVSGFSLTNYAVHWVRQSPGKGLEW |
| (1J21) | LGAIWSDGSTDYNAAFISRLSISKDNSKSQVFFKMNSLQADDTAIYYC |
| with Ig6 | ARKDYSNYVFDYWGQGTTLTVSSGGGGSGGGGSGGGGSGGEIVLT |
| hinge | QSPAITAASLGQKVTISCSASSSVSYMHWYQQKSGTSPKPWIYDISKL |
| | ASGVPARFSGSGSGTSYSLTISSMEAEDAAIYYCQQWSYPLITFGAGT |
| | KLELKGGGGPKPSISSNNSKPVEDKDAVAFTCEPETQDATYLWWVN |
| | NQSLPVSPRLQLSNGNRTLTLFNVTRNDTASYKCETQNPVSARRSDS |
| | VILNVLYGPDAPTISPLNTSYRSGENLNLSCHAASNPPAQYSWFVNGT |
| | FQQSTQELFIPNITVNNSGSYTCQAHNSDTGLNRTTVTTITVYAEPPKP |
| | FITSNNSNPVEDEDAVALTCEPEIQNTTYLWWVNNQSLPVSPRLQLS |
| | NDNRTLTLLSVTRNDVGPYECGIQNELSVDHSDPVILNVLYGPDDPTI |
| | SPSYTYYRPGVNLSLSCHAASNPPAQYSWLIDGNIQQHTQELFISNITE |
| | KNSGLYTCQANNSASGHSRTTVKTITVSAELPKPSISSNNSKPVEDKD |
| | AVAFTCEPEAQNTTYLWWVNGQSLPVSPRLQLSNGNRTLTLFNVTR |
| | NDARAYVCGIQNSVSANRSDPVTLDVLYGPDTPIISPPDSSYLSGANL |
| | NLSCHSASNPSPQYSWRINGIPQQHTQVLFIAKITPNNNGTYACFVSN |
| | LATGRNNSIVKSITVSVVIGILVAVILLLLLLLLLFLILRHRRQGKHWT |
| | STQRKADFQHPAGAVGPEPTDRGLQWRSSPAADAQEENLYAAVKH |
| | TQPEDGVEMDTRSPHDEDPQAVTYAEVKHSRPRREMASPPSPLSGEF |
| | LDTKDRQAEEDRQMDTEAAASEAPQDVTYAQLHSLTLRREATEPPP |
| | SQEGPSPAVPSIYATLAIH (SEQ ID NO: 64) |
| aMSLN | QVQLVQSGAEVKKPGASVKVSCKASGYSFTGYTMNWVRQAPGQRL |
| (SS1) with | EWMGLITPYNGASSYNQKFRGRVTITRDTSASTAYMELSSLRSEDTA |
| EGF1 | VYYCARGGYDGRGFDYWGQGTTVTVSSGGGGSGGGGSGGGGSGG |
| hinge | DIQMTQSPSSLSASVGDRVTITCSASSSVSYMHWYQQKPGKAPKRLI |
| | YDTSKLASGVPSRFSGSGSGTEFTLTISSLQPEDFATYYCQQWSGYPL |
| | TFGQGTKLEIKGGGGLTCVGHCSNGGSCTMNSKMMPECQCPPHMT |
| | GPRCEVVIGILVAVILLLLLLLLLFLILRHRRQGKHWTSTQRKADFQH |
| | PAGAVGPEPTDRGLQWRSSPAADAQEENLYAAVKHTQPEDGVEMD |
| | TRSPHDEDPQAVTYAEVKHSRPRREMASPPSPLSGEFLDTKDRQAEE |
| | DRQMDTEAAASEAPQDVTYAQLHSLTLRREATEPPPSQEGPSPAVPSI |
| | YATLAIH (SEQ ID NO: 65) |
| aMSLN | QVQLVQSGAEVKKPGASVKVSCKASGYSFTGYTMNWVRQAPGQRL |
| (SS1) with | EWMGLITPYNGASSYNQKFRGRVTITRDTSASTAYMELSSLRSEDTA |
| EGF2 | VYYCARGGYDGRGFDYWGQGTTVTVSSGGGGSGGGGSGGGGSGG |
| hinge | DIQMTQSPSSLSASVGDRVTITCSASSSVSYMHWYQQKPGKAPKRLI |
| | YDTSKLASGVPSRFSGSGSGTEFTLTISSLQPEDFATYYCQQWSGYPL |
| | TFGQGTKLEIKGGGGEVNKCSRCLEGACVVNKQSGDVTCNCTDGRV |
| | APSCLTCVGHCSNGGSCTMNSKMMPECQCPPHMTGPRCEVVIGILV |
| | AVILLLLLLLLLFLILRHRRQGKHWTSTQRKADFQHPAGAVGPEPTD |
| | RGLQWRSSPAADAQEENLYAAVKHTQPEDGVEMDTRSPHDEDPQA |
| | VTYAEVKHSRPRREMASPPSPLSGEFLDTKDRQAEEDRQMDTEAAA |
| | SEAPQDVTYAQLHSLTLRREATEPPPSQEGPSPAVPSIYATLAIH (SEQ |
| | ID NO: 66) |
| aMSLN | QVQLVQSGAEVKKPGASVKVSCKASGYSFTGYTMNWVRQAPGQRL |
| (SS1) with | EWMGLITPYNGASSYNQKFRGRVTITRDTSASTAYMELSSLRSEDTA |
| EGF3 | VYYCARGGYDGRGFDYWGQGTTVTVSSGGGGSGGGGSGGGGSGG |
| hinge | DIQMTQSPSSLSASVGDRVTITCSASSSVSYMHWYQQKPGKAPKRLI |
| | YDTSKLASGVPSRFSGSGSGTEFTLTISSLQPEDFATYYCQQWSGYPL |
| | TFGQGTKLEIKGGGGQYRQCSGYCENFGTCQMAADGSRQCRCTAYF |
| | EGSRCEVNKCSRCLEGACVVNKQSGDVTCNCTDGRVAPSCLTCVGH |
| | CSNGGSCTMNSKMMPECQCPPHMTGPRCEVVIGILVAVILLLLLLLL |
| | LFLILRHRRQGKHWTSTQRKADFQHPAGAVGPEPTDRGLQWRSSPA |
| | ADAQEENLYAAVKHTQPEDGVEMDTRSPHDEDPQAVTYAEVKHSR |
| | PRREMASPPSPLSGEFLDTKDRQAEEDRQMDTEAAASEAPQDVTYA |
| | QLHSLTLRREATEPPPSQEGPSPAVPSIYATLAIH (SEQ ID NO: 67) |
| aMSLN | QVQLVQSGAEVKKPGASVKVSCKASGYSFTGYTMNWVRQAPGQRL |
| (SS1) with | EWMGLITPYNGASSYNQKFRGRVTITRDTSASTAYMELSSLRSEDTA |
| EGF4 | VYYCARGGYDGRGFDYWGQGTTVTVSSGGGGSGGGGSGGGGSGG |
| hinge | DIQMTQSPSSLSASVGDRVTITCSASSSVSYMHWYQQKPGKAPKRLI |
| | YDTSKLASGVPSRFSGSGSGTEFTLTISSLQPEDFATYYCQQWSGYPL |
| | TFGQGTKLEIKGGGGTQQVCAGYCANNSTCTVNQGNQPQCRCLPGF |
| | LGDRCQYRQCSGYCENFGTCQMAADGSRQCRCTAYFEGSRCEVNK |
| | CSRCLEGACVVNKQSGDVTCNCTDGRVAPSCLTCVGHCSNGGSCT |
| | MNSKMMPECQCPPHMTGPRCEVVIGILVAVILLLLLLLLLFLILRHRR |
| | QGKHWTSTQRKADFQHPAGAVGPEPTDRGLQWRSSPAADAQEENL |
| | YAAVKHTQPEDGVEMDTRSPHDEDPQAVTYAEVKHSRPRREMASPP |
| | SPLSGEFLDTKDRQAEEDRQMDTEAAASEAPQDVTYAQLHSLTLRR |
| | EATEPPPSQEGPSPAVPSIYATLAIH (SEQ ID NO: 68) |
| aMSLN | QVQLVQSGAEVKKPGASVKVSCKASGYSFTGYTMNWVRQAPGQRL |
| (SS1) with | EWMGLITPYNGASSYNQKFRGRVTITRDTSASTAYMELSSLRSEDTA |
| EGF5 | VYYCARGGYDGRGFDYWGQGTTVTVSSGGGGSGGGGSGGGGSGG |
| hinge | DIQMTQSPSSLSASVGDRVTITCSASSSVSYMHWYQQKPGKAPKRLI |
| | YDTSKLASGVPSRFSGSGSGTEFTLTISSLQPEDFATYYCQQWSGYPL |
| | TFGQGTKLEIKGGGGELDQCWEHCRNGGTCAASPSGMPTCRCPTGF |
| | TGPKCTQQVCAGYCANNSTCTVNQGNQPQCRCLPGFLGDRCQYRQ |
| | CSGYCENFGTCQMAADGSRQCRCTAYFEGSRCEVNKCSRCLEGACV |
| | VNKQSGDVTCNCTDGRVAPSCLTCVGHCSNGGSCTMNSKMMPECQ |
| | CPPHMTGPRCEVVIGILVAVILLLLLLLLLFLILRHRRQGKHWTSTQR |
| | KADFQHPAGAVGPEPTDRGLQWRSSPAADAQEENLYAAVKHTQPE |
| | DGVEMDTRSPHDEDPQAVTYAEVKHSRPRREMASPPSPLSGEFLDTK |
| | DRQAEEDRQMDTEAAASEAPQDVTYAQLHSLTLRREATEPPPSQEGP |
| | SPAVPSIYATLAIH (SEQ ID NO: 69) |
| aMSLN | QVQLVQSGAEVKKPGASVKVSCKASGYSFTGYTMNWVRQAPGQRL |
| (SS1) with | EWMGLITPYNGASSYNQKFRGRVTITRDTSASTAYMELSSLRSEDTA |
| EGF6 | VYYCARGGYDGRGFDYWGQGTTVTVSSGGGGSGGGGSGGGGSGG |
| hinge | DIQMTQSPSSLSASVGDRVTITCSASSSVSYMHWYQQKPGKAPKRLI |
| | YDTSKLASGVPSRFSGSGSGTEFTLTISSLQPEDFATYYCQQWSGYPL |
| | TFGQGTKLEIKGGGGRPGTCNLQCFNGGSCFLNARRQPKCRCQPRYT |
| | GDKCELDQCWEHCRNGGTCAASPSGMPTCRCPTGFTGPKCTQQVCA |
| | GYCANNSTCTVNQGNQPQCRCLPGFLGDRCQYRQCSGYCENFGTCQ |
| | MAADGSRQCRCTAYFEGSRCEVNKCSRCLEGACVVNKQSGDVTCN |
| | CTDGRVAPSCLTCVGHCSNGGSCTMNSKMMPECQCPPHMTGPRCE |
| | VVIGILVAVILLLLLLLLLFLILRHRRQGKHWTSTQRKADFQHPAGAV |
| | GPEPTDRGLQWRSSPAADAQEENLYAAVKHTQPEDGVEMDTRSPHD |
| | EDPQAVTYAEVKHSRPRREMASPPSPLSGEFLDTKDRQAEEDRQMD |
| | TEAAASEAPQDVTYAQLHSLTLRREATEPPPSQEGPSPAVPSIYATLAI |
| | H (SEQ ID NO: 70) |
| aMSLN | QVQLVQSGAEVKKPGASVKVSCKASGYSFTGYTMNWVRQAPGQRL |
| (SS1) with | EWMGLITPYNGASSYNQKFRGRVTITRDTSASTAYMELSSLRSEDTA |
| EGF7 | VYYCARGGYDGRGFDYWGQGTTVTVSSGGGGSGGGGSGGGGSGG |
| hinge | DIQMTQSPSSLSASVGDRVTITCSASSSVSYMHWYQQKPGKAPKRLI |
| | YDTSKLASGVPSRFSGSGSGTEFTLTISSLQPEDFATYYCQQWSGYPL |
| | TFGQGTKLEIKGGGGVTNPCDRKKCEWLCLLSPSGPVCTCPNGKRLD |
| | NGTCVPVPSPTPPPDAPRPGTCNLQCFNGGSCFLNARRQPKCRCQPR |
| | YTGDKCELDQCWEHCRNGGTCAASPSGMPTCRCPTGFTGPKCTQQV |
| | CAGYCANNSTCTVNQGNQPQCRCLPGFLGDRCQYRQCSGYCENFGT |
| | CQMAADGSRQCRCTAYFEGSRCEVNKCSRCLEGACVVNKQSGDVT |
| | CNCTDGRVAPSCLTCVGHCSNGGSCTMNSKMMPECQCPPHMTGPR |
| | CEVVIGILVAVILLLLLLLLLFLILRHRRQGKHWTSTQRKADFQHPAG |
| | AVGPEPTDRGLQWRSSPAADAQEENLYAAVKHTQPEDGVEMDTRSP |
| | HDEDPQAVTYAEVKHSRPRREMASPPSPLSGEFLDTKDRQAEEDRQ |
| | MDTEAAASEAPQDVTYAQLHSLTLRREATEPPPSQEGPSPAVPSIYAT |
| | LAIH (SEQ ID NO: 71) |
| aMSLN | QVQLVQSGAEVKKPGASVKVSCKASGYSFTGYTMNWVRQAPGQRL |
| (SS1) with | EWMGLITPYNGASSYNQKFRGRVTITRDTSASTAYMELSSLRSEDTA |
| LIR hinge | VYYCARGGYDGRGFDYWGQGTTVTVSSGGGGSGGGGSGGGGSGG |
| | DIQMTQSPSSLSASVGDRVTITCSASSSVSYMHWYQQKPGKAPKRLI |
| | YDTSKLASGVPSRFSGSGSGTEFTLTISSLQPEDFATYYCQQWSGYPL |
| | TFGQGTKLEIKYGSQSSKPYLLTHPSDPLELVVSGPSGGPSSPTTGPTS |
| | TSGPEDQPLTPTGSDPQSGLGRHLGVVIGILVAVILLLLLLLLLFLILRH |
| | RRQGKHWTSTQRKADFQHPAGAVGPEPTDRGLQWRSSPAADAQEE |
| | NLYAAVKHTQPEDGVEMDTRSPHDEDPQAVTYAEVKHSRPRREMA |
| | SPPSPLSGEFLDTKDRQAEEDRQMDTEAAASEAPQDVTYAQLHSLTL |
| | RREATEPPPSQEGPSPAVPSIYATLAIH (SEQ ID NO: 72) |

### Differentially Expressed Inhibitor Ligands

The disclosure provides inhibitor ligands (second antigens) that are differentially expressed between cancer cells and normal cells.

Activation of the inhibitory receptor is mediated by the presence of the second antigen on the surface of a cell. A cell that expresses the second antigen will activate the inhibitory receptor based on the level of expression of the second antigen. In some embodiments, the second antigen is expressed by both target and non-target cells. However, in these embodiments, the second antigen is expressed by non-target cells at a higher level than the target cells. The higher levels of second antigen expressed by the non-target cells activate the inhibitory receptor, thereby preventing activation of the immune cell. In contrast, the lower levels of second antigen expressed by the target are not sufficient to activate the inhibitory receptor, leading to activation of the immune cell.

In alternative embodiments, the second antigen is expressed by non-target cells but not by target cells. In the absence of expression of the second antigen, the target cells activate the target receptor, thereby activating the immune cells.

Differential expression can be determined by any techniques known in the art used to measure expression. These include, inter alia, techniques for measuring mRNA and/or protein levels of a target gene in a cell. Methods of measuring protein levels in samples include immunohistochemistry, enzyme-linked immunosorbent assays (ELISA), and analytical methods such as liquid chromatography-mass spectrometry (LC-MS). Methods of measuring mRNA levels include real time quantitative reverse transcription PCR (qRT-PCR), as well as high throughput sequencing. Expression differences can be observed between, for example, a normal cell and a diseased cell, for example a cancer cell.

Activation of the inhibitory receptor by a second antigen can occur according to various modalities known in the art. Activation of the inhibitory receptor by a second antigen can be determined by methods known in the art. For example, the level of downstream intracellular signaling in a cell expressing the inhibitory receptor can be measured through the use of a reporter gene.

Without wishing to be bound by theory, whether or not expression of a second antigen inhibits activation of an immune cell via activation of the inhibitory receptor can occur according to the ratio of the second antigen to the inhibitor receptor. The expression levels of the second antigen and the inhibitory receptor, and the ratio thereof, can be determined by methods known in the art, including, inter alia, immunohistochemistry and fluorescence activated cell sorting (FACS). Analysis of the expression levels of the non-target antigen on target and non-target cells can be used to predict selective targeting of the immune cells expressing the inhibitory receptor. Low or no expression of the second antigen on a target or non-target cell can indicate, for example, that the inhibitory receptor will not be activated in an immune cell of the disclosure.

Alternatively, or in addition, and without wishing to be bound by theory, inhibition of immune cell activation by a second antigen via activation of the inhibitory receptor can depend on the affinity of the second antigen for the inhibitory receptor. Methods of measuring affinity are known in the art, and include, inter alia, enzyme-linked immunosorbent assay or radioimmunoassay methods.

Alternatively, or in addition, and without wishing to be bound by theory, inhibition of immune cell activation by a second antigen via activation of the inhibitory receptor can occur according to cross talk between the inhibitory receptor and the activator receptor, leading to down-regulation of the activity of the activator receptor. For example, activation of the inhibitory receptor by the second antigen can lead to reduced expression of the activator receptor on the surface of the immune cell.

In some embodiments, the second antigen is expressed at a lower level in a target cell than a normal cell. In some embodiments, the second antigen is expressed by healthy cells, i.e., cells that are not cancer cells. In some embodiments, the second antigen expression level is at least about 10 times less, at least about 30 times less, at least about 50 times less, at least about 70 times less, at least about 90 times less, at least about 100 times less, at least about 110 times less, at least about 150 times less, at least about 200 times less, at least about 250 times less, at least about 300 times less, at least about 350 times less, at least about 400 times less, at least about 450 times less, at least about 500 times less, at least about 600 times less, at least about 700 times less, at least about 800 times less, at least about 900 times less or at least about 1000 times less in the target cell than in the non-target cell. In some embodiments, the second antigen expression level is about 10 times less, about 30 times less, about 50 times less, about 70 times less, about 90 times less, about 100 times less, or about 110 times less than the plurality of healthy cells. In some embodiments, the second antigen expression level is at least about 5 times less in the plurality of cancer cells than in the plurality of healthy cells. In some embodiments, the second antigen expression level is at least about 5 times less in a target cell than a non-target cell. In some embodiments, the target cells are a plurality of cancer cells that have low or no expression of the second antigen.

Any cell surface molecule expressed by the non-target cells that is not expressed by target cells (or expressed at a low level) may be a suitable second antigen for the second receptor extracellular ligand binding domain. For example, a cell adhesion molecule, a cell-cell signaling molecule, an extracellular domain, a molecule involved in chemotaxis, a glycoprotein, a G protein-coupled receptor, a transmembrane protein, a receptor for a neurotransmitter or a voltage gated ion channel can be used as a non-target antigen.

In some embodiments, the second antigen is selected from the group consisting of leucine rich repeat neuronal 4 (LRRN4) and uroplakin B3 (UPKB3), or a peptide antigen of any of these in a complex with a major histocompatibility complex class I (MHC-I). In some embodiments, the second antigen is LRRN4 or a peptide antigen thereof in a complex with MHC-I. In some embodiments, the second antigen is UPKB3 or a peptide antigen thereof in a complex with MHC-I.

In some embodiments, the second antigen is a peptide antigen of a cancer cell-specific antigen in a complex with a major histocompatibility complex class I (MHC-I).

Second MHC-I (pNMC) antigens comprising any of HLA-A, HLA-B or HLA-C are envisaged as within the scope of the disclosure. In some embodiments, the second antigen comprises HLA-A. In some embodiments, the second antigen comprises HLA-B. In some embodiments, the second antigen comprises HLA-C.

Non-target antigens comprise proteins that have low or no expression in cancer cells, for example lung cancer cells, but are expressed in normal tissues, such as normal lung tissue.

In some embodiments, the second antigen comprises LRRN4 or an antigen peptide thereof in a complex with MHC-I. A human LRRN4 is described in NCBI record number NP_689824.2, the contents of which are incorporated by reference herein in their entirety. In some embodiments, LRRN4 comprises an amino acid sequence of:

| (SEQ ID NO: 73) | |
|---|---|
| 1 | MRQTLPLLLL TVLRPSWADP PQEKVPLFRV TQQGPWGSSG SNATDSPCEG LPAADATALT |
| 61 | LANRNLERLP GCLPRTLRSL DASHNLLRAL STSELGHLEQ LQVLTLRHNR IAALRWGPGG |
| 121 | PAGLHTLDLS YNQLAALPPC TGPALSSLRA LALAGNPLRA LQPRAFACFP ALQLLNLSCT |
| 181 | ALGRGAQGGI AEAAFAGEDG APLVTLEVLD LSGTFLERVE SGWIRDLPKL TSLYLRKMPR |
| 241 | LTTLEGDIFK MTPNLQQLDC QDSPALASVA THIFQDTPHL QVLLFQNCNL SSFPPWTLDS |
| 301 | SQVLSINLFG NPLTCSCDLS WLLTDAKRTV LSRAADTMCA PAAGSSGPFS ASLSLSQLPG |
| 361 | VCQSDQSTTL GASHPPCFNR STYAQGTTVA PSAAPATRPA GDQQSVSKAP NVGSRTIAAW |
| 421 | PHSDAREGTA PSTTNSVAGH SNSSVFPRAA STTRTQHRGE HAPELVLEPD ISAASTPLAS |
| 481 | KLLGPFPTSW DRSISSPQPG QRTHATPQAP NPSLSEGEIP VLLLDDYSEE EEGRKEEVGT |
| 541 | PHQDVPCDYH PCKHLQTPCA ELQRRWRCRC PGLSGEDTIP DPPRLQGVTE TTDTSALVHW |
| 601 | CAPNSVVHGY QIRYSAEGWA GNQSVVGVIY ATARQHPLYG LSPGTTYRVC VLAANRAGLS |
| 661 | RSSGWRSPCA AFTTKPSFAL LLSGLCAASG LLLASTVVLS ACLCRRGQTL GLQRCDTHLV |
| 721 | AYKNPAFDDY PLGLQTVS. |

In some embodiments, LRRN4 comprises a sequence that shares at least 80%, at least 85%, at least 90%, at least 95%, at least 96%, at least 97%, at least 98%, or at least 99% identity to SEQ ID NO: 73. In some embodiments, LRRN4 comprises a sequence identical to SEQ ID NO: 73.

In some embodiments, the second antigen comprises UPK3B or an antigen peptide thereof in a complex with MHC-I. All isoforms of UPK3B are envisaged as within the scope of the instant disclosure. A human UPK3B isoform a precursor is described in NCBI record number NP_085047.1, the contents of which are incorporated by reference herein in their entirety. In some embodiments, UPK3B isoform a precursor comprises an amino acid sequence of

| (SEQ ID NO: 74) | |
|---|---|
| 1 | MGLPWGQPHL GLQMLLLALN CLRPSLSLGE WGSWMDASSQ TQGAGGPAGV IGPWAPAPLR |
| 61 | LGEAAPGTPT PVSVAHLLSP VATELVPYTP QITAWDLEGK VTATTFSLEQ PRCVFDGLAS |
| 121 | ASDTVWLVVA FSNASRGFQN PETLADIPAS PQLLTDGHYM TLPLSPDQLP CGDPMAGSGG |
| 181 | APVLRVGHDH GCHQQPFCNA PLPGPGPYRE DPRIHRHLAR AAKWQHDRHY LHPLFSGRPP |
| 241 | TLGLLGSLYH ALLQPVVAGG GPGAAADRLL HGQALHDPPH PTQRGRHTAG GLQAWPGPPP |
| 301 | QPQPLAWPLC MGLGEMGRRE. |

A human UPK3B isoform b precursor is described in NCBI record number NP_872625.1, the contents of which are incorporated by reference herein in their entirety. In some embodiments, UPK3B isoform b precursor comprises an amino acid sequence of

| (SEQ ID NO: 75) | |
|---|---|
| 1 | MGLPWGQPHL GLQMLLLALN CLRPSLSLEL VPYTPQITAW DLEGKVTATT FSLEQPRCVF |
| 61 | DGLASASDTV WLVVAFSNAS RGFQNPETLA DIPASPQLLT DGHYMTLPLS PDQLPCGDPM |
| 121 | AGSGGAPVLR VGHDHGCHQQ PFCNAPLPGP GPYRVKFLLM DTRGSPRAET KWSDPITLHQ |
| 181 | GKTPGSIDTW PGRRSGSMIV ITSILSSLAG LLLLAFLAAS TMRFSSLWWP EEAPEQLRIG |
| 241 | SFMGKRYMTH HIPPSEAATL PVGCKPGLDP LPSLSP. |

A human UPK3B isoform c precursor is described in NCBI record number NP_872624.1, the contents of which are incorporated by reference herein in their entirety. In some embodiments, UPK3B isoform c precursor comprises an amino acid sequence of:

| (SEQ ID NO: 76) | |
|---|---|
| 1 | MGLPWGQPHL GLQMLLLALN CLRPSLSLEL VPYTPQITAW DLEGKVTATT FSLEQPRCVF |
| 61 | DGLASASDTV WLVVAFSNAS RGFQNPETLA DIPASPQLLT DGHYMTLPLS PDQLPCGDPM |
| 121 | AGSGGAPVLR VGHDHGCHQQ PFCNAPLPGP GPYREDPRIH RHLARAAKWQ HDRHYLHPLF |
| 181 | SGRPPTLGLL GSLYHALLQP VVAGGGPGAA ADRLLHGQAL HDPPHPTQRG RHTAGGLQAW |
| 241 | PGPPPQPQPL AWPLCMGLGE MGRRE. |

A human UPK3B isoform d precursor is described in NCBI record number NP_001334613.1, the contents of which are incorporated by reference herein in their entirety. In some embodiments, UPK3B isoform c precursor comprises an amino acid sequence of

| (SEQ ID NO: 77) | |
|---|---|
| 1 | MGLPWGQPHL GLQMLLLALN CLRPSLSLEL VPYTPQITAW DLEGKVTATT FSLEQPRCVF |
| 61 | DGLASASDTV WLVVAFSNAS RGFQNPETLA DIPASPQLLT DGHYMTLPLS PDQLPCGDPM |
| 121 | AGSGGAPVLR VGHDHGCHQQ PFCNAPLPGP GPYRVKFLLM DTRGSPRAET KWSDPITLHQ |
| 181 | GKTPGSIDTW PGRRSGSMIV ITSILSSLAG LLLLAFLAAS TMRFSSLWWP EEAPEQLRIG |
| 241 | SFMGKRYMTH HIPPREAATL PVGCKPGLDP LPSLSP. |

In some embodiments, UPKB3 comprises a sequence or subsequence that shares at least 80%, at least 85%, at least 90%, at least 95%, at least 96%, at least 97%, at least 98%, or at least 99% identity to any one of SEQ ID NOs: 74-77. In some embodiments, UPKB3 comprises a sequence or subsequence identical to SEQ ID NOs: 74-77.

### Inhibitory Chimeric Antigen Receptors

The disclosure provides an inhibitory receptor that is an inhibitory chimeric antigen receptor. The inhibitory receptor may comprise an extracellular ligand binding domain that binds to and recognizes the second antigen or a peptide derivative thereof in an MHC-I complex.

The term “inhibitory receptor” as used herein refers to a ligand-binding domain that is fused to an intracellular signaling domain capable of transducing an inhibitory signal that inhibits or suppresses the immune activity of an immune cell. Inhibitory receptors have immune cell inhibitory potential, and are distinct and distinguishable from CARs, which are receptors with immune cell activating potential. For example, CARs are activating receptors as they include intracellular stimulatory and/or co-stimulatory domains. Inhibitory receptors are inhibiting receptors that contain intracellular inhibitory domains.

As used herein “inhibitory signal” refers to signal transduction or changes in protein expression in an immune cell resulting in suppression of an immune response (e.g., decrease in cytokine production or reduction of immune cell activation). Inhibition or suppression of an immune cell can selective and/or reversible, or not selective and/or reversible.

Inhibitory receptors of the disclosure may comprise an extracellular ligand binding domain. Any type of ligand binding domain that can regulate the activity of a receptor in a ligand dependent manner is envisaged as within the scope of the instant disclosure. Inhibitory receptors are responsive to non-target antigens (e.g., HLA-A*02). For example, when a non-target antigen (e.g., HLA-A*02) binds to or contacts the inhibitory receptor, the inhibitory receptor is responsive and activates an inhibitory signal in the immune cell expressing the inhibitory receptor upon binding of the non-target antigen by the extracellular ligand binding domain of the inhibitory receptor.

Inhibitory receptors of the disclosure may comprise an extracellular ligand binding domain. Any type of ligand binding domain that can regulate the activity of a receptor in a ligand dependent manner is envisaged as within the scope of the instant disclosure.

In some embodiments, the ligand binding domain is an antigen binding domain. Exemplary antigen binding domains include, inter alia, scFv, SdAb, Vβ-only domains, and TCR antigen binding domains derived from the TCR α and β chain variable domains.

Any type of antigen binding domain is envisaged as within the scope of the instant disclosure.

In some embodiments, the extracellular ligand binding domain of the second receptor is an scFv.

In some embodiments, the extracellular ligand binding domain of the second receptor binds to and recognizes a polymorphic variant of intercellular adhesion molecule 1 (ICAM1), catechol-O-methyltransferase (COMT), C—X—C motif chemokine ligand 16 (CXCL16), leucine rich repeat neuronal 4 (LRRN4) and uroplakin 3B UPK3B, or an antigen peptide thereof in a complex with a major histocompatibility complex class I (MHC-I), or HLA-A*02. In some embodiments, the extracellular ligand binding domain of the second receptor is an scFv.

In some embodiments, the extracellular ligand binding domain of the second receptor is fused to the extracellular domain of an inhibitory receptor.

In some embodiments, the CARs of the present disclosure comprise an extracellular hinge region. Hinge elements contemplated for use in the present disclosure are described in detail above.

The inhibitory receptors of the present disclosure can be designed to comprise a transmembrane domain that is fused to the extracellular domain of the inhibitory receptor. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein. Transmembrane regions may be isolated or derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, or from an immunoglobulin such as IgG4. Alternatively, the transmembrane domain may be synthetic, in which case it will comprise predominantly hydrophobic residues such as leucine and valine. In some embodiments, a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the intracellular domain of the inhibitory receptor. A glycine-serine doublet provides a particularly suitable linker.

The disclosure provides an inhibitory receptor comprising an intracellular domain. The intracellular domain of the inhibitory receptors of the instant disclosure is responsible for inhibiting activation of the immune cells comprising the inhibitory receptor, which would otherwise be activated in response to activation signals by the first receptor. In some embodiments, the inhibitory intracellular domain comprises an immunoreceptor tyrosine-based inhibitory motif (ITIM). In some embodiments, the inhibitory intracellular domain comprising an ITIM can be isolated or derived from an immune checkpoint inhibitor such as CTLA-4 and PD-1. CTLA-4 and PD-1 are immune inhibitory receptors expressed on the surface of T cells and play a pivotal role in attenuating or terminating T cell responses.

In some embodiments, an inhibitory intracellular domain is isolated from human tumor necrosis factor related apoptosis inducing ligand (TRAIL) receptor and CD200 receptor 1. In some embodiments, the TRAIL receptor comprises TR10A, TR10B or TR10D.

In some embodiments, an inhibitory intracellular domain is isolated from phosphoprotein membrane anchor with glycosphingolipid microdomains 1 (PAGI). In some embodiments, an inhibitory intracellular domain is isolated from leukocyte immunoglobulin like receptor B1 (LILRB1).

In some embodiments, the inhibitory domain is isolated or derived from a human protein, for example a human TRAIL receptor, CTLA-4, PD-1, PAGI or LILRB1 protein.

In some embodiments, the inhibitory domain comprises an intracellular domain, a transmembrane or a combination thereof. In some embodiments, the inhibitory domain comprises an intracellular domain, a transmembrane domain, a hinge region or a combination thereof.

In some embodiments, the inhibitory domain is isolated or derived from killer cell immunoglobulin like receptor, three Ig domains and long cytoplasmic tail 2 (KIR3DL2), killer cell immunoglobulin like receptor, three Ig domains and long cytoplasmic tail 3 (KIR3DL3), leukocyte immunoglobulin like receptor B1 (LIR1, also called LIR-1 and LILRB1), programmed cell death 1 (PD-1), Fc gamma receptor IIB (FcgRIIB), killer cell lectin like receptor K1 (NKG2D), CTLA-4, a domain containing a synthetic consensus ITIM, a ZAP70 SH2 domain (e.g., one or both of the N and C terminal SH2 domains), or ZAP70 KI_K369A (kinase inactive ZAP70).

In some embodiments, the inhibitory domain is isolated or derived from a human protein.

In some embodiments, the inhibitory receptor comprises an inhibitory domain. In some embodiments, the inhibitory receptor comprises an inhibitory intracellular domain and/or an inhibitory transmembrane domain. In some embodiments, the inhibitory intracellular domain is fused to an intracellular domain of an inhibitory receptor. In some embodiments, the inhibitory intracellular domain is fused to the transmembrane domain of an inhibitory receptor.

In some embodiments, the inhibitory receptor comprises a cytoplasmic domain, a transmembrane domain, and an extracellular domain or a portion thereof isolated or derived isolated or derived from the same protein, for example an ITIM containing protein. In some embodiments, the inhibitory receptor comprises a hinge region isolated or derived from isolated or derived from the same protein as the intracellular domain and/or transmembrane domain, for example an ITIM containing protein.

In some embodiments, the inhibitory receptor is a TCR comprising an inhibitory domain (an inhibitory TCR). In some embodiments, the inhibitory TCR comprises an inhibitory intracellular domain and/or an inhibitory transmembrane domain. In some embodiments, the inhibitory intracellular domain is fused to the intracellular domain of TCR alpha, TCR beta, CD3 delta, CD3 gamma or CD3 epsilon or a portion thereof a TCR. In some embodiments, the inhibitory intracellular domain is fused to the transmembrane domain of TCR alpha, TCR beta, CD3 delta, CD3 gamma or CD3 epsilon. In some embodiments, the second receptor is a TCR comprising an inhibitory domain (an inhibitory TCR). In some embodiments, the inhibitory domain is isolated or derived from LILRB1.

### LILRB1 Inhibitory Receptors

The disclosure provides an inhibitory receptor comprising a LILRB1 inhibitory domain, and optionally, a LILRB1 transmembrane and/or hinge domain, or functional variants thereof. The inclusion of the LILRB1 transmembrane domain and/or the LILRB1 hinge domain in the inhibitory receptor may increase the inhibitory signal generated by the inhibitory receptor compared to a reference inhibitory receptor having another transmembrane domain or another hinge domains. The inhibitory receptor comprising the LILRB1 inhibitory domain may be a CAR or TCR, as described herein. Any suitable ligand binding domain may be fused to an extracellular domain, hinge domain or transmembrane of the TCRs described herein. For example, the ligand binding domain can be an antigen binding domain of an antibody or TCR, or comprise an antibody fragment, a Vβ only domain, a linear antibody, a single-chain variable fragment (scFv), or a single domain antibody (sdAb).

Leukocyte immunoglobulin-like receptor subfamily B member 1 (LILRB1), also known as Leukocyte immunoglobulin-like receptor B1, as well as ILT2, LIR1, MIR7, PIRB, CD85J, ILT-2 LIR-1, MIR-7 and PIR-B, is a member of the leukocyte immunoglobulin-like receptor (LIR) family. The LILRB1 protein belongs to the subfamily B class of LIR receptors. These receptors contain two to four extracellular immunoglobulin domains, a transmembrane domain, and two to four cytoplasmic immunoreceptor tyrosine-based inhibitory motifs (ITIMs). The LILRB1 receptor is expressed on immune cells, where it binds to MHC class I molecules on antigen-presenting cells and transduces a negative signal that inhibits stimulation of an immune response. LILRB1 is thought to regulate inflammatory responses, as well as cytotoxicity, and to play a role in limiting auto-reactivity. Multiple transcript variants encoding different isoforms of LILRB1 exist, all of which are contemplated as within the scope of the instant disclosure.

LILRB1 sequences useful in the inhibitory receptors disclosed herein are described in PCT/US2021/060607 and PCT/US2023/011698, which are incorporated herein by reference in their entirety.

### Inhibitory Receptors Comprising Combinations of LILRB1 Domains

In some embodiments, the LILRB1-based inhibitory receptors of the disclosure comprise more than one LILRB1 domain or functional equivalent thereof. For example, in some embodiments, the inhibitory receptor comprises an LILRB1 transmembrane domain and intracellular domain, or an LILRB1 hinge domain, transmembrane domain and intracellular domain.

LILRB1 domain combinations and sequences useful in the inhibitory receptors disclosed herein are described in PCT/US2021/060607 and PCT/US2023/011698, which are incorporated herein by reference in their entirety.

Exemplary inhibitory receptors of the disclosure comprise the scFv specific to any of HLA-A*03, fused to the N terminus a LILRB1 hinge, transmembrane and intracellular domain. In some embodiments, the LILRB1 hinge comprises a sequence of SEQ ID NO: 145, the LILRB1 transmembrane domain comprises a sequence of SEQ ID NO: 146, and the LILRB1 intracellular domain comprises a sequence of SEQ ID NO: 142.

In some embodiments, the non-target antigen comprises HLA-A*03, and the inhibitory receptor comprises a sequence of:

EVQLVESGGGLVKPGGSLRLSCAASGFTFSNYWMNWVRQAPGKGLEWVGE IRLKSTNYATHYAESVKGRFTISRDDSKNTLYLQMNSLKTEDTAVYYCTTLITPDYW GQGTTVTVSSGGGGSGGGGSGGGGSGGDIQMTQSPSSVSASVGDRVTITCKASQDV STTVAWYQQKPGKAPKLLIYSASYRYTGVPSRFSGSGSGTDFTLTISSLQPEDFATYY CQQHYSTPPTFGGGTKVEIKYGSQSSKPYLLTHPSDPLELVVSGPSGGPSSPTTGPTST SGPEDQPLTPTGSDPQSGLGRHLGVVIGILVAVILLLLLLLLLFLILRHRRQGKHWTST QRKADFQHPAGAVGPEPTDRGLQWRSSPAADAQEENLYAAVKHTQPEDGVEMDT RSPHDEDPQAVTYAEVKHSRPRREMASPPSPLSGEFLDTKDRQAEEDRQMDTEAAA SEAPQDVTYAQLHSLTLRREATEPPPSQEGPSPAVPSIYATLAIHGSGEGRGSLLTCG DVEENPGPMEFGLSWLFLVAILKGVQCQVQLQESGPGLVKPSQTLSLTCTVSGGSIS SGDYYWSWIRQPPGKGLEWIGYIYYSGSTYYNPSLKSRVTISVDTSKNQFSLKLSSV TAADTAVYYCAREDVVKGAFDIWGQGTMVTVSSGGGGSGGGGSGGGGSGGDIQM TQSPSSLSASVGDRVTITCRASQSISSYLNWYQQKPGKAPKLLIYAASSLQSGVPSRF SGSGSGTDFTLTISSLQPEDFATYYCQQSYSTPLTFGGGTKVEIKTTTPAPRPPTPAPTI ASQPLSLRPEACRPAAGGAVHTRGLDFACDFWVLVVVGGVLACYSLLVTVAFIIFW VRSKRSRLLHSDYMNMTPRRPGPTRKHYQPYAPPRDFAAYRSKRGRKKLLYIFKQP FMRPVQTTQEEDGCSCRFPEEEEGGCELRVKFSRSADAPAYKQGQNQLYNELNLGR REEYDVLDKRRGRDPEMGGKPRRKNPQEGLYNELQKDKMAEAYSEIGMKGERRR GKGHDGLYQGLSTATKDTYDALHMQALPPR (SEQ ID NO: 220), or a sequence having at least 90%, at least 95%, at least 97% or at least 99% identity thereto. In some embodiments, the non-target antigen comprises HLA-A*03 and the inhibitory receptor comprises a sequence of SEQ ID NO: 220.

In some embodiments, the non-target antigen comprises HLA-A*02 and the inhibitory receptor comprises a sequence of:

MDMRVPAQLLGLLLLWLRGARCDVLMTQTPLSLPVSLGDQASISCRSSQSIV HSNGNTYLEWYLQKPGQSPKLLIYKVSNRFSGVPDRFSGSGSGTDFTLKISRVEAED LGVYYCFQGSHVPRTSGGGTKLEIKGGGGSGGGGSGGGGSGGQVQLQQSGPELVK PGASVRISCKASGYTFTSYHIHWVKQRPGQGLEWIGWIYPGNVNTEYNEKFKGKAT LTADKSSSTAYMHLSSLTSEDSAVYFCAREEITYAMDYWGQGTSVTVSSYGSQSSK PYLLTHPSDPLELVVSGPSGGPSSPTTGPTSTSGPEDQPLTPTGSDPQSGLGRHLGVVI GILVAVILLLLLLLLLFLILRHRRQGKHWTSTQRKADFQHPAGAVGPEPTDRGLQWR SSPAADAQEENLYAAVKHTQPEDGVEMDTRSPHDEDPQAVTYAEVKHSRPRREMA SPPSPLSGEFLDTKDRQAEEDRQMDTEAAASEAPQDVTYAQLHSLTLRREATEPPPS QEGPSPAVPSIYATLAIH (SEQ ID NO: 110) or a sequence having at least 90%, at least 95%, at least 97% or at least 99% identity thereto. In some embodiments, the non-target antigen comprises HLA-A*02 and the inhibitory receptor comprises a sequence of SEQ ID NO: 110.

In some embodiments, the non-target antigen comprises HLA-A*02 and the inhibitory receptor comprises a sequence of:

DVLMTQTPLSLPVSLGDQASISCRSSQSIVHSNGNTYLEWYLQKPGQSPKLLI YKVSNRFSGVPDRFSGSGSGTDFTLKISRVEAEDLGVYYCFQGSHVPRTSGGGTKLE IKGGGGSGGGGSGGGGSGGQVQLQQSGPELVKPGASVRISCKASGYTF TSYHIHWV KQRPGQGLEWIGWIYPGNVNTEYNEKFKGKATLTADKSSSTAYMHLSSLTSEDSAV YFCAREEITYAMDYWGQGTSVTVSSYGSQSSKPYLLTHPSDPLELVVSGPSGGPSSP TTGPTSTSGPEDQPLTPTGSDPQSGLGRHLGVVIGILVAVILLLLLLLLLFLILRHRRQ GKHWTSTQRKADFQHPAGAVGPEPTDRGLQWRSSPAADAQEENLYAAVKHTQPE DGVEMDTRSPHDEDPQAVTYAEVKHSRPRREMASPPSPLSGEFLDTKDRQAEEDRQ MDTEAAASEAPQDVTYAQLHSLTLRREATEPPPSQEGPSPAVPSIYATLAIH (SEQ ID NO: 111) or a sequence having at least 90%, at least 95%, at least 97% or at least 99% identity thereto. In some embodiments, the non-target antigen comprises HLA-A*02 and the inhibitory receptor comprises a sequence of SEQ ID NO: 111.

### Immune Checkpoint Inhibitory Receptors

The disclosure provides an inhibitory receptor comprising an immune checkpoint inhibitory domain. In some embodiments, the inhibitory domain is homologous to a signal transduction element of an immune checkpoint protein, such as an immune checkpoint protein selected from the group consisting of PD1; CTLA4; BTLA; 2B4; CD 160; CEACAM, such as CEACAM1; KIRs, such as KIR2DL1, KIR2DL2, KIR2DL3, KIR2DL4, KIR2DL5A, KIR2DL5B, KIR3DL1, KIR3DL2, KIR3DL3, LIR1, LIR2, LIR3, LIR5, LIR8 and CD94-KG2A; LAG3; TIM3; V-domain Ig suppressor of T cell activation (VISTA); STimulator of INterferon Genes (STING); immunoreceptor tyrosine-based inhibitory motif (ITIM)-containing proteins, T cell immunoglobulin and ITIM domain (TIGIT), and adenosine receptor (e.g., A2aR). In some embodiments, the immune checkpoint protein is a negative immune regulator. In some embodiments, the negative immune regulator is selected from the group consisting of 2B4, LAG-3 and BTLA-4.

In some embodiments, immune checkpoint protein is a natural killer cell inhibitory receptor, e.g., KIRs, such as KIR2DL1, KTR2DL2, KIR2DL3, KIR2DL4, KIR2DL5A, KIR2DL5B, KIR3DL1, KIR3DL2, KTR3DL3; or a Leukocyte Ig-like receptor, such as LIR1, LIR2, LIR3, LIR5, LIR8; and CD94-NKG2A, a C-type lectin receptor which forms heterodimers with CD94 and contains 2 ITIMs.

### Inhibitory Chimeric Antigen Receptors

The disclosure provides an inhibitory receptor that is an inhibitory chimeric antigen receptor. The inhibitory receptor may comprise an extracellular ligand binding domain that binds to and recognizes HLA-A*02 or a peptide derivative thereof in an MHC-I complex.

The term “inhibitory receptor” as used herein refers to a ligand-binding domain that is fused to an intracellular signaling domain capable of transducing an inhibitory signal that inhibits or suppresses the immune activity of an immune cell. Inhibitory receptors have immune cell inhibitory potential, and are distinct and distinguishable from CARs, which are receptors with immune cell activating potential. For example, CARs are activating receptors as they include intracellular stimulatory and/or co-stimulatory domains. Inhibitory receptors are inhibiting receptors that contain intracellular inhibitory domains.

As used herein “inhibitory signal” refers to signal transduction or changes in protein expression in an immune cell resulting in suppression of an immune response (e.g., decrease in cytokine production or reduction of immune cell activation). Inhibition or suppression of an immune cell can selective and/or reversible, or not selective and/or reversible.

Inhibitory receptors of the disclosure may comprise an extracellular ligand binding domain. Any type of ligand binding domain that can regulate the activity of a receptor in a ligand dependent manner is envisaged as within the scope of the instant disclosure. Inhibitory receptors are responsive to non-target antigens (e.g., HLA-A*02). For example, when a non-target antigen (e.g., HLA-A*02) binds to or contacts the inhibitory receptor, the inhibitory receptor is responsive and activates an inhibitory signal in the immune cell expressing the inhibitory receptor upon binding of the non-target antigen by the extracellular ligand binding domain of the inhibitory receptor.

Inhibitory receptors of the disclosure may comprise an extracellular ligand binding domain. Any type of ligand binding domain that can regulate the activity of a receptor in a ligand dependent manner is envisaged as within the scope of the instant disclosure.

In some embodiments, the ligand binding domain is an antigen binding domain. Exemplary antigen binding domains include, inter alia, scFv, SdAb, Vβ-only domains, and TCR antigen binding domains derived from the TCR α and β chain variable domains.

Any type of antigen binding domain is envisaged as within the scope of the instant disclosure.

In some embodiments, the extracellular ligand binding domain of the inhibitory receptor is an scFv.

In some embodiments, the extracellular ligand binding domain of the inhibitory receptor binds to and recognizes HLA-A*02. In some embodiments, the extracellular ligand binding domain of the inhibitory receptor is an scFv.

In some embodiments, the extracellular ligand binding domain of the inhibitory receptor is fused to the extracellular domain of an inhibitory receptor.

In some embodiments, the inhibitory receptors of the present disclosure comprise an extracellular hinge region. Exemplary hinges can be isolated or derived from IgD and CD8 domains, for example IgG1. In some embodiments, the hinge is isolated or derived from CD8α or CD28.

The inhibitory receptors of the present disclosure can be designed to comprise a transmembrane domain that is fused to the extracellular domain of the inhibitory receptor. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein. Transmembrane regions may be isolated or derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, or from an immunoglobulin such as IgG4. Alternatively, the transmembrane domain may be synthetic, in which case it will comprise predominantly hydrophobic residues such as leucine and valine. In some embodiments, a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the intracellular domain of the inhibitory receptor. A glycine-serine doublet provides a particularly suitable linker.

The disclosure provides an inhibitory receptor comprising an intracellular domain. The intracellular domain of the inhibitory receptors of the instant disclosure is responsible for inhibiting activation of the immune cells comprising the inhibitory receptor, which would otherwise be activated in response to activation signals by the activator receptor. In some embodiments, the inhibitory intracellular domain comprises an immunoreceptor tyrosine-based inhibitory motif (ITIM). In some embodiments, the inhibitory intracellular domain comprising an ITIM can be isolated or derived from an immune checkpoint inhibitor such as CTLA-4 and PD-1. CTLA-4 and PD-1 are immune inhibitory receptors expressed on the surface of T cells and play a pivotal role in attenuating or terminating T cell responses.

In some embodiments, an inhibitory intracellular domain is isolated from human tumor necrosis factor related apoptosis inducing ligand (TRAIL) receptor and CD200 receptor 1. In some embodiments, the TRAIL receptor comprises TR10A, TR10B or TR10D.

In some embodiments, an inhibitory intracellular domain is isolated from phosphoprotein membrane anchor with glycosphingolipid microdomains 1 (PAGI). In some embodiments, an inhibitory intracellular domain is isolated from leukocyte immunoglobulin like receptor B1 (LILRB1).

In some embodiments, the inhibitory domain is isolated or derived from a human protein, for example a human TRAIL receptor, CTLA-4, PD-1, PAGI or LILRB1 protein.

In some embodiments, the inhibitory domain comprises an intracellular domain, a transmembrane or a combination thereof. In some embodiments, the inhibitory domain comprises an intracellular domain, a transmembrane domain, a hinge region or a combination thereof.

In some embodiments, the inhibitory domain is isolated or derived from killer cell immunoglobulin like receptor, three Ig domains and long cytoplasmic tail 2 (KIR3DL2), killer cell immunoglobulin like receptor, three Ig domains and long cytoplasmic tail 3 (KIR3DL3), leukocyte immunoglobulin like receptor B1 (LIR1, also called LIR-1 and LILRB1), programmed cell death 1 (PD-1), Fc gamma receptor IIB (FcgRIIB), killer cell lectin like receptor K1 (NKG2D), CTLA-4, a domain containing a synthetic consensus ITIM, a ZAP70 SH2 domain (e.g., one or both of the N and C terminal SH2 domains), or ZAP70 KI_K369A (kinase inactive ZAP70).

In some embodiments, the inhibitory domain is isolated or derived from a human protein.

In some embodiments, the inhibitory receptor comprises an inhibitory domain. In some embodiments, the inhibitory receptor comprises an inhibitory intracellular domain and/or an inhibitory transmembrane domain. In some embodiments, the inhibitory intracellular domain is fused to an intracellular domain of an inhibitory receptor. In some embodiments, the inhibitory intracellular domain is fused to the transmembrane domain of an inhibitory receptor.

In some embodiments, the inhibitory receptor comprises a cytoplasmic domain, a transmembrane domain, and an extracellular domain or a portion thereof isolated or derived isolated or derived from the same protein, for example an ITIM containing protein. In some embodiments, the inhibitory receptor comprises a hinge region isolated or derived from isolated or derived from the same protein as the intracellular domain and/or transmembrane domain, for example an ITIM containing protein.

In some embodiments, the inhibitory receptor is a TCR comprising an inhibitory domain (an inhibitory TCR). In some embodiments, the inhibitory TCR comprises an inhibitory intracellular domain and/or an inhibitory transmembrane domain. In some embodiments, the inhibitory intracellular domain is fused to the intracellular domain of TCR alpha, TCR beta, CD3 delta, CD3 gamma or CD3 epsilon or a portion thereof a TCR. In some embodiments, the inhibitory intracellular domain is fused to the transmembrane domain of TCR alpha, TCR beta, CD3 delta, CD3 gamma or CD3 epsilon.

In some embodiments, the inhibitory receptor is a TCR comprising an inhibitory domain (an inhibitory TCR). In some embodiments, the inhibitory domain is isolated or derived from LILRB1.

### Inhibitor Ligands

In some embodiments, the HLA (for example HLA-A*02) is not expressed by the target cells and is expressed by non-target cells. In some embodiments, the HLA (for example HLA-A*02) is expressed by healthy cells, i.e., cells that are not cancer cells. In some embodiments, the target cells are a plurality of cancer cells that have lost expression of the non-target antigen through loss of heterozygosity (LOH). In some embodiments, the non-target cells are a plurality of healthy cells (i.e., non-cancer cells), that express both the target and the non-target antigen.

In some embodiments, the HLA (for example HLA-A*02) is lost in the cancer cells due to loss of heterozygosity.

Non-target major histocompatibility complex class I MHC-I (or pMHC-I) antigens comprising any of HLA-A are envisaged as within the scope of the disclosure. In some embodiments, the non-target antigen comprises a Major Histocompatibility Complex (MHC) protein. In some embodiments, the MHC is MHC class I. In some embodiments, the MHC class I protein comprises a human leukocyte antigen (HLA) protein. In some embodiments, the non-target antigen comprises an allele of an HLA Class I protein selected from the group consisting of HLA-A. In some embodiments, the HLA-A allele comprises HLA-A*03. In some embodiments, the HLA-A allele comprises HLA-A*02.

In some embodiments, the ligand binding domain of the inhibitory receptor comprises an scFv. In some embodiments, the scFv binds to HLA-A*03. In some embodiments, the scFv binds to HLA-A*02.

In some embodiments, the non-target antigen comprises HLA-A*03, and the extracellular ligand binding domain of the inhibitory receptor comprises an HLA-A*03 ligand binding domain. In some embodiments, the HLA-A*03 ligand binding domain comprises an scFv domain.

In some embodiments, the non-target antigen comprises HLA-A*03, and the ligand binding domain of the inhibitory receptor comprises an HLA-A*03 ligand binding domain. In some embodiments, the ligand binding domain binds HLA-A*03 independent of the peptide in a pMHC complex comprising HLA-A*03. In some embodiments, the HLA-A*03 ligand binding domain comprises an scFv domain.

In some embodiments, the HLA-A*03 antigen binding domain comprises a heavy chain and a light chain.

In some embodiments, the antigen binding domain comprises a variable light chain region (VL) comprising a sequence set forth in 4A or 4B. In some embodiments, the antigen binding domain comprises a VL comprising a sequence having at least 90%, at least 95%, at least 97%, at least 98% or at least 99% identity a sequence set forth in 4A or 4B.

In each case, the VH may be paired with any of the VLs, as the heavy chains and light chains share similarity, with routine testing to confirm desired expression and binding activity.

In some embodiments, the VH and VL are separated by a linker, for example GGGGSGGGGSGGGGSGG (SEQ ID NO: 152). In some embodiments, the VH and VL are ordered, from N to C terminal, VH, linker and VL. In some embodiments, the VH and VL are ordered, from N to C terminal, VL, linker and VH.

In some embodiments, the non-target antigen comprises HLA-A*02, and the extracellular ligand binding domain of the inhibitory receptor comprises an HLA-A*02 ligand binding domain. In some embodiments, the HLA-A*02 ligand binding domain comprises an scFv domain. Such scFv's include, for example and without limitation the following mouse and humanized scFv antibodies that bind HLA-A*02 in a peptide-independent way (complementarity determining regions (CDRs) underlined):

HLA-A*02 ScFv: C-001765
(SEQ ID NO: 112)
MMTQTPLSLPVSLGDQASISCRSSQSIVHSNGNTYLEWYLQKPGQSPKLLIYKVSNRFSGVPDRFSGSGSGT
DFTLKISRVEAEDLGVYYCFQGSHVPRTSGGGTKLEIKGGGGSGGGGSGGGGSGGQVQLQQSGPELVKPGAS
VRISCKASGYTFTSYHIHWVKQRPGQGLEWIGWIYPGNVNTEYNEKFKGKATLTADKSSSTAYMHLSSLTSE
DSAVYFCAREEITYAMDYWGQGTSVTVSSYG;
or
(SEQ ID NO: 113)
DVLMTQTPLSLPVSLGDQASISCRSSQSIVHSNGNTYLEWYLQKPGQSPKLLIYKVSNRFSGVPDRFSGSGS
GTDFTLKISRVEAEDLGVYYCFQGSHVPRTSGGGTKLEIKGGGGSGGGGSGGGGSGGQVQLQQSGPELVKPG
ASVRISCKASGYTFTSYHIHWVKQRPGQGLEWIGWIYPGNVNTEYNEKFKGKATLTADKSSSTAYMHLSSLT
SEDSAVYFCAREEITYAMDYWGQGTSVTVSS
HLA-A*02 ScFv: C-002159
(SEQ ID NO: 114)
QLVQSGAEVKKPGSSVKVSCKASGYTFTSYHIHWVRQAPGQGLEWMGWIYPGNVNTEYNEKFKGKATITADK
STSTAYMELSSLRSEDTAVYYCAREEITYAMDYWGQGTTVTVSSGGGGSGGGGSGGGGSGGEIVLTQSPGTL
SLSPGERATLSCRSSQSIVHSNGNTYLEWYQQKPGQAPRLLIYKVSNRFSGIPDRFSGSGSGTDFTLTISRL
EPEDFAVYYCFQGSHVPRTFGGGTKVEIK
HLA-A*02 ScFv: C-002160
(SEQ ID NO: 115)
QLVQSGAEVKKPGSSVKVSCKASGYTFTSYHIHWVRQAPGQGLEWMGWIYPGNVNTEYNEKFKGKATITADK
STSTAYMELSSLRSEDTAVYYCAREEITYAMDYWGQGTTVTVSSGGGGSGGGGSGGGGSGGDIVMTQTPLSL
PVTPGEPASISCRSSQSIVHSNGNTYLEWYLQKPGQSPQLLIYKVSNRFSGVPDRFSGSGSGTDFTLKISRV
EAEDVGVYYCFQGSHVPRTFGGGTKVEIK
HLA-A*02 ScFv: C-002161
(SEQ ID NO: 116)
QLVESGGGLVKPGGSLRLSCAASGYTFTSYHIHWVRQAPGKGLEWVGWIYPGNVNTEYNEKFKGRFTISRDD
SKNTLYLQMNSLKTEDTAVYYCAREEITYAMDYWGQGTTVTVSSGGGGSGGGGSGGGGSGGDIQMTQSPSSL
SASVGDRVTITCRSSQSIVHSNGNTYLEWYQQKPGKAPKLLIYKVSNRFSGVPSRFSGSGSGTDFTLTISSL
QPEDFATYYCFQGSHVPRTFGGGTKVEIK
HLA-A*02 ScFv: C-002162
(SEQ ID NO: 117)
QLVQSGAEVKKPGSSVKVSCKASGYTFTSYHIHWVRQAPGQGLEWIGWIYPGNVNTEYNEKFKGKATITADE
STNTAYMELSSLRSEDTAVYYCAREEITYAMDYWGQGTLVTVSSGGGGSGGGGSGGGGSGGDIQMTQSPSTL
SASVGDRVTITCRSSQSIVHSNGNTYLEWYQQKPGKAPKLLIYKVSNRFSGVPARFSGSGSGTEFTLTISSL
QPDDFATYYCFQGSHVPRTFGQGTKVEVK
HLA-A*02 ScFv: C-002163
(SEQ ID NO: 118)
QLVQSGAEVKKPGSSVKVSCKASGYTFTSYHMHWVRQAPGQGLEWIGYIYPGNVNTEYNEKFKGKATLTADK
STNTAYMELSSLRSEDTAVYFCAREEITYAMDYWGQGTLVTVSSGGGGSGGGGSGGGGSGGDVQMTQSPSTL
SASVGDRVTITCSSSQSIVHSNGNTYMEWYQQKPGKAPKLLIYKVSNRFSGVPDRFSGSGSGTEFTLTISSL
QPDDFATYYCHQGSHVPRTFGQGTKVEVK
HLA-A*02 ScFv: C-002164
(SEQ ID NO: 119)
QVQLQQSGPELVKPGASVKMSCKASGYTFTSYHIQWVKQRPGQGLEWIGWIYPGDGSTQYNEKFKGKTTLTA
DKSSSTAYMLLSSLTSEDSAIYFCAREGTYYAMDYWGQGTSVTVSSGGGGSGGGGSGGGGSGGDVLMTQTPL
SLPVSLGDQVSISCRSSQSIVHSNGNTYLEWYLQKPGQSPKLLIYKVSNRFSGVPDRFSGSGSGTDFTLKIS
RVEAEDLGVYYCFQGSHVPRTFGGGTKLEIK
HLA-A*02 ScFv: C-002165
(SEQ ID NO: 120)
QLQLQESGPGLVKPSETLSLTCTVSGYTFTSYHIQWIRQPPGKGLEWIGWIYPGDGSTQYNEKFKGRATISV
DTSKNQFSLNLDSVSAADTAIYYCAREGTYYAMDYWGKGSTVTVSSGGGGSGGGGSGGGGSGGDIQMTQSPS
SLSASVGDRVTITCRSSQSIVHSNGNTYLEWYQQKPGKAPKLLIYKVSNRFSGVPSRFSGSGSGTDFTFTIS
SLQPEDIATYYCFQGSHVPRTFGPGTKVDIK
HLA-A*02 ScFv: C-002166
(SEQ ID NO: 121)
EVQLVQSGAELKKPGSSVKVSCKASGYTFTSYHIQWVKQAPGQGLEWIGWIYPGDGSTQYNEKFKGKATLTV
DKSTNTAYMELSSLRSEDTAVYYCAREGTYYAMDYWGQGTLVTVSSGGGGSGGGGSGGGGSGGDIQMTQSPS
TLSASVGDRVTITCRSSQSIVHSNGNTYLEWYQQKPGKAPKLLIYKVSNRFSGVPSRFSGSGSGTDFTLTIS
SLQPDDFATYYCFQGSHVPRTFGQGTKVEVK
HLA-A*02 ScFv: C-002167
(SEQ ID NO: 122)
QVQLVQSGAEVKKPGSSVKVSCKASGYTFTSYHIQWVRQAPGQGLEWMGWIYPGDGSTQYNEKFKGRVTITA
DKSTSTAYMELSSLRSEDTAVYYCAREGTYYAMDYWGQGTTVTVSSGGGGSGGGGSGGGGSGGEIVLTQSPG
TLSLSPGERATLSCRSSQSIVHSNGNTYLEWYQQKPGQAPRLLIYKVSNRFSGIPDRFSGSGSGTDFTLTIS
RLEPEDFAVYYCFQGSHVPRTFGGGTKVEIK
HLA-A*02 ScFv: C-002168
(SEQ ID NO: 123)
QVTLKQSGAEVKKPGSSVKVSCTASGYTFTSYHVSWVRQAPGQGLEWLGRIYPGDGSTQYNEKFKGKVTITA
DKSMDTSFMELTSLTSEDTAVYYCAREGTYYAMDLWGQGTLVTVSSGGGGSGGGGSGGGGSGGEIVLTQSPG
TLSLSPGERATLSCRSSQSIVHSNGNTYLAWYQQKPGQAPRLLISKVSNRFSGVPDRFSGSGSGTDFTLTIS
RLEPEDFAVYYCQQGSHVPRTFGGGTKVEIK
HLA-A*02 ScFv: C-002169
(SEQ ID NO: 124)
QVQLVQSGAEVKKPGASVKVSCKASGYTFTSYHMHWVRQAPGQRLEWMGWIYPGDGSTQYNEKFKGKVTITR
DTSASTAYMELSSLRSEDTAVYYCAREGTYYAMDYWGQGTLVTVSSGGGGSGGGGSGGGGSGGDIVMTQTPL
SLPVTPGEPASISCRSSQSIVHSNGNTYLDWYLQKPGQSPQLLIYKVSNRFSGVPDRFSGSGSGTDFTLKIS
RVEAEDVGVYYCMQGSHVPRTFGGGTKVEIK

### Differentially Expressed Inhibitor Ligands

The disclosure provides inhibitor ligands (non-target antigens, e.g., HLA-A*03 or HLA-A*02) that are differentially expressed between cancer cells and normal cells.

Activation of the inhibitory receptor is mediated by the presence of HLA-A*03 on the surface of a cell. A cell that expresses HLA-A*03 will activate the inhibitory receptor based on the level of expression of the HLA-A*03. In some embodiments, the HLA-A*03 is expressed by both target and non-target cells. However, in these embodiments, the HLA-A*03 is expressed by non-target cells at a higher level than the target cells. The higher levels of HLA-A*03 expressed by the non-target cells activate the inhibitory receptor, thereby preventing activation of the immune cell. In contrast, the lower levels of HLA-A*03 expressed by the target are not sufficient to activate the inhibitory receptor, leading to activation of the immune cell.

In alternative embodiments, the HLA-A*03 is expressed by non-target cells but not by target cells. In the absence of expression of the HLA-A*03, the target cells activate the target receptor, thereby activating the immune cells.

Differential expression can be determined by any techniques known in the art used to measure expression. These include, inter alia, techniques for measuring mRNA and/or protein levels of a target gene in a cell. Methods of measuring protein levels in samples include immunohistochemistry, enzyme-linked immunosorbent assays (ELISA), and analytical methods such as liquid chromatography-mass spectrometry (LC-MS). Methods of measuring mRNA levels include real time quantitative reverse transcription PCR (qRT-PCR), as well as high throughput sequencing. Expression differences can be observed between, for example, a normal cell and a diseased cell, for example a cancer cell.

Activation of the inhibitory receptor by HLA-A*03 can occur according to various modalities known in the art. Activation of the inhibitory receptor by HLA-A*03 can be determined by methods known in the art. For example, the level of downstream intracellular signaling in a cell expressing the inhibitory receptor can be measured through the use of a reporter gene.

Without wishing to be bound by theory, whether or not expression of HLA-A*03 inhibits activation of an immune cell via activation of the inhibitory receptor can occur according to the ratio of the HLA-A*03 to the inhibitor receptor. The expression levels of the HLA-A*03 and the inhibitory receptor, and the ratio thereof, can be determined by methods known in the art, including, inter alia, immunohistochemistry and fluorescence activated cell sorting (FACS). Analysis of the expression levels of the HLA-A*03 on target and non-target cells can be used to predict selective targeting of the immune cells expressing the inhibitory receptor. Low or no expression of the HLA-A*03 on a target or non-target cell can indicate, for example, that the inhibitory receptor will not be activated in an immune cell of the disclosure.

Alternatively, or in addition, and without wishing to be bound by theory, inhibition of immune cell activation by HLA-A*03 via activation of the inhibitory receptor can depend on the affinity of the HLA-A*03 for the inhibitory receptor. Methods of measuring affinity are known in the art, and include, inter alia, enzyme-linked immunosorbent assay or radioimmunoassay methods.

Alternatively, or in addition, and without wishing to be bound by theory, inhibition of immune cell activation by HLA-A*03 via activation of the inhibitory receptor can occur according to cross talk between the inhibitory receptor and the activator receptor, leading to down-regulation of the activity of the activator receptor. For example, activation of the inhibitory receptor by HLA-A*03 can lead to reduced expression of the activator receptor on the surface of the immune cell.

In some embodiments, HLA-A*03 is expressed at a lower level in a target cell than a normal cell. In some embodiments, the HLA-A*03 is expressed by healthy cells, i.e., cells that are not cancer cells. In some embodiments, HLA-A*03 expression level is at least about 10 times less, at least about 30 times less, at least about 50 times less, at least about 70 times less, at least about 90 times less, at least about 100 times less, at least about 110 times less, at least about 150 times less, at least about 200 times less, at least about 250 times less, at least about 300 times less, at least about 350 times less, at least about 400 times less, at least about 450 times less, at least about 500 times less, at least about 600 times less, at least about 700 times less, at least about 800 times less, at least about 900 times less or at least about 1000 times less in the target cell than in the non-target cell. In some embodiments, the HLA-A*03 expression level is about 10 times less, about 30 times less, about 50 times less, about 70 times less, about 90 times less, about 100 times less, or about 110 times less than the plurality of healthy cells. In some embodiments, the HLA-A*03 expression level is at least about 5 times less in the plurality of cancer cells than in the plurality of healthy cells. In some embodiments, the HLA-A*03 expression level is at least about 5 times less in a target cell than a non-target cell. In some embodiments, the target cells are a plurality of cancer cells that have low or no expression of HLA-A*03.

### Polynucleotides and Vectors

The disclosure provides polynucleotides encoding the sequence(s) of the activator and inhibitory receptors of the disclosure. The disclosure provides cells comprising the polynucleotides and vectors described herein.

In some embodiments, the sequence of the activator and/or inhibitory receptor is operably linked to a promoter. In some embodiments, the sequence encoding the activator receptor is operably linked to a first promoter, and the sequence encoding the inhibitory receptor is operably linked to a second promoter.

The disclosure provides vectors comprising the polynucleotides described herein.

In some embodiments, the activator receptor is encoded by a first vector and the inhibitory receptor is encoded by a second vector. In some embodiments, both receptors are encoded by a single vector.

In some embodiments, both receptors are encoded by a single vector. In some embodiments the vector comprises an shRNA, for example a B2M shRNA.

In some embodiments, the activator and inhibitory receptors are encoded by a single vector. Methods of encoding multiple polypeptides using a single vector will be known to persons of ordinary skill in the art, and include, inter alia, encoding multiple polypeptides under control of different promoters, or, if a single promoter is used to control transcription of multiple polypeptides, use of sequences encoding internal ribosome entry sites (IRES) and/or self-cleaving peptides. Exemplary self-cleaving peptides include T2A, P2A, E2A and F2A self-cleaving peptides. In some embodiments, the T2A self-cleaving peptide comprises a sequence of EGRGSLLTCGDVEENPGP (SEQ ID NO: 78). In some embodiments, the P2A self-cleaving peptide comprises a sequence of ATNFSLLKQAGDVEENPGP (SEQ ID NO: 79). In some embodiments, the E2A self-cleaving peptide comprises a sequence of QCTNYALLKLAGDVESNPGP (SEQ ID NO: 80). In some embodiments, the F2A self-cleaving peptide comprises a sequence of VKQTLNFDLLKLAGDVESNPGP (SEQ ID NO: 81). In some embodiments, the T2A self-cleaving peptide comprises a sequence of EGRGSLLTCGDVEENPGP (SEQ ID NO: 78). Any of the foregoing can also include an N terminal GSG linker. For example, a T2A self-cleaving peptide can also comprise a sequence of GSGEGRGSLLTCGDVEENPGP (SEQ ID NO: 82), which can be encoded by a sequence of:

(SEQ ID NO: 83)
GGATCCGGAGAGGGCAGAGGCAGCCTGCTGACATGTGGCGACGTGGAAG
AGAACCCTGGCCCC.

In some embodiments, the vector is an expression vector, i.e., for the expression of the activator and/or inhibitory receptor in a suitable cell.

Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

The expression of natural or synthetic nucleic acids encoding receptors is typically achieved by operably linking a nucleic acid encoding the receptor or portions thereof to a promoter and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

The polynucleotides encoding the receptors can be cloned into a number of types of vectors. For example, the polynucleotides can be cloned into a vector including, but not limited to a plasmid, a phagemid, a phage derivative, an animal virus, and cosmid. Vectors of particular interest include expression vectors, replication vectors, probe generation vectors, and sequencing vectors.

Further, the expression vector may be provided to cells, such as immune cells, in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcriptional initiation. Typically, these are located in the region 30-110 base pairs (bp) upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, a U6 promoter, as well as human gene promoters such as, but not limited to, the actin promoter, the myosin promoter, the hemoglobin promoter, and the creatine kinase promoter. Further, the disclosure should not be limited to the use of constitutive promoters. Inducible promoters are also contemplated as part of the disclosure. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In order to assess the expression of a receptor, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected or transduced cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). One method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Regardless of the method used to introduce exogenous nucleic acids into a host cell or otherwise expose a cell to the inhibitor of the present disclosure, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the disclosure.

### Cells

The disclosure provides cells comprising the receptors, vectors and polynucleotides described herein.

In some embodiments, the cell is an immune cell or a cell capable of differentiating into an immune cell.

As used herein, the term “immune cell” refers to a cell involved in the innate or adaptive (acquired) immune systems. Exemplary innate immune cells include phagocytic cells such as neutrophils, monocytes and macrophages, Natural Killer (NK) cells, polymorphonuclear leukocytes such as neutrophils eosinophils and basophils and mononuclear cells such as monocytes, macrophages and mast cells. Immune cells with roles in acquired immunity include lymphocytes such as T-cells and B-cells.

As used herein, a “T-cell” refers to a type of lymphocyte that originates from a bone marrow precursor that develops in the thymus gland. There are several distinct types of T-cells which develop upon migration to the thymus, which include, helper CD4+ T-cells, cytotoxic CD8+ T cells, memory T cells, regulatory CD4+ T-cells and stem memory T-cells. Different types of T-cells can be distinguished by the ordinarily skilled artisan based on their expression of markers. Methods of distinguishing between T-cell types will be readily apparent to the ordinarily skilled artisan.

In some embodiments, the activator receptor and the inhibitory receptor together specifically activate the immune cell in the presence of the target cell.

In some embodiments, the immune cell is CD4+, CD8+, a gamma delta T cell, an invariant T cells, an iNK cell, a NK cell, a macrophage, or combinations thereof. In some embodiments, the immune cell is a gamma delta (γδ) T cell. In some embodiments, the immune cell is an invariant T cell. In some embodiments, the immune cell is an invariant natural killer T cell (iNKT cell). In some embodiments, the immune cell is a T cell. In some embodiments, the immune cell is a B cell. In some embodiments, the immune cell is a Natural Killer (NK) cell. In some embodiments, the immune cell is CD8−. In some embodiments, the immune cell is CD8+. In some embodiments, the immune cell is CD4+. In some embodiments, the immune cell is CD4−. In some embodiments, the immune cell is CD8−/CD4+. In some embodiments, the immune cell is a CD8+CD4− T cell.

In some embodiments, the immune cell is non-natural. In some embodiments, the immune cell is isolated.

Methods transforming populations of immune cells, such as T cells, with the vectors of the instant disclosure will be readily apparent to the person of ordinary skill in the art. For example, CD3+ T cells can be isolated from PBMCs using a CD3+ T cell negative isolation kit (Miltenyi), according to manufacturer's instructions. T cells can be cultured at a density of 1×10\hat{ }6 cells/mL in X-Vivo 15 media supplemented with 5% human A/B serum and 1% Pen/strep in the presence of CD3/28 Dynabeads (1:1 cell to bead ratio) and 300 Units/mL of IL-2 (Miltenyi). After 2 days, T cells can be transduced with viral vectors, such as lentiviral vectors using methods known in the art. In some embodiments, the viral vector is transduced at a multiplicity of infection (MOI) of 5. Cells can then be cultured in IL-2 or other cytokines such as combinations of IL-7/15/21 for an additional 5 days prior to enrichment. Methods of isolating and culturing other populations of immune cells, such as B cells, or other populations of T cells, will be readily apparent to the person of ordinary skill in the art. Although this method outlines a potential approach it should be noted that these methodologies are rapidly evolving. For example, excellent viral transduction of peripheral blood mononuclear cells can be achieved after 5 days of growth to generate a >99% CD3+ highly transduced cell population.

Methods of activating and culturing populations of T cells comprising the TCRs, CARs, inhibitory receptors, receptors or vectors encoding same, will be readily apparent to the person of ordinary skill in the art.

Whether prior to or after genetic modification of T cells to express a TCR, the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041, 10040846; and U.S. Pat. Appl. Pub. No. 2006/0121005.

In some embodiments, T cells of the instant disclosure are expanded and activated in vitro. Generally, the T cells of the instant disclosure are expanded in vitro by contact with a surface having attached thereto an agent that stimulates a CD3/TCR complex associated signal and a ligand that stimulates a co-stimulatory molecule on the surface of the T cells. In particular, T cell populations may be stimulated as described herein, such as by contact with an anti-CD3 antibody. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody can be used. Examples of an anti-CD28 antibody include 9.3, B-T3, XR-CD28 (Diaclone, Besangon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

In some embodiments, the primary stimulatory signal and the co-stimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In some embodiments, the agent providing the co-stimulatory signal is bound to a cell surface and the agent providing the primary activation signal is in solution or coupled to a surface. In certain embodiments, both agents can be in solution. In another embodiment, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present disclosure.

In some embodiments, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the co-stimulatory signal is an anti-CD28 antibody or antigen-binding fragment thereof, and both agents are co-immobilized to the same bead in equivalent molecular amounts. In one embodiment, a 1:1 ratio of each antibody bound to the beads for CD4+ T cell expansion and T cell growth is used. In some embodiments, the ratio of CD3:CD28 antibody bound to the beads ranges from 100:1 to 1:100 and all integer values there between. In one aspect of the present disclosure, more anti-CD28 antibody is bound to the particles than anti-CD3 antibody, i.e., the ratio of CD3:CD28 is less than one. In certain embodiments of the disclosure, the ratio of anti CD28 antibody to anti CD3 antibody bound to the beads is greater than 2:1.

Ratios of particles to cells from 1:500 to 500:1 and any integer values in between may be used to stimulate T cells or other target cells. As those of ordinary skill in the art can readily appreciate, the ratio of particles to cells may depend on particle size relative to the target cell. For example, small sized beads could only bind a few cells, while larger beads could bind many. In certain embodiments the ratio of cells to particles ranges from 1:100 to 100:1 and any integer values in-between and in further embodiments the ratio comprises 1:9 to 9:1 and any integer values in between, can also be used to stimulate T cells. In some embodiments, a ratio of 1:1 cells to beads is used. One of skill in the art will appreciate that a variety of other ratios may be suitable for use in the present disclosure. In particular, ratios will vary depending on particle size and on cell size and type.

In further embodiments of the present disclosure, the cells, such as T cells, are combined with agent-coated beads, the beads and the cells are subsequently separated, and then the cells are cultured. In an alternative embodiment, prior to culture, the agent-coated beads and cells are not separated but are cultured together. In a further embodiment, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached to contact the T cells. In one embodiment the cells (for example, CD4+ T cells) and beads (for example, DYNABEADS CD3/CD28 T paramagnetic beads at a ratio of 1:1) are combined in a buffer. Again, those of ordinary skill in the art can readily appreciate any cell concentration may be used. In certain embodiments, it may be desirable to significantly decrease the volume in which particles and cells are mixed together (i.e., increase the concentration of cells), to ensure maximum contact of cells and particles. For example, in one embodiment, a concentration of about 2 billion cells/ml is used. In another embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. In some embodiments, cells that are cultured at a density of 1×10⁶ cells/mL are used.

In some embodiments, the mixture may be cultured for several hours (about 3 hours) to about 14 days or any hourly integer value in between. In another embodiment, the beads and T cells are cultured together for 2-3 days. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. In some embodiments, the media comprises X-VIVO-15 media supplemented with 5% human A/B serum, 1% penicillin/streptomycin (pen/strep) and 300 Units/ml of IL-2 (Miltenyi).

The T cells are maintained under conditions necessary to support growth, for example, an appropriate temperature (e.g., 37 °C) and atmosphere (e.g., air plus 5% CO2).

In some embodiments, the T cells comprising TCRs, CARs and inhibitory receptors of the disclosure are autologous. Prior to expansion and genetic modification, a source of T cells is obtained from a subject. Immune cells such as T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present disclosure, any number of T cell lines available in the art, may be used. In certain embodiments of the present disclosure, T cells can be obtained from a unit of blood collected from a subject using any number of techniques known to the skilled artisan, such as Ficoll™ separation.

In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing steps. In some embodiments, the cells are washed with phosphate buffered saline (PBS). In alternative embodiments, the wash solution lacks calcium and may lack magnesium or may lack many if not all divalent cations. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca2+-free, Mg2+-free PBS, PlasmaLyte A, or other saline solution with or without buffer. Alternatively, the undesirable components of the apheresis sample may be removed, and the cells directly resuspended in culture media.

In some embodiments, immune cells such as T cells are isolated from peripheral blood lymphocytes by lysing the red blood cells and depleting the monocytes, for example, by centrifugation through a PERCOLL™ gradient or by counterflow centrifugal elutriation. Specific subpopulations of immune cells, such as T cells, B cells, or CD4+ T cells can be further isolated by positive or negative selection techniques. For example, in one embodiment, T cells are isolated by incubation with anti-CD4-conjugated beads, for a time period sufficient for positive selection of the desired T cells.

Enrichment of an immune cell population, such as a T cell population, by negative selection can be accomplished with a combination of antibodies directed to surface markers unique to the negatively selected cells. One method is cell sorting and/or selection via negative magnetic immune-adherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD 14, CD20, CD 11b, CD 16, HLA-DR, and CD8.

For isolation of a desired population of immune cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads.

In some embodiments, the cells may be incubated on a rotator for varying lengths of time at varying speeds at either 2-10 °C or at room temperature.

T cells for stimulation, or PBMCs from which immune cells such as T cells are isolated, can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80 °C at a rate of 1 per minute and stored in the vapor phase of a liquid nitrogen storage tank. Other methods of controlled freezing may be used as well as uncontrolled freezing immediately at −20 °C or in liquid nitrogen.

The disclosure provides an immune cell expressing the activator and/or blocker receptors described herein, wherein the immune cell has reduced expression and/or function the major histocompatibility (MHC) class I complex.

In some embodiments, the immune cell is autologous. For example, the immune cells are isolated or derived from same subject who will receive the cell as part of a therapeutic regimen. It can be advantageous to modify autologous immune cells to have reduced expression and/or function of MHC class I with the blocker receptor is specific to an MHC class I antigen. Without wishing to be bound by theory, modification of autologous immune cells to have reduced expression and/or function of MHC class I reduces binding of the blocker receptor by MHC class I expressed by the immune cells, either in cis or in trans.

In some embodiments, the immune cell is all allogeneic. Allogeneic immune cells can be derived from a donor other than the subject to which the immune cells will be administered. Allogeneic immune cells have been commonly referred to in cell therapy as “off-the-shelf” or “universal” because of the possibility for allogeneic cells to be prepared and stored for use in subjects of a variety of genotypes.

Any suitable methods of reducing expression and/or function the MHC class I complex are envisaged as within the scope of the instant disclosure, and include, inter alia, expression of interfering RNAs that knock down one or more RNAs encoding MHC class I components, or modifications of genes encoding MHC class I components.

The major histocompatibility complex (NMC) is a locus on the vertebrate genome that encodes a set of polypeptides required for the adaptive immune system. Among these are MHC class I polypeptides that include HLA-A, HLA-B, and HLA-C and alleles thereof. MHC class I alleles are highly polymorphic and expressed in all nucleated cells. MHC class I polypeptides encoded by HLA-A, HLA-B, and HLA-C and alleles thereof form heterodimers with β2 microglobulin (B2M) and present in complex with antigens on the surface of cells. As referred to herein, an MHC class I gene or polypeptide may refer to any polypeptide found in the MHC, or the corresponding gene encoding said polypeptide. In some embodiments, the immune cells of the disclosure are inactivated by an inhibitor ligand comprising an MHC class I polypeptide, e.g., HLA-A, HLA-B, and HLA-C and alleles thereof. HLA-A alleles can be, for example and without limitation, HLA-A*02, HLA-A*02:01, HLA-A*02:01:01, HLA-A*02:01:01:01, and/or any gene that encodes protein identical or similar to HLA-A*02 protein. Thus, to prevent autocrine signaling/binding as described herein, it is desirable to eliminate or reduce expression of polypeptides encoded by HLA-A, HLA-B, and HLA-C and alleles thereof in the immune cells.

Immune Cells with Reduced MHC Class I Polypeptide Expression

In some embodiments, the genetically engineered immune cells described herein are modified to reduce or eliminate expression of the B2M gene product. The beta-2 microglobulin (B2M) gene encodes a protein that associates with the major histocompatibility complex (MHC) class I, i.e., MHC-I complexes. The MHC-I complex is required for presentation of antigens on the cell surface. The MHC-I complex is disrupted and non-functional when the B2M is deleted (Wang D et al. Stem Cells Transl Med. 4:1234-1245 (2015)). Furthermore, the B2M gene can be disrupted with high efficiency using gene editing techniques known in the art (Ren et al. Clin. Cancer Res. 23:2255-2266 (2017)). Reducing or eliminating B2M can reduce or eliminate functional MHC I on the surface of the immune cell.

In some embodiments, the expression and of function of B2M is reduced using gene editing systems. The disclosure provides gene editing systems for editing an endogenous target gene in an immune cell. The disclosure provides interfering RNAs specific to sequences of target genes. Gene editing systems such as CRISPR/Cas systems, TALENs and zinc fingers can be used to generate double strand breaks, which, through gene repair mechanisms such as homology directed repair or non-homologous end joining (NHEJ), can be used to introduce mutations. NHEJ after resection of the ends of the break, or improper end joining, can be used to introduce deletions. In some embodiments, the target gene comprises a gene encoding a subunit of the MIC-I complex.

Target gene sequences include, but are not limited to, promoters, enhancers, introns, exons, intron/exon junctions, transcription products (pre-mRNA, mRNA, and splice variants), and/or 3′ and 5′ untranslated regions (UTRs). Any gene element or combination of gene elements may be targeted for the purpose of genetic editing in the immune cells described herein. Modifications to the target genes can be accomplished using any method known in the art to edit the target gene that results in altered or disrupted expression or function the target gene or gene product.

In some embodiments, a target gene is edited in the immune cells described herein using a nucleic acid guided endonuclease. Exemplary nucleic acid guided endonucleases include Class 2 Type II endonucleases, such as CRISPR/Cas9.

In some embodiments, the expression and of function of B2M is reduced using RNA interference. “RNAi” or “RNA interference” refers to the process of sequence-specific post-transcriptional gene silencing, mediated by double-stranded RNA (dsRNA). Duplex RNAs such as siRNA (small interfering RNA), miRNA (micro-RNA), shRNA (short hairpin RNA), ddRNA (DNA-directed RNA), piRNA (Piwi-interacting RNA), or rasiRNA (repeat associated siRNA) and modified forms thereof are all capable of mediating RNA interference. These dsRNA molecules may be commercially available or may be designed and prepared based on known sequence information.

### Pharmaceutical Compositions

The disclosure provides pharmaceutical compositions comprising immune cells comprising the first and second receptors of the disclosure and a pharmaceutically acceptable diluent, carrier or excipient.

Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; and preservatives.

In some embodiments, the cell expresses both the activator receptor and the inhibitory receptor. In some embodiments, at least about 50%, about 55%, about 60%, about 65%, about 70%, about 75%, about 80%, about 85%, about 90%, or about 95% of the cells express both the activator receptor and the inhibitory receptor. In some embodiments, at least 90% of the cells express both the activator receptor and the inhibitory receptor.

### Treating Cancer

Provided herein are methods of killing a plurality of cancer cells, or treating cancer, in a subject, comprising administering to the subject a therapeutically effective amount of a composition comprising immune cells comprising the first and second receptors of the disclosure. The immune cells express both receptors in the same cell.

Cancer is a disease in which abnormal cells divide without control and spread to nearby tissue. In some embodiments, the cancer comprises a liquid tumor or a solid tumor. Exemplary liquid tumors include leukemias and lymphomas. Cancers can arise in virtually an organ in the body, including epithelial tissues. Any cancer wherein a plurality of the cancer cells expresses the first, activator, ligand and do not express the second, inhibitor ligand is envisaged as within the scope of the instant disclosure. For example, MSLN positive cancers that can be treated using the methods described herein include mesothelioma, ovarian cancer, cervical cancer, uterine cancer, gastric cancer, pancreatic cancer, lung cancers such as lung adenocarcinomas, colorectal cancer and cholangiocarcinoma.

In some embodiments, the plurality of cancer cells express the target antigen. In some embodiments, the plurality cancer cells of the subject express MSLN. MSNL positive cancers include mesothelioma cancer, ovarian cancer, cervical cancer, colorectal cancer, esophageal cancer, head and neck cancer, kidney cancer, uterine cancer, gastric cancer, pancreatic cancer, lung cancer, lung adenocarcinomas, colorectal cancer, or cholangiocarcinoma, as well as other solid epithelial tumors. Further cancers that express MSLN include relapsed, refractory or metastatic gastric, esophageal, head and neck and kidney cancers. In some embodiments, the MSLN positive cancer comprises an epithelial tumor, for example a carcinoma.

Provided herein are methods of treating MSLN+ cancer in a subject having a MSLN+ tumor, the tumor having loss of heterozygosity at an MHC class I locus. In some embodiments, the methods comprise administering to the subject an effective amount of the immune cells or pharmaceutical compositions described herein.

In some embodiments, a plurality of cancer cells do not express a polymorphic allele of ICAM1, COMT or CXCL16. For example, the cancer cells have lost an allele of CAM1, COMT or CXCL16 through loss of heterozygosity at that locus.

In some embodiments, the plurality of cancer cells do not express, or have lower expression than normal cells, of LRRN4 or UPK3B.

Methods of genotyping cancer cells and normal cells from a subject for the presence or absence of SNPs will be readily apparent to persons of ordinary skill in the art. SNP genotyping methods include, inter alia, PCR based methods such as dual-probe TaqMan assays, array-based hybridization methods and sequencing.

Methods of measuring the expression of the target antigen in cancer or normal cells from a subject will be readily apparent to persons of ordinary skill in the art. These include, inter alia, methods of measuring RNA expression such as RNA sequencing and reverse transcription polymerase chain reaction (RT-PCR), as well as methods of measuring protein expression such as immunohistochemistry-based methods. Methods of measuring loss of heterozygosity in a plurality of cancer cells, include, inter alia, high throughput sequencing of genomic DNA extracted from cancer cells using methods known in the art.

The disclosure provides methods of treating a cancer in a subject comprising measuring the expression level of the non-target antigen in a plurality of cancer cells, and treating the subject when the expression level of the non-target antigen in the plurality of cancer cells is less than the expression level of the non-target antigen in the plurality of cancer cells is less than the expression level of the non-target antigen a plurality of healthy cells. In some embodiments, the non-target antigen comprises LRRN4 or UPKB3, or a peptide antigen of LRRN4 or UPKB3. In some embodiments, the methods comprise determining the expression of MSLN in a plurality of cancer cells; and administering a plurality of immune cells to the subject if the plurality of cancer cells have low or no expression of the non-target antigen, and the plurality of cancer cells are MSLN positive.

Methods of measuring the expression of the target antigen in cancer or cells from a subject will be readily apparent to persons of ordinary skill in the art. These include, inter alia, methods of measuring RNA expression such as RNA sequencing and reverse transcription polymerase chain reaction (RT-PCR), as well as methods of measuring protein expression such as immunohistochemistry-based methods.

In some embodiments, the immune cells are T cells.

In some embodiments, the immune cells are allogeneic or autologous.

In some embodiments, the second receptor increases the specificity of the immune cells for the MSLN positive cancer cells compared to immune cells that express the first receptor but do not express the second receptor. In some embodiments, the immune cells have reduced side effects compared to immune cells that express the first receptor but do not express the second receptor.

Administration of the immune cells or pharmaceutical compositions described herein can arrest the growth of a tumor in the subject. For example, the immune cells or pharmaceutical compositions can kill tumor cells, so that the tumor stops growing, or is reduced in size. In some cases, immune cells or pharmaceutical compositions can prevent formation of additional tumors or reduce the total number of tumors in the subject.

Administration of the immune cells or pharmaceutical compositions described herein can result in selective killing of a cancer cell but not a wild-type cell in the subject. In some embodiments, about 60% of the cells killed are cancer cells, about 65% of the cells killed are cancer cells, about 70% of the cells killed are cancer cells, about 75% of the cells killed are cancer cells, about 80% of the cells killed are cancer cells, about 85% of the cells killed are cancer cells, about 90% of the cells killed are cancer cells, about 95% of the cells killed are cancer cells, or about 100% of the cells killed are cancer cells.

Administration of the immune cells or pharmaceutical compositions described herein can result in the killing of about 40%, about 50%, about 60%, about 70%, about 80%, about 90% or all of the cancer cells of the subject.

Administration of the immune cells or pharmaceutical compositions described herein can result in fewer side effects for the subject than administration of an otherwise equivalent immune cell comprising the first activator receptor but no second inhibitory receptor. For example, administering the immune cells or pharmaceutical compositions described herein can reduce dose limited toxicity relative to the MSLN CAR, or MSLN TCR administered without the second inhibitory receptor.

Treating cancer can result in a reduction in size of a tumor. A reduction in size of a tumor may also be referred to as “tumor regression”. Preferably, after treatment, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. The size of a tumor may be measured as a diameter of the tumor.

Treating cancer can result in a reduction in tumor volume. Preferably, after treatment, tumor volume is reduced by 5% or greater relative to its size prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

Administration of the immune cells or pharmaceutical compositions described herein can reduce the size of a tumor in the subject. In some embodiments, the size of the tumor is reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, relative to the size of the tumor before administration of the immune cells or pharmaceutical compositions. In some embodiments, the tumor is eliminated.

Treating cancer results in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

Treating cancer can result in a decrease in number of metastatic lesions in other tissues or organs distant from the primary tumor site. Preferably, after treatment, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

Treating cancer can result in an increase in average survival time of a population of treated subjects in comparison to a population receiving carrier alone. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in an increase in average survival time of a population of treated subjects in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in increase in average survival time of a population of treated subjects in comparison to a population receiving monotherapy with a drug that is not a compound of the present disclosure, or a pharmaceutically acceptable salt, prodrug, metabolite, analog or derivative thereof. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in a decrease in the mortality rate of a population of treated subjects in comparison to a population receiving carrier alone. Treating cancer can result in a decrease in the mortality rate of a population of treated subjects in comparison to an untreated population. Treating cancer can result in a decrease in the mortality rate of a population of treated subjects in comparison to a population receiving monotherapy with a drug that is not a compound of the present disclosure, or a pharmaceutically acceptable salt, prodrug, metabolite, analog or derivative thereof. Preferably, the mortality rate is decreased by more than 2%; more preferably, by more than 5%; more preferably, by more than 10%; and most preferably, by more than 25%. A decrease in the mortality rate of a population of treated subjects may be measured by any reproducible means. A decrease in the mortality rate of a population may be measured, for example, by calculating for a population the average number of disease-related deaths per unit time following initiation of treatment with an active compound. A decrease in the mortality rate of a population may also be measured, for example, by calculating for a population the average number of disease-related deaths per unit time following completion of a first round of treatment with an active compound.

Treating cancer can result in a decrease in tumor growth rate. Preferably, after treatment, tumor growth rate is reduced by at least 5% relative to number prior to treatment; more preferably, tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. Tumor growth rate can be measured according to a change in tumor diameter per unit time.

Treating cancer can result in a decrease in tumor regrowth. Preferably, after treatment, tumor regrowth is less than 5%; more preferably, tumor regrowth is less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

Treating or preventing a cancer can result in a reduction in the rate of cellular proliferation. Preferably, after treatment, the rate of cellular proliferation is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The rate of cellular proliferation may be measured by any reproducible means of measurement. The rate of cellular proliferation is measured, for example, by measuring the number of dividing cells in a tissue sample per unit time.

Treating or preventing cancer can result in a reduction in the proportion of proliferating cells. Preferably, after treatment, the proportion of proliferating cells is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The proportion of proliferating cells may be measured by any reproducible means of measurement. Preferably, the proportion of proliferating cells is measured, for example, by quantifying the number of dividing cells relative to the number of nondividing cells in a tissue sample. The proportion of proliferating cells can be equivalent to the mitotic index.

Treating or preventing cancer can result in a decrease in size of an area or zone of cellular proliferation. Preferably, after treatment, size of an area or zone of cellular proliferation is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Size of an area or zone of cellular proliferation may be measured by any reproducible means of measurement. The size of an area or zone of cellular proliferation may be measured as a diameter or width of an area or zone of cellular proliferation.

Treating or preventing cancer can result in a decrease in the number or proportion of cells having an abnormal appearance or morphology. Preferably, after treatment, the number of cells having an abnormal morphology is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. An abnormal cellular appearance or morphology may be measured by any reproducible means of measurement. An abnormal cellular morphology can be measured by microscopy, e.g., using an inverted tissue culture microscope. An abnormal cellular morphology can take the form of nuclear pleiomorphism.

### Dosage and Administration

The cells and of the present disclosure may be administered in a number of ways depending upon whether local or systemic treatment is desired.

In general, administration may be parenteral.

Methods for administration of cells for adoptive cell therapy are known and may be used in connection with the provided methods and compositions. For example, adoptive T cell therapy methods are described, e.g., in US Patent Application Publication No. 2003/0170238 to Gruenberg et al and U.S. Pat. No. 4,690,915 to Rosenberg.

The compositions of the disclosure are suitable for parenteral administration. As used herein, “parenteral administration” of a pharmaceutical composition includes any route of administration characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal, intravenous, intraarterial, intrathecal, intraventricular, intraurethral, intracranial, intratumoral, intrasynovial injection or infusions, and kidney dialytic infusion techniques. In some embodiments, parenteral administration of the compositions of the present disclosure comprises intravenous or intraarterial administration.

The disclosure provides pharmaceutical compositions comprising a plurality of immune cells of the disclosure, and a pharmaceutically acceptable carrier, diluent or excipient.

Formulations of a pharmaceutical composition suitable for parenteral administration typically generally comprise of immune cells combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for parenteral administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. Parenteral formulations also include aqueous solutions which may contain excipients such as salts, carbohydrates and buffering agents. Exemplary parenteral administration forms include solutions or suspensions in sterile aqueous solutions, for example, aqueous propylene glycol or dextrose solutions. Such dosage forms can be suitably buffered, if desired. Formulations for parenteral administration may be formulated to be immediate and/or modified release. Modified release formulations include delayed-, sustained-, pulsed-, controlled-, targeted and programmed release.

In some embodiments, the formulated composition comprising the immune cells is suitable for administration via injection. In some embodiments, the formulated composition comprising the immune cells is suitable for administration via infusion.

The pharmaceutical compositions of the present disclosure, which may conveniently be presented in unit dosage form, may be prepared according to conventional techniques well known in the pharmaceutical industry. Such techniques include the step of bringing into association the immune cells with the pharmaceutical carrier(s) or excipient(s), such as liquid carriers.

Aqueous suspensions may further contain substances that increase the viscosity of the suspension including, for example, sodium carboxymethylcellulose, sorbitol and/or dextran. The suspension may also contain stabilizers.

The compositions of the present disclosure may additionally contain other adjunct components conventionally found in pharmaceutical compositions. Thus, for example, the compositions may contain additional, compatible, pharmaceutically active materials such as, for example, antipruritic, astringents, local anesthetics or anti-inflammatory agents, or may contain additional materials useful in physically formulating various dosage forms of the compositions of the present disclosure, such as dyes, preservatives, antioxidants, opacifiers, thickening agents and stabilizers. However, such materials, when added, should not unduly interfere with the biological activities of the immune cells of the compositions of the present disclosure.

The formulation or composition may also contain more than one active ingredient useful for the particular indication, disease, or condition being treated with the immune cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents.

The pharmaceutical composition in some aspects can employ time-released, delayed release, and sustained release delivery systems such that the delivery of the composition occurs prior to, and with sufficient time to cause, sensitization of the site to be treated. Many types of release delivery systems are available and known. Such systems can avoid repeated administrations of the composition, thereby increasing convenience to the subject and the physician.

Administration can be effected in one dose, continuously or intermittently throughout the course of treatment. Single or multiple administrations can be carried out with the dose level and pattern being selected by the treating physician.

The pharmaceutical composition in some embodiments contains the immune cells in amounts effective to treat or prevent a cancer, such as a therapeutically effective or prophylactically effective amount. Therapeutic or prophylactic efficacy in some embodiments is monitored by periodic assessment of treated subjects. For repeated administrations over days, weeks or months, depending on the condition, the treatment can be repeated until a desired suppression of cancer signs or symptoms occurs. However, other dosage regimens may be useful and can be determined. The desired dosage can be delivered by a single bolus administration or infusion of the composition or by multiple bolus administrations or infusions of the composition.

The cells or population of cells can be administrated in one or more doses. In some embodiments, an effective amount of cells can be administrated as a single dose. In some embodiments, an effective amount of cells can be administrated as more than one doses over a period time. Timing of administration is within the judgment of a managing physician and depends on the clinical condition of the patient.

The cells or population of cells may be obtained from any source, such as a blood bank or a donor, or the patient themselves.

An effective amount means an amount which provides a therapeutic or prophylactic benefit. The dosage administered will be dependent upon the age, health and weight of the recipient, kind of concurrent treatment, if any, frequency of treatment and the nature of the effect desired. In some embodiments, an effective amount of cells or composition comprising those cells are administrated parenterally. In some embodiments, administration can be an intravenous administration. In some embodiments, administration can be directly done by injection within a tumor.

For purposes of the disclosure, an assay, which comprises, for example, comparing the extent to which target cells are lysed or one or more cytokines are secreted by immune cells expressing the receptors, upon administration of a given dose of such immune cells to a mammal, among a set of mammals of which is each given a different dose of the immune cells, can be used to determine a starting dose to be administered to a mammal.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The immune cells of the disclosure are in some embodiments are co-administered with one or more additional therapeutic agents or in connection with another therapeutic intervention, either simultaneously or sequentially in any order. In some contexts, the immune cells are co-administered with another therapy sufficiently close in time such that the immune cell populations enhance the effect of one or more additional therapeutic agents, or vice versa. In some embodiments, the immune cells are administered prior to the one or more additional therapeutic agents. In some embodiments, the immune cells are administered after to the one or more additional therapeutic agents.

In embodiments, a lymphodepleting chemotherapy is administered to the subject prior to, concurrently with, or after administration (e.g., infusion) of adoptive immune cells. In an example, the lymphodepleting chemotherapy is administered to the subject prior to administration of the immune cells. For example, the lymphodepleting chemotherapy ends 1-4 days (e.g., 1, 2, 3, or 4 days) prior to adoptive cell infusion. In embodiments, multiple doses of adoptive cells are administered, e.g., as described herein. In embodiments, a lymphodepleting chemotherapy is administered to the subject prior to, concurrently with, or after administration (e.g., infusion) of the immune cells described herein. Examples of lymphodepletion include, but may not be limited to, nonmyeloablative lymphodepleting chemotherapy, myeloablative lymphodepleting chemotherapy, total body irradiation, etc. Examples of lymphodepleting agents include, but are not limited to, antithymocyte globulin, anti-CD3 antibodies, anti-CD4 antibodies, anti-CD8 antibodies, anti-CD52 antibodies, anti-CD2 antibodies, TCRaP blockers, anti-CD20 antibodies, anti-CD19 antibodies, Bortezomib, rituximab, anti-CD 154 antibodies, rapamycin, CD3 immunotoxin, fludarabine, cyclophosphamide, busulfan, melphalan, Mabthera, Tacrolimus, alefacept, alemtuzumab, OKT3, OKT4, OKT8, OKT11, fngolimod, anti-CD40 antibodies, anti-BR3 antibodies, Campath-1H, anti-CD25 antibodies, calcineurin inhibitors, mycophenolate, and steroids, which may be used alone or in combination. As a further example, a lymphodepletion regimen can include, administration of alemtuzumab, cyclophosphamide, benduamustin, rituximab, pentostatin, and/or fludarabine. Lymphodepletion regimen can be administered in one or more cycles until the desired outcome of reduced circulating immune cells. In some embodiments, the lymphodepletion comprises administering an agent that specifically targets, and reduces or eliminates CD52+ cells in the subject, and the immune cells are modified to reduce or eliminate CD52 expression.

In some embodiments, an immune stimulating therapy is administered to the subject prior to, concurrently with, or after administration (e.g., infusion) of adoptive immune cells. In some embodiments, the immune stimulating therapy comprises homeostatic cytokines. In some embodiments, the immune stimulating therapy comprises immune-stimulatory molecules. In some embodiments, the immune stimulating therapy comprises IL-2, IL-7, IL-12, IL-15, IL-21, IL-9, or a functional fragment thereof. In some embodiments, the immune stimulating therapy comprises IL-2, IL-7, IL-12, IL-15, IL-21, IL-9, or combinations thereof. In some embodiments, the immune stimulating therapy comprises IL-2, or a functional fragment thereof.

Methods for adoptive cell therapy using autologous cells includes isolating immune cells from patient blood, performing a series of modifications on the isolated cells including transducing the cells with one or more vectors encoding the dual receptor system described herein, and administering the cells to a patient. Providing immune cells from a subject suffering from or at risk for cancer or a hematological malignancy requires isolation of immune cell from the patient's blood and can be accomplished through methods known in the art, for example, by leukapheresis. During leukapheresis, blood from a subject is extracted and the peripheral blood mononuclear cells (PBMCs) are separated, and the remainder of the blood is returned to the subject's circulation. The PBMCs are stored either frozen or cryopreserved as a sample of immune cells and provided for further processing steps, such as, e.g., the modifications described herein.

In some embodiments, the method of treating a subject described herein comprises modifications to immune cells from the subject comprising a series of modifications comprising enrichment and/or depletion, activation, genetic modification, expansion, formulation, and cryopreservation.

The disclosure provides enrichment and/or depletion steps that can be, for example, washing and fractionating methods known in the art for preparation of subject PBMCs for downstream procedures, e.g., the modifications described herein. For example, without limitation, methods can include devices to remove gross red blood cells and platelet contaminants, systems for size-based cell fractionation for the depletion of monocytes and the isolation of lymphocytes, and/or systems that allow the enrichment of specific subsets of T cells, such as, e.g. CD4+, CD8+, CD25+, or CD62L+ T cells. Following the enrichment steps, a target sub-population of immune cells will be isolated from the subject PMBCs for further processing. Those skilled in the art will appreciate that enrichment steps, as provided herein, may also encompass any newly discovered method, device, reagent or combination thereof.

The disclosure provides activation steps that can be any method known in the art to induce activation of immune cells, e.g., T cells, required for their ex vivo expansion. Immune cell activation can be achieved, for example, by culturing the subject immune cells in the presence of dendritic cells, culturing the subject immune cells in the presence of artificial antigen-presenting cells (AAPCs), or culturing the immune cells in the presence of irradiated K562-derived AAPCs. Other methods for activating subject immune cells can be, for example, culturing the immune cells in the presence of isolated activating factors and compositions, e.g., beads, surfaces, or particles functionalized with activating factors. Activating factors can include, for example, antibodies, e.g., anti-CD3 and/or anti-CD28 antibodies. Activating factors can also be, for example, cytokines, e.g., interleukin (IL)-2 or IL-21. Activating factors can also be costimulatory molecules, such as, for example, CD40, CD40L, CD70, CD80, CD83, CD86, CD137L, ICOSL, GITRL, and CD134L. Those skilled in the art will appreciate that activating factors, as provided herein, may also encompass any newly discovered activating factor, reagent, composition, or combination thereof that can activate immune cells.

In some embodiments, a therapeutically effective dose of the immune cells described herein are administered. In some embodiments, the immune cells of the disclosure are administered by intravenous injection. In some embodiments, the immune cells of the disclosure are administered by intraperitoneal injection. In some embodiments, a therapeutically effective dose comprises about 0.5×10⁶ cells, about 1×10⁶ cells, about 2×10⁶ cells, about 3×10⁶ cells, 4×10⁶ cells, about 5×10⁶ cells, about 6×10⁶ cells, about 7×10⁶ cells, about 8×10⁶ cells, about 9×10⁶ cells, about 1×10⁷, about 2×10⁷, about 3×10⁷, about 4×10⁷, about 5×10⁷, about 6×10⁷, about 7×10⁷, about 8×10⁷, about 9×10⁷, about 1×10⁸ cells, about 2×10⁸ cells, about 3×10′ cells, about 4×10⁸ cells, about 5×10⁸ cells, about 6×10⁸ cells, about 7×10⁸ cells, about 8×10⁸ cells, about 9×10⁸ cells, about 1×10⁹ cells, about 2×10⁹ cells, about 3×10⁹ cells, about 3×10⁹ cells, about 4×10⁹ cells, about 5×10⁹ cells, about 5×10⁹ cells, about 6×10⁹ cells, about 7×10⁹ cells, about 8×10⁹ cells, about 9×10⁹ cells, about 1×10¹⁰ cells, about 2×10¹⁰ cells, about 3×10¹⁰ cells, about 4×10¹⁰ cells, about 5×10¹⁰ cells, about 6×10¹⁰ cells, about 7×10¹⁰ cells, about 8×10¹⁰ cells, or about 9×10¹⁰ cells.

In some embodiments, a therapeutically effective dose comprises about 0.5×10⁶ cells to about 9×10¹⁰ cells, about 1×10⁶ cells to about 5×10¹⁰ cells, about 2×10⁶ cells to about 5×10⁹ cells, about 3×10⁶ cells to about 5×10⁹ cells, about 4×10⁶ cells to about 3×10⁹ cells, about 5×10⁶ cells to about 2×10⁹ cells, about 6×10⁶ cells to about 1×10⁹ cells, 0.5×10⁶ cells to about 6×10⁹ cells, about 1×10⁶ cells to about 5×10⁹ cells, about 2×10⁶ cells to about 5×10⁹ cells, about 3×10⁶ cells to about 4×10⁹ cells, about 4×10⁶ cells to about 3×10⁹ cells, about 5×10⁶ cells to about 2×10⁹ cells, about 6×10⁶ cells to about 1×10⁹ cells, 0.5×10⁶ cells to about 6×10⁸ cells, about 1×10⁶ cells to about 5×10⁸ cells, about 2×10⁶ cells to about 5×10⁸ cells, about 3×10⁶ cells to about 4×10⁸ cells, about 4×10⁶ cells to about 3×10⁸ cells, about 5×10⁶ cells to about 2×10⁸ cells, about 6×10⁶ cells to about 1×10⁸ cells, about 7×10⁶ cells to about 9×10⁸ cells, about 8×10⁶ cells to about 8×10⁸ cells, about 9×10⁶ cells to about 7×10⁸ cells, about 1×10⁷ cells to about 6×10⁸ cells, about 2×10⁷ cells to about 5×10⁸ cells, about 7×10⁶ cells to about 9×10⁷ cells, about 8×10⁶ cells to about 8×10⁷ cells, about 9×10⁶ cells to about 7×10⁷ cells, about 1×10⁷ cells to about 6×10⁷ cells, or about 2×10⁷ cells to about 5×10⁷ cells.

In some embodiments, a therapeutically effective dose comprises about 0.5×10⁵ cells to about 9×10¹⁰ cells. In some embodiments, a therapeutically effective dose comprises about 0.5×10⁶ cells to about 1×10¹⁰ cells. In some embodiments, a therapeutically effective dose comprises about 0.5×10⁶ cells to about 5×10⁹ cells. In some embodiments, a therapeutically effective dose comprises about 0.5×10⁶ cells to about 1×10⁹ cells. In some embodiments, a therapeutically effective dose comprises about 0.5×10⁶ cells to about 6×10⁸ cells. In some embodiments, a therapeutically effective dose comprises about 0.5×10⁶ cells to about 9×10¹⁰ cells. In some embodiments, a therapeutically effective dose comprises about 0.5×10⁷ cells to about 1×10¹⁰ cells. In some embodiments, a therapeutically effective dose comprises about 0.5×10⁷ cells to about 5×10⁹ cells. In some embodiments, a therapeutically effective dose comprises about 0.5×10⁷ cells to about 1×10⁹ cells. In some embodiments, a therapeutically effective dose comprises about 0.5×10⁷ cells to about 6×10⁸ cells. In some embodiments, a therapeutically effective dose comprises about 0.5×10⁸ cells to about 9×10¹⁰ cells. In some embodiments, a therapeutically effective dose comprises about 0.5×10⁸ cells to about 1×10¹⁰ cells. In some embodiments, a therapeutically effective dose comprises about 0.5×10⁸ cells to about 5×10⁹ cells. In some embodiments, a therapeutically effective dose comprises about 0.5×10⁸ cells to about 1×10⁹ cells. The term “about” as referred to in a therapeutically dose, can be, for example, ±0.5×10⁶ cells, ±0.5×10⁷ cells, or 0.5×10⁸ cells.

### Kits and Articles of Manufacture

The disclosure provides kits and articles of manufacture comprising the polynucleotides and vectors encoding the receptors described herein, and cells comprising the receptors described herein. In some embodiments, the kit comprises articles such as vials, syringes and instructions for use.

In some embodiments, the kit comprises a polynucleotide or vector comprising a sequence encoding one or more receptors of the disclosure.

In some embodiments, the kit comprises a plurality of cells comprising the receptors as described herein. In some embodiments, the plurality of cells comprises a plurality of T cells.

In some embodiments, the kit further comprises instructions for use.

### ENUMERATED EMBODIMENTS

Embodiment 1: A method of producing an engineered cell responsive to targets cells expressing a first antigen and not a second antigen, the method comprising: identifying a first antigen and a second antigen having epitopes of defined heights; selecting one or more hinge elements based on the relative heights of the epitopes of the first antigen and the second antigen; generating polynucleotide sequences encoding: an activator receptor with a defined receptor height that specifically binds the epitope of the first antigen, and an inhibitory receptor with a defined receptor height that specifically binds the epitope of the second antigen, wherein either the activator receptor or the inhibitor receptor, or both, comprise at least one hinge element such that the combined height of the inhibitory receptor and second antigen epitope is less than or approximately equal to the combined height of the activator receptor and first antigen epitope; and introducing the polynucleotide sequences into a cell; thereby generating the engineered cell responsive to targets cells expressing the first antigen and not the second antigen, and nonresponsive to cells expressing both the first and second antigen.

Embodiment 2: A method of rescuing activity of paired inhibitory and activating receptors in an engineered cell, comprising: generating polynucleotide sequences encoding: an activator receptor that specifically binds an epitope of a first antigen, and an inhibitory receptor that specifically binds an epitope of a second antigen, wherein the activator receptor comprises at least one hinge element such that the combined height of the activator receptor and first antigen epitope is greater than the combined height of the inhibitory receptor and second antigen epitope; and introducing the polynucleotide sequences into a cell, thereby rescuing the activity of inhibitory and activating receptors in the engineered cell.

Embodiment 3: The method of Embodiment 1 or 2, wherein the cell is an immune cell or a cell capable of differentiating into an immune cell.

Embodiment 4: The method of any one of Embodiments 1 to 3, wherein the activator receptor and the inhibitory receptor each comprises at least one selected hinge element such that the combined height of the inhibitory receptor and second antigen epitope is approximately equal to the combined height of the activator receptor and first antigen.

Embodiment 5: The method of any one of Embodiments 1 to 3, wherein the activator receptor comprises a plurality of selected hinge elements such that the combined height of the activator receptor and the first antigen epitope is larger than the combined height of the inhibitory receptor and second antigen epitope.

Embodiment 6: The method of any one of Embodiments 1 to 3, wherein the height of the inhibitory receptor is reduced such that the combined height of the inhibitory receptor and second antigen epitope is less than the combined height of the activator receptor and first antigen epitope.

Embodiment 7: An engineered cell selectively responsive to target cells expressing a first antigen and not a second antigen, but nonresponsive to cells expressing both the first and second antigen, the engineered cell comprising: an activator receptor that specifically binds the first antigen, and an inhibitory receptor that specifically binds the second antigen, wherein the receptor that binds the shorter of the epitopes of two antigens comprises one or more hinge elements such that said receptor has a greater height than the other receptor.

Embodiment 8: The engineered cell of Embodiment 7, wherein the cell is an immune cell or a cell capable of differentiating into an immune cell.

Embodiment 9: The engineered cell of Embodiment 7 or 8, wherein the activator receptor and the inhibitory receptor each comprises one or more hinge elements.

Embodiment 10: The engineered cell of any one of Embodiments 7-9, wherein the one or more hinge elements are derived from polypeptides encoding a G4Q hinge, a thrombospondin (TSP) hinge, an Immunoglobulin (Ig) hinge, an Epidermal Growth Factor (EGF) hinge, a fibronectin III (FNIII) hinge, or combinations thereof.

Embodiment 11: The engineered cell of Embodiment 10, wherein the one or more hinge elements comprises a hinge element comprising a sequence of any one of SEQ ID NOs: 1-22, or a sequence having at least 85%, at least 90%, at least 95%, at least 97% or at least 99% identity thereto.

Embodiment 12: The engineered cell of any one of Embodiments 7-11, wherein the height of the inhibitory receptor is approximately equal to or less than the epitope height of the activator receptor.

Embodiment 13: The engineered cell of any one of Embodiments 7-12, wherein the first antigen is selected from MSLN, CD19, and CEA.

Embodiment 14: The engineered cell of any one of Embodiments 7-12, wherein the second antigen is selected from MSLN, LRRN4, and HLA-A2.

Embodiment 15: The engineered cell of any one of Embodiments 7-14, wherein the activator receptor is a chimeric antigen receptor (CAR).

Embodiment 16: The engineered cell of Embodiment 15, wherein the activator receptor comprises an scFv.

Embodiment 17: The engineered cell of Embodiment 16 wherein the scFv comprises a sequence of any one of SEQ ID NO: 29-31, or a sequence having at least 85%, at least 90%, at least 95%, at least 97% or at least 99% identity thereto.

Embodiment 18: The engineered cell of any one of Embodiments 15-17, wherein the CAR comprises a CD8 or CD28 transmembrane domain.

Embodiment 19: The engineered cell of any one of Embodiments 15-18, wherein the CAR comprises CD28 and/or 4-1BB intracellular domains and a CD3z intracellular domain.

Embodiment 20: The engineered cell of any one of Embodiments 15-19, wherein the activator receptor comprises a sequence having at least 85%, at least 90%, at least 95%, at least 97%, at least 99% identity to any one of SEQ ID NOs. 84-104.

Embodiment 21: The engineered cell of any one of Embodiments 7-20, wherein the inhibitory receptor is a chimeric antigen receptor (CAR).

Embodiment 22: The engineered cell of Embodiment 21, wherein the inhibitory receptor comprises an scFv.

Embodiment 23: The engineered cell of Embodiment 22, wherein the scFv comprises a sequence of SEQ ID NO: 28, 29, or 31, or a sequence having at least 85%, at least 90%, at least 95%, at least 97% or at least 99% identity thereto.

Embodiment 24: The engineered cell of any one of Embodiments 7-23, wherein the inhibitory receptor comprises a LILRB1 intracellular domain or a functional variant thereof.

Embodiment 25: The engineered cell of any one of Embodiments 7-24, wherein the inhibitory receptor comprises one or more EGF hinge elements.

Embodiment 26: The engineered cell of Embodiment 25, wherein the inhibitory receptor comprises 1, 2, 3, or 4 EGF hinge elements.

Embodiment 27: The engineered cell of any one of Embodiments 7-26, wherein the inhibitory receptor comprises a sequence having at least 85%, at least 90%, at least 95%, at least 97%, at least 99% identity to any one of SEQ ID NOs. 50-72.

Embodiment 28: An engineered cell comprising: an activator receptor and an inhibitory receptor wherein the activator receptor comprises one or more hinge elements.

Embodiment 29: The engineered cell of Embodiment 28, wherein the hinge element is derived from polypeptides encoding a G4Q hinge, a thrombospondin (TSP) hinge, an Immunoglobulin (Ig) hinge, an Epidermal Growth Factor (EGF) hinge, a fibronectin III (FNIII) hinge, or combinations thereof.

Embodiment 30: The engineered cell of Embodiment 29, wherein the one or more hinge elements comprises a hinge element comprising a sequence of any one of SEQ ID NOs: 1-22, or a sequence having at least 85%, at least 90%, at least 95%, at least 97% or at least 99% identity thereto.

Embodiment 31: An engineered immune cell made by the process of any one of Embodiments 1-6.

Embodiment 32: A pharmaceutical composition, comprising a therapeutically effective amount of the engineered cells of any one of Embodiments 7-30.

Embodiment 33: The pharmaceutical composition of Embodiment 32 further comprising a pharmaceutically acceptable carrier, diluent, or excipient.

Embodiment 34: A method of selectively killing targets cells expressing a first antigen and not a second antigen, comprising contacting target cells with the engineered immune cells of any one of Embodiments 7-30.

Embodiment 35: A method of treating or preventing cancer in a subject, comprising administering to the subject the engineered cells of any one of Embodiments 7-30.

Embodiment 36: An engineered cell selectively responsive to target cells expressing a first antigen and not LRRN4, the engineered cell comprising: an activator receptor that specifically binds the first antigen, optionally MSLN, and an inhibitory receptor that specifically binds LRRN4.

Embodiment 37: The engineered cell of Embodiment 36, wherein the cell is an immune cell or a cell capable of differentiating into an immune cell.

Embodiment 38: The engineered cell of any one of Embodiments 36 or 37, wherein the inhibitory receptor comprises one or more hinge elements, such that the combined height of the inhibitory receptor and LRRN4 epitope is less than or approximately equivalent to the combined height of the activator receptor and first antigen epitope.

Embodiment 39: The engineered cell of any one of Embodiments 36-38, wherein the activator receptor and the inhibitory receptor each comprises one or more hinge elements, such that the combined height of the inhibitory receptor and LRRN4 epitope is less than or approximately equal to the combined height of the activator receptor and first antigen epitope.

Embodiment 40: The engineered cell of Embodiment 38 or Embodiment 39, wherein the one or more hinge elements are selected from a G4Q hinge, a TSP hinge, an Ig hinge, an EGF hinge, a FNIII hinge, or combinations thereof.

Embodiment 41: The engineered cell of Embodiment 40, wherein the one or more hinge elements comprises a hinge element comprising a sequence of any one of SEQ ID NOs: 1-22, or a sequence having at least 85%, at least 90%, at least 95%, at least 97% or at least 99% identity thereto.

Embodiment 42: The engineered cell of any one of Embodiments 38-41, wherein the activator receptor is a chimeric antigen receptor (CAR).

Embodiment 43: The engineered cell of Embodiment 42, wherein the activator receptor comprises an scFv.

Embodiment 44: The engineered cell of Embodiment 43, wherein the scFv comprises a sequence of any one of SEQ ID NO: 29-31, or a sequence having at least 85%, at least 90%, at least 95%, at least 97% or at least 99% identity thereto.

Embodiment 45: The engineered cell of any one of Embodiments 42-44, wherein the CAR comprises a CD8 or CD28 transmembrane domain.

Embodiment 46: The engineered cell of any one of Embodiments 42-45, wherein the CAR comprises CD28 and 4-1BB intracellular domains and a CD3z intracellular domain.

Embodiment 47: The engineered cell of any one of Embodiments 42-46, wherein the activator receptor comprises a sequence having at least 85%, at least 90%, at least 95%, at least 97%, at least 99% identity to any one of SEQ ID NOs. 84-104.

Embodiment 48: The engineered cell of any one of Embodiments 36-47, wherein the inhibitory receptor is a chimeric antigen receptor (CAR).

Embodiment 49: The engineered cell of Embodiment 48, wherein the inhibitory receptor comprises an scFv.

Embodiment 50: The engineered cell of Embodiment 49, wherein the scFv comprises a sequence of SEQ ID NO: 29, or a sequence having at least 85%, at least 90%, at least 95%, at least 97% or at least 99% identity thereto.

Embodiment 51: The engineered cell of any one of Embodiments 36-50, wherein the inhibitory receptor comprises a LILRB1 intracellular domain or a functional variant thereof.

Embodiment 52: The engineered cell of any one of Embodiments 36-51, wherein the inhibitory receptor comprises one or more EGF hinge elements.

Embodiment 53: The engineered cell of any one of Embodiments 36-52, wherein the inhibitory receptor comprises 5, 6, or 7 EGF hinge elements.

Embodiment 54: The engineered cell of any one of Embodiments 36-53, wherein the inhibitory receptor comprises a sequence having at least 85%, at least 90%, at least 95%, at least 97%, at least 99% identity to any one of SEQ ID NO: 50.

Embodiment 55: A pharmaceutical composition, comprising a therapeutically effective amount of the engineered cell of any one of Embodiments 36-54.

Embodiment 56: The pharmaceutical composition of Embodiment 55 further comprising a pharmaceutically acceptable carrier, diluent, or excipient.

Embodiment 57: A method of selectively killing targets cells expressing a first antigen, optionally MSLN and not LRRN4, comprising contacting target cells with the engineered immune cell of any one of Embodiments 36-54.

Embodiment 58: A method of treating or preventing cancer in a subject, comprising administering to the subject the engineered cells of any one of Embodiments 36-54.

Embodiment 59: A method of increasing the selective inhibition of an immune cell by an inhibitory receptor, comprising: generating an immune cell comprising: an inhibitory receptor that specifically binds an inhibitor antigen, and an activator receptor that specifically binds an activator antigen, wherein the activator receptor comprises one or more hinge elements; wherein selective inhibition by the inhibitory receptor is increased compared to an immune cell comprising the same inhibitor receptor and a reference activator receptor lacking the hinge element.

Embodiment 60: A method of increasing the selective inhibition activity of an inhibitor receptor in an immune cell comprising an activator receptor, comprising inserting into the extracellular domain of the activator receptor one or more hinge elements until selective inhibition is maximized.

Embodiment 61: A method of increasing the selective inhibition activity of an inhibitor receptor in an immune cell comprising an activator receptor, comprising removing polypeptide segments from the inhibitor receptor until selective inhibition is maximized.

Embodiment 62: The method of one of Embodiments 59 to 61, wherein selective inhibition is maximized when RA≥[TA−EA].

Any engineered cell, pharmaceutical composition, or method disclosed herein is applicable to any herein-disclosed engineered cell, pharmaceutical composition, or method. In other words, any aspect or embodiment described herein can be combined with any other aspect or embodiment as disclosed herein.

### EXAMPLES

The following Examples are intended for illustration only and do not limit the scope of the invention. Throughout the examples, the term “blocker antigen” is used to describe embodiments of a non-target antigen.

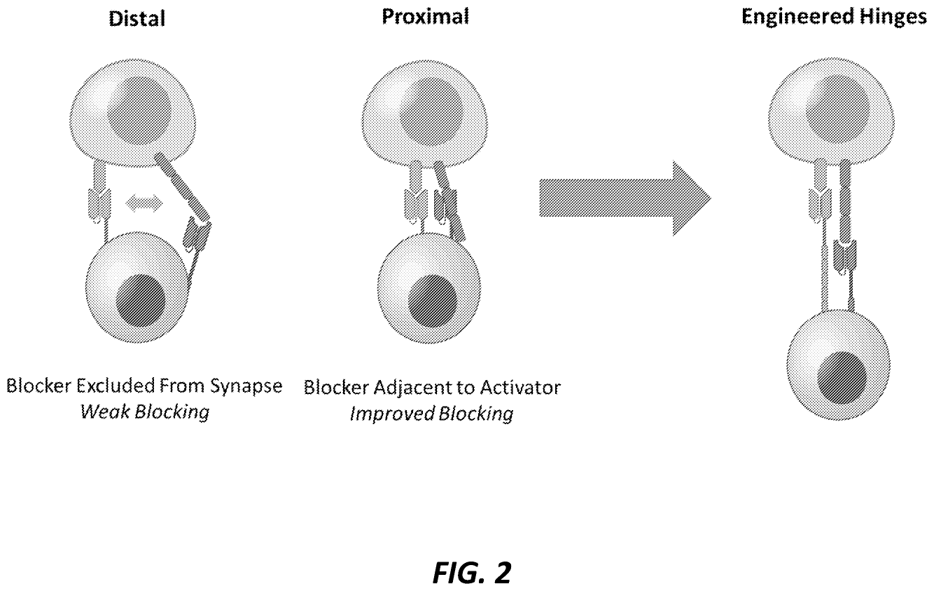

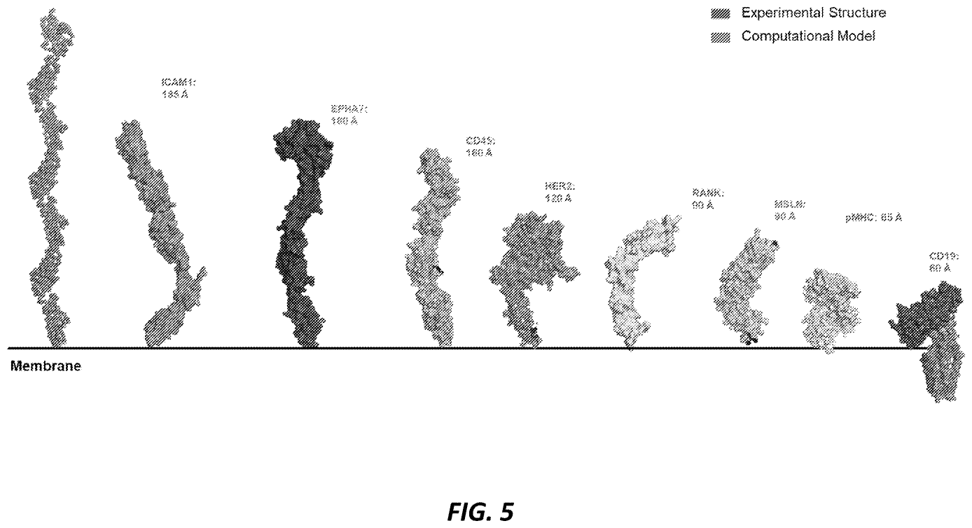

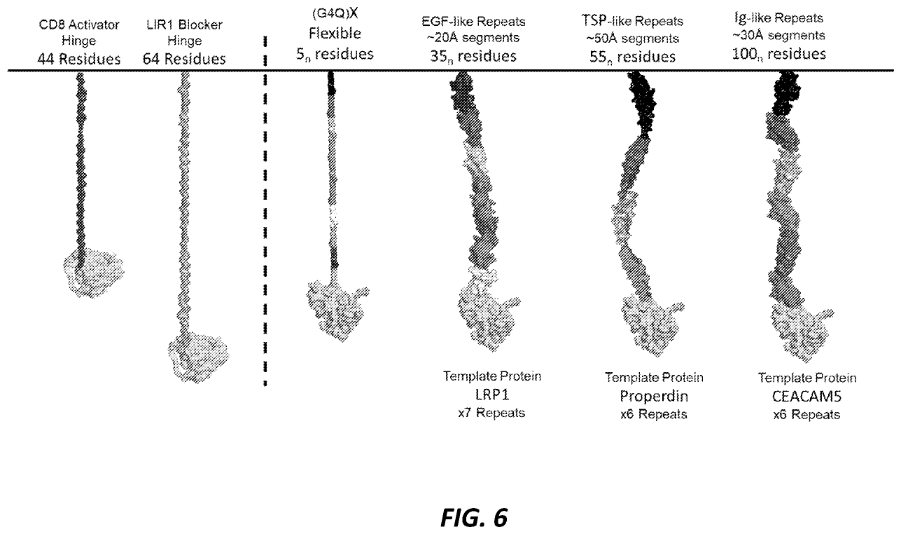

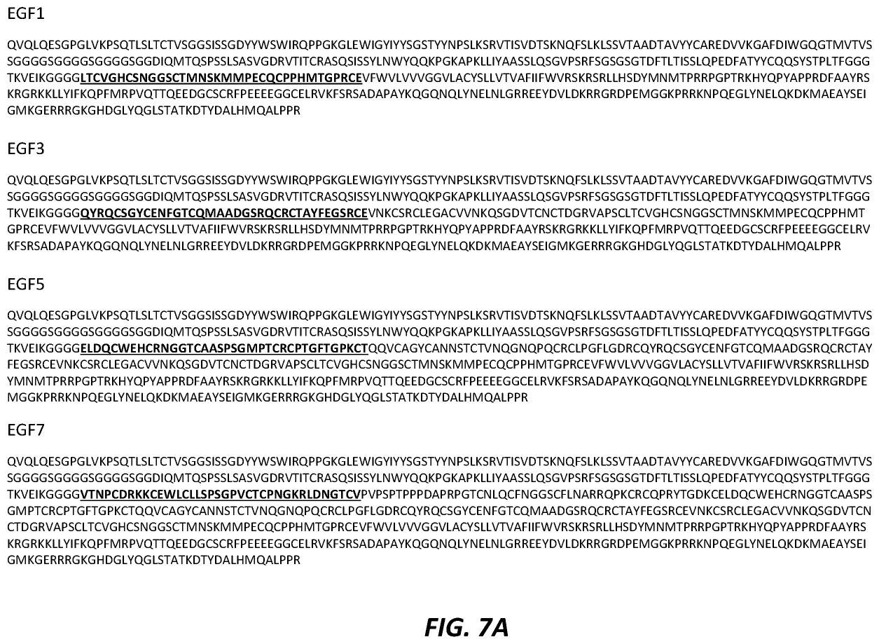

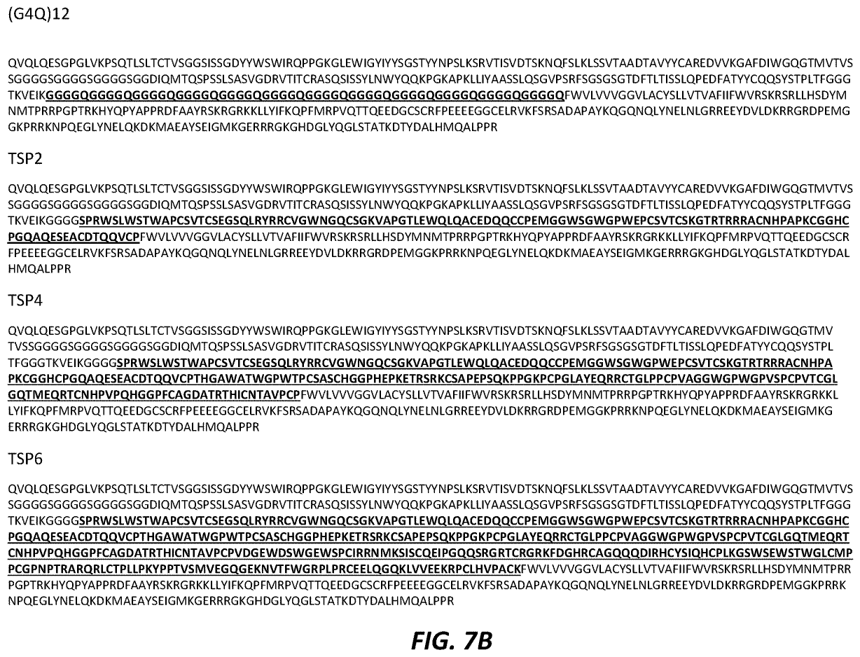

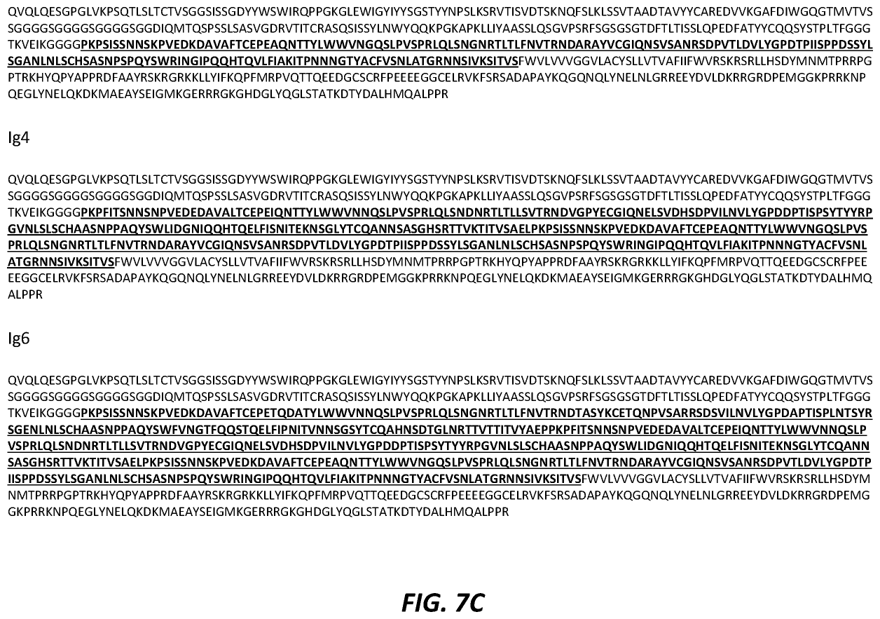

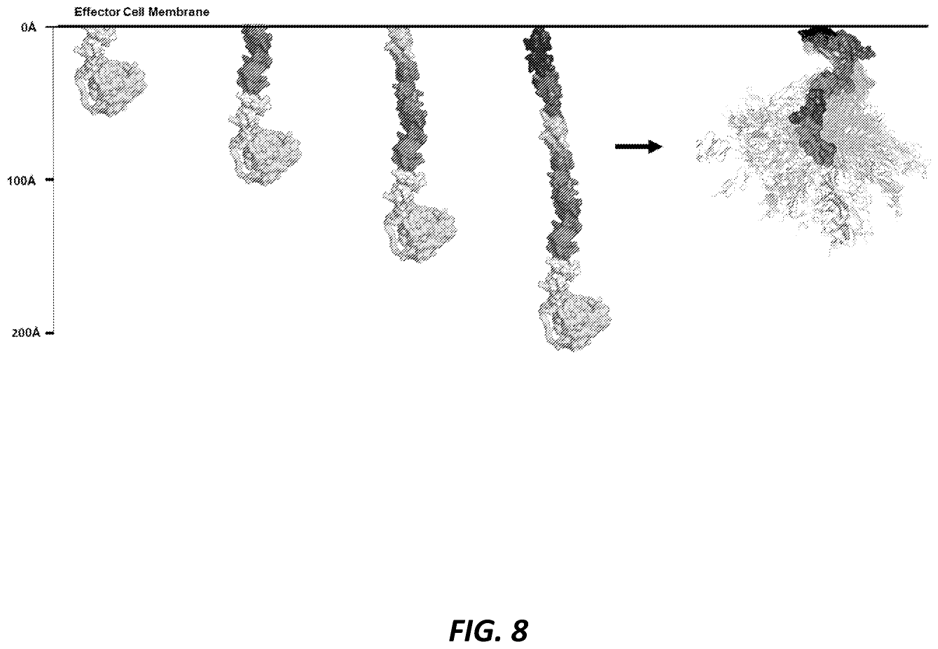

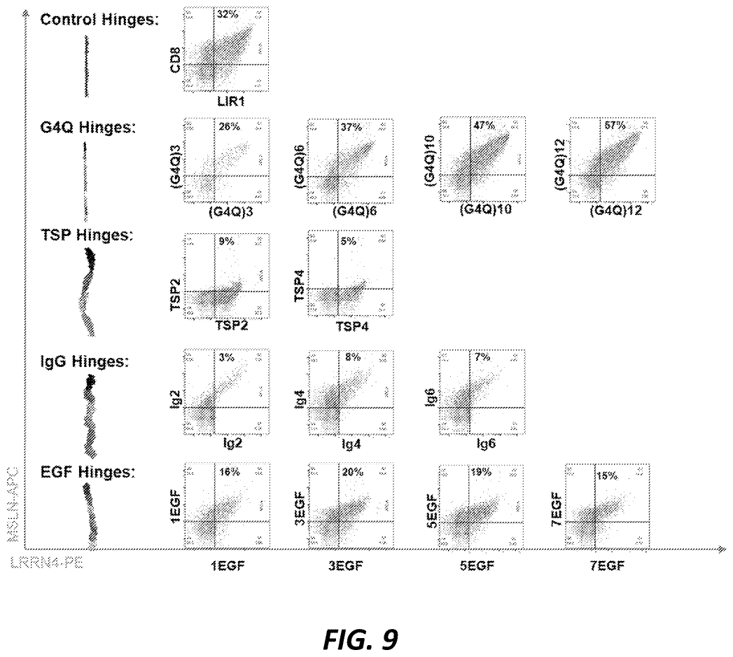

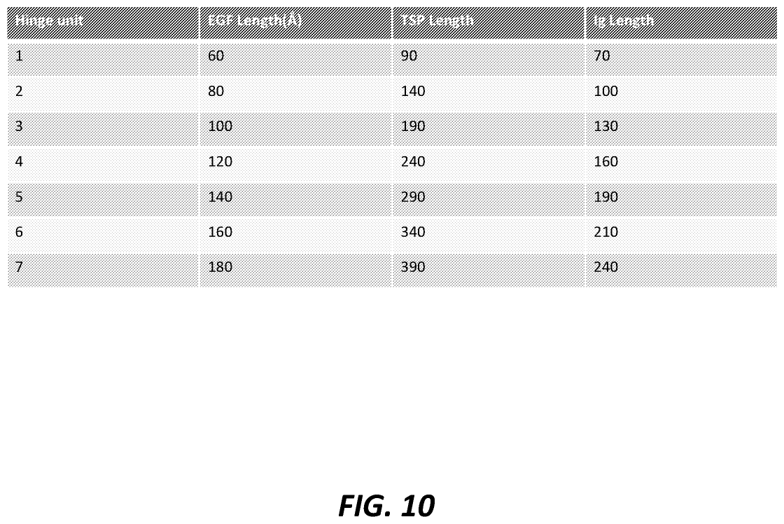

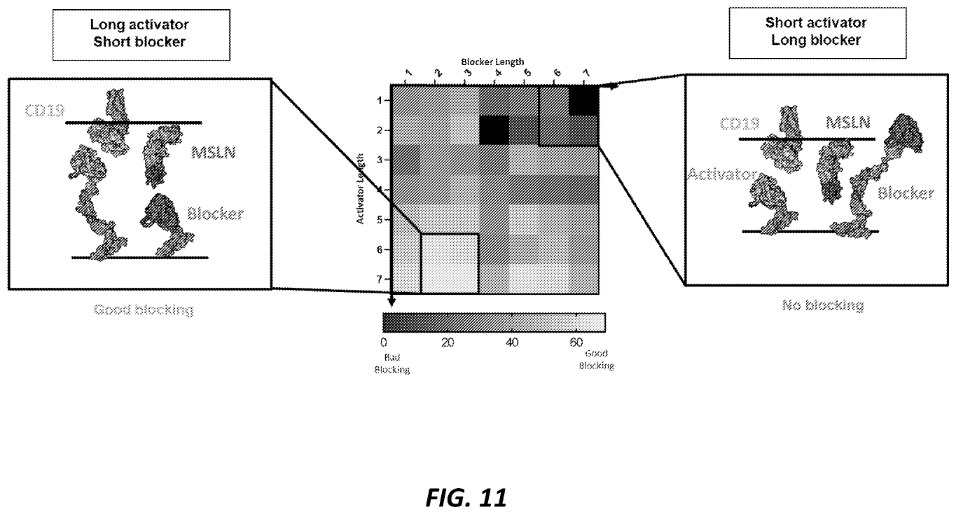

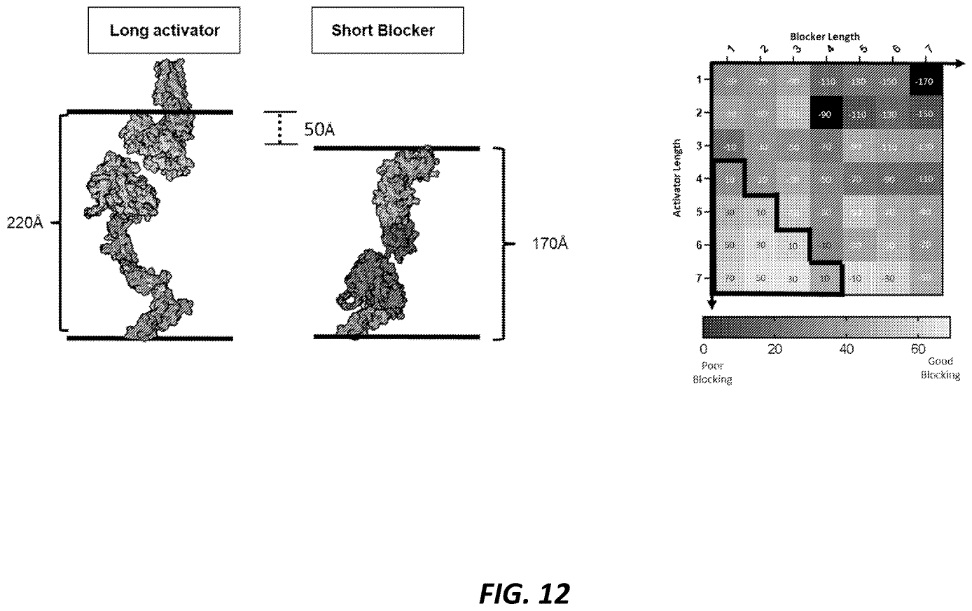

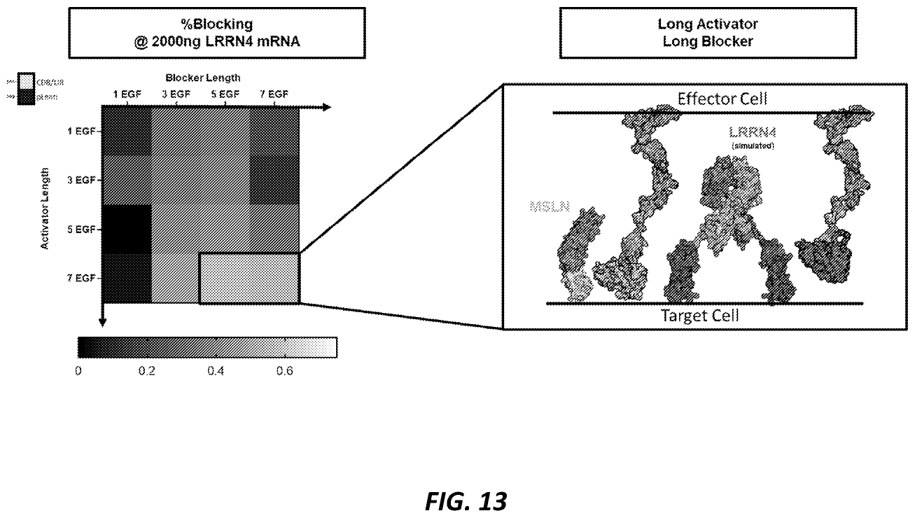

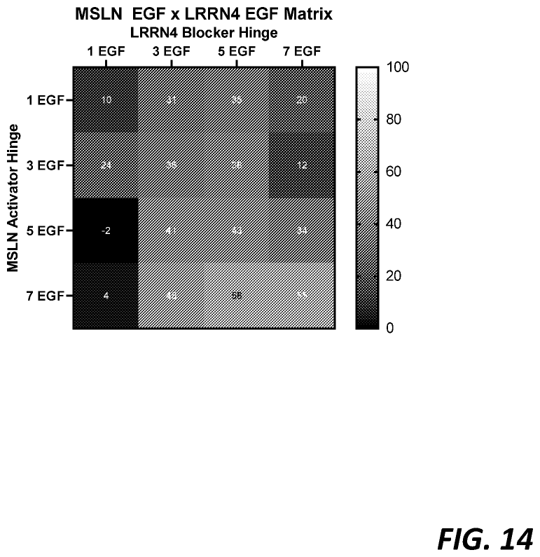

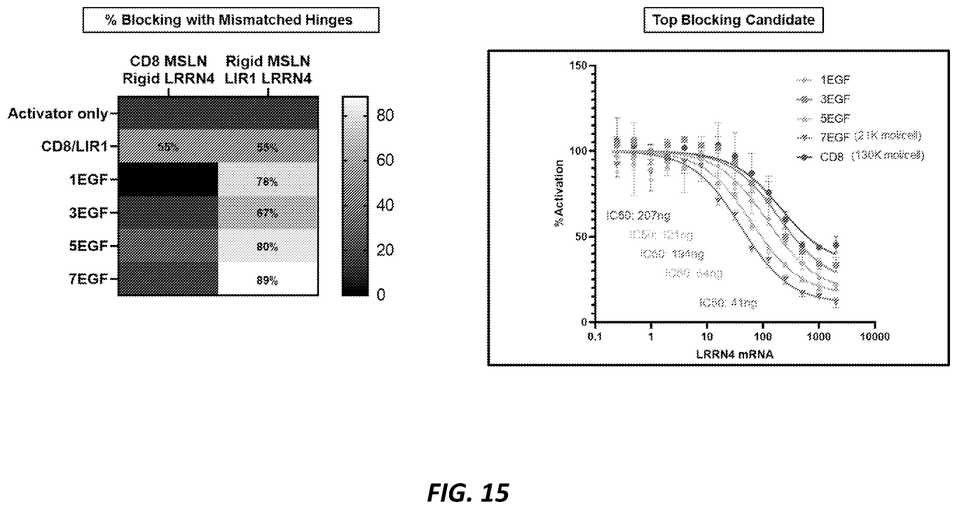

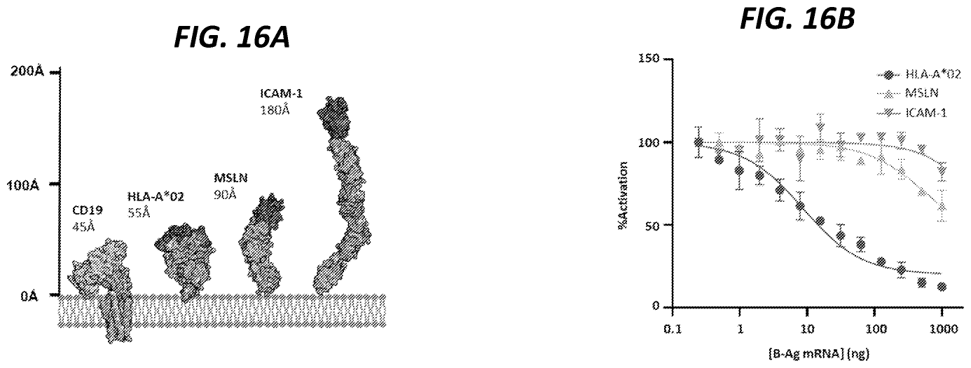

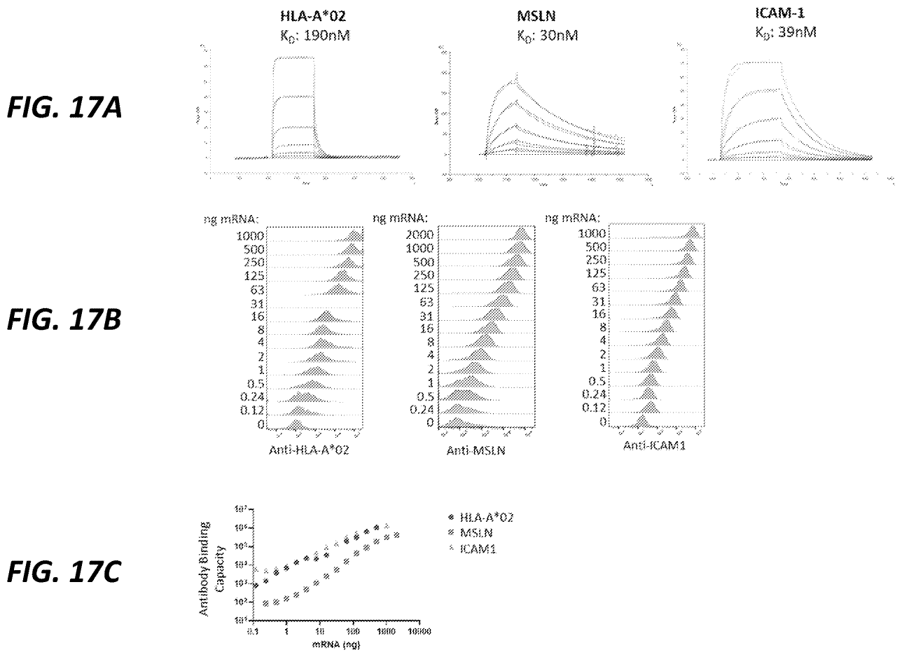

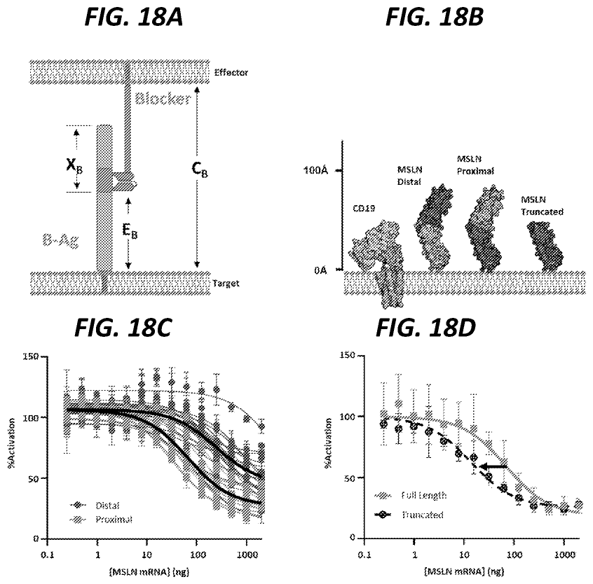

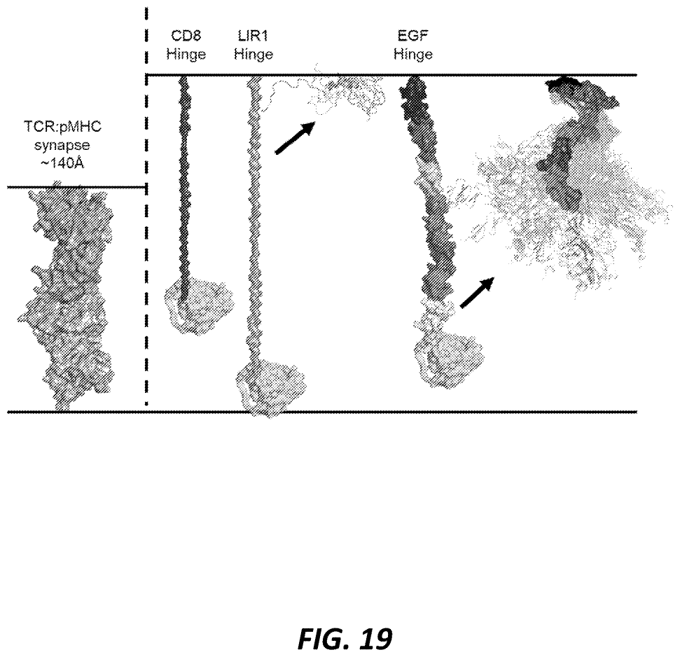

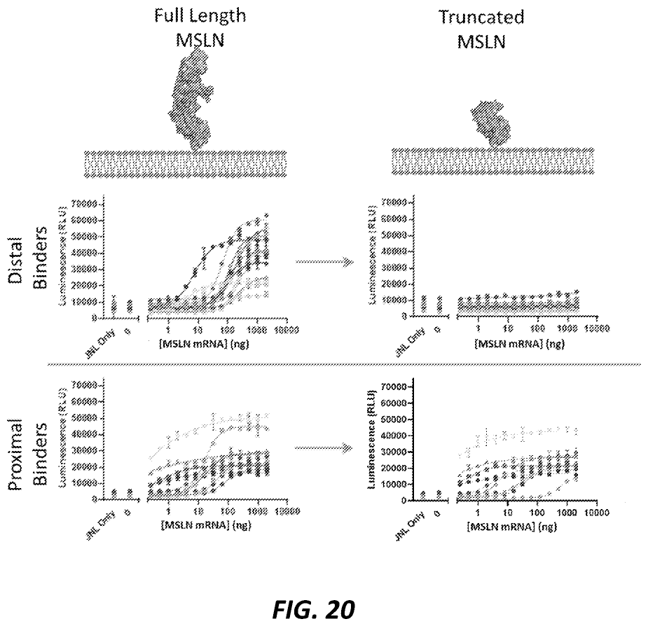

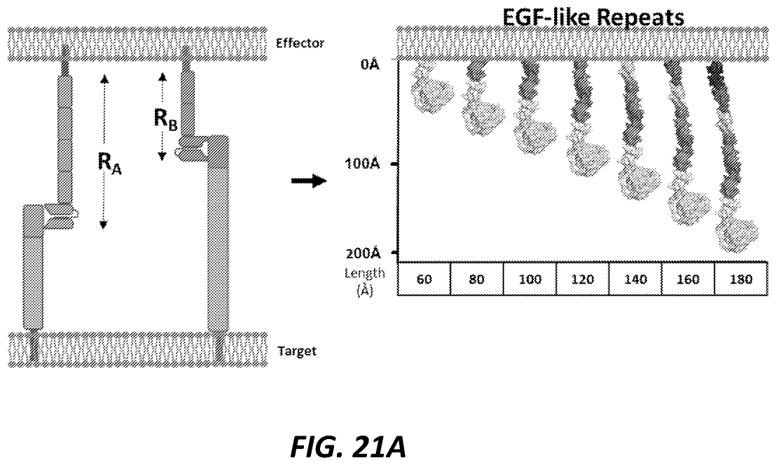

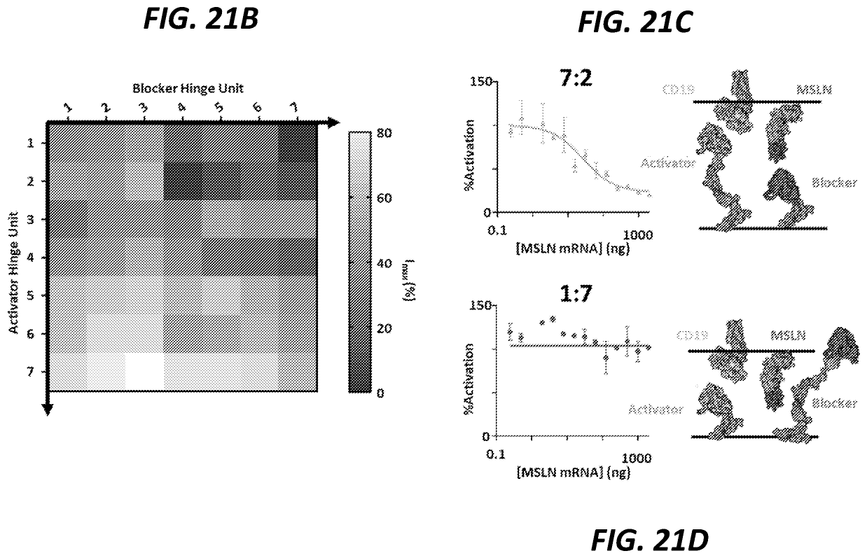

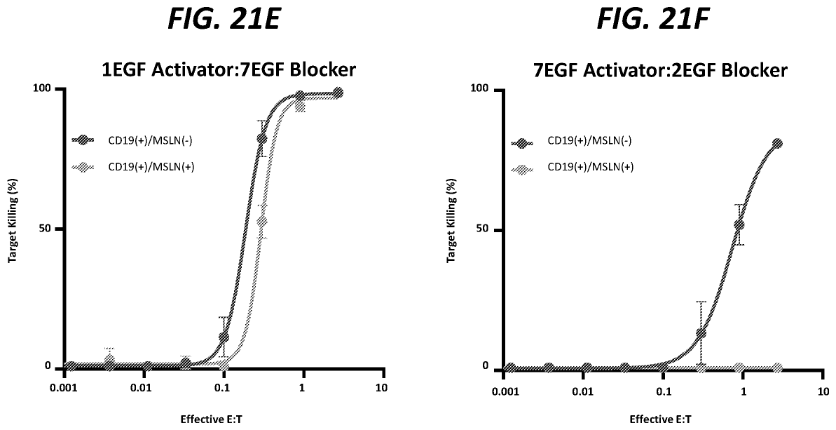

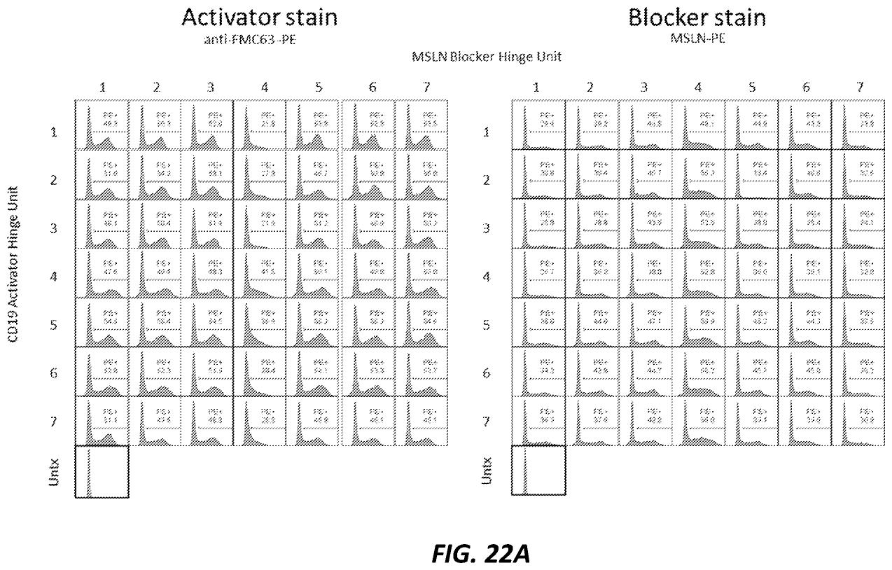

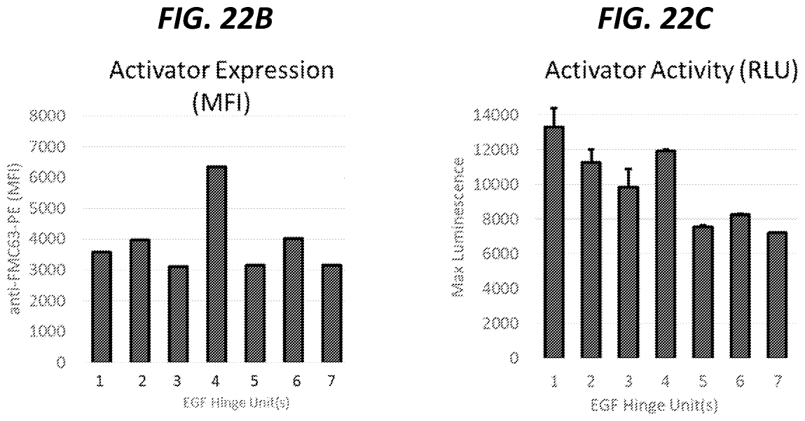

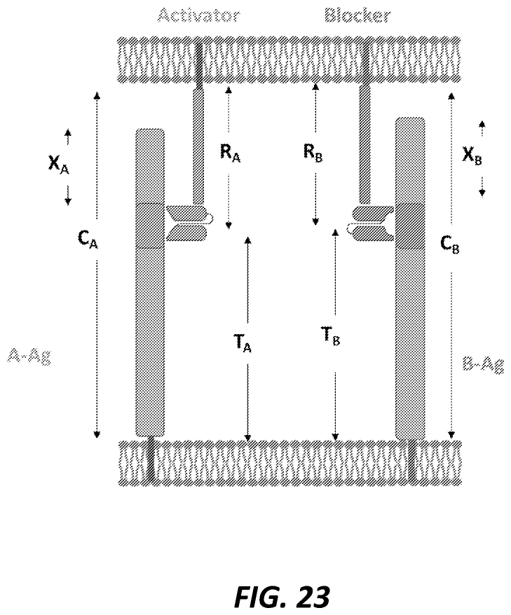

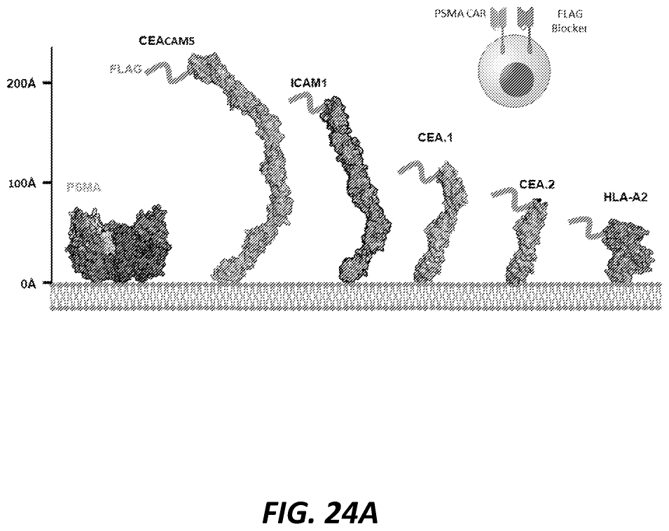

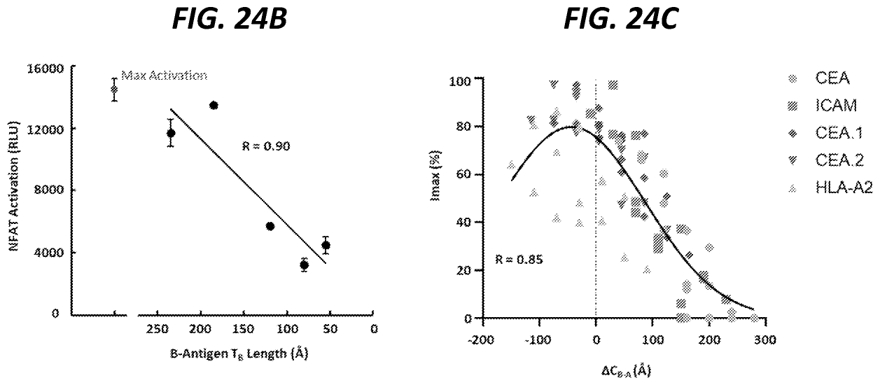

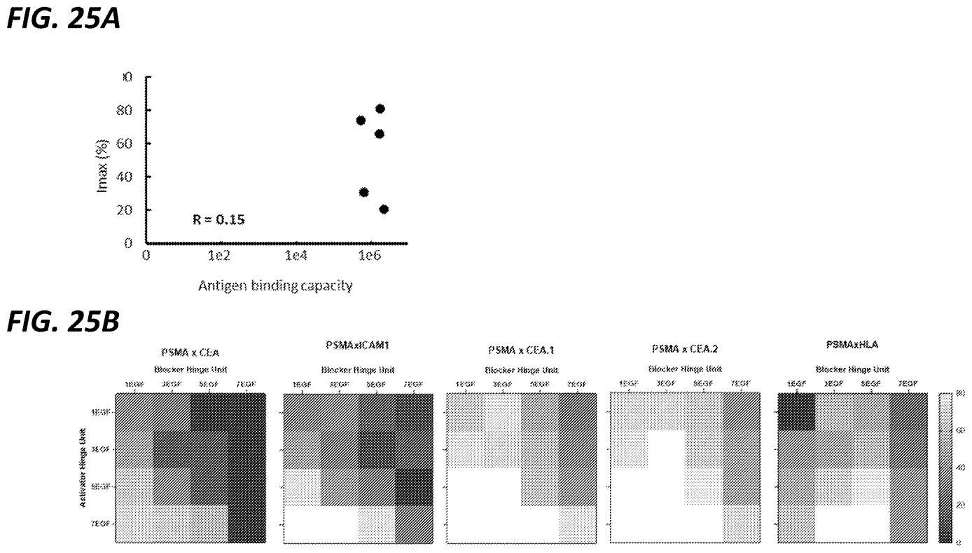

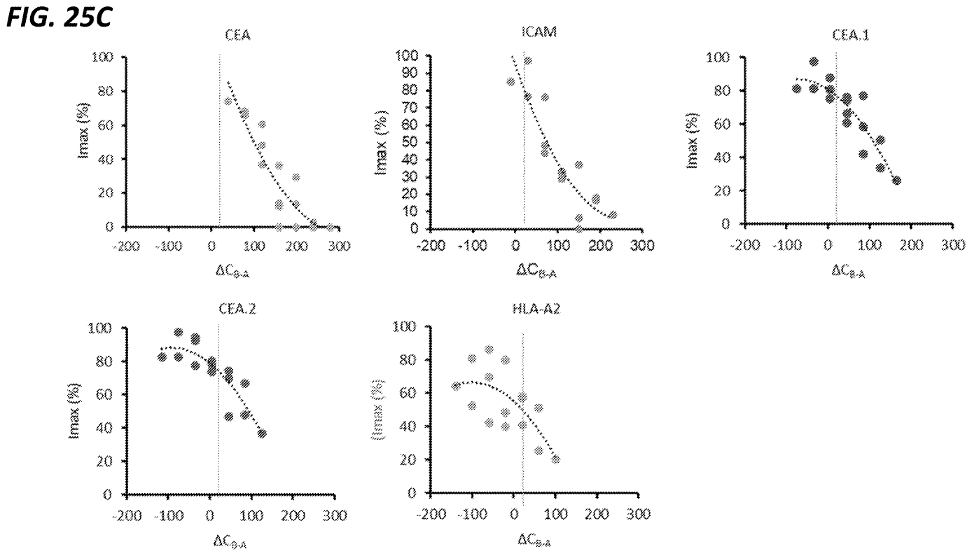

### Example 1: Methods for FIGS. 1-28

Target Cell mRNA Transfection. On the day of transfection, target cells (HeLA or K562, depending on experiment) were counted, washed with 1×PBS and resuspended to 1.1e7 vc/mL in SE (for HeLa cells) or SF (for K562 cells) transfection buffer (Lonza). Target antigen mRNA was serially diluted 2-fold across 15 points in SE/SF transfection buffer in 96-well v-bottom plates. Target cells were added to each well containing the mRNA at a concentration of 1.33e7 vc/mL. The mRNA/cell mixture was transferred to a 16-well Lonza 4D cuvette and electroporated according to the manufacturer's protocol established for target cell line. Post-transfection, the cells were immediately placed into MEM growth media containing serum and seeded into rows of 384-well culture plates at a density of 5000-10,000 cells/well, depending on experiment. Remaining transfected cells were seeded into separate 96-well plates specifically for fluorescence-activated cell sorting (FACS) expression testing. Plates were cultured for >16 hrs at 37 C and 5% CO₂.

Jurkat Coculture Assay. Jurkat-NFAT luciferase cells were counted, washed with 1×PBS and 2e6 viable cells were resuspended in 120 ml of R2 buffer (ThermoFisherScientific) containing 4-8 ug of a 1:1 DNA mixture encoding appropriate activator and blocker receptor constructs. Cells were transfected with the Neon Transfection System (Invitrogen) using the 100 μl format with E2 Buffer (1500 Volts, 10 width, 3 pulses). Immediately after transfection, cells were cultured overnight at 37 C, 5% CO₂ in RPMI containing 20% FBS for co-culture assays the following day. 5,000-10,000 activating and/or blocker receptor expressing Jurkat-NFAT luciferase cells were combined with 5,000-10,000 transfected target cells expressing a fixed amount of activator antigen as described above in triplicate wells of a 96-well plate. The co-culture plates were incubated at 37 C, 5% CO₂ for 6 h. Twenty microliters per well of luciferase substrate (BPS Biosciences) was added to each well of the plate. The plate was then incubated at room temp for 15 min and read on a Tecan M1000 luminescent plate reader with 100 ms integration time/well. % Inhibition was interpreted as the ratio of luminescence between Jurkat cells co-cultured with target cells treated with the highest mRNA concentration of blocker target and Jurkat cells cocultured with target cells expressing no blocker target.

Primary T Killing Assay. Primary human T cells from three donors were transfected with bicistronic constructs encoding the MSLN CAR and LRRN4 blocker using transposase mRNA from the piggybac transfection platform. Cells were grown in GREX according to manufacturer's instructions in X-VIVO 15 supplemented with 1% human serum and 300 IU/mL IL-2. After 14 days, samples were taken from each well, counted, and stained with recombinant MSLN and LRRN4 to estimate transfection efficiency. Luciferase (+) GFP (+) HeLa target cells were used to enable IXM/Incuycyte visualization and terminal luminescence measurements. Rluc GFP HeLA targets were cocultured with Tmod T cells at effective E:T ratios ranging from 0.001-1 (0.01-10 actual) for 48 hours. Using Renilla luciferase substrate (Promega), relative luminescence values were captured, and a specific blocking percentage was calculated. IXM software was used to calculate GFP (+) surface area between conditions.

Structural Modeling. Antigens were modeled and measured in Pymol using coordinates derived from PDB IDs 7JIC (CD19), 6AMT (HLA-A*02), or 1Z8L (PSMA). For antigens without a complete experimental structure (CEA, ICAM1, MSLN), starting coordinates were derived from Alphafold. All structures were relaxed and re-modeled using Rosetta with the all-atom score function and the Pyrosetta interface.

Construct Design and Cloning. scFvs were designed using flexible (G4S)3-GGlinker to connect the VH and VL domains. All third-generation activator CAR constructs contained the CD8a, or EGF-like hinges fused to CD28 TM, as well as CD28, 4-1BB, and CD3z ICDs. All inhibitory receptor constructs contained either the LILRB1 or EGF-like hinges fused to the LILRB1 TM and ICD. Templates used for target protein mRNA synthesis contained 5′ T7 promoter followed by the V kappa 1 signal peptide and codons encoding either HLA, MSLN, ICAM-1, CEA, or PSMA. For FLAG-based blocking assays, a FLAG sequence (DYKDDDDK) was inserted N-terminally to the mature protein sequence. All DNA constructs were assembled using Golden Gate Assembly. DNA templates were either amplified by PCR or linearized by restriction enzyme digest, then mRNA was synthesized using the HiScribe T7 ARCA mRNA kit (New England Biolabs). The in vitro synthesized mRNA was purified using the Monarch RNA Cleanup Kit (New England Biolabs), eluted in 1 mM sodium acetate, and stored at −80 °C.

Surface Plasmon Resonance Binding Data and Analysis. Surface plasmon resonance experiments were performed with a Biacore 3000 instrument using CM5 sensor chips. In all experiments, recombinantly expressed scFvs were immobilized on the sensor chip via standard amine coupling and monovalent antigens were injected as analyte. Experiments were performed at 25 °C in 20 mM HEPES (pH7.4), 150 mM NaCl, and 0.005% surfactant P-20. Injected analyte concentrations spanned 1-1000 nM. Data were processed using BiaEvaluation 4.

Flow Cytometry. Approximately 100,000 cells were collected from each transfected group for flow cytometric analysis. The cells were washed three times in cold PBS containing 1% BSA, then incubated with the corresponding primary antibody for 45 minutes on ice using the manufacturers' recommended staining concentrations. To prepare antigen probes, biotinylated proteins were incubated with PE-conjugated streptavidin at a 4:1 ratio for 20 minutes at room temperature and quenched using RPMI media containing 2% v/v FBS and diluted to 10 g/mL using PBS/BSA. The cells were then washed 3 times in PBS/BSA and incubated with secondary antibody (see below) using the manufacturers' recommended staining concentrations. Cells were then loaded for flow cytometry data acquisition using a BD FACSCanto (BD Biosciences) and FACSDiva software (BD Biosciences). Forward scatter (FSC) vs side scatter (SSC) measurements were used to gate for single cells. Flow cytometric analysis was performed using FlowJo software.

Quantitative analysis of antibody-binding capacity was performed using the QIFIKIT (Dako) according to manufacturer's instructions. Briefly, during flow cytometry sample preparation, 50 μL of resuspended QIFI bead slurry was incubated with secondary antibody using the same protocol as the sample cells. After 3 washes in PBS/BSA, the QIFI beads were loaded on the FACSCanto, using the same parameters as the sample cells being interrogated. Using FlowJo, median fluorescence intensity (MFI) was calculated for each of 6 species of QIFI beads containing different densities of antibody-binding sites, then converted into antibody-binding capacity using values provided by the manufacturer. A calibration curve was generated from these values, which was then used to convert sample cell MFI values into antibody-binding capacities.

Antibodies used in this study: Mouse anti-huHLA-A*02 (BD Biosciences, Cat #551230); F(ab′)2-Goat anti-Mouse IgG (H+L) cross-adsorbed secondary antibody, Alexa Fluor 647 (Invitrogen, Cat #A21237); Mouse anti-huMSLN (R&D Systems, Cat #MAB32653); Mouse anti-huICAM-1 (R&D Systems, Cat #MAB720); Mouse anti-FMC63, PE (Acro Biosystems, Cat #FM3-HPY53); biotinylated human Mesothelin (296-580) protein, His, Avitag (Acro Biosystems, Cat #MSN-H82E9); PE Streptavidin (BioLegend, Cat #405245).

The methods and examples described in PCT/US2021/060607, PCT/US2023/011698, US 20230029341, US 20220370497, US 20220273721, and US 20220054551, are incorporated herein by reference in their entirety.

### Example 2: Characterization of Antigen Height as a Functional Parameter

CAR-T platforms have generally been more successful when targeting small proteins over large and bulky antigens, however it was unknown whether this principle applied to the inhibitory receptors (B-ligands). The first iterations of the engineered blocker system were developed to target the relatively small (55 Å axial length; FIG. 1 and FIG. 16A) extracellular domain of HLA-A*02, which can trigger exceptional inhibitory behavior. However, expansion into larger patient populations independent of HLA LOH status requires the ability to engage non-HLA targets.

To investigate the effectiveness of a blocker module reprogrammed to target B-antigens of different heights, two blockers directed at non-HLA antigens were compared to the established HLA-A*02 blocker. The established CD19 CAR with the FMC63 scFv was used as the activator, paired with three different blocker constructs targeting membrane-distal epitopes of three distinct B-antigens (HLA-A*02, MSLN, and ICAM-1; FIG. 16B) (SEQ ID NO: 30). The affinities of the scFvs for their cognate B-antigens ranged from 30 to 200 nM (FIG. 17) (SEQ ID NOs: 158, 31, and 1334-1349). These model B-antigens cover a range of ~100 Å axial lengths and were intended to explore the modularity of the Tmod platform regarding B-antigen height.

The effectiveness of each blocker was measured by the B-ligand-dependent inhibition of the CD19-mediated CAR activation. The genes for each receptor combination were co-transfected into Jurkat cells expressing an NFAT-luciferase reporter. Jurkat cells were co-cultured with target cell lines expressing a fixed level of CD19 and increasing levels of B-antigen (generated by transfection with increasing amounts of B-antigen synthetic mRNA) to generate dose-inhibition curves. The level of inhibition with two parameters: IC50 (B-antigen concentration at half-maximal inhibition) and Imax (maximum percentage of inhibition) were measured.

The inhibitory receptor targeting the HLA-A*02 B-antigen efficiently inhibited Jurkat cell activation in a concentration-dependent manner. The highest HLA-A*02 mRNA concentration resulted in approximately 90% inhibition of the CD19 CAR activation signal (FIG. 17C). In contrast, inhibitory receptors targeting MSLN and ICAM-1 were less effective at inhibiting the CD19 CAR (40% and 20%, respectively). The relative amount of inhibition was anti-correlated with the height of the blocker target, with the small HLA-A*02 (55 Å) outperforming both the larger MSLN (90 Å) and ICAM-1 (185 Å). Taken together, in the dual receptor engineered immune cell system inhibition signaling (Imax/Imin) is improved when the Blocker+B-Ag epitope length is less that Activator+A-ag epitope length (FIG. 1).

This example shows that the height of the blocker antigen has a direct impact on the blocking function of the engineered immune cell.

### Example 3: Targeting Membrane-Proximal Epitopes Results in More Effective Blocking

Next, it was considered how the proximity of the B-antigen epitope height (E_{B}) relative to the target cell membrane may also influence blocking function, presumably by altering C_{B} (FIG. 18A). However, targeting membrane-proximal epitopes of long or bulky antigens may also limit epitope accessibility, and therefore receptor engagement, by creating the potential for steric interference of the B-antigen distal region with the effector cell membrane. The length of the epitope distal region on the blocker antigen (X_{B}) was also considered.

The height of a CAR: ICAM-1 complex was compared to that of a TCR: pMHC complex (FIG. 16B; FIG. 19), and it was predicted that the X_{B} values of membrane-proximal ICAM binders would far exceed the tolerance of C_{B}. Based on this prediction, the mid-sized MSLN B-antigen from above was selected to explore these variables. 36 anti-MSLN LBDs were screened with the intention to diversify E_{B} and the MSLN truncations were used to functionally define and classify the membrane-distal and membrane-proximal binders (FIG. 20). After conversion into the blocker format, the blocking potencies were assessed with the Jurkat activation assay described above. Clustering anti-MSLN blockers by epitope revealed that blockers targeting membrane-proximal MSLN epitopes were generally more effective at blocking the CAR activation signal compared to those targeting membrane-distal epitopes (p<0.001, FIG. 18B, C). On average, blockers generated from membrane proximal MSLN binders inhibited over 70% of the maximum activation signal when co-cultured with CD19(+) target cells transfected with MSLN mRNA. This contrasted with the reduced potencies of blockers derived from membrane distal MSLN binders (Imax~70% vs 50% inhibition; IC50~60 ng vs 250 ng MSLN mRNA) (FIG. 18C). Although blocker function in these experiments was not normalized for expression level and affinity, the data here imply that the inhibitory function of the blocker can be dependent on the epitope distance from the membrane.

As mentioned above, targeting membrane-proximal epitopes of large antigens must consider the extracellular antigen bulk beyond the epitope. Indeed, the full-length MSLN protein extends ~50% further from the target cell membrane than HLA-A*02 (FIG. 16B), and this additional mass of extracellular domain (X_{B}, FIG. 18A) may interfere with blocking, regardless of epitope position. To directly interrogate the effect of the excess MSLN extracellular domain (X_{B}), the most sensitive MSLN blocker (SEQ ID NO: 28) from above was selected and the inhibition experiment with a truncated form of MSLN was repeated (FIG. 18B). Although the truncated MSLN variant was expressed at comparable levels relative to the full-length construct (data not shown), the selected blocker was more sensitive to the truncated form (FIG. 18D). Together, these data suggest that, in addition to the height of the B-antigen (T_{B}), the location of the epitope (E_{B}) may also influence blocking function (FIG. 16A, 18A). However, although targeting membrane-proximal epitopes shortens E_{B}, it also leads to a proportionally larger X_{B} value that may sterically prevent the formation of the blocker complex.

### Example 4: Controlling Blocking with Receptor Hinge Lengths

To thoroughly define the relationship between receptor length (R_{B}) and blocker function, modular domain repeats were used for estimation of the distance of the ligand binding domain (LBD) from the T cell membrane surface based on the number of repeats (FIG. 21A). Epidermal growth factor (EGF)-like domains (SEQ ID NOS: 14-20), characterized by three intradomain disulfide bonds, were used due to the high and uniform expression levels when incorporated as hinge subunits (FIG. 22). Thus, a series of blocker hinges were designed to include up to 7 EGF-like domain repeats (extracted from LRP-1; uniprotID: Q07954, aa 4147-4409) to increase the distance between the MSLN ligand-binding domain and the T cell surface in 20-30 Å increments (FIG. 21A; TABLE 5 below) (SEQ ID NOS: 65-72). To characterize the geometric relationship between CAR and blocker the same rigid hinge series was also incorporated into the CD19 CAR (SEQ ID NOS: 98-104). Blocking efficiencies of each hinge combination were used to generate a 7×7 functional matrix, enabling a systematic evaluation of different activator/blocker geometries.

TABLE 5

| EGF-like Hinge Unit | R Estimate(Å) |
|---|---|
| 1 | 60 |
| 2 | 80 |
| 3 | 100 |
| 4 | 120 |
| 5 | 140 |
| 6 | 160 |
| 7 | 180 |

To compare blocking strength, a simplified inhibition assay was used in which each hinge combination was transfected into Jurkat cells and co-cultured with either MSLN (+) or MSLN (−)K562 target cells engineered to overexpress CD19. The percent decrease in activation signal associated with MSLN expression was then calculated. Populating a matrix with these values revealed that short blocker hinges paired with long activator hinges led to significantly improved blocking, whereas short activators paired with long blockers completely abrogated blocker function (FIG. 21B). Comparing two distinct hinge configurations in a MSLN titration co-culture assay yielded the full span of blocking profiles (FIG. 21C, 21D). Furthermore, structural models suggest that the short CD19 activators sterically prevent the blocker from engaging the MSLN B-antigen (i.e., the blocker complex is too large; FIG. 21D), whereas the hinge combination that best compensated for differences in the target antigens led to the most efficient blocking profile (FIG. 21C). Additionally, PBMCs expressing a short CD19 activator and long MSLN blocker kill target cells expressing CD19 independently of MSLN expression status (FIG. 21E). However, although blocking was most effective with long activators, extended activators were also associated with a decrease in activation sensitivity (FIG. 24) PBMCs expressing a long activator and short blocker require higher E:T ratios to effectively kill target cells expressing CD19, but completely spare target cells expressing both CD19 and MSLN (FIG. 21F).

In summary, the model CD19/MSLN target system explored above suggests that several geometric parameters (height of the receptor, antigen, and overall complex) can be directly related to T cell function. These predictors contextualize the data for the engineered immune cells antigen pairs examined in Example 5(CD19/HLA-A*02, CD19/MSLN, CD19/ICAM-1). Furthermore, the observations suggest a set of design principles to predict and improve blocker performance: (i) the blocker complex should be smaller than the activator complex and (ii): the B-antigen epitope must be accessible to the blocker on the cell surface, following engagement of A-Antigen by the activating CAR (FIG. 23).

### Example 5: Prospective Test of Geometric Rules for Engineered Immune Cell Design: Applications to Other Targets

Next, the generalizability of these design principles and engineering strategies when targeting a more extreme size disparity with different activator and blocker antigens was tested. PSMA was selected as the A-antigen and HLA-A2, ICAM-1, and a series of CEACAM5 variants were selected as the B-antigen (SEQ ID NOs: 133-1357). The different B-antigens allow for the impact of different axial lengths to be tested. To control variability between distinct blocker LBDs due to differences in expression, affinity, and epitope accessibility a blocker directed towards an engineered FLAG tag present on the N-terminus of each B-antigen target was developed (FIG. 24A). Thus, any differences observed in Jurkat activation should be a direct result of differences in the target antigen construct.

Consistent with the observation in FIG. 1C, the ability of the FLAG-blocker to inhibit the PSMA activation signal was closely correlated with B-antigen axial length (R=0.9; FIG. 24B). Jurkat cells expressing the PSMA activator and FLAG-directed blocker only inhibited ~30% of the activation signal when co-cultured with PSMA (+)/FLAG-CEA (+) HeLa target cells (relative to co-culture with PSMA (+)/FLAG (−) target cells). In contrast, the same Jurkat cells inhibited over 80% of the maximum activation signal when co-cultured with target cells expressing the smallest FLAG-tagged B-antigens (FIG. 24B). Differences in B-antigen expression did not explain these differences in function, as each B-antigen expressed at similar, high levels (>500,000 antigen-binding capacity (ABC) quantified by QIFI) (FIG. 25A).

Based on the diverse functional behavior in response to antigen height observed in FIG. 24A and FIG. 24B, the modular EFG hinges were used to determine the optimal hinge size for both the activator and blocker. A series of modular hinges were incorporated into both the PSMA activator and FLAG blocker to generate a 4×4 matrix of Jurkat cells expressing both receptors (SEQ ID NOs: 1350-1365). The Jurkat cells expressing long activators and short blockers were generally more capable of inhibiting the maximum activation signal when co-cultured with PSMA (+)/FLAG (+) HeLa target cell lines, regardless of B-antigen length (FIG. 25B). Indeed, the hinge extensions improved the engineered immune cell's function with even the largest FLAG-CEA and FLAG-ICAM-1 B-antigens, although the resultant long activator hinge reduced the baseline signal.

To compare these datasets in a single analytical framework the length of each receptor-ligand complex was estimated using a conversion table (Table 6 below) that sums the axial length of the receptor with the axial length of the target antigen. After calculating the maximum percentage of inhibition (Imax), the measured values were plotted against the difference in estimated length between the activation and blocking complexes (ΔCB-A, FIG. 25C). Despite the cumulative inaccuracies associated with estimating the length of both receptor and antigen, the data followed a general nonlinear trend with peak inhibition occurring when ΔCB-A approaches zero). Plotting all the data together revealed an approximate Gaussian distribution, even with limited data points on the lefthand (i.e., activator smaller than blocker) side (FIG. 24C). Thus, any gross mismatch between activator and blocker complex has the potential to limit the engineered immune cell function, but rational receptor design can correct for suboptimal conditions.

TABLE 6

| Target Combinations | | | | | |
|---|---|---|---|---|---|
| A-Antigen | B-Antigen | T_{A} | T_{B} | E_{A} | E_{B} |
| CD19 | MSLN | 45 | 90 | 45 | 90 |
| PSMA | CEA | 75 | 235 | 75 | 235 |
| PSMA | CEA.1 | 75 | 120 | 75 | 120 |
| PSMA | CEA.2 | 75 | 80 | 75 | 80 |

### ABBREVIATIONS

RA=Height of CAR extracellular region from effector cell membrane
RB=Height of Blocker extracellular region from effector cell membrane
TB=Total height of B-Antigen extracellular region from target cell membrane
EA=A-Antigen epitope distance from target cell membrane
EB=B-Antigen epitope distance from target cell membrane
(TA−EA)=Minimum RA required to access A-Antigen epitope
(TB−EB)=Minimum RB required to access B-Antigen epitope
(RB+EB)≤(RA+EA)=Ideal relationship between blocker and activator

### Activation Parameters

Emax=Maximum signal
EC50=Antigen mRNA concentration at half-maximum signal
Emin=Baseline signal; effector and target cells, no activator mRNA transfected
E0=Tonic signaling of effector cells only

### Inhibition Parameters

Imax=Maximum inhibition signal
IC50=Antigen mRNA concentration at half-maximum inhibition signal
Imin=Minimum inhibition value (also where signal is maximum)
E0=Tonic signaling; the same measurement as on the left graph

What is claimed is:

1. A method of producing an engineered cell responsive to targets cells expressing a first antigen and not a second antigen, the method comprising:
identifying a first antigen and a second antigen having epitopes of defined heights;
selecting one or more hinge elements based on the relative heights of the epitopes of the first antigen and the second antigen;
generating polynucleotide sequences encoding:
an activator receptor with a defined receptor height that specifically binds the epitope of the first antigen, and
an inhibitory receptor with a defined receptor height that specifically binds the epitope of the second antigen,
wherein either the activator receptor or the inhibitor receptor, or both, comprise at least one hinge element such that the combined height of the inhibitory receptor and second antigen epitope is less than or approximately equal to the combined height of the activator receptor and first antigen epitope; and
introducing the polynucleotide sequences into a cell;
thereby generating the engineered cell responsive to targets cells expressing the first antigen and not the second antigen, and nonresponsive to cells expressing both the first and second antigen.

2. A method of rescuing activity of paired inhibitory and activating receptors in an engineered cell, comprising:
generating polynucleotide sequences encoding:
an activator receptor that specifically binds an epitope of a first antigen, and
an inhibitory receptor that specifically binds an epitope of a second antigen,
wherein the activator receptor comprises at least one hinge element such that the combined height of the activator receptor and first antigen epitope is greater than the combined height of the inhibitory receptor and second antigen epitope; and
introducing the polynucleotide sequences into a cell,
thereby rescuing the activity of inhibitory and activating receptors in the engineered cell.

3. The method of claim 1 or 2, wherein the cell is an immune cell or a cell capable of differentiating into an immune cell.

4. The method of claim 1 or claim 2, wherein the activator receptor and the inhibitory receptor each comprises at least one selected hinge element such that the combined height of the inhibitory receptor and second antigen epitope is approximately equal to the combined height of the activator receptor and first antigen.

5. The method of claim 1 or 2, wherein the activator receptor comprises a plurality of selected hinge elements such that the combined height of the activator receptor and the first antigen epitope is larger than the combined height of the inhibitory receptor and second antigen epitope.

6. The method of claim 1 or 2, wherein the height of the inhibitory receptor is reduced such that the combined height of the inhibitory receptor and second antigen epitope is less than the combined height of the activator receptor and first antigen epitope.

7. An engineered cell selectively responsive to target cells expressing a first antigen and not a second antigen, but nonresponsive to cells expressing both the first and second antigen, the engineered cell comprising:
an activator receptor that specifically binds the first antigen, and
an inhibitory receptor that specifically binds the second antigen,
wherein the receptor that binds the shorter of the epitopes of two antigens comprises one or more hinge elements such that said receptor has a greater height than the other receptor.

8. The engineered cell of claim 7, wherein the cell is an immune cell or a cell capable of differentiating into an immune cell.

9. The engineered cell of claim 7, wherein the activator receptor and the inhibitory receptor each comprises one or more hinge elements.

10. The engineered cell of claim 7, wherein the one or more hinge elements are derived from polypeptides encoding a G4Q hinge, a thrombospondin (TSP) hinge, an Immunoglobulin (Ig) hinge, an Epidermal Growth Factor (EGF) hinge, a fibronectin III (FNIII) hinge, or combinations thereof.

11. The engineered cell of claim 10, wherein the one or more hinge elements comprises a hinge element comprising a sequence of any one of SEQ ID NOs: 1-22, or a sequence having at least 85%, at least 90%, at least 95%, at least 97% or at least 99% identity thereto.

12. The engineered cell of claim 7, wherein the height of the inhibitory receptor is approximately equal to or less than the epitope height of the activator receptor.

13. The engineered cell of claim 7, wherein the first antigen is selected from MSLN, CD19, and CEA.

14. The engineered cell of claim 7, wherein the second antigen is selected from MSLN, LRRN4, and HLA-A2.

15. The engineered cell of claim 7, wherein the activator receptor is a chimeric antigen receptor (CAR).

16. The engineered cell of claim 15, wherein the activator receptor comprises an scFv.

17. The engineered cell of claim 16 wherein the scFv comprises a sequence of any one of SEQ ID NO: 29-31, or a sequence having at least 85%, at least 90%, at least 95%, at least 97% or at least 99% identity thereto.

18. The engineered cell of claim 15, wherein the CAR comprises a CD8 or CD28 transmembrane domain.

19. The engineered cell of claim 15, wherein the CAR comprises CD28 and/or 4-1BB intracellular domains and a CD3z intracellular domain.

20. The engineered cell of claim 15, wherein the activator receptor comprises a sequence having at least 85%, at least 90%, at least 95%, at least 97%, at least 99% identity to any one of SEQ ID NOs. 84-104.

21. The engineered cell of claim 7, wherein the inhibitory receptor is a chimeric antigen receptor (CAR).

22. The engineered cell of claim 21, wherein the inhibitory receptor comprises an scFv.

23. The engineered cell of claim 22, wherein the scFv comprises a sequence of SEQ ID NO: 28, 29, or 31, or a sequence having at least 85%, at least 90%, at least 95%, at least 97% or at least 99% identity thereto.

24. The engineered cell of claim 7, wherein the inhibitory receptor comprises a LILRB1 intracellular domain or a functional variant thereof.

25. The engineered cell of claim 7, wherein the inhibitory receptor comprises one or more EGF hinge elements.

26. The engineered cell of claim 25, wherein the inhibitory receptor comprises 1, 2, 3, or 4 EGF hinge elements.

27. The engineered cell of claim 7, wherein the inhibitory receptor comprises a sequence having at least 85%, at least 90%, at least 95%, at least 97%, at least 99% identity to any one of SEQ ID NOs. 50-72.

28. An engineered cell comprising:
an activator receptor and
an inhibitory receptor
wherein the activator receptor comprises one or more hinge elements.

29. The engineered cell of claim 28, wherein the hinge element is derived from polypeptides encoding a G4Q hinge, a thrombospondin (TSP) hinge, an Immunoglobulin (Ig) hinge, an Epidermal Growth Factor (EGF) hinge, a fibronectin III (FNIII) hinge, or combinations thereof.

30. The engineered cell of claim 29, wherein the one or more hinge elements comprises a hinge element comprising a sequence of any one of SEQ ID NOs: 1-22, or a sequence having at least 85%, at least 90%, at least 95%, at least 97% or at least 99% identity thereto.

31. An engineered immune cell made by the process of claim 1 or claim 2.

32. A pharmaceutical composition, comprising a therapeutically effective amount of the engineered cells of claim 7.

33. The pharmaceutical composition of claim 32 further comprising a pharmaceutically acceptable carrier, diluent, or excipient.

34. A method of selectively killing targets cells expressing a first antigen and not a second antigen, comprising contacting target cells with the engineered immune cells of claim 7.

35. A method of treating or preventing cancer in a subject, comprising administering to the subject the engineered cells of claim 7.

36. An engineered cell selectively responsive to target cells expressing a first antigen and not LRRN4, the engineered cell comprising:
an activator receptor that specifically binds the first antigen, optionally MSLN, and
an inhibitory receptor that specifically binds LRRN4.

37. The engineered cell of claim 36, wherein the cell is an immune cell or a cell capable of differentiating into an immune cell.

38. The engineered cell of claim 37, wherein the inhibitory receptor comprises one or more hinge elements, such that the combined height of the inhibitory receptor and LRRN4 epitope is less than or approximately equivalent to the combined height of the activator receptor and first antigen epitope.

39. The engineered cell of claim 37, wherein the activator receptor and the inhibitory receptor each comprises one or more hinge elements, such that the combined height of the inhibitory receptor and LRRN4 epitope is less than or approximately equal to the combined height of the activator receptor and first antigen epitope.

40. The engineered cell of claim 38 or claim 39, wherein the one or more hinge elements are selected from a G4Q hinge, a TSP hinge, an Ig hinge, an EGF hinge, a FNIII hinge, or combinations thereof.

41. The engineered cell of claim 40, wherein the one or more hinge elements comprises a hinge element comprising a sequence of any one of SEQ ID NOs: 1-22, or a sequence having at least 85%, at least 90%, at least 95%, at least 97% or at least 99% identity thereto.

42. The engineered cell of claim 38 or claim 39, wherein the activator receptor is a chimeric antigen receptor (CAR).

43. The engineered cell of claim 42, wherein the activator receptor comprises an scFv.

44. The engineered cell of claim 43, wherein the scFv comprises a sequence of any one of SEQ ID NO: 29-31, or a sequence having at least 85%, at least 90%, at least 95%, at least 97% or at least 99% identity thereto.

45. The engineered cell of claim 42, wherein the CAR comprises a CD8 or CD28 transmembrane domain.

46. The engineered cell of claim 42, wherein the CAR comprises CD28 and 4-1BB intracellular domains and a CD3z intracellular domain.

47. The engineered cell of claim 42, wherein the activator receptor comprises a sequence having at least 85%, at least 90%, at least 95%, at least 97%, at least 99% identity to any one of SEQ ID NOs. 84-104.

48. The engineered cell of claim 38 or claim 39, wherein the inhibitory receptor is a chimeric antigen receptor (CAR).

49. The engineered cell of claim 48, wherein the inhibitory receptor comprises an scFv.

50. The engineered cell of claim 49, wherein the scFv comprises a sequence of SEQ ID NO: 29, or a sequence having at least 85%, at least 90%, at least 95%, at least 97% or at least 99% identity thereto.

51. The engineered cell of claim 36 or claim 37, wherein the inhibitory receptor comprises a LILRB1 intracellular domain or a functional variant thereof.

52. The engineered cell of claim 36 or claim 37, wherein the inhibitory receptor comprises one or more EGF hinge elements.

53. The engineered cell of claim 36 or claim 37, wherein the inhibitory receptor comprises 5, 6, or 7 EGF hinge elements.

54. The engineered cell of claim 36 or claim 37, wherein the inhibitory receptor comprises a sequence having at least 85%, at least 90%, at least 95%, at least 97%, at least 99% identity to any one of SEQ ID NO: 50.

55. A pharmaceutical composition, comprising a therapeutically effective amount of the engineered cell of claim 36 or claim 37.

56. The pharmaceutical composition of claim 55 further comprising a pharmaceutically acceptable carrier, diluent, or excipient.

57. A method of selectively killing targets cells expressing a first antigen, optionally MSLN and not LRRN4, comprising contacting target cells with the engineered immune cell of claim 36 or claim 37.

58. A method of treating or preventing cancer in a subject, comprising administering to the subject the engineered cells of claim 36 or claim 37.

59. A method of increasing the selective inhibition of an immune cell by an inhibitory receptor, comprising:
generating an immune cell comprising:
an inhibitory receptor that specifically binds an inhibitor antigen, and
an activator receptor that specifically binds an activator antigen,
wherein the activator receptor comprises one or more hinge elements;
wherein selective inhibition by the inhibitory receptor is increased compared to an immune cell comprising the same inhibitor receptor and a reference activator receptor lacking the hinge element.

60. A method of increasing the selective inhibition activity of an inhibitor receptor in an immune cell comprising an activator receptor, comprising inserting into the extracellular domain of the activator receptor one or more hinge elements until selective inhibition is maximized.

61. A method of increasing the selective inhibition activity of an inhibitor receptor in an immune cell comprising an activator receptor, comprising removing polypeptide segments from the inhibitor receptor until selective inhibition is maximized.

62. The method of one of claims 59 to 61, wherein selective inhibition is maximized when R_{A}≥[T_{A}−E_{A}].


SEQUENCE LISTING (540 sequences)

SEQ ID NO: 1 - protein, 5 aa, synthetic construct
GGGGQ

SEQ ID NO: 2 - protein, 49 aa, synthetic construct
DPVLCFTQYEESSGKCKGLLGGGVSVEDCCLNTAFAYQKRSGGLCQPCR

SEQ ID NO: 3 - protein, 58 aa, synthetic construct
SPRWSLWSTWAPCSVTCSEGSQLRYRRCVGWNGQCSGKVAPGTLEWQLQACEDQQCCP

SEQ ID NO: 4 - protein, 63 aa, synthetic construct
HGAWATWGPWTPCSASCHGGPHEPKETRSRKCSAPEPSQKPPGKPCPGLAYEQRRCTGLPPCP

SEQ ID NO: 5 - protein, 57 aa, synthetic construct
AGGWGPWGPVSPCPVTCGLGQTMEQRTCNHPVPQHGGPFCAGDATRTHICNTAVPCP

SEQ ID NO: 6 - protein, 63 aa, synthetic construct
DGEWDSWGEWSPCIRRNMKSISCQEIPGQQSRGRTCRGRKFDGHRCAGQQQDIRHCYSIQHCP

SEQ ID NO: 7 - protein, 84 aa, synthetic construct
KGSWSEWSTWGLCMPPCGPNPTRARQRLCTPLLPKYPPTVSMVEGQGEKNVTFWGRPLPRCEELQGQKLVVEEKRPCLHVPACK

SEQ ID NO: 8 - protein, 83 aa, synthetic construct
PDTPIISPPDSSYLSGANLNLSCHSASNPSPQYSWRINGIPQQHTQVLFIAKITPNNNGTYACFVSNLATGRNNSIVKSITVS

SEQ ID NO: 9 - protein, 88 aa, synthetic construct
PKPSISSNNSKPVEDKDAVAFTCEPEAQNTTYLWWVNGQSLPVSPRLQLSNGNRTLTLFNVTRNDARAYVCGIQNSVSANRSDPVTLD

SEQ ID NO: 10 - protein, 78 aa, synthetic construct
PTISPSYTYYRPGVNLSLSCHAASNPPAQYSWLIDGNIQQHTQELFISNITEKNSGLYTCQANNSASGHSRTTVKTIT

SEQ ID NO: 11 - protein, 88 aa, synthetic construct
PKPFITSNNSNPVEDEDAVALTCEPEIQNTTYLWWVNNQSLPVSPRLQLSNDNRTLTLLSVTRNDVGPYECGIQNELSVDHSDPVILN

SEQ ID NO: 12 - protein, 76 aa, synthetic construct
PTISPLNTSYRSGENLNLSCHAASNPPAQYSWFVNGTFQQSTQELFIPNITVNNSGSYTCQAHNSDTGLNRTTVTT

SEQ ID NO: 13 - protein, 88 aa, synthetic construct
PKPSISSNNSKPVEDKDAVAFTCEPETQDATYLWWVNNQSLPVSPRLQLSNGNRTLTLFNVTRNDTASYKCETQNPVSARRSDSVILN

SEQ ID NO: 14 - protein, 37 aa, synthetic construct
SCLTCVGHCSNGGSCTMNSKMMPECQCPPHMTGPRCE

SEQ ID NO: 15 - protein, 36 aa, synthetic construct
EVNKCSRCLEGACVVNKQSGDVTCNCTDGRVAPSCL

SEQ ID NO: 16 - protein, 37 aa, synthetic construct
QYRQCSGYCENFGTCQMAADGSRQCRCTAYFEGSRCE

SEQ ID NO: 17 - protein, 37 aa, synthetic construct
TQQVCAGYCANNSTCTVNQGNQPQCRCLPGFLGDRCQ

SEQ ID NO: 18 - protein, 37 aa, synthetic construct
ELDQCWEHCRNGGTCAASPSGMPTCRCPTGFTGPKCT

SEQ ID NO: 19 - protein, 37 aa, synthetic construct
RPGTCNLQCFNGGSCFLNARRQPKCRCQPRYTGDKCE

SEQ ID NO: 20 - protein, 37 aa, synthetic construct
VTNPCDRKKCEWLCLLSPSGPVCTCPNGKRLDNGTCV

SEQ ID NO: 21 - protein, 93 aa, synthetic construct
PPSQVWNMTVSMTSDNSMHVKCRPPRDRNGPHERYHLEVEAGNTLVRNESHKNCDFRVKDLQYSTDYTFKAYFHNGDYPGEPFILHHSTSYNS

SEQ ID NO: 22 - protein, 93 aa, synthetic construct
SPGEPQIIFCRSEAAHQGVITWNPPQRSFHNFTLCYIKETEKDCLNLDKNLIKYDLQNLKPYTKYVLSLHAYIIAKVQRNGSAAMCHFTTKSA

SEQ ID NO: 23 - protein, 622 aa, synthetic construct
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 24 - protein, 630 aa, synthetic construct
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQAPRRPLPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 25 - protein, 17 aa, synthetic construct
GGGGSGGGGSGGGGSGG

SEQ ID NO: 26 - protein, 22 aa, synthetic construct
MDMRVPAQLLGLLLLWLRGARC

SEQ ID NO: 27 - DNA, 66 bp, synthetic construct
  source 1..66: mol_type = other DNA
atggacatgagggtccccgctcagctcctggggctcctgctactctggctccgaggtgccagatgt

SEQ ID NO: 28 - protein, 244 aa, synthetic construct
QVQLQESGPGLVKPSQTLSLTCTVSGGSISSGDYYWSWIRQPPGKGLEWIGYIYYSGSTYYNPSLKSRVTISVDTSKNQFSLKLSSVTAADTAVYYCAREDVVKGAFDIWGQGTMVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 29 - protein, 241 aa, synthetic construct
QVQLKESGPGLVAPSQSLSITCTVSGFSLTNYAVHWVRQSPGKGLEWLGAIWSDGSTDYNAAFISRLSISKDNSKSQVFFKMNSLQADDTAIYYCARKDYSNYVFDYWGQGTTLTVSSGGGGSGGGGSGGGGSGGEIVLTQSPAITAASLGQKVTISCSASSSVSYMHWYQQKSGTSPKPWIYDISKLASGVPARFSGSGSGTSYSLTISSMEAEDAAIYYCQQWSYPLITFGAGTKLELK

SEQ ID NO: 30 - protein, 244 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSGGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 31 - protein, 242 aa, synthetic construct
QVQLVQSGAEVKKPGASVKVSCKASGYSFTGYTMNWVRQAPGQRLEWMGLITPYNGASSYNQKFRGRVTITRDTSASTAYMELSSLRSEDTAVYYCARGGYDGRGFDYWGQGTTVTVSSGGGGSGGGGSGGGGSGGDIQMTQSPSSLSASVGDRVTITCSASSSVSYMHWYQQKPGKAPKRLIYDTSKLASGVPSRFSGSGSGTEFTLTISSLQPEDFATYYCQQWSGYPLTFGQGTKLEIK

SEQ ID NO: 32 - protein, 27 aa, synthetic construct
FWVLVVVGGVLACYSLLVTVAFIIFWV

SEQ ID NO: 33 - DNA, 81 bp, synthetic construct
  source 1..81: mol_type = other DNA
ttctgggtgctggtcgttgtgggcggcgtgctggcctgctacagcctgctggtgacagtggccttcatcatcttttgggtg

SEQ ID NO: 34 - protein, 25 aa, synthetic construct
IPWLGHLLVGLSGAFGFIILVYLLI

SEQ ID NO: 35 - protein, 75 aa, synthetic construct
ATTCCGTGGCTCGGCCACCTCCTCGTGGGCCTCAGCGGGGCTTTTGGCTTCATCATCTTAGTGTACTTGCTGATC

SEQ ID NO: 36 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - DNA, 336 bp, synthetic construct
  source 1..336: mol_type = other DNA
agagtgaagttcagcaggagcgcagacgcccccgcgtacaagcagggccagaaccagctctataacgagctcaatctaggacgaagagaggagtacgatgttttggacaagcgtagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 38 - skipped

SEQ ID NO: 39 - protein, 43 aa, synthetic construct
RVKFSRSADAPAYQQGQNQLYNELNLGRREEYDVLHMQALPPR

SEQ ID NO: 40 - DNA, 129 bp, synthetic construct
  source 1..129: mol_type = other DNA
agagtgaagttcagcaggagcgcagacgcccccgcgtaccagcagggccagaaccagctctataacgagctcaatctaggacgaagagaggagtacgatgttttgcacatgcaggccctgccccctcgc

SEQ ID NO: 41 - protein, 41 aa, synthetic construct
RSKRSRLLHSDYMNMTPRRPGPTRKHYQPYAPPRDFAAYRS

SEQ ID NO: 42 - DNA, 123 bp, synthetic construct
  source 1..123: mol_type = other DNA
aggagcaagcggagcagactgctgcacagcgactacatgaacatgaccccccggaggcctggccccacccggaagcactaccagccctacgcccctcccagggatttcgccgcctaccggagc

SEQ ID NO: 43 - protein, 42 aa, synthetic construct
KRGRKKLLYIFKQPFMRPVQTTQEEDGCSCRFPEEEEGGCEL

SEQ ID NO: 44 - DNA, 126 bp, synthetic construct
  source 1..126: mol_type = other DNA
aaacggggcagaaagaaactcctgtatatattcaaacaaccatttatgaggccagtacaaactactcaagaggaagatggctgtagctgccgatttccagaagaagaagaaggaggatgtgaactg

SEQ ID NO: 45 - protein, 195 aa, synthetic construct
RSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - protein, 17 aa, synthetic construct
GGGGSGGGGSGGGGSGG

SEQ ID NO: 47 - protein, 532 aa, synthetic construct
MAPSSPRPALPALLVLLGALFPGPGNAQTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTRKVTVNVLSPRYEIVIITVVAAAVIMGTAGLSTYLYNRQRKIKKYRLQQAQKGTPMKPNTQATPP

SEQ ID NO: 48 - protein, 271 aa, synthetic construct
MPEAPPLLLAAVLLGLVLLVVLLLLLRHWGWGLCLIGWNEFILQPIHNLLMGDTKEQRILNHVLQHAEPGNAQSVLEAIDTYCEQKEWAMNVGDKKGKIVDAVIQEHQPSVLLELGAYCGYSAVRMARLLSPGARLITIEINPDCAAITQRMVDFAGVKDKVTLVVGASQDIIPQLKKKYDVDTLDMVFLDHWKDRYLPDTLLLEECGLLRKGTVLLADNVICPGAPDFLAHVRGSSCFECTHYQSFLEYREVVDGLEKAIYKGPGSEAGP

SEQ ID NO: 49 - protein, 273 aa, synthetic construct
MSGSQSEVAPSPQSPRSPEMGRDLRPGSRVLLLLLLLLLVYLTQPGNGNEGSVTGSCYCGKRISSDSPPSVQFMNRLRKHLRAYHRCLYYTRFQLLSWSVCGGNKDPWVQELMSCLDLKECGHAYSGIVAHQKHLLPTSPPISQASEGASSDIHTPAQMLLSTLQSTQRPTLPVGSLSSDKELTRPNETTIHTAGHSLAAGPEAGENQKQPEKNAGPTARTSATVPVLCLLAIIFILTAALSYVLCKRRRGQSPQSSPDLPVHYIPVAPDSNT

SEQ ID NO: 50 - protein, 494 aa, synthetic construct
QVQLKESGPGLVAPSQSLSITCTVSGFSLTNYAVHWVRQSPGKGLEWLGAIWSDGSTDYNAAFISRLSISKDNSKSQVFFKMNSLQADDTAIYYCARKDYSNYVFDYWGQGTTLTVSSGGGGSGGGGSGGGGSGGEIVLTQSPAITAASLGQKVTISCSASSSVSYMHWYQQKSGTSPKPWIYDISKLASGVPARFSGSGSGTSYSLTISSMEAEDAAIYYCQQWSYPLITFGAGTKLELKYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 51 - protein, 470 aa, synthetic construct
QVQLKESGPGLVAPSQSLSITCTVSGFSLTNYAVHWVRQSPGKGLEWLGAIWSDGSTDYNAAFISRLSISKDNSKSQVFFKMNSLQADDTAIYYCARKDYSNYVFDYWGQGTTLTVSSGGGGSGGGGSGGGGSGGEIVLTQSPAITAASLGQKVTISCSASSSVSYMHWYQQKSGTSPKPWIYDISKLASGVPARFSGSGSGTSYSLTISSMEAEDAAIYYCQQWSYPLITFGAGTKLELKGGGGLTCVGHCSNGGSCTMNSKMMPECQCPPHMTGPRCEVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 52 - protein, 541 aa, synthetic construct
QVQLKESGPGLVAPSQSLSITCTVSGFSLTNYAVHWVRQSPGKGLEWLGAIWSDGSTDYNAAFISRLSISKDNSKSQVFFKMNSLQADDTAIYYCARKDYSNYVFDYWGQGTTLTVSSGGGGSGGGGSGGGGSGGEIVLTQSPAITAASLGQKVTISCSASSSVSYMHWYQQKSGTSPKPWIYDISKLASGVPARFSGSGSGTSYSLTISSMEAEDAAIYYCQQWSYPLITFGAGTKLELKGGGGQYRQCSGYCENFGTCQMAADGSRQCRCTAYFEGSRCEVNKCSRCLEGACVVNKQSGDVTCNCTDGRVAPSCLTCVGHCSNGGSCTMNSKMMPECQCPPHMTGPRCEVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 53 - protein, 613 aa, synthetic construct
QVQLKESGPGLVAPSQSLSITCTVSGFSLTNYAVHWVRQSPGKGLEWLGAIWSDGSTDYNAAFISRLSISKDNSKSQVFFKMNSLQADDTAIYYCARKDYSNYVFDYWGQGTTLTVSSGGGGSGGGGSGGGGSGGEIVLTQSPAITAASLGQKVTISCSASSSVSYMHWYQQKSGTSPKPWIYDISKLASGVPARFSGSGSGTSYSLTISSMEAEDAAIYYCQQWSYPLITFGAGTKLELKGGGGELDQCWEHCRNGGTCAASPSGMPTCRCPTGFTGPKCTQQVCAGYCANNSTCTVNQGNQPQCRCLPGFLGDRCQYRQCSGYCENFGTCQMAADGSRQCRCTAYFEGSRCEVNKCSRCLEGACVVNKQSGDVTCNCTDGRVAPSCLTCVGHCSNGGSCTMNSKMMPECQCPPHMTGPRCEVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 54 - protein, 698 aa, synthetic construct
QVQLKESGPGLVAPSQSLSITCTVSGFSLTNYAVHWVRQSPGKGLEWLGAIWSDGSTDYNAAFISRLSISKDNSKSQVFFKMNSLQADDTAIYYCARKDYSNYVFDYWGQGTTLTVSSGGGGSGGGGSGGGGSGGEIVLTQSPAITAASLGQKVTISCSASSSVSYMHWYQQKSGTSPKPWIYDISKLASGVPARFSGSGSGTSYSLTISSMEAEDAAIYYCQQWSYPLITFGAGTKLELKGGGGVTNPCDRKKCEWLCLLSPSGPVCTCPNGKRLDNGTCVPVPSPTPPPDAPRPGTCNLQCFNGGSCFLNARRQPKCRCQPRYTGDKCELDQCWEHCRNGGTCAASPSGMPTCRCPTGFTGPKCTQQVCAGYCANNSTCTVNQGNQPQCRCLPGFLGDRCQYRQCSGYCENFGTCQMAADGSRQCRCTAYFEGSRCEVNKCSRCLEGACVVNKQSGDVTCNCTDGRVAPSCLTCVGHCSNGGSCTMNSKMMPECQCPPHMTGPRCEVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 55 - protein, 446 aa, synthetic construct
QVQLKESGPGLVAPSQSLSITCTVSGFSLTNYAVHWVRQSPGKGLEWLGAIWSDGSTDYNAAFISRLSISKDNSKSQVFFKMNSLQADDTAIYYCARKDYSNYVFDYWGQGTTLTVSSGGGGSGGGGSGGGGSGGEIVLTQSPAITAASLGQKVTISCSASSSVSYMHWYQQKSGTSPKPWIYDISKLASGVPARFSGSGSGTSYSLTISSMEAEDAAIYYCQQWSYPLITFGAGTKLELKGGGGQGGGGQGGGGQVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 56 - protein, 461 aa, synthetic construct
QVQLKESGPGLVAPSQSLSITCTVSGFSLTNYAVHWVRQSPGKGLEWLGAIWSDGSTDYNAAFISRLSISKDNSKSQVFFKMNSLQADDTAIYYCARKDYSNYVFDYWGQGTTLTVSSGGGGSGGGGSGGGGSGGEIVLTQSPAITAASLGQKVTISCSASSSVSYMHWYQQKSGTSPKPWIYDISKLASGVPARFSGSGSGTSYSLTISSMEAEDAAIYYCQQWSYPLITFGAGTKLELKGGGGQGGGGQGGGGQGGGGQGGGGQGGGGQVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 57 - protein, 481 aa, synthetic construct
QVQLKESGPGLVAPSQSLSITCTVSGFSLTNYAVHWVRQSPGKGLEWLGAIWSDGSTDYNAAFISRLSISKDNSKSQVFFKMNSLQADDTAIYYCARKDYSNYVFDYWGQGTTLTVSSGGGGSGGGGSGGGGSGGEIVLTQSPAITAASLGQKVTISCSASSSVSYMHWYQQKSGTSPKPWIYDISKLASGVPARFSGSGSGTSYSLTISSMEAEDAAIYYCQQWSYPLITFGAGTKLELKGGGGQGGGGQGGGGQGGGGQGGGGQGGGGQGGGGQGGGGQGGGGQGGGGQVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 58 - protein, 491 aa, synthetic construct
QVQLKESGPGLVAPSQSLSITCTVSGFSLTNYAVHWVRQSPGKGLEWLGAIWSDGSTDYNAAFISRLSISKDNSKSQVFFKMNSLQADDTAIYYCARKDYSNYVFDYWGQGTTLTVSSGGGGSGGGGSGGGGSGGEIVLTQSPAITAASLGQKVTISCSASSSVSYMHWYQQKSGTSPKPWIYDISKLASGVPARFSGSGSGTSYSLTISSMEAEDAAIYYCQQWSYPLITFGAGTKLELKGGGGQGGGGQGGGGQGGGGQGGGGQGGGGQGGGGQGGGGQGGGGQGGGGQGGGGQGGGGQVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 59 - protein, 550 aa, synthetic construct
QVQLKESGPGLVAPSQSLSITCTVSGFSLTNYAVHWVRQSPGKGLEWLGAIWSDGSTDYNAAFISRLSISKDNSKSQVFFKMNSLQADDTAIYYCARKDYSNYVFDYWGQGTTLTVSSGGGGSGGGGSGGGGSGGEIVLTQSPAITAASLGQKVTISCSASSSVSYMHWYQQKSGTSPKPWIYDISKLASGVPARFSGSGSGTSYSLTISSMEAEDAAIYYCQQWSYPLITFGAGTKLELKGGGGSPRWSLWSTWAPCSVTCSEGSQLRYRRCVGWNGQCSGKVAPGTLEWQLQACEDQQCCPEMGGWSGWGPWEPCSVTCSKGTRTRRRACNHPAPKCGGHCPGQAQESEACDTQQVCPVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 60 - protein, 672 aa, synthetic construct
QVQLKESGPGLVAPSQSLSITCTVSGFSLTNYAVHWVRQSPGKGLEWLGAIWSDGSTDYNAAFISRLSISKDNSKSQVFFKMNSLQADDTAIYYCARKDYSNYVFDYWGQGTTLTVSSGGGGSGGGGSGGGGSGGEIVLTQSPAITAASLGQKVTISCSASSSVSYMHWYQQKSGTSPKPWIYDISKLASGVPARFSGSGSGTSYSLTISSMEAEDAAIYYCQQWSYPLITFGAGTKLELKGGGGSPRWSLWSTWAPCSVTCSEGSQLRYRRCVGWNGQCSGKVAPGTLEWQLQACEDQQCCPEMGGWSGWGPWEPCSVTCSKGTRTRRRACNHPAPKCGGHCPGQAQESEACDTQQVCPTHGAWATWGPWTPCSASCHGGPHEPKETRSRKCSAPEPSQKPPGKPCPGLAYEQRRCTGLPPCPVAGGWGPWGPVSPCPVTCGLGQTMEQRTCNHPVPQHGGPFCAGDATRTHICNTAVPCPVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 61 - protein, 821 aa, synthetic construct
QVQLKESGPGLVAPSQSLSITCTVSGFSLTNYAVHWVRQSPGKGLEWLGAIWSDGSTDYNAAFISRLSISKDNSKSQVFFKMNSLQADDTAIYYCARKDYSNYVFDYWGQGTTLTVSSGGGGSGGGGSGGGGSGGEIVLTQSPAITAASLGQKVTISCSASSSVSYMHWYQQKSGTSPKPWIYDISKLASGVPARFSGSGSGTSYSLTISSMEAEDAAIYYCQQWSYPLITFGAGTKLELKGGGGSPRWSLWSTWAPCSVTCSEGSQLRYRRCVGWNGQCSGKVAPGTLEWQLQACEDQQCCPEMGGWSGWGPWEPCSVTCSKGTRTRRRACNHPAPKCGGHCPGQAQESEACDTQQVCPTHGAWATWGPWTPCSASCHGGPHEPKETRSRKCSAPEPSQKPPGKPCPGLAYEQRRCTGLPPCPVAGGWGPWGPVSPCPVTCGLGQTMEQRTCNHPVPQHGGPFCAGDATRTHICNTAVPCPVDGEWDSWGEWSPCIRRNMKSISCQEIPGQQSRGRTCRGRKFDGHRCAGQQQDIRHCYSIQHCPLKGSWSEWSTWGLCMPPCGPNPTRARQRLCTPLLPKYPPTVSMVEGQGEKNVTFWGRPLPRCEELQGQKLVVEEKRPCLHVPACKVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 62 - protein, 610 aa, synthetic construct
QVQLKESGPGLVAPSQSLSITCTVSGFSLTNYAVHWVRQSPGKGLEWLGAIWSDGSTDYNAAFISRLSISKDNSKSQVFFKMNSLQADDTAIYYCARKDYSNYVFDYWGQGTTLTVSSGGGGSGGGGSGGGGSGGEIVLTQSPAITAASLGQKVTISCSASSSVSYMHWYQQKSGTSPKPWIYDISKLASGVPARFSGSGSGTSYSLTISSMEAEDAAIYYCQQWSYPLITFGAGTKLELKGGGGPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 63 - protein, 788 aa, synthetic construct
QVQLKESGPGLVAPSQSLSITCTVSGFSLTNYAVHWVRQSPGKGLEWLGAIWSDGSTDYNAAFISRLSISKDNSKSQVFFKMNSLQADDTAIYYCARKDYSNYVFDYWGQGTTLTVSSGGGGSGGGGSGGGGSGGEIVLTQSPAITAASLGQKVTISCSASSSVSYMHWYQQKSGTSPKPWIYDISKLASGVPARFSGSGSGTSYSLTISSMEAEDAAIYYCQQWSYPLITFGAGTKLELKGGGGPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 64 - protein, 966 aa, synthetic construct
QVQLKESGPGLVAPSQSLSITCTVSGFSLTNYAVHWVRQSPGKGLEWLGAIWSDGSTDYNAAFISRLSISKDNSKSQVFFKMNSLQADDTAIYYCARKDYSNYVFDYWGQGTTLTVSSGGGGSGGGGSGGGGSGGEIVLTQSPAITAASLGQKVTISCSASSSVSYMHWYQQKSGTSPKPWIYDISKLASGVPARFSGSGSGTSYSLTISSMEAEDAAIYYCQQWSYPLITFGAGTKLELKGGGGPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 65 - protein, 471 aa, synthetic construct
QVQLVQSGAEVKKPGASVKVSCKASGYSFTGYTMNWVRQAPGQRLEWMGLITPYNGASSYNQKFRGRVTITRDTSASTAYMELSSLRSEDTAVYYCARGGYDGRGFDYWGQGTTVTVSSGGGGSGGGGSGGGGSGGDIQMTQSPSSLSASVGDRVTITCSASSSVSYMHWYQQKPGKAPKRLIYDTSKLASGVPSRFSGSGSGTEFTLTISSLQPEDFATYYCQQWSGYPLTFGQGTKLEIKGGGGLTCVGHCSNGGSCTMNSKMMPECQCPPHMTGPRCEVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 66 - protein, 506 aa, synthetic construct
QVQLVQSGAEVKKPGASVKVSCKASGYSFTGYTMNWVRQAPGQRLEWMGLITPYNGASSYNQKFRGRVTITRDTSASTAYMELSSLRSEDTAVYYCARGGYDGRGFDYWGQGTTVTVSSGGGGSGGGGSGGGGSGGDIQMTQSPSSLSASVGDRVTITCSASSSVSYMHWYQQKPGKAPKRLIYDTSKLASGVPSRFSGSGSGTEFTLTISSLQPEDFATYYCQQWSGYPLTFGQGTKLEIKGGGGEVNKCSRCLEGACVVNKQSGDVTCNCTDGRVAPSCLTCVGHCSNGGSCTMNSKMMPECQCPPHMTGPRCEVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 67 - protein, 542 aa, synthetic construct
QVQLVQSGAEVKKPGASVKVSCKASGYSFTGYTMNWVRQAPGQRLEWMGLITPYNGASSYNQKFRGRVTITRDTSASTAYMELSSLRSEDTAVYYCARGGYDGRGFDYWGQGTTVTVSSGGGGSGGGGSGGGGSGGDIQMTQSPSSLSASVGDRVTITCSASSSVSYMHWYQQKPGKAPKRLIYDTSKLASGVPSRFSGSGSGTEFTLTISSLQPEDFATYYCQQWSGYPLTFGQGTKLEIKGGGGQYRQCSGYCENFGTCQMAADGSRQCRCTAYFEGSRCEVNKCSRCLEGACVVNKQSGDVTCNCTDGRVAPSCLTCVGHCSNGGSCTMNSKMMPECQCPPHMTGPRCEVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 68 - protein, 578 aa, synthetic construct
QVQLVQSGAEVKKPGASVKVSCKASGYSFTGYTMNWVRQAPGQRLEWMGLITPYNGASSYNQKFRGRVTITRDTSASTAYMELSSLRSEDTAVYYCARGGYDGRGFDYWGQGTTVTVSSGGGGSGGGGSGGGGSGGDIQMTQSPSSLSASVGDRVTITCSASSSVSYMHWYQQKPGKAPKRLIYDTSKLASGVPSRFSGSGSGTEFTLTISSLQPEDFATYYCQQWSGYPLTFGQGTKLEIKGGGGTQQVCAGYCANNSTCTVNQGNQPQCRCLPGFLGDRCQYRQCSGYCENFGTCQMAADGSRQCRCTAYFEGSRCEVNKCSRCLEGACVVNKQSGDVTCNCTDGRVAPSCLTCVGHCSNGGSCTMNSKMMPECQCPPHMTGPRCEVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 69 - protein, 614 aa, synthetic construct
QVQLVQSGAEVKKPGASVKVSCKASGYSFTGYTMNWVRQAPGQRLEWMGLITPYNGASSYNQKFRGRVTITRDTSASTAYMELSSLRSEDTAVYYCARGGYDGRGFDYWGQGTTVTVSSGGGGSGGGGSGGGGSGGDIQMTQSPSSLSASVGDRVTITCSASSSVSYMHWYQQKPGKAPKRLIYDTSKLASGVPSRFSGSGSGTEFTLTISSLQPEDFATYYCQQWSGYPLTFGQGTKLEIKGGGGELDQCWEHCRNGGTCAASPSGMPTCRCPTGFTGPKCTQQVCAGYCANNSTCTVNQGNQPQCRCLPGFLGDRCQYRQCSGYCENFGTCQMAADGSRQCRCTAYFEGSRCEVNKCSRCLEGACVVNKQSGDVTCNCTDGRVAPSCLTCVGHCSNGGSCTMNSKMMPECQCPPHMTGPRCEVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 70 - protein, 650 aa, synthetic construct
QVQLVQSGAEVKKPGASVKVSCKASGYSFTGYTMNWVRQAPGQRLEWMGLITPYNGASSYNQKFRGRVTITRDTSASTAYMELSSLRSEDTAVYYCARGGYDGRGFDYWGQGTTVTVSSGGGGSGGGGSGGGGSGGDIQMTQSPSSLSASVGDRVTITCSASSSVSYMHWYQQKPGKAPKRLIYDTSKLASGVPSRFSGSGSGTEFTLTISSLQPEDFATYYCQQWSGYPLTFGQGTKLEIKGGGGRPGTCNLQCFNGGSCFLNARRQPKCRCQPRYTGDKCELDQCWEHCRNGGTCAASPSGMPTCRCPTGFTGPKCTQQVCAGYCANNSTCTVNQGNQPQCRCLPGFLGDRCQYRQCSGYCENFGTCQMAADGSRQCRCTAYFEGSRCEVNKCSRCLEGACVVNKQSGDVTCNCTDGRVAPSCLTCVGHCSNGGSCTMNSKMMPECQCPPHMTGPRCEVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 71 - protein, 699 aa, synthetic construct
QVQLVQSGAEVKKPGASVKVSCKASGYSFTGYTMNWVRQAPGQRLEWMGLITPYNGASSYNQKFRGRVTITRDTSASTAYMELSSLRSEDTAVYYCARGGYDGRGFDYWGQGTTVTVSSGGGGSGGGGSGGGGSGGDIQMTQSPSSLSASVGDRVTITCSASSSVSYMHWYQQKPGKAPKRLIYDTSKLASGVPSRFSGSGSGTEFTLTISSLQPEDFATYYCQQWSGYPLTFGQGTKLEIKGGGGVTNPCDRKKCEWLCLLSPSGPVCTCPNGKRLDNGTCVPVPSPTPPPDAPRPGTCNLQCFNGGSCFLNARRQPKCRCQPRYTGDKCELDQCWEHCRNGGTCAASPSGMPTCRCPTGFTGPKCTQQVCAGYCANNSTCTVNQGNQPQCRCLPGFLGDRCQYRQCSGYCENFGTCQMAADGSRQCRCTAYFEGSRCEVNKCSRCLEGACVVNKQSGDVTCNCTDGRVAPSCLTCVGHCSNGGSCTMNSKMMPECQCPPHMTGPRCEVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 72 - protein, 495 aa, synthetic construct
QVQLVQSGAEVKKPGASVKVSCKASGYSFTGYTMNWVRQAPGQRLEWMGLITPYNGASSYNQKFRGRVTITRDTSASTAYMELSSLRSEDTAVYYCARGGYDGRGFDYWGQGTTVTVSSGGGGSGGGGSGGGGSGGDIQMTQSPSSLSASVGDRVTITCSASSSVSYMHWYQQKPGKAPKRLIYDTSKLASGVPSRFSGSGSGTEFTLTISSLQPEDFATYYCQQWSGYPLTFGQGTKLEIKYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 73 - protein, 738 aa, synthetic construct
MRQTLPLLLLTVLRPSWADPPQEKVPLFRVTQQGPWGSSGSNATDSPCEGLPAADATALTLANRNLERLPGCLPRTLRSLDASHNLLRALSTSELGHLEQLQVLTLRHNRIAALRWGPGGPAGLHTLDLSYNQLAALPPCTGPALSSLRALALAGNPLRALQPRAFACFPALQLLNLSCTALGRGAQGGIAEAAFAGEDGAPLVTLEVLDLSGTFLERVESGWIRDLPKLTSLYLRKMPRLTTLEGDIFKMTPNLQQLDCQDSPALASVATHIFQDTPHLQVLLFQNCNLSSFPPWTLDSSQVLSINLFGNPLTCSCDLSWLLTDAKRTVLSRAADTMCAPAAGSSGPFSASLSLSQLPGVCQSDQSTTLGASHPPCFNRSTYAQGTTVAPSAAPATRPAGDQQSVSKAPNVGSRTIAAWPHSDAREGTAPSTTNSVAGHSNSSVFPRAASTTRTQHRGEHAPELVLEPDISAASTPLASKLLGPFPTSWDRSISSPQPGQRTHATPQAPNPSLSEGEIPVLLLDDYSEEEEGRKEEVGTPHQDVPCDYHPCKHLQTPCAELQRRWRCRCPGLSGEDTIPDPPRLQGVTETTDTSALVHWCAPNSVVHGYQIRYSAEGWAGNQSVVGVIYATARQHPLYGLSPGTTYRVCVLAANRAGLSRSSGWRSPCAAFTTKPSFALLLSGLCAASGLLLASTVVLSACLCRRGQTLGLQRCDTHLVAYKNPAFDDYPLGLQTVS

SEQ ID NO: 74 - protein, 320 aa, synthetic construct
MGLPWGQPHLGLQMLLLALNCLRPSLSLGEWGSWMDASSQTQGAGGPAGVIGPWAPAPLRLGEAAPGTPTPVSVAHLLSPVATELVPYTPQITAWDLEGKVTATTFSLEQPRCVFDGLASASDTVWLVVAFSNASRGFQNPETLADIPASPQLLTDGHYMTLPLSPDQLPCGDPMAGSGGAPVLRVGHDHGCHQQPFCNAPLPGPGPYREDPRIHRHLARAAKWQHDRHYLHPLFSGRPPTLGLLGSLYHALLQPVVAGGGPGAAADRLLHGQALHDPPHPTQRGRHTAGGLQAWPGPPPQPQPLAWPLCMGLGEMGRRE

SEQ ID NO: 75 - protein, 276 aa, synthetic construct
MGLPWGQPHLGLQMLLLALNCLRPSLSLELVPYTPQITAWDLEGKVTATTFSLEQPRCVFDGLASASDTVWLVVAFSNASRGFQNPETLADIPASPQLLTDGHYMTLPLSPDQLPCGDPMAGSGGAPVLRVGHDHGCHQQPFCNAPLPGPGPYRVKFLLMDTRGSPRAETKWSDPITLHQGKTPGSIDTWPGRRSGSMIVITSILSSLAGLLLLAFLAASTMRFSSLWWPEEAPEQLRIGSFMGKRYMTHHIPPSEAATLPVGCKPGLDPLPSLSP

SEQ ID NO: 76 - protein, 265 aa, synthetic construct
MGLPWGQPHLGLQMLLLALNCLRPSLSLELVPYTPQITAWDLEGKVTATTFSLEQPRCVFDGLASASDTVWLVVAFSNASRGFQNPETLADIPASPQLLTDGHYMTLPLSPDQLPCGDPMAGSGGAPVLRVGHDHGCHQQPFCNAPLPGPGPYREDPRIHRHLARAAKWQHDRHYLHPLFSGRPPTLGLLGSLYHALLQPVVAGGGPGAAADRLLHGQALHDPPHPTQRGRHTAGGLQAWPGPPPQPQPLAWPLCMGLGEMGRRE

SEQ ID NO: 77 - protein, 276 aa, synthetic construct
MGLPWGQPHLGLQMLLLALNCLRPSLSLELVPYTPQITAWDLEGKVTATTFSLEQPRCVFDGLASASDTVWLVVAFSNASRGFQNPETLADIPASPQLLTDGHYMTLPLSPDQLPCGDPMAGSGGAPVLRVGHDHGCHQQPFCNAPLPGPGPYRVKFLLMDTRGSPRAETKWSDPITLHQGKTPGSIDTWPGRRSGSMIVITSILSSLAGLLLLAFLAASTMRFSSLWWPEEAPEQLRIGSFMGKRYMTHHIPPREAATLPVGCKPGLDPLPSLSP

SEQ ID NO: 78 - protein, 18 aa, synthetic construct
EGRGSLLTCGDVEENPGP

SEQ ID NO: 79 - protein, 19 aa, synthetic construct
ATNFSLLKQAGDVEENPGP

SEQ ID NO: 80 - protein, 20 aa, synthetic construct
QCTNYALLKLAGDVESNPGP

SEQ ID NO: 81 - protein, 22 aa, synthetic construct
VKQTLNFDLLKLAGDVESNPGP

SEQ ID NO: 82 - protein, 21 aa, synthetic construct
GSGEGRGSLLTCGDVEENPGP

SEQ ID NO: 83 - DNA, 63 bp, synthetic construct
  source 1..63: mol_type = other DNA
ggatccggagagggcagaggcagcctgctgacatgtggcgacgtggaagagaaccctggcccc

SEQ ID NO: 84 - protein, 513 aa, synthetic construct
QVQLQESGPGLVKPSQTLSLTCTVSGGSISSGDYYWSWIRQPPGKGLEWIGYIYYSGSTYYNPSLKSRVTISVDTSKNQFSLKLSSVTAADTAVYYCAREDVVKGAFDIWGQGTMVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKGGGGLTCVGHCSNGGSCTMNSKMMPECQCPPHMTGPRCEVFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSEQIDNO

SEQ ID NO: 85 - protein, 584 aa, synthetic construct
QVQLQESGPGLVKPSQTLSLTCTVSGGSISSGDYYWSWIRQPPGKGLEWIGYIYYSGSTYYNPSLKSRVTISVDTSKNQFSLKLSSVTAADTAVYYCAREDVVKGAFDIWGQGTMVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKGGGGQYRQCSGYCENFGTCQMAADGSRQCRCTAYFEGSRCEVNKCSRCLEGACVVNKQSGDVTCNCTDGRVAPSCLTCVGHCSNGGSCTMNSKMMPECQCPPHMTGPRCEVFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSEQIDNO

SEQ ID NO: 86 - protein, 656 aa, synthetic construct
QVQLQESGPGLVKPSQTLSLTCTVSGGSISSGDYYWSWIRQPPGKGLEWIGYIYYSGSTYYNPSLKSRVTISVDTSKNQFSLKLSSVTAADTAVYYCAREDVVKGAFDIWGQGTMVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKGGGGELDQCWEHCRNGGTCAASPSGMPTCRCPTGFTGPKCTQQVCAGYCANNSTCTVNQGNQPQCRCLPGFLGDRCQYRQCSGYCENFGTCQMAADGSRQCRCTAYFEGSRCEVNKCSRCLEGACVVNKQSGDVTCNCTDGRVAPSCLTCVGHCSNGGSCTMNSKMMPECQCPPHMTGPRCEVFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSEQIDNO

SEQ ID NO: 87 - protein, 741 aa, synthetic construct
QVQLQESGPGLVKPSQTLSLTCTVSGGSISSGDYYWSWIRQPPGKGLEWIGYIYYSGSTYYNPSLKSRVTISVDTSKNQFSLKLSSVTAADTAVYYCAREDVVKGAFDIWGQGTMVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKGGGGVTNPCDRKKCEWLCLLSPSGPVCTCPNGKRLDNGTCVPVPSPTPPPDAPRPGTCNLQCFNGGSCFLNARRQPKCRCQPRYTGDKCELDQCWEHCRNGGTCAASPSGMPTCRCPTGFTGPKCTQQVCAGYCANNSTCTVNQGNQPQCRCLPGFLGDRCQYRQCSGYCENFGTCQMAADGSRQCRCTAYFEGSRCEVNKCSRCLEGACVVNKQSGDVTCNCTDGRVAPSCLTCVGHCSNGGSCTMNSKMMPECQCPPHMTGPRCEVFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSEQIDNO

SEQ ID NO: 88 - protein, 488 aa, synthetic construct
QVQLQESGPGLVKPSQTLSLTCTVSGGSISSGDYYWSWIRQPPGKGLEWIGYIYYSGSTYYNPSLKSRVTISVDTSKNQFSLKLSSVTAADTAVYYCAREDVVKGAFDIWGQGTMVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKGGGGQGGGGQGGGGQFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSEQIDNO

SEQ ID NO: 89 - protein, 503 aa, synthetic construct
QVQLQESGPGLVKPSQTLSLTCTVSGGSISSGDYYWSWIRQPPGKGLEWIGYIYYSGSTYYNPSLKSRVTISVDTSKNQFSLKLSSVTAADTAVYYCAREDVVKGAFDIWGQGTMVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKGGGGQGGGGQGGGGQGGGGQGGGGQGGGGQFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSEQIDNO

SEQ ID NO: 90 - protein, 523 aa, synthetic construct
QVQLQESGPGLVKPSQTLSLTCTVSGGSISSGDYYWSWIRQPPGKGLEWIGYIYYSGSTYYNPSLKSRVTISVDTSKNQFSLKLSSVTAADTAVYYCAREDVVKGAFDIWGQGTMVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKGGGGQGGGGQGGGGQGGGGQGGGGQGGGGQGGGGQGGGGQGGGGQGGGGQFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSEQIDNO

SEQ ID NO: 91 - protein, 533 aa, synthetic construct
QVQLQESGPGLVKPSQTLSLTCTVSGGSISSGDYYWSWIRQPPGKGLEWIGYIYYSGSTYYNPSLKSRVTISVDTSKNQFSLKLSSVTAADTAVYYCAREDVVKGAFDIWGQGTMVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKGGGGQGGGGQGGGGQGGGGQGGGGQGGGGQGGGGQGGGGQGGGGQGGGGQGGGGQGGGGQFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSEQIDNO

SEQ ID NO: 92 - protein, 592 aa, synthetic construct
QVQLQESGPGLVKPSQTLSLTCTVSGGSISSGDYYWSWIRQPPGKGLEWIGYIYYSGSTYYNPSLKSRVTISVDTSKNQFSLKLSSVTAADTAVYYCAREDVVKGAFDIWGQGTMVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKGGGGSPRWSLWSTWAPCSVTCSEGSQLRYRRCVGWNGQCSGKVAPGTLEWQLQACEDQQCCPEMGGWSGWGPWEPCSVTCSKGTRTRRRACNHPAPKCGGHCPGQAQESEACDTQQVCPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSEQIDNO

SEQ ID NO: 93 - protein, 714 aa, synthetic construct
QVQLQESGPGLVKPSQTLSLTCTVSGGSISSGDYYWSWIRQPPGKGLEWIGYIYYSGSTYYNPSLKSRVTISVDTSKNQFSLKLSSVTAADTAVYYCAREDVVKGAFDIWGQGTMVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKGGGGSPRWSLWSTWAPCSVTCSEGSQLRYRRCVGWNGQCSGKVAPGTLEWQLQACEDQQCCPEMGGWSGWGPWEPCSVTCSKGTRTRRRACNHPAPKCGGHCPGQAQESEACDTQQVCPTHGAWATWGPWTPCSASCHGGPHEPKETRSRKCSAPEPSQKPPGKPCPGLAYEQRRCTGLPPCPVAGGWGPWGPVSPCPVTCGLGQTMEQRTCNHPVPQHGGPFCAGDATRTHICNTAVPCPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSEQIDNO

SEQ ID NO: 94 - protein, 863 aa, synthetic construct
QVQLQESGPGLVKPSQTLSLTCTVSGGSISSGDYYWSWIRQPPGKGLEWIGYIYYSGSTYYNPSLKSRVTISVDTSKNQFSLKLSSVTAADTAVYYCAREDVVKGAFDIWGQGTMVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKGGGGSPRWSLWSTWAPCSVTCSEGSQLRYRRCVGWNGQCSGKVAPGTLEWQLQACEDQQCCPEMGGWSGWGPWEPCSVTCSKGTRTRRRACNHPAPKCGGHCPGQAQESEACDTQQVCPTHGAWATWGPWTPCSASCHGGPHEPKETRSRKCSAPEPSQKPPGKPCPGLAYEQRRCTGLPPCPVAGGWGPWGPVSPCPVTCGLGQTMEQRTCNHPVPQHGGPFCAGDATRTHICNTAVPCPVDGEWDSWGEWSPCIRRNMKSISCQEIPGQQSRGRTCRGRKFDGHRCAGQQQDIRHCYSIQHCPLKGSWSEWSTWGLCMPPCGPNPTRARQRLCTPLLPKYPPTVSMVEGQGEKNVTFWGRPLPRCEELQGQKLVVEEKRPCLHVPACKFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSEQIDNO

SEQ ID NO: 95 - protein, 652 aa, synthetic construct
QVQLQESGPGLVKPSQTLSLTCTVSGGSISSGDYYWSWIRQPPGKGLEWIGYIYYSGSTYYNPSLKSRVTISVDTSKNQFSLKLSSVTAADTAVYYCAREDVVKGAFDIWGQGTMVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKGGGGPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSEQIDNO

SEQ ID NO: 96 - protein, 830 aa, synthetic construct
QVQLQESGPGLVKPSQTLSLTCTVSGGSISSGDYYWSWIRQPPGKGLEWIGYIYYSGSTYYNPSLKSRVTISVDTSKNQFSLKLSSVTAADTAVYYCAREDVVKGAFDIWGQGTMVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKGGGGPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSEQIDNO

SEQ ID NO: 97 - protein, 1008 aa, synthetic construct
QVQLQESGPGLVKPSQTLSLTCTVSGGSISSGDYYWSWIRQPPGKGLEWIGYIYYSGSTYYNPSLKSRVTISVDTSKNQFSLKLSSVTAADTAVYYCAREDVVKGAFDIWGQGTMVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKGGGGPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSEQIDNO

SEQ ID NO: 98 - protein, 513 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSGGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGLTCVGHCSNGGSCTMNSKMMPECQCPPHMTGPRCEVFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSEQIDNO

SEQ ID NO: 99 - protein, 548 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSGGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGEVNKCSRCLEGACVVNKQSGDVTCNCTDGRVAPSCLTCVGHCSNGGSCTMNSKMMPECQCPPHMTGPRCEVFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSEQIDNO

SEQ ID NO: 100 - protein, 584 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSGGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGQYRQCSGYCENFGTCQMAADGSRQCRCTAYFEGSRCEVNKCSRCLEGACVVNKQSGDVTCNCTDGRVAPSCLTCVGHCSNGGSCTMNSKMMPECQCPPHMTGPRCEVFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSEQIDNO

SEQ ID NO: 101 - protein, 620 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSGGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGTQQVCAGYCANNSTCTVNQGNQPQCRCLPGFLGDRCQYRQCSGYCENFGTCQMAADGSRQCRCTAYFEGSRCEVNKCSRCLEGACVVNKQSGDVTCNCTDGRVAPSCLTCVGHCSNGGSCTMNSKMMPECQCPPHMTGPRCEVFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSEQIDNO

SEQ ID NO: 102 - protein, 656 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSGGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGELDQCWEHCRNGGTCAASPSGMPTCRCPTGFTGPKCTQQVCAGYCANNSTCTVNQGNQPQCRCLPGFLGDRCQYRQCSGYCENFGTCQMAADGSRQCRCTAYFEGSRCEVNKCSRCLEGACVVNKQSGDVTCNCTDGRVAPSCLTCVGHCSNGGSCTMNSKMMPECQCPPHMTGPRCEVFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSEQIDNO

SEQ ID NO: 103 - protein, 692 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSGGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGRPGTCNLQCFNGGSCFLNARRQPKCRCQPRYTGDKCELDQCWEHCRNGGTCAASPSGMPTCRCPTGFTGPKCTQQVCAGYCANNSTCTVNQGNQPQCRCLPGFLGDRCQYRQCSGYCENFGTCQMAADGSRQCRCTAYFEGSRCEVNKCSRCLEGACVVNKQSGDVTCNCTDGRVAPSCLTCVGHCSNGGSCTMNSKMMPECQCPPHMTGPRCEVFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSEQIDNO

SEQ ID NO: 104 - protein, 741 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSGGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGVTNPCDRKKCEWLCLLSPSGPVCTCPNGKRLDNGTCVPVPSPTPPPDAPRPGTCNLQCFNGGSCFLNARRQPKCRCQPRYTGDKCELDQCWEHCRNGGTCAASPSGMPTCRCPTGFTGPKCTQQVCAGYCANNSTCTVNQGNQPQCRCLPGFLGDRCQYRQCSGYCENFGTCQMAADGSRQCRCTAYFEGSRCEVNKCSRCLEGACVVNKQSGDVTCNCTDGRVAPSCLTCVGHCSNGGSCTMNSKMMPECQCPPHMTGPRCEVFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSEQIDNO

SEQ ID NO: 105 - protein, 45 aa, synthetic construct
TTTPAPRPPTPAPTIASQPLSLRPEACRPAAGGAVHTRGLDFACD

SEQ ID NO: 106 - DNA, 135 bp, synthetic construct
  source 1..135: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 107 - protein, 41 aa, synthetic construct
CTIEVMYPPPYLDNEKSNGTIIHVKGKHLCPSPLFPGPSKP

SEQ ID NO: 108 - protein, 11 aa, synthetic construct
SIVLXYXXIVL

SEQ ID NO: 109 - protein, 20 aa, synthetic construct
YGSQSSKPYLLTHPSDPLEL

SEQ ID NO: 110 - protein, 522 aa, synthetic construct
MDMRVPAQLLGLLLLWLRGARCDVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPRTSGGGTKLEIKGGGGSGGGGSGGGGSGGQVQLQQSGPELVKPGASVRISCKASGYTFTSYHIHWVKQRPGQGLEWIGWIYPGNVNTEYNEKFKGKATLTADKSSSTAYMHLSSLTSEDSAVYFCAREEITYAMDYWGQGTSVTVSSYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 111 - protein, 500 aa, synthetic construct
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPRTSGGGTKLEIKGGGGSGGGGSGGGGSGGQVQLQQSGPELVKPGASVRISCKASGYTFTSYHIHWVKQRPGQGLEWIGWIYPGNVNTEYNEKFKGKATLTADKSSSTAYMHLSSLTSEDSAVYFCAREEITYAMDYWGQGTSVTVSSYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 112 - protein, 247 aa, synthetic construct
MMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPRTSGGGTKLEIKGGGGSGGGGSGGGGSGGQVQLQQSGPELVKPGASVRISCKASGYTFTSYHIHWVKQRPGQGLEWIGWIYPGNVNTEYNEKFKGKATLTADKSSSTAYMHLSSLTSEDSAVYFCAREEITYAMDYWGQGTSVTVSSYG

SEQ ID NO: 113 - protein, 247 aa, synthetic construct
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPRTSGGGTKLEIKGGGGSGGGGSGGGGSGGQVQLQQSGPELVKPGASVRISCKASGYTFTSYHIHWVKQRPGQGLEWIGWIYPGNVNTEYNEKFKGKATLTADKSSSTAYMHLSSLTSEDSAVYFCAREEITYAMDYWGQGTSVTVSS

SEQ ID NO: 114 - protein, 245 aa, synthetic construct
QLVQSGAEVKKPGSSVKVSCKASGYTFTSYHIHWVRQAPGQGLEWMGWIYPGNVNTEYNEKFKGKATITADKSTSTAYMELSSLRSEDTAVYYCAREEITYAMDYWGQGTTVTVSSGGGGSGGGGSGGGGSGGEIVLTQSPGTLSLSPGERATLSCRSSQSIVHSNGNTYLEWYQQKPGQAPRLLIYKVSNRFSGIPDRFSGSGSGTDFTLTISRLEPEDFAVYYCFQGSHVPRTFGGGTKVEIK

SEQ ID NO: 115 - protein, 245 aa, synthetic construct
QLVQSGAEVKKPGSSVKVSCKASGYTFTSYHIHWVRQAPGQGLEWMGWIYPGNVNTEYNEKFKGKATITADKSTSTAYMELSSLRSEDTAVYYCAREEITYAMDYWGQGTTVTVSSGGGGSGGGGSGGGGSGGDIVMTQTPLSLPVTPGEPASISCRSSQSIVHSNGNTYLEWYLQKPGQSPQLLIYKVSNRFSGVPDRFSGSGSGTDFTLKISRVEAEDVGVYYCFQGSHVPRTFGGGTKVEIK

SEQ ID NO: 116 - protein, 245 aa, synthetic construct
QLVESGGGLVKPGGSLRLSCAASGYTFTSYHIHWVRQAPGKGLEWVGWIYPGNVNTEYNEKFKGRFTISRDDSKNTLYLQMNSLKTEDTAVYYCAREEITYAMDYWGQGTTVTVSSGGGGSGGGGSGGGGSGGDIQMTQSPSSLSASVGDRVTITCRSSQSIVHSNGNTYLEWYQQKPGKAPKLLIYKVSNRFSGVPSRFSGSGSGTDFTLTISSLQPEDFATYYCFQGSHVPRTFGGGTKVEIK

SEQ ID NO: 117 - protein, 245 aa, synthetic construct
QLVQSGAEVKKPGSSVKVSCKASGYTFTSYHIHWVRQAPGQGLEWIGWIYPGNVNTEYNEKFKGKATITADESTNTAYMELSSLRSEDTAVYYCAREEITYAMDYWGQGTLVTVSSGGGGSGGGGSGGGGSGGDIQMTQSPSTLSASVGDRVTITCRSSQSIVHSNGNTYLEWYQQKPGKAPKLLIYKVSNRFSGVPARFSGSGSGTEFTLTISSLQPDDFATYYCFQGSHVPRTFGQGTKVEVK

SEQ ID NO: 118 - protein, 245 aa, synthetic construct
QLVQSGAEVKKPGSSVKVSCKASGYTFTSYHMHWVRQAPGQGLEWIGYIYPGNVNTEYNEKFKGKATLTADKSTNTAYMELSSLRSEDTAVYFCAREEITYAMDYWGQGTLVTVSSGGGGSGGGGSGGGGSGGDVQMTQSPSTLSASVGDRVTITCSSSQSIVHSNGNTYMEWYQQKPGKAPKLLIYKVSNRFSGVPDRFSGSGSGTEFTLTISSLQPDDFATYYCHQGSHVPRTFGQGTKVEVK

SEQ ID NO: 119 - protein, 247 aa, synthetic construct
QVQLQQSGPELVKPGASVKMSCKASGYTFTSYHIQWVKQRPGQGLEWIGWIYPGDGSTQYNEKFKGKTTLTADKSSSTAYMLLSSLTSEDSAIYFCAREGTYYAMDYWGQGTSVTVSSGGGGSGGGGSGGGGSGGDVLMTQTPLSLPVSLGDQVSISCRSSQSIVHSNGNTYLEWYLQKPGQSPKLLIYKVSNRFSGVPDRFSGSGSGTDFTLKISRVEAEDLGVYYCFQGSHVPRTFGGGTKLEIK

SEQ ID NO: 120 - protein, 247 aa, synthetic construct
QLQLQESGPGLVKPSETLSLTCTVSGYTFTSYHIQWIRQPPGKGLEWIGWIYPGDGSTQYNEKFKGRATISVDTSKNQFSLNLDSVSAADTAIYYCAREGTYYAMDYWGKGSTVTVSSGGGGSGGGGSGGGGSGGDIQMTQSPSSLSASVGDRVTITCRSSQSIVHSNGNTYLEWYQQKPGKAPKLLIYKVSNRFSGVPSRFSGSGSGTDFTFTISSLQPEDIATYYCFQGSHVPRTFGPGTKVDIK

SEQ ID NO: 121 - protein, 247 aa, synthetic construct
EVQLVQSGAELKKPGSSVKVSCKASGYTFTSYHIQWVKQAPGQGLEWIGWIYPGDGSTQYNEKFKGKATLTVDKSTNTAYMELSSLRSEDTAVYYCAREGTYYAMDYWGQGTLVTVSSGGGGSGGGGSGGGGSGGDIQMTQSPSTLSASVGDRVTITCRSSQSIVHSNGNTYLEWYQQKPGKAPKLLIYKVSNRFSGVPSRFSGSGSGTDFTLTISSLQPDDFATYYCFQGSHVPRTFGQGTKVEVK

SEQ ID NO: 122 - protein, 247 aa, synthetic construct
QVQLVQSGAEVKKPGSSVKVSCKASGYTFTSYHIQWVRQAPGQGLEWMGWIYPGDGSTQYNEKFKGRVTITADKSTSTAYMELSSLRSEDTAVYYCAREGTYYAMDYWGQGTTVTVSSGGGGSGGGGSGGGGSGGEIVLTQSPGTLSLSPGERATLSCRSSQSIVHSNGNTYLEWYQQKPGQAPRLLIYKVSNRFSGIPDRFSGSGSGTDFTLTISRLEPEDFAVYYCFQGSHVPRTFGGGTKVEIK

SEQ ID NO: 123 - protein, 247 aa, synthetic construct
QVTLKQSGAEVKKPGSSVKVSCTASGYTFTSYHVSWVRQAPGQGLEWLGRIYPGDGSTQYNEKFKGKVTITADKSMDTSFMELTSLTSEDTAVYYCAREGTYYAMDLWGQGTLVTVSSGGGGSGGGGSGGGGSGGEIVLTQSPGTLSLSPGERATLSCRSSQSIVHSNGNTYLAWYQQKPGQAPRLLISKVSNRFSGVPDRFSGSGSGTDFTLTISRLEPEDFAVYYCQQGSHVPRTFGGGTKVEIK

SEQ ID NO: 124 - protein, 247 aa, synthetic construct
QVQLVQSGAEVKKPGASVKVSCKASGYTFTSYHMHWVRQAPGQRLEWMGWIYPGDGSTQYNEKFKGKVTITRDTSASTAYMELSSLRSEDTAVYYCAREGTYYAMDYWGQGTLVTVSSGGGGSGGGGSGGGGSGGDIVMTQTPLSLPVTPGEPASISCRSSQSIVHSNGNTYLDWYLQKPGQSPQLLIYKVSNRFSGVPDRFSGSGSGTDFTLKISRVEAEDVGVYYCMQGSHVPRTFGGGTKVEIK

SEQ ID NO: 125 - DNA, 123 bp, synthetic construct
  source 1..123: mol_type = other DNA
tgtaccattgaagttatgtatcctcctccttacctagacaatgagaagagcaatggaaccattatccatgtgaaagggaaacacctttgtccaagtcccctatttcccggaccttctaagccc

SEQ ID NO: 126 - protein, 23 aa, synthetic construct
VIGFRILLLKVAGFNLLMTLRLW

SEQ ID NO: 127 - DNA, 69 bp, synthetic construct
  source 1..69: mol_type = other DNA
gtgattgggttccgaatcctcctcctgaaagtggccgggtttaatctgctcatgacgctgcggctgtgg

SEQ ID NO: 128 - protein, 23 aa, synthetic construct
TILYEILLGKATLYAVLVSALVL

SEQ ID NO: 129 - DNA, 69 bp, synthetic construct
  source 1..69: mol_type = other DNA
accatcctctatgagatcttgctagggaaggccaccttgtatgccgtgctggtcagtgccctcgtgctg

SEQ ID NO: 130 - protein, 10 aa, synthetic construct
MAMVKRKDSR

SEQ ID NO: 131 - DNA, 30 bp, synthetic construct
  source 1..30: mol_type = other DNA
atggccatggtcaagagaaaggattccaga

SEQ ID NO: 132 - protein, 6 aa, synthetic construct
NLYAAV

SEQ ID NO: 133 - protein, 6 aa, synthetic construct
VTYAEV

SEQ ID NO: 134 - protein, 6 aa, synthetic construct
VTYAQL

SEQ ID NO: 135 - protein, 6 aa, synthetic construct
SIYATL

SEQ ID NO: 136 - protein, 35 aa, synthetic construct
NLYAAVKHTQPEDGVEMDTRSPHDEDPQAVTYAEV

SEQ ID NO: 137 - protein, 58 aa, synthetic construct
VTYAEVKHSRPRREMASPPSPLSGEFLDTKDRQAEEDRQMDTEAAASEAPQDVTYAQL

SEQ ID NO: 138 - protein, 36 aa, synthetic construct
VTYAQLHSLTLRREATEPPPSQEGPSPAVPSIYATL

SEQ ID NO: 139 - protein, 87 aa, synthetic construct
NLYAAVKHTQPEDGVEMDTRSPHDEDPQAVTYAEVKHSRPRREMASPPSPLSGEFLDTKDRQAEEDRQMDTEAAASEAPQDVTYAQL

SEQ ID NO: 140 - protein, 88 aa, synthetic construct
VTYAEVKHSRPRREMASPPSPLSGEFLDTKDRQAEEDRQMDTEAAASEAPQDVTYAQLHSLTLRREATEPPPSQEGPSPAVPSIYATL

SEQ ID NO: 141 - protein, 117 aa, synthetic construct
NLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATL

SEQ ID NO: 142 - protein, 167 aa, synthetic construct
RHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 143 - protein, 20 aa, synthetic construct
YGSQSSKPYLLTHPSDPLEL

SEQ ID NO: 144 - protein, 43 aa, synthetic construct
VVSGPSGGPSSPTTGPTSTSGPEDQPLTPTGSDPQSGLGRHLG

SEQ ID NO: 145 - protein, 63 aa, synthetic construct
YGSQSSKPYLLTHPSDPLELVVSGPSGGPSSPTTGPTSTSGPEDQPLTPTGSDPQSGLGRHLG

SEQ ID NO: 146 - protein, 23 aa, synthetic construct
VVIGILVAVILLLLLLLLLFLIL

SEQ ID NO: 147 - protein, 86 aa, synthetic construct
YGSQSSKPYLLTHPSDPLELVVSGPSGGPSSPTTGPTSTSGPEDQPLTPTGSDPQSGLGRHLGVVIGILVAVILLLLLLLLLFLIL

SEQ ID NO: 148 - protein, 190 aa, synthetic construct
VVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 149 - protein, 253 aa, synthetic construct
YGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 150 - protein, 233 aa, synthetic construct
VVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 151 - protein, 670 aa, synthetic construct
MTPILTVLICLGLSLGPRTHVQAGHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIHPSQEGPSPAVPSIYATLAIH

SEQ ID NO: 152 - protein, 64 aa, synthetic construct
YGSQSSKPYLLTHPSDPLELVVSGPSGGPSSPTTGPTSTSGPEDQPLTPTGSDPQSGLGRHLGV

SEQ ID NO: 153 - protein, 44 aa, synthetic construct
VVSGPSGGPSSPTTGPTSTSGPEDQPLTPTGSDPQSGLGRHLGV

SEQ ID NO: 154 - protein, 67 aa, synthetic construct
AGSGGSGGSGGSPVPSTPPTPSPSTPPTPSPSGGSGNSSGSGGSPVPSTPPTPSPSTPPTPSPSASV

SEQ ID NO: 155 - protein, 75 aa, synthetic construct
AGSGGSGGSGGSPVPSTPPTNSSSTPPTPSPSPVPSTPPTNSSSTPPTPSPSPVPSTPPTNSSSTPPTPSPSASV

SEQ ID NO: 156 - protein, 85 aa, synthetic construct
VVSGPSGGPSSPTTGPTSTSGPEDQPLTPTGSDPQSGLGRHVVSGPSGGPSSPTTGPTSTSGPEDQPLTPTGSDPQSGLGRHLGV

SEQ ID NO: 157 - protein, 32 aa, synthetic construct
TTGPTSTSGPEDQPLTPTGSDPQSGLGRHLGV

SEQ ID NO: 158 - protein, 512 aa, synthetic construct
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTDLPKLRNFHIWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 159 - protein, 515 aa, synthetic construct
EVQLVESGGGLVQPGGSLRLSCAASGFTFSSYAMHWVRQAPGKGLEYVSAISSNGGSTYYANSVKGRFTISRDNSKNTLYLQMGSLRAEDMAVYYCASLEYHGFRQYGLRYWHWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 160 - protein, 514 aa, synthetic construct
QLQLQESGSGLVKPSQTLSLTCAVSGGSISSGGYSWSWIRQPPGKGLEWIGYIYHSGSTYYNPSLKSRVTISVDRSKNQFSLKLSSVTAADTAVYYCASIKFWFAGINYFFPWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 161 - protein, 510 aa, synthetic construct
EVQLVESGGGLVQPGGSLRLSCAASGFTFSSYWMHWVRQAPGKGLVWVSRINSDGSSTSYADSVKGRFTISRDNAKNTLYLQMNSLRAEDTAVYYCASGFLGMGSNFIWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 162 - protein, 510 aa, synthetic construct
QVQLQESGPGLVKPSQTLSLTCTVSGGSISSGGYYWSWIRQHPGKGLEWIGYIYYSGSTYYNPSLKSLVTISVDTSKNQFSLKLSSVTAADTAVYYCASGDRARYFDLWGRGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 163 - protein, 514 aa, synthetic construct
EVQLVESGGGLVQPGGSLRLSCAASGFTFSSYWMHWVRQAPGKGLVWVSRINSDGSSTSYADSVKGRFTISRDNAKNTLYLQMNSLRAEDTAVYYCARYPRGYHQMVDAFDIWGQGTMVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 164 - protein, 517 aa, synthetic construct
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARVRFLAARTTIPEANFLWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 165 - protein, 509 aa, synthetic construct
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCARVLSRARFDYWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 166 - protein, 508 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARLRGRVFDPWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 167 - protein, 514 aa, synthetic construct
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARIKFTSFLYVHGFLWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 168 - protein, 513 aa, synthetic construct
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCARGQRWLYLGGIRRHWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 169 - protein, 511 aa, synthetic construct
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAREWIPSRPYYFDYWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 170 - protein, 511 aa, synthetic construct
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARESTGTGAFDIWGQGTMVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 171 - protein, 513 aa, synthetic construct
EVQLVESGGGVVRPGGSLRLSCAASGFTFDDYGMSWVRQAPGKGLEWVSGINWNGGSTGYADSVKGRFTISRDNAKNSLYLQMNSLRAEDTALYHCARERYRRVLHWYFDLWGRGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 172 - protein, 507 aa, synthetic construct
QVQLVQSGAEVKKPGASVKVSCKASGYTFTSYDINWVRQATGQGLEWMGWMNPNSGNTGYAQKFQGRVTMTRNTSISTAYMELSSLRSEDTAVYYCAREPDAFDIWGQGTMVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 173 - protein, 513 aa, synthetic construct
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCAREHMGTIPYYFDYWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 174 - protein, 511 aa, synthetic construct
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCAREEFGYGDVLYWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 175 - protein, 511 aa, synthetic construct
QVQLQESGPGLVKPSQTLSLTCTVSGGSISSGDYYWSWIRQPPGKGLEWIGYIYYSGSTYYNPSLKSRVTISVDTSKNQFSLKLSSVTAADTAVYYCAREDVVKGAFDIWGQGTMVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 176 - protein, 512 aa, synthetic construct
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCAREDFSHKLGYFQHWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 177 - protein, 505 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARDYDYVWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 178 - protein, 511 aa, synthetic construct
QVQLQESGPGLVKPSQTLSLTCTVSGGSISSGDYYWSWIRQPPGKGLEWIGYIYYSGSTYYNPSLKSRVTISVDTSKNQFSLKLSSVTAADTAVYYCARDRRDWDWFDPWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 179 - protein, 510 aa, synthetic construct
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARDQQALKYRVDWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 180 - protein, 509 aa, synthetic construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDLTLGCFDYWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 181 - protein, 509 aa, synthetic construct
EVQLVESGGGLVQPGGSLRLSCAASGFTVSSNYMSWVRQAPGKGLEWVSVIYSGGSTYYADSVKGRFTISRDNSKNTLYLQMNSLRAEDTAVYYCARDGSNSWYFDLWGRGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 182 - protein, 509 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARAFLFLSFSVWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 183 - protein, 512 aa, synthetic construct
EVQLVESGGVVVQPGGSLRLSCAASGFTFDDYTMHWVRQAPGKGLEWVSLISWDGGSTYYADSVKGRFTISRDNSKNSLYLQMNSLRTEDTALYYCAKGIFYSSKEDFDYWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 184 - protein, 515 aa, synthetic construct
EVQLVESGGVVVQPGGSLRLSCAASGFTFDDYTMHWVRQAPGKGLEWVSLISWDGGSTYYADSVKGRFTISRDNSKNSLYLQMNSLRTEDTALYYCAKDIWIFYSSNPKPTVYWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 185 - protein, 513 aa, synthetic construct
QVQLVQSGSELKKPGASVKVSCKASGYTFTSYAMNWVRQAPGQGLEWMGWINTNTGNPTYAQGFTGRFVFSFDTSVSTAYLQICSLKAEDTAVYYCARKDQTLTYGNWFDPWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 186 - protein, 509 aa, synthetic construct
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCARDHYERGLYWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 187 - protein, 509 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARYMYNWYFDLWGRGTLVTVSSGGGGSGGGGSGGGGSGGDIQMTQSPSSVSASVGDRVTITCRASQGISSWLAWYQQKPGKAPKLLIYAASSLQSGVPSRFSGSGSGTDFTLTISSLQPEDFATYYCQQANSFPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 188 - protein, 508 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARDRRPAFDIWGQGTMVTVSSGGGGSGGGGSGGGGSGGDIQMTQSPSSVSASVGDRVTITCRASQGISSWLAWYQQKPGKAPKLLIYAASSLQSGVPSRFSGSGSGTDFTLTISSLQPEDFATYYCQQANSFPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 189 - protein, 510 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAVHLKRRPYFDYWGQGTLVTVSSGGGGSGGGGSGGGGSGGAIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 190 - protein, 509 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCASVHKKPIFDYWGQGTLVTVSSGGGGSGGGGSGGGGSGGAIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 191 - protein, 509 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCASTSRRCTFQHWGQGTLVTVSSGGGGSGGGGSGGGGSGGAIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 192 - protein, 509 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCASTSPRPLFQHWGQGTLVTVSSGGGGSGGGGSGGGGSGGAIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 193 - protein, 511 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCASPYQVRGVYFDYWGQGTLVTVSSGGGGSGGGGSGGGGSGGAIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 194 - protein, 511 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCASPYKKRRTVFDYWGQGTLVTVSSGGGGSGGGGSGGGGSGGAIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 195 - protein, 510 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCASLQRGLALFQHWGQGTLVTVSSGGGGSGGGGSGGGGSGGAIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 196 - protein, 509 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCASILSVPYFDLWGRGTLVTVSSGGGGSGGGGSGGGGSGGAIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 197 - protein, 514 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCASGWIRVPLRLPLFQHWGQGTLVTVSSGGGGSGGGGSGGGGSGGAIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 198 - protein, 509 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARVTIFAIFDIWGQGTMVTVSSGGGGSGGGGSGGGGSGGAIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 199 - protein, 510 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARVGRGFVHFDLWGRGTLVTVSSGGGGSGGGGSGGGGSGGAIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 200 - protein, 511 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARTSRGLCVLFDYWGQGTLVTVSSGGGGSGGGGSGGGGSGGAIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 201 - protein, 511 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARSGPSSYWYFDLWGRGTLVTVSSGGGGSGGGGSGGGGSGGAIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 202 - protein, 511 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARNIYMGGIWFDPWGQGTLVTVSSGGGGSGGGGSGGGGSGGAIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 203 - protein, 510 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARLTVRTGAFDIWGQGTMVTVSSGGGGSGGGGSGGGGSGGAIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 204 - protein, 510 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARLRTAHLDFDLWGRGTLVTVSSGGGGSGGGGSGGGGSGGAIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 205 - protein, 510 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARDLIFPVVFDYWGQGTLVTVSSGGGGSGGGGSGGGGSGGAIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 206 - protein, 513 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARDGYRKYGYVFFDIWGQGTMVTVSSGGGGSGGGGSGGGGSGGAIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 207 - protein, 513 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARDGRYRRFWHAFDIWGQGTMVTVSSGGGGSGGGGSGGGGSGGAIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 208 - protein, 509 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARAHIRGYFDLWGRGTLVTVSSGGGGSGGGGSGGGGSGGAIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 209 - protein, 512 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCATWMGGGGRWYFDLWGRGTLVTVSSGGGGSGGGGSGGGGSGGAIQLTQSPSSLSASVGDRVTITCRASQGISSALAWYQQKPGKAPKLLIYDASSLESGVPSRFSGSGSGTDFTLTISSLQPEDFATYYCQQFNNYPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 210 - protein, 510 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARTSRTTWYFDLWGRGTLVTVSSGGGGSGGGGSGGGGSGGAIQLTQSPSSLSASVGDRVTITCRASQGISSALAWYQQKPGKAPKLLIYDASSLESGVPSRFSGSGSGTDFTLTISSLQPEDFATYYCQQFNNYPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 211 - protein, 512 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKWMGGGGRLYFDLWGRGTLVTVSSGGGGSGGGGSGGGGSGGAIQLTQSPSSLSASVGDRVTITCRASQGISSALAWYQQKPGKAPKLLIYDASSLESGVPSRFSGSGSGTDFTLTISSLQPEDFATYYCQQFNNYPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 212 - protein, 511 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKWGGRLYWYFDLWGRGTLVTVSSGGGGSGGGGSGGGGSGGAIQLTQSPSSLSASVGDRVTITCRASQGISSALAWYQQKPGKAPKLLIYDASSLESGVPSRFSGSGSGTDFTLTISSLQPEDFATYYCQQFNNYPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 213 - protein, 510 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKVIRQLWYFDLWGRGTLVTVSSGGGGSGGGGSGGGGSGGAIQLTQSPSSLSASVGDRVTITCRASQGISSALAWYQQKPGKAPKLLIYDASSLESGVPSRFSGSGSGTDFTLTISSLQPEDFATYYCQQFNNYPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 214 - protein, 510 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKVFANSWYFDLWGRGTLVTVSSGGGGSGGGGSGGGGSGGAIQLTQSPSSLSASVGDRVTITCRASQGISSALAWYQQKPGKAPKLLIYDASSLESGVPSRFSGSGSGTDFTLTISSLQPEDFATYYCQQFNNYPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 215 - protein, 510 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKVDRTTWYFDLWGRGTLVTVSSGGGGSGGGGSGGGGSGGAIQLTQSPSSLSASVGDRVTITCRASQGISSALAWYQQKPGKAPKLLIYDASSLESGVPSRFSGSGSGTDFTLTISSLQPEDFATYYCQQFNNYPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 216 - protein, 513 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKRWGKDGPYWYFDLWGRGTLVTVSSGGGGSGGGGSGGGGSGGAIQLTQSPSSLSASVGDRVTITCRASQGISSALAWYQQKPGKAPKLLIYDASSLESGVPSRFSGSGSGTDFTLTISSLQPEDFATYYCQQFNNYPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 217 - protein, 513 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKRRDSYGPYWYFDLWGRGTLVTVSSGGGGSGGGGSGGGGSGGAIQLTQSPSSLSASVGDRVTITCRASQGISSALAWYQQKPGKAPKLLIYDASSLESGVPSRFSGSGSGTDFTLTISSLQPEDFATYYCQQFNNYPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 218 - protein, 512 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKNRPPPGYWYFDLWGRGTLVTVSSGGGGSGGGGSGGGGSGGAIQLTQSPSSLSASVGDRVTITCRASQGISSALAWYQQKPGKAPKLLIYDASSLESGVPSRFSGSGSGTDFTLTISSLQPEDFATYYCQQFNNYPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 219 - protein, 510 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGRRFSWYFDLWGRGTLVTVSSGGGGSGGGGSGGGGSGGAIQLTQSPSSLSASVGDRVTITCRASQGISSALAWYQQKPGKAPKLLIYDASSLESGVPSRFSGSGSGTDFTLTISSLQPEDFATYYCQQFNNYPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 220 - protein, 1045 aa, synthetic construct
EVQLVESGGGLVKPGGSLRLSCAASGFTFSNYWMNWVRQAPGKGLEWVGEIRLKSTNYATHYAESVKGRFTISRDDSKNTLYLQMNSLKTEDTAVYYCTTLITPDYWGQGTTVTVSSGGGGSGGGGSGGGGSGGDIQMTQSPSSVSASVGDRVTITCKASQDVSTTVAWYQQKPGKAPKLLIYSASYRYTGVPSRFSGSGSGTDFTLTISSLQPEDFATYYCQQHYSTPPTFGGGTKVEIKYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIHGSGEGRGSLLTCGDVEENPGPMEFGLSWLFLVAILKGVQCQVQLQESGPGLVKPSQTLSLTCTVSGGSISSGDYYWSWIRQPPGKGLEWIGYIYYSGSTYYNPSLKSRVTISVDTSKNQFSLKLSSVTAADTAVYYCAREDVVKGAFDIWGQGTMVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 221 - protein, 116 aa, synthetic construct
QVQLVQSGAEVKKPGASVKVSCKASGYTFTSYDINWVRQATGQGLEWMGWMNPNSGNTGYAQKFQGRVTMTRNTSISTAYMELSSLRSEDTAVYYCAREPDAFDIWGQGTMVTVSS

SEQ ID NO: 222 - protein, 107 aa, synthetic construct [sequence omitted]

SEQ ID NO: 223 - protein, 5 aa, synthetic construct
SYDIN

SEQ ID NO: 224 - protein, 17 aa, synthetic construct
WMNPNSGNTGYAQKFQG

SEQ ID NO: 225 - protein, 7 aa, synthetic construct
EPDAFDI

SEQ ID NO: 226 - protein, 11 aa, synthetic construct
RASQSISSYLN

SEQ ID NO: 227 - protein, 7 aa, synthetic construct
AASSLQS

SEQ ID NO: 228 - protein, 9 aa, synthetic construct
QQSYSTPLT

SEQ ID NO: 229 - protein, 118 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCASTSRRCTFQHWGQGTLVTVSS

SEQ ID NO: 230 - protein, 106 aa, synthetic construct
IQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIK

SEQ ID NO: 231 - protein, 5 aa, synthetic construct
DYYMS

SEQ ID NO: 232 - protein, 17 aa, synthetic construct
YISSSGSTIYYADSVKG

SEQ ID NO: 233 - protein, 9 aa, synthetic construct
TSRRCTFQH

SEQ ID NO: 234 - protein, 11 aa, synthetic construct
RASQGISSALA

SEQ ID NO: 235 - protein, 7 aa, synthetic construct
DASSLES

SEQ ID NO: 236 - protein, 9 aa, synthetic construct
QQFNSYPLT

SEQ ID NO: 237 - protein, 122 aa, synthetic construct
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCAREHMGTIPYYFDYWGQGTLVTVSS

SEQ ID NO: 238 - protein, 107 aa, synthetic construct [sequence omitted]

SEQ ID NO: 239 - protein, 7 aa, synthetic construct
SGGYYWS

SEQ ID NO: 240 - protein, 16 aa, synthetic construct
YIYYSGSTYYNPSLKS

SEQ ID NO: 241 - protein, 12 aa, synthetic construct
EHMGTIPYYFDY

SEQ ID NO: 242 - protein, 11 aa, synthetic construct
RASQSISSYLN

SEQ ID NO: 243 - protein, 7 aa, synthetic construct
AASSLQS

SEQ ID NO: 244 - protein, 9 aa, synthetic construct
QQSYSTPLT

SEQ ID NO: 245 - protein, 120 aa, synthetic construct
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCAREEFGYGDVLYWGQGTLVTVSS

SEQ ID NO: 246 - protein, 107 aa, synthetic construct [sequence omitted]

SEQ ID NO: 247 - protein, 7 aa, synthetic construct
SGGYYWS

SEQ ID NO: 248 - protein, 16 aa, synthetic construct
YIYYSGSTYYNPSLKS

SEQ ID NO: 249 - protein, 10 aa, synthetic construct
EEFGYGDVLY

SEQ ID NO: 250 - protein, 11 aa, synthetic construct
RASQSISSYLN

SEQ ID NO: 251 - protein, 7 aa, synthetic construct
AASSLQS

SEQ ID NO: 252 - protein, 9 aa, synthetic construct
QQSYSTPLT

SEQ ID NO: 253 - protein, 122 aa, synthetic construct
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCARGQRWLYLGGIRRHWGQGTLVTVSS

SEQ ID NO: 254 - protein, 107 aa, synthetic construct [sequence omitted]

SEQ ID NO: 255 - protein, 5 aa, synthetic construct
SYAMH

SEQ ID NO: 256 - protein, 17 aa, synthetic construct
WINAGNGNTKYSQKFQG

SEQ ID NO: 257 - protein, 13 aa, synthetic construct
GQRWLYLGGIRRH

SEQ ID NO: 258 - protein, 11 aa, synthetic construct
RASQSISSYLN

SEQ ID NO: 259 - protein, 7 aa, synthetic construct
AASSLQS

SEQ ID NO: 260 - protein, 9 aa, synthetic construct
QQSYSTPLT

SEQ ID NO: 261 - protein, 119 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAVHLKRRPYFDYWGQGTLVTVSS

SEQ ID NO: 262 - protein, 106 aa, synthetic construct; identical to SEQ ID NO: 230

SEQ ID NO: 263 - protein, 5 aa, synthetic construct
DYYMS

SEQ ID NO: 264 - protein, 17 aa, synthetic construct
YISSSGSTIYYADSVKG

SEQ ID NO: 265 - protein, 10 aa, synthetic construct
HLKRRPYFDY

SEQ ID NO: 266 - protein, 11 aa, synthetic construct
RASQGISSALA

SEQ ID NO: 267 - protein, 7 aa, synthetic construct
DASSLES

SEQ ID NO: 268 - protein, 9 aa, synthetic construct
QQFNSYPLT

SEQ ID NO: 269 - protein, 118 aa, synthetic construct
EVQLVESGGGLVQPGGSLRLSCAASGFTVSSNYMSWVRQAPGKGLEWVSVIYSGGSTYYADSVKGRFTISRDNSKNTLYLQMNSLRAEDTAVYYCARDGSNSWYFDLWGRGTLVTVSS

SEQ ID NO: 270 - protein, 107 aa, synthetic construct [sequence omitted]

SEQ ID NO: 271 - protein, 5 aa, synthetic construct
SNYMS

SEQ ID NO: 272 - protein, 16 aa, synthetic construct
VIYSGGSTYYADSVKG

SEQ ID NO: 273 - protein, 10 aa, synthetic construct
DGSNSWYFDL

SEQ ID NO: 274 - protein, 11 aa, synthetic construct
RASQSISSYLN

SEQ ID NO: 275 - protein, 7 aa, synthetic construct
AASSLQS

SEQ ID NO: 276 - protein, 9 aa, synthetic construct
QQSYSTPLT

SEQ ID NO: 277 - protein, 118 aa, synthetic construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDLTLGCFDYWGQGTLVTVSS

SEQ ID NO: 278 - protein, 107 aa, synthetic construct [sequence omitted]

SEQ ID NO: 279 - protein, 5 aa, synthetic construct
SYGIS

SEQ ID NO: 280 - protein, 17 aa, synthetic construct
WISAYNGNTNYAQKLQG

SEQ ID NO: 281 - protein, 9 aa, synthetic construct
DLTLGCFDY

SEQ ID NO: 282 - protein, 11 aa, synthetic construct
RASQSISSYLN

SEQ ID NO: 283 - protein, 7 aa, synthetic construct
AASSLQS

SEQ ID NO: 284 - protein, 9 aa, synthetic construct
QQSYSTPLT

SEQ ID NO: 285 - protein, 121 aa, synthetic construct
EVQLVESGGVVVQPGGSLRLSCAASGFTFDDYTMHWVRQAPGKGLEWVSLISWDGGSTYYADSVKGRFTISRDNSKNSLYLQMNSLRTEDTALYYCAKGIFYSSKEDFDYWGQGTLVTVSS

SEQ ID NO: 286 - protein, 107 aa, synthetic construct [sequence omitted]

SEQ ID NO: 287 - protein, 5 aa, synthetic construct
DYTMH

SEQ ID NO: 288 - protein, 17 aa, synthetic construct
LISWDGGSTYYADSVKG

SEQ ID NO: 289 - protein, 12 aa, synthetic construct
GIFYSSKEDFDY

SEQ ID NO: 290 - protein, 11 aa, synthetic construct
RASQSISSYLN

SEQ ID NO: 291 - protein, 7 aa, synthetic construct
AASSLQS

SEQ ID NO: 292 - protein, 9 aa, synthetic construct
QQSYSTPLT

SEQ ID NO: 293 - protein, 122 aa, synthetic construct
QVQLVQSGSELKKPGASVKVSCKASGYTFTSYAMNWVRQAPGQGLEWMGWINTNTGNPTYAQGFTGRFVFSFDTSVSTAYLQICSLKAEDTAVYYCARKDQTLTYGNWFDPWGQGTLVTVSS

SEQ ID NO: 294 - protein, 107 aa, synthetic construct [sequence omitted]

SEQ ID NO: 295 - protein, 5 aa, synthetic construct
SYAMN

SEQ ID NO: 296 - protein, 17 aa, synthetic construct
WINTNTGNPTYAQGFTG

SEQ ID NO: 297 - protein, 13 aa, synthetic construct
KDQTLTYGNWFDP

SEQ ID NO: 298 - protein, 11 aa, synthetic construct
RASQSISSYLN

SEQ ID NO: 299 - protein, 7 aa, synthetic construct
AASSLQS

SEQ ID NO: 300 - protein, 9 aa, synthetic construct
QQSYSTPLT

SEQ ID NO: 301 - protein, 120 aa, synthetic construct
QVQLQESGPGLVKPSQTLSLTCTVSGGSISSGDYYWSWIRQPPGKGLEWIGYIYYSGSTYYNPSLKSRVTISVDTSKNQFSLKLSSVTAADTAVYYCAREDVVKGAFDIWGQGTMVTVSS

SEQ ID NO: 302 - protein, 107 aa, synthetic construct [sequence omitted]

SEQ ID NO: 303 - protein, 7 aa, synthetic construct
SGDYYWS

SEQ ID NO: 304 - protein, 16 aa, synthetic construct
YIYYSGSTYYNPSLKS

SEQ ID NO: 305 - protein, 10 aa, synthetic construct
EDVVKGAFDI

SEQ ID NO: 306 - protein, 11 aa, synthetic construct
RASQSISSYLN

SEQ ID NO: 307 - protein, 7 aa, synthetic construct
AASSLQS

SEQ ID NO: 308 - protein, 9 aa, synthetic construct
QQSYSTPLT

SEQ ID NO: 309 - protein, 119 aa, synthetic construct
EVQLVESGGGLVQPGGSLRLSCAASGFTFSSYWMHWVRQAPGKGLVWVSRINSDGSSTSYADSVKGRFTISRDNAKNTLYLQMNSLRAEDTAVYYCASGFLGMGSNFIWGQGTLVTVSS

SEQ ID NO: 310 - protein, 107 aa, synthetic construct [sequence omitted]

SEQ ID NO: 311 - protein, 5 aa, synthetic construct
SYWMH

SEQ ID NO: 312 - protein, 17 aa, synthetic construct
RINSDGSSTSYADSVKG

SEQ ID NO: 313 - protein, 10 aa, synthetic construct
GFLGMGSNFI

SEQ ID NO: 314 - protein, 11 aa, synthetic construct
RASQSISSYLN

SEQ ID NO: 315 - protein, 7 aa, synthetic construct
AASSLQS

SEQ ID NO: 316 - protein, 9 aa, synthetic construct
QQSYSTPLT

SEQ ID NO: 317 - protein, 114 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARDYDYVWGQGTLVTVSS

SEQ ID NO: 318 - protein, 107 aa, synthetic construct [sequence omitted]

SEQ ID NO: 319 - protein, 5 aa, synthetic construct
DYYMS

SEQ ID NO: 320 - protein, 17 aa, synthetic construct
YISSSGSTIYYADSVKG

SEQ ID NO: 321 - protein, 5 aa, synthetic construct
DYDYV

SEQ ID NO: 322 - protein, 11 aa, synthetic construct
RASQSISSYLN

SEQ ID NO: 323 - protein, 7 aa, synthetic construct
AASSLQS

SEQ ID NO: 324 - protein, 9 aa, synthetic construct
QQSYSTPLT

SEQ ID NO: 325 - protein, 118 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCASTSPRPLFQHWGQGTLVTVSS

SEQ ID NO: 326 - protein, 106 aa, synthetic construct; identical to SEQ ID NO: 230

SEQ ID NO: 327 - protein, 5 aa, synthetic construct
DYYMS

SEQ ID NO: 328 - protein, 17 aa, synthetic construct
YISSSGSTIYYADSVKG

SEQ ID NO: 329 - protein, 9 aa, synthetic construct
TSPRPLFQH

SEQ ID NO: 330 - protein, 11 aa, synthetic construct
RASQGISSALA

SEQ ID NO: 331 - protein, 7 aa, synthetic construct
DASSLES

SEQ ID NO: 332 - protein, 9 aa, synthetic construct
QQFNSYPLT

SEQ ID NO: 333 - protein, 120 aa, synthetic construct
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARESTGTGAFDIWGQGTMVTVSS

SEQ ID NO: 334 - protein, 107 aa, synthetic construct [sequence omitted]

SEQ ID NO: 335 - protein, 7 aa, synthetic construct
SGGYYWS

SEQ ID NO: 336 - protein, 16 aa, synthetic construct
YIYYSGSTYYNPSLKS

SEQ ID NO: 337 - protein, 10 aa, synthetic construct
ESTGTGAFDI

SEQ ID NO: 338 - protein, 11 aa, synthetic construct
RASQSISSYLN

SEQ ID NO: 339 - protein, 7 aa, synthetic construct
AASSLQS

SEQ ID NO: 340 - protein, 9 aa, synthetic construct
QQSYSTPLT

SEQ ID NO: 341 - protein, 118 aa, synthetic construct
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCARVLSRARFDYWGQGTLVTVSS

SEQ ID NO: 342 - protein, 107 aa, synthetic construct [sequence omitted]

SEQ ID NO: 343 - protein, 5 aa, synthetic construct
SYSMN

SEQ ID NO: 344 - protein, 17 aa, synthetic construct
YISSSSSTIYYADSVKG

SEQ ID NO: 345 - protein, 9 aa, synthetic construct
VLSRARFDY

SEQ ID NO: 346 - protein, 11 aa, synthetic construct
RASQSISSYLN

SEQ ID NO: 347 - protein, 7 aa, synthetic construct
AASSLQS

SEQ ID NO: 348 - protein, 9 aa, synthetic construct
QQSYSTPLT

SEQ ID NO: 349 - protein, 117 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARDRRPAFDIWGQGTMVTVSS

SEQ ID NO: 350 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQANSFPLTFGGGTKVEIK

SEQ ID NO: 351 - protein, 5 aa, synthetic construct
DYYMS

SEQ ID NO: 352 - protein, 17 aa, synthetic construct
YISSSGSTIYYADSVKG

SEQ ID NO: 353 - protein, 8 aa, synthetic construct
DRRPAFDI

SEQ ID NO: 354 - protein, 11 aa, synthetic construct
RASQGISSWLA

SEQ ID NO: 355 - protein, 7 aa, synthetic construct
AASSLQS

SEQ ID NO: 356 - protein, 9 aa, synthetic construct
QQANSFPLT

SEQ ID NO: 357 - protein, 124 aa, synthetic construct
EVQLVESGGVVVQPGGSLRLSCAASGFTFDDYTMHWVRQAPGKGLEWVSLISWDGGSTYYADSVKGRFTISRDNSKNSLYLQMNSLRTEDTALYYCAKDIWIFYSSNPKPTVYWGQGTLVTVSS

SEQ ID NO: 358 - protein, 107 aa, synthetic construct [sequence omitted]

SEQ ID NO: 359 - protein, 5 aa, synthetic construct
DYTMH

SEQ ID NO: 360 - protein, 17 aa, synthetic construct
LISWDGGSTYYADSVKG

SEQ ID NO: 361 - protein, 15 aa, synthetic construct
DIWIFYSSNPKPTVY

SEQ ID NO: 362 - protein, 11 aa, synthetic construct
RASQSISSYLN

SEQ ID NO: 363 - protein, 7 aa, synthetic construct
AASSLQS

SEQ ID NO: 364 - protein, 9 aa, synthetic construct
QQSYSTPLT

SEQ ID NO: 365 - protein, 120 aa, synthetic construct
QVQLQESGPGLVKPSQTLSLTCTVSGGSISSGDYYWSWIRQPPGKGLEWIGYIYYSGSTYYNPSLKSRVTISVDTSKNQFSLKLSSVTAADTAVYYCARDRRDWDWFDPWGQGTLVTVSS

SEQ ID NO: 366 - protein, 107 aa, synthetic construct [sequence omitted]

SEQ ID NO: 367 - protein, 7 aa, synthetic construct
SGDYYWS

SEQ ID NO: 368 - protein, 16 aa, synthetic construct
YIYYSGSTYYNPSLKS

SEQ ID NO: 369 - protein, 10 aa, synthetic construct
DRRDWDWFDP

SEQ ID NO: 370 - protein, 11 aa, synthetic construct
RASQSISSYLN

SEQ ID NO: 371 - protein, 7 aa, synthetic construct
AASSLQS

SEQ ID NO: 372 - protein, 9 aa, synthetic construct
QQSYSTPLT

SEQ ID NO: 373 - protein, 124 aa, synthetic construct
EVQLVESGGGLVQPGGSLRLSCAASGFTFSSYAMHWVRQAPGKGLEYVSAISSNGGSTYYANSVKGRFTISRDNSKNTLYLQMGSLRAEDMAVYYCASLEYHGFRQYGLRYWHWGQGTLVTVSS

SEQ ID NO: 374 - protein, 107 aa, synthetic construct [sequence omitted]

SEQ ID NO: 375 - protein, 5 aa, synthetic construct
SYAMH

SEQ ID NO: 376 - protein, 17 aa, synthetic construct
AISSNGGSTYYANSVKG

SEQ ID NO: 377 - protein, 15 aa, synthetic construct
LEYHGFRQYGLRYWH

SEQ ID NO: 378 - protein, 11 aa, synthetic construct
RASQSISSYLN

SEQ ID NO: 379 - protein, 7 aa, synthetic construct
AASSLQS

SEQ ID NO: 380 - protein, 9 aa, synthetic construct
QQSYSTPLT

SEQ ID NO: 381 - protein, 123 aa, synthetic construct
EVQLVESGGGLVQPGGSLRLSCAASGFTFSSYWMHWVRQAPGKGLVWVSRINSDGSSTSYADSVKGRFTISRDNAKNTLYLQMNSLRAEDTAVYYCARYPRGYHQMVDAFDIWGQGTMVTVSS

SEQ ID NO: 382 - protein, 107 aa, synthetic construct [sequence omitted]

SEQ ID NO: 383 - protein, 5 aa, synthetic construct
SYWMH

SEQ ID NO: 384 - protein, 17 aa, synthetic construct
RINSDGSSTSYADSVKG

SEQ ID NO: 385 - protein, 14 aa, synthetic construct
YPRGYHQMVDAFDI

SEQ ID NO: 386 - protein, 11 aa, synthetic construct
RASQSISSYLN

SEQ ID NO: 387 - protein, 7 aa, synthetic construct
AASSLQS

SEQ ID NO: 388 - protein, 9 aa, synthetic construct
QQSYSTPLT

SEQ ID NO: 389 - protein, 120 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCASPYQVRGVYFDYWGQGTLVTVSS

SEQ ID NO: 390 - protein, 106 aa, synthetic construct; identical to SEQ ID NO: 230

SEQ ID NO: 391 - protein, 5 aa, synthetic construct
DYYMS

SEQ ID NO: 392 - protein, 17 aa, synthetic construct
YISSSGSTIYYADSVKG

SEQ ID NO: 393 - protein, 11 aa, synthetic construct
PYQVRGVYFDY

SEQ ID NO: 394 - protein, 11 aa, synthetic construct
RASQGISSALA

SEQ ID NO: 395 - protein, 7 aa, synthetic construct
DASSLES

SEQ ID NO: 396 - protein, 9 aa, synthetic construct
QQFNSYPLT

SEQ ID NO: 397 - protein, 118 aa, synthetic construct
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCARDHYERGLYWGQGTLVTVSS

SEQ ID NO: 398 - protein, 107 aa, synthetic construct [sequence omitted]

SEQ ID NO: 399 - protein, 7 aa, synthetic construct
SGSYYWS

SEQ ID NO: 400 - protein, 16 aa, synthetic construct
YIYYSGSTNYNPSLKS

SEQ ID NO: 401 - protein, 8 aa, synthetic construct
DHYERGLY

SEQ ID NO: 402 - protein, 11 aa, synthetic construct
RASQSISSYLN

SEQ ID NO: 403 - protein, 7 aa, synthetic construct
AASSLQS

SEQ ID NO: 404 - protein, 9 aa, synthetic construct
QQSYSTPLT

SEQ ID NO: 405 - protein, 123 aa, synthetic construct
QLQLQESGSGLVKPSQTLSLTCAVSGGSISSGGYSWSWIRQPPGKGLEWIGYIYHSGSTYYNPSLKSRVTISVDRSKNQFSLKLSSVTAADTAVYYCASIKFWFAGINYFFPWGQGTLVTVSS

SEQ ID NO: 406 - protein, 107 aa, synthetic construct [sequence omitted]

SEQ ID NO: 407 - protein, 7 aa, synthetic construct
SGGYSWS

SEQ ID NO: 408 - protein, 16 aa, synthetic construct
YIYHSGSTYYNPSLKS

SEQ ID NO: 409 - protein, 13 aa, synthetic construct
IKFWFAGINYFFP

SEQ ID NO: 410 - protein, 11 aa, synthetic construct
RASQSISSYLN

SEQ ID NO: 411 - protein, 7 aa, synthetic construct
AASSLQS

SEQ ID NO: 412 - protein, 9 aa, synthetic construct
QQSYSTPLT

SEQ ID NO: 413 - protein, 121 aa, synthetic construct
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCAREDFSHKLGYFQHWGQGTLVTVSS

SEQ ID NO: 414 - protein, 107 aa, synthetic construct [sequence omitted]

SEQ ID NO: 415 - protein, 5 aa, synthetic construct
SYSMN

SEQ ID NO: 416 - protein, 17 aa, synthetic construct
YISSSSSTIYYADSVKG

SEQ ID NO: 417 - protein, 12 aa, synthetic construct
EDFSHKLGYFQH

SEQ ID NO: 418 - protein, 11 aa, synthetic construct
RASQSISSYLN

SEQ ID NO: 419 - protein, 7 aa, synthetic construct
AASSLQS

SEQ ID NO: 420 - protein, 9 aa, synthetic construct
QQSYSTPLT

SEQ ID NO: 421 - protein, 123 aa, synthetic construct
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARIKFTSFLYVHGFLWGQGTLVTVSS

SEQ ID NO: 422 - protein, 107 aa, synthetic construct [sequence omitted]

SEQ ID NO: 423 - protein, 7 aa, synthetic construct
SSSYYWG

SEQ ID NO: 424 - protein, 16 aa, synthetic construct
SIYYSGSTYYNPSLKS

SEQ ID NO: 425 - protein, 13 aa, synthetic construct
IKFTSFLYVHGFL

SEQ ID NO: 426 - protein, 11 aa, synthetic construct
RASQSISSYLN

SEQ ID NO: 427 - protein, 7 aa, synthetic construct
AASSLQS

SEQ ID NO: 428 - protein, 9 aa, synthetic construct
QQSYSTPLT

SEQ ID NO: 429 - protein, 118 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARYMYNWYFDLWGRGTLVTVSS

SEQ ID NO: 430 - protein, 107 aa, synthetic construct; identical to SEQ ID NO: 350

SEQ ID NO: 431 - protein, 5 aa, synthetic construct
DYYMS

SEQ ID NO: 432 - protein, 17 aa, synthetic construct
YISSSGSTIYYADSVKG

SEQ ID NO: 433 - protein, 9 aa, synthetic construct
YMYNWYFDL

SEQ ID NO: 434 - protein, 11 aa, synthetic construct
RASQGISSWLA

SEQ ID NO: 435 - protein, 7 aa, synthetic construct
AASSLQS

SEQ ID NO: 436 - protein, 9 aa, synthetic construct
QQANSFPLT

SEQ ID NO: 437 - protein, 118 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARAFLFLSFSVWGQGTLVTVSS

SEQ ID NO: 438 - protein, 107 aa, synthetic construct [sequence omitted]

SEQ ID NO: 439 - protein, 5 aa, synthetic construct
SYGMH

SEQ ID NO: 440 - protein, 17 aa, synthetic construct
VIWYDGSNKYYADSVKG

SEQ ID NO: 441 - protein, 9 aa, synthetic construct
AFLFLSFSV

SEQ ID NO: 442 - protein, 11 aa, synthetic construct
RASQSISSYLN

SEQ ID NO: 443 - protein, 7 aa, synthetic construct
AASSLQS

SEQ ID NO: 444 - protein, 9 aa, synthetic construct
QQSYSTPLT

SEQ ID NO: 445 - protein, 126 aa, synthetic construct
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARVRFLAARTTIPEANFLWGQGTLVTVSS

SEQ ID NO: 446 - protein, 107 aa, synthetic construct [sequence omitted]

SEQ ID NO: 447 - protein, 7 aa, synthetic construct
SSSYYWG

SEQ ID NO: 448 - protein, 16 aa, synthetic construct
SIYYSGSTYYNPSLKS

SEQ ID NO: 449 - protein, 16 aa, synthetic construct
VRFLAARTTIPEANFL

SEQ ID NO: 450 - protein, 11 aa, synthetic construct
RASQSISSYLN

SEQ ID NO: 451 - protein, 7 aa, synthetic construct
AASSLQS

SEQ ID NO: 452 - protein, 9 aa, synthetic construct
QQSYSTPLT

SEQ ID NO: 453 - protein, 120 aa, synthetic construct
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAREWIPSRPYYFDYWGQGTLVTVSS

SEQ ID NO: 454 - protein, 107 aa, synthetic construct [sequence omitted]

SEQ ID NO: 455 - protein, 5 aa, synthetic construct
SYYWS

SEQ ID NO: 456 - protein, 16 aa, synthetic construct
YIYYSGSTNYNPSLKS

SEQ ID NO: 457 - protein, 12 aa, synthetic construct
EWIPSRPYYFDY

SEQ ID NO: 458 - protein, 11 aa, synthetic construct
RASQSISSYLN

SEQ ID NO: 459 - protein, 7 aa, synthetic construct
AASSLQS

SEQ ID NO: 460 - protein, 9 aa, synthetic construct
QQSYSTPLT

SEQ ID NO: 461 - protein, 119 aa, synthetic construct
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARDQQALKYRVDWGQGTLVTVSS

SEQ ID NO: 462 - protein, 107 aa, synthetic construct [sequence omitted]

SEQ ID NO: 463 - protein, 6 aa, synthetic construct
SSNWWS

SEQ ID NO: 464 - protein, 16 aa, synthetic construct
EIYHSGSTNYNPSLKS

SEQ ID NO: 465 - protein, 10 aa, synthetic construct
DQQALKYRVD

SEQ ID NO: 466 - protein, 11 aa, synthetic construct
RASQSISSYLN

SEQ ID NO: 467 - protein, 7 aa, synthetic construct
AASSLQS

SEQ ID NO: 468 - protein, 9 aa, synthetic construct
QQSYSTPLT

SEQ ID NO: 469 - protein, 117 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARLRGRVFDPWGQGTLVTVSS

SEQ ID NO: 470 - protein, 107 aa, synthetic construct [sequence omitted]

SEQ ID NO: 471 - protein, 5 aa, synthetic construct
SYSMN

SEQ ID NO: 472 - protein, 17 aa, synthetic construct
SISSSSSYIYYADSVKG

SEQ ID NO: 473 - protein, 8 aa, synthetic construct
LRGRVFDP

SEQ ID NO: 474 - protein, 11 aa, synthetic construct
RASQSISSYLN

SEQ ID NO: 475 - protein, 7 aa, synthetic construct
AASSLQS

SEQ ID NO: 476 - protein, 9 aa, synthetic construct
QQSYSTPLT

SEQ ID NO: 477 - protein, 118 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCASVHKKPIFDYWGQGTLVTVSS

SEQ ID NO: 478 - protein, 106 aa, synthetic construct; identical to SEQ ID NO: 230

SEQ ID NO: 479 - protein, 5 aa, synthetic construct
DYYMS

SEQ ID NO: 480 - protein, 17 aa, synthetic construct
YISSSGSTIYYADSVKG

SEQ ID NO: 481 - protein, 9 aa, synthetic construct
VHKKPIFDY

SEQ ID NO: 482 - protein, 11 aa, synthetic construct
RASQGISSALA

SEQ ID NO: 483 - protein, 7 aa, synthetic construct
DASSLES

SEQ ID NO: 484 - protein, 9 aa, synthetic construct
QQFNSYPLT

SEQ ID NO: 485 - protein, 122 aa, synthetic construct
EVQLVESGGGVVRPGGSLRLSCAASGFTFDDYGMSWVRQAPGKGLEWVSGINWNGGSTGYADSVKGRFTISRDNAKNSLYLQMNSLRAEDTALYHCARERYRRVLHWYFDLWGRGTLVTVSS

SEQ ID NO: 486 - protein, 107 aa, synthetic construct [sequence omitted]

SEQ ID NO: 487 - protein, 5 aa, synthetic construct
DYGMS

SEQ ID NO: 488 - protein, 17 aa, synthetic construct
GINWNGGSTGYADSVKG

SEQ ID NO: 489 - protein, 13 aa, synthetic construct
ERYRRVLHWYFDL

SEQ ID NO: 490 - protein, 11 aa, synthetic construct
RASQSISSYLN

SEQ ID NO: 491 - protein, 7 aa, synthetic construct
AASSLQS

SEQ ID NO: 492 - protein, 9 aa, synthetic construct
QQSYSTPLT

SEQ ID NO: 493 - protein, 121 aa, synthetic construct
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTDLPKLRNFHIWGQGTLVTVSS

SEQ ID NO: 494 - protein, 107 aa, synthetic construct [sequence omitted]

SEQ ID NO: 495 - protein, 5 aa, synthetic construct
NAWMS

SEQ ID NO: 496 - protein, 19 aa, synthetic construct
RIKSKTDGGTTDYAAPVKG

SEQ ID NO: 497 - protein, 10 aa, synthetic construct
DLPKLRNFHI

SEQ ID NO: 498 - protein, 11 aa, synthetic construct
RASQSISSYLN

SEQ ID NO: 499 - protein, 7 aa, synthetic construct
AASSLQS

SEQ ID NO: 500 - protein, 9 aa, synthetic construct
QQSYSTPLT

SEQ ID NO: 501 - protein, 119 aa, synthetic construct
QVQLQESGPGLVKPSQTLSLTCTVSGGSISSGGYYWSWIRQHPGKGLEWIGYIYYSGSTYYNPSLKSLVTISVDTSKNQFSLKLSSVTAADTAVYYCASGDRARYFDLWGRGTLVTVSS

SEQ ID NO: 502 - protein, 107 aa, synthetic construct [sequence omitted]

SEQ ID NO: 503 - protein, 7 aa, synthetic construct
SGGYYWS

SEQ ID NO: 504 - protein, 16 aa, synthetic construct
YIYYSGSTYYNPSLKS

SEQ ID NO: 505 - protein, 9 aa, synthetic construct
GDRARYFDL

SEQ ID NO: 506 - protein, 11 aa, synthetic construct
RASQSISSYLN

SEQ ID NO: 507 - protein, 7 aa, synthetic construct
AASSLQS

SEQ ID NO: 508 - protein, 9 aa, synthetic construct
QQSYSTPLT

SEQ ID NO: 509 - protein, 123 aa, synthetic construct
QVQLQQSGPGLVKPSETLSLTCTVSGYSISSGYFWGWIRQPPGKGLEWIGSIYQSGSTYYNPSLKSRVTISLDTSKNQFSLKLSSVTAADTAVYYCARDGYCSGGSCYPFDYWGQGTTVTVSS

SEQ ID NO: 510 - protein, 110 aa, synthetic construct
QSVLTQPPSASGTPGQRVTISCSGSSSNIGSNTVNWYQRLPGAAPQLLIYNNDQRPSGIPDRFSGSKSGTSGSLVISGLQSEDEADYYCASWDDSLNGRVFGGGTKLTVL

SEQ ID NO: 511 - protein, 6 aa, synthetic construct
SGYFWG

SEQ ID NO: 512 - protein, 16 aa, synthetic construct
SIYQSGSTYYNPSLKS

SEQ ID NO: 513 - protein, 14 aa, synthetic construct
DGYCSGGSCYPFDY

SEQ ID NO: 514 - protein, 13 aa, synthetic construct
SGSSSNIGSNTVN

SEQ ID NO: 515 - protein, 7 aa, synthetic construct
NNDQRPS

SEQ ID NO: 516 - protein, 11 aa, synthetic construct
ASWDDSLNGRV

SEQ ID NO: 517 - protein, 124 aa, synthetic construct
QVQLQQSGAEVKMPGSSVKVSCKAFGNSFSSYATNWVRQAPGQGLEWLGWISAANGITNYGQKFQGRVTMTTDKSTDTAYMELRSLRSDDTAVYYCARTPFSENRGYYYYMDVWGKGTMVTVSS

SEQ ID NO: 518 - protein, 110 aa, synthetic construct
QSVLTQPPSASGTPGQRVTISCSGSGSNIGSNTVNWYQRLPGAAPQLLIYNNGQRPSGIPDRFSGSKSGTSGSLVISGLQSEDEADYYCASWDDSLNGRVFGGGTKLTVL

SEQ ID NO: 519 - protein, 5 aa, synthetic construct
SYATN

SEQ ID NO: 520 - protein, 17 aa, synthetic construct
WISAANGITNYGQKFQG

SEQ ID NO: 521 - protein, 15 aa, synthetic construct
TPFSENRGYYYYMDV

SEQ ID NO: 522 - protein, 13 aa, synthetic construct
SGSGSNIGSNTVN

SEQ ID NO: 523 - protein, 7 aa, synthetic construct
NNGQRPS

SEQ ID NO: 524 - protein, 11 aa, synthetic construct
ASWDDSLNGRV

SEQ ID NO: 525 - protein, 115 aa, synthetic construct
EVQLQQSGPELKKPGTSVRISCKTSGYTFTEYTIHWVKQSHGKSLEWIGNINPNNGGTTYNQKFEDKATLTVDKSSSTAYMELRSLTSEDSAVYYCAAGWNFDYWGQGTTLTVSS

SEQ ID NO: 526 - protein, 107 aa, synthetic construct
DIVMTQSHKFMSTSVGDRVSIICKASQDVGTAVDWYQQKPGQSPKLLIYWASTRHTGVPDRFTGSGSGTDFTLTITNVQSEDLADYFCQQYNSYPLTFGGGTKLEIK

SEQ ID NO: 527 - protein, 5 aa, synthetic construct
EYTIH

SEQ ID NO: 528 - protein, 17 aa, synthetic construct
NINPNNGGTTYNQKFED

SEQ ID NO: 529 - protein, 6 aa, synthetic construct
GWNFDY

SEQ ID NO: 530 - protein, 11 aa, synthetic construct
KASQDVGTAVD

SEQ ID NO: 531 - protein, 7 aa, synthetic construct
WASTRHT

SEQ ID NO: 532 - protein, 9 aa, synthetic construct
QQYNSYPLT

SEQ ID NO: 533 - protein, 117 aa, synthetic construct
QVQLQQSAAELARPGASVKMSCKASGYSFTTYTIHWVKQRPGQGLEWIGYINPSSGYAAYNQNFKDETTLTADPSSSTAYMELNSLTSEDSAVYYCAREKFYGYDYWGQGATLTVSS

SEQ ID NO: 534 - protein, 112 aa, synthetic construct
DVLMTQIPLSLPVSLGDQASISCRSSQSIVHRNGNTYLEWYLLKPGQSPKLLIYKVSNRFSGVPDRFSGSGSGTDFTLKISRVEAEDLGVYYCFQGSHVPYTFGGGTKLEIR

SEQ ID NO: 535 - protein, 5 aa, synthetic construct
TYTIH

SEQ ID NO: 536 - skipped

SEQ ID NO: 537 - protein, 8 aa, synthetic construct
EKFYGYDY

SEQ ID NO: 538 - protein, 16 aa, synthetic construct
RSSQSIVHRNGNTYLE

SEQ ID NO: 539 - protein, 7 aa, synthetic construct
KVSNRFS

SEQ ID NO: 540 - protein, 9 aa, synthetic construct
FQGSHVPYT